(19) United States
(12) Patent Application Publication
(10) Pub. No.: US 2024/0327503 A1
(43) Pub. Date: Oct. 3, 2024

(54) SARS-COV-2 NEUTRALIZING ANTIBODIES AND USES THEREOF

(71) Applicants: Jimmy Dale GOLLIHAR, Hewitt, TX (US); BOARD OF REGENTS, THE UNIVERSITY OF TEXAS SYSTEM, Austin, TX (US); THE UNITED STATES OF AMERICA AS REPRESENTED BY THE SECRETARY OF THE ARMY, Washington, DC (US)

(72) Inventors: Jimmy Dale GOLLIHAR, Hewitt, TX (US); Jule GOIKE, Austin, TX (US); Ching-Lin HSIEH, Austin, TX (US); Andrew P. HORTON, Austin, TX (US); Elizabeth GARDNER, Austin, TX (US); Daniel BOUTZ, Austin, TX (US); Andrew ELLINGTON, Austin, TX (US); Edward MARCOTTE, Austin, TX (US); Jason S. MCLELLAN, Austin, TX (US); George GEORGIOU, Austin, TX (US); Gregory C. IPPOLITO, Austin, TX (US)

(21) Appl. No.: 18/550,194

(22) PCT Filed: Mar. 11, 2022
(86) PCT No.: PCT/US2022/019934

§ 371 Date: Sep. 12, 2023

Related U.S. Application Data
(60) Provisional application No. 63/160,373, filed on Mar. 12, 2021.

(51) Int. Cl. C07K 16/10; A61P 31/14; G01N 33/569

(52) U.S. Cl. CPC C07K 16/1003; A61P 31/14; G01N 33/56983; C07K 2317/24; C07K 2317/31; G01N 2333/165; G01N 2469/10

(57) ABSTRACT

Provided herein are antibodies and antibody fragments that bind to SARS-COV-2 spike protein. Methods of using these antibodies in in vitro methods are provided. Methods of using these antibodies in vivo to treat or prevent SARS-COV-2 infections are also provided.

### REFERENCE TO RELATED APPLICATIONS

The present application claims the priority benefit of U.S. provisional application No. 63/160,373, filed Mar. 12, 2021, the entire contents of which is incorporated herein by reference.

### STATEMENT REGARDING FEDERALLY SPONSORED RESEARCH

This invention was made with government support under Grant nos. R01 AI127521, R01 AI158177 and R35 GM122480 awarded by the National Institutes of Health, Grant no. W911NF-17-2-0091 awarded by the Army Research Office, Grant no. HDTRA1-20-1-0011 awarded by the Department of Defense/Department of Threat Reduction and Grant no. 70NANB20H037 awarded by the National Institute of Standards and Technology. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 9, 2022, is named UTFBP1262WO_ST25.txt and is 270,107 bytes in size.

### BACKGROUND

### 1. Field

The present invention relates generally to the fields of medicine, immunology, and virology. More particularly, it concerns human antibodies that bind the SARS-COV-2 spike protein and methods of their use.

### 2. Description of Related Art

The rapid global dissemination of the severe acute respiratory syndrome coronavirus 2 (SARS-COV-2) (Zhou et al., 2020), the cause of coronavirus disease 19 (COVID-19) (Wu et al., 2020), has highlighted our extreme vulnerability to novel microbial threats. The speed of SARS-COV-2 transmission and absence of widespread adaptive immunity created a pandemic that overwhelmed the international medical community. This situation was exacerbated by the scarcity of treatment options, especially early in the pandemic. Functional immune repertoire analysis has the potential to efficiently address this scarcity. By analyzing primary immune responses directed towards emerging pathogens, newly elicited antibodies can be identified and rapidly deployed to treat patients (Ju et al., 2020).

The COVID-19 pandemic has stimulated global research efforts to identify SARS-COV-2-neutralizing antibodies for therapeutic and prophylactic applications. Initial attempts to identify neutralizing antibodies focused on screening extant antibodies elicited against previous SARS-COV strains. These efforts were largely unsuccessful owing to limited cross-reactivity (Tian et al., 2020), primarily due to SARS-COV-2's sequence divergence in the receptor-binding domain (RBD) of its trimeric spike protein (Wrapp et al., 2020).

The ectodomain (ECD) of the SARS-COV-2 spike(S) protein is essential for initial binding and subsequent entry of the virus into human cells and has been the primary target for therapeutics and vaccine formulations. The ECD consists of the S1 subunit, containing an N-terminal domain (NTD) and RBD, and the S2 subunit, containing the fusion machinery that mediates entry into host cells. The RBD initiates attachment through interaction with angiotensin converting enzyme2 (ACE2) (Tai et al., 2020; Li et al., 2003; Yan et al., 2020). The functional significance of the ACE2-RBD interaction makes the RBD a prime target for neutralizing antibodies (Shi et al., 2020; Yuan et al., 2020; Yi et al., 2020). Targeting this domain increases the selective pressure on the RBD, which may promote the emergence of escape mutants that maintain virulence. Indeed, neutralizing antibodies targeting a single epitope can induce virus escape in cell culture, quickly rendering antibodies ineffective (Baum et al., 2020).

The FDA approved antibody cocktail of REGN10933 and REGN10987 (Hansen et al., 2020) targets distinct epitopes of the RBD and sustains some neutralization activity against new SARS-COV-2 strains B.1.1.7 and B.1.351, originally identified in the UK and South Africa, respectively (Shen et al., 2021; Wang et al., 2021; Ferrareze et al., 2021). Recently, a deep mutational scan of the RBD found that the single amino acid mutation E406W increased the IC₅₀ of REGN10933 by more than 300-fold and also detrimentally affected REGN10987 binding (Starr et al., 2021). The scan also identified amino acid changes that compromised the epitope of Eli Lilly's monoclonal LY-CoV016 (Starr et al., 2021). Moreover, the mass sequencing of 5,085 SARS-COV-2 genomes from the Houston metropolitan area published in September 2020 already identified numerous spike protein mutations that affect the existing neutralizing antibodies' abilities to bind to their epitopes (Long et al., 2020).

The Alpha (B.1.1.7; UK), B.1.1.248 (Brazil), Beta (B.1.351; South Africa), Gamma (P.1) and Delta (B.1.617.2) are variants of concern because they have each accumulated multiple spike protein mutations and have shown increased transmissibility (Volz et al., 2021; Tegally et al., 2021; Coutinho et al., 2021; Liu et al., 2021; Luo et al., 2021). These mutations are located in the NTD, RBD, and furin cleavage site. NTD-directed antibodies in particular show reduced or abolished binding to these strains (Wang et al., 2021). More concerning than the loss of binding ability, however, is the reduced neutralizing activity of convalescent plasma from patients infected in the spring of 2020 (Wang et al., 2021; Hoffmann et al., 2021).

Furthermore, currently available vaccines are largely based on an earlier prefusion-stabilized spike variant (Wuhan-Hu-1) (Baum et al., 2020). When assayed, sera from patients vaccinated with the Moderna and Pfizer/BioNTech vaccines showed a roughly 1-fold decrease in in vitro neutralization activity towards strain Beta (B.1.351), while Alpha (B.1.1.7) was only mildly affected (Wang et al., 2021). Arguably most alarming, is the ability of the Delta variant to cause vaccine breakthrough infections (Farinholt et al., 2021). Fortunately, studies have shown that both prior infection and, to a larger degree, vaccination confer protection against hospitalization, severe disease, and death for patients infected with currently circulating variants (Bian et al., 2021). Nevertheless, it is critical to identify neutralizing antibodies to a broad spectrum of non-overlapping epitopes on the spike protein to achieve highly potent and persistent neutralization (Baum et al., 2020).

### SUMMARY

To understand antibody-mediated immunity and gain insight into how vaccination campaigns based on Wuhan-Hu-1 might protect against VOCs, the inventors developed a multi-pronged antibody discovery and informatics strategy involving both proteomic analysis of donor sera and selection of combinatorially paired variable heavy-variable light chain (VH-VL) libraries from donor B-cell receptor repertoires. Specifically, Ig-Seq proteomics were used to identify candidate variable heavy chains (VHs) of serum antibodies binding SARS-COV-2 RBD or ECD. Productive light chain pairs for these heavy chains were identified either from a bioinformatically derived set of “public light chains” (PLCs) or through combinatorial pairing with donor light chain repertoires via yeast surface display (YSD) selection. In addition to Ig-Seq interrogation of circulating IgGs, YSD was used to select high-affinity antibodies by combinatorial display of all donor VH-VL pairs as Fab fragments through multiple rounds of selection.

Together this holistic strategy allowed the inventors to probe the secreted, circulating antibody repertoire and the nascent cellular repertoire of a primary immune response. By interrogating the combined repertoire, valuable insight was gained into the antibody response elicited by SARS-COV-2 and neutralizing antibodies to multiple distinct domains of the spike protein were discovered. The structural characterization of two such neutralizers, including a highly potent neutralizing antibody (N3-1) that binds a quaternary epitope of the trimeric spike protein via a novel binding modality, are provided herein. Finally, binding to emerging spike variants of SARS-COV-2 was tested and N3-1 was demonstrated to maintain robust binding to these variants.

In one embodiment, provided herein are monoclonal antibodies or antibody fragments, wherein the antibodies or antibody fragments comprise a heavy chain variable region (VH) comprising VHCDR1, VHCDR2, and VHCDR3 amino acid sequences and a light chain variable region (VL) comprising VLCDR1, VLCDR2, and VLCDR3 amino acid sequences derived from clone-paired VH and VL sequence from Table 1. In some aspects, the antibodies or antibody fragments comprise a heavy chain variable region (VH) comprising VHCDR1, VHCDR2, and VHCDR3 amino acid sequences derived from SEQ ID NO: 1 and a light chain variable region (VL) comprising VLCDR1, VLCDR2, and VLCDR3 amino acid sequences derived from SEQ ID NO: 2. In some aspects, the antibodies or antibody fragments comprises clone-paired heavy and light chain CDR sequences from Table 2.

In some aspects, the antibodies or antibody fragments comprise clone-paired heavy chain and light chain variable sequences having, independently, at least 70%, 75%, 80%, 85%, 90%, 91%, 92%, 93%, 94%, 95%, 96%, 97%, 98%, 99%, or 100% identity to sequences from Table 1. In some aspects, the antibodies or antibody fragments bind to a coronavirus spike protein and are derived, by way of one or more amino acid substitutions, deletions or insertions, from clone-paired heavy chain and light chain variable polypeptide sequences of Table 1. In some aspects, the antibodies or antibody fragments comprise clone-paired heavy chain and light chain variable sequences from Table 1.

In one embodiment, provided herein are monoclonal antibodies or antibody fragments, wherein the antibodies or antibody fragments comprise a heavy chain variable region (VH) comprising VHCDR1, VHCDR2, and VHCDR3 amino acid sequences derived from a VH sequence from Table 1 and light chain variable region (VL) comprising VLCDR1, VLCDR2, and VLCDR3 amino acid sequences derived from VL sequence from Table 8. In other words, each heavy chain disclosed herein is specifically contemplated as pairing with each of the public light chains disclosed herein.

In some aspects, the antibody fragment is a monovalent scFv (single chain fragment variable) antibody, divalent scFv, Fab fragment, F(ab′)₂ fragment, F(ab′)₃ fragment, Fv fragment, or single chain antibody. In some aspects, the antibody is a chimeric antibody, bispecific antibody, or BiTE. In some aspects, the antibody is an IgG antibody or a recombinant IgG antibody or antibody fragment.

In some aspects, the antibody is capable of binding to a coronavirus spike protein. In some aspects, the coronavirus spike protein is from SARS-COV, SARS-COV-2, or MERS. In some aspects, the antibody is capable of binding to the spike protein from SARS-CoV-2. In some aspects, the antibody is capable of binding to SARS-COV-2 B.1.1.7, B.1.1.248, and/or B.1.351 spike protein variants.

In some aspects, the antibody or antibody fragment is fused to an imaging agent. In some aspects, the antibody or antibody fragment is labeled. In some aspects, the label is a fluorescent label, an enzymatic label, or a radioactive label.

In one embodiment, provided herein are monoclonal antibodies or antibody fragments, which compete for binding to the same epitope of a coronavirus spike protein as the monoclonal antibody or an antibody fragment according to any one of the present embodiments. In one embodiment, provided herein are monoclonal antibodies or antibody fragments that bind to an epitope on a coronavirus spike protein recognized by the monoclonal antibody or antibody fragment of any one of the present embodiments.

In some aspects, the antibody or antibody fragment that binds to an epitope on a coronavirus spike protein recognized by the N3-1 monoclonal antibody or a fragment thereof. In some aspects, the antibody or antibody fragment simultaneously binds to epitopes on two receptor binding domain (RBD) subunits of a coronavirus spike trimer. In some aspects, one of the RBDs is in the “up” position and one of the RBDs is in the “down” position. In some aspects, the epitope on the RBD “up” subunit comprises residues that correspond to amino acids 369, 377, 378, and 384 of SEQ ID NO: 298. In some aspects, the epitope on the RBD “down” subunit comprises residues that correspond to amino acids 486, 487, 489, and 493 of SEQ ID NO: 298. In some aspects, when bound to a coronavirus spike protein trimer, the monoclonal antibody binds to residues of one subunit of the coronavirus spike protein trimer that correspond to positions 369, 377, 378, and 384 of SEQ ID NO: 298 and to residues of a second subunit of the coronavirus spike protein trimer that correspond to position 486, 487, 489, and 493 of SEQ ID NO: 298. In some aspects, when bound to a coronavirus spike protein, the monoclonal antibody additionally binds to at least one residue of a coronavirus spike protein that corresponds to position 379 and 408 of SEQ ID NO: 298. In some aspects, the antibody is capable of binding to the spike proteins from SARS-COV, SARS-COV-2, and MERS. In some aspects, the antibody is capable of binding to SARS-CoV-2 B.1.1.7, B.1.1.248, and/or B.1.351 spike protein variants.

In one embodiment, provided herein are isolated nucleic acids encoding the antibody heavy and/or light chain variable region of the antibody or antibody fragment of any one of the present embodiments. In one embodiment, provided herein are expression vectors comprising the nucleic acid.

In one embodiment, provided herein are hybridomas or engineered cells comprising a nucleic acid encoding an antibody or antibody fragment of any one of the present embodiments. In one embodiment, provided herein are methods of making the monoclonal antibody or antibody fragment of any one of the present embodiments, the method comprising culturing the hybridoma or engineered cell under conditions that allow expression of the antibody or antibody fragment and optionally isolating the antibody or antibody fragment from the culture.

In one embodiment, provided herein are pharmaceutical formulations comprising one or more antibody or antibody fragment of any one of the present embodiments. In one embodiment, provided herein are pharmaceutical formulations comprising one or more expression vector encoding a first antibody or antibody fragment of any one of the present embodiments.

In one embodiment, provided herein are methods of reducing the likelihood of a pathogenic coronavirus infection in a patient at risk of contracting the pathogenic coronavirus, the methods comprising delivering to the patient an antibody or antibody fragment of any one of the present embodiments. In some aspects, the methods prevent a SARS-COV-2 infection in the patient. In some aspects, the patient has been exposed to a coronavirus. In some aspects, the antibody or antibody fragment is delivered to the patient prior to infection or after infection. In some aspects, delivering comprises antibody or antibody fragment administration, or genetic delivery with an RNA or DNA sequence or vector encoding the antibody or antibody fragment.

In one embodiment, provided herein are methods of treating a patient infected with a pathogenic coronavirus, the methods comprising delivering to the patient an antibody or antibody fragment of any one of the present embodiments. In some aspects, delivering comprises antibody or antibody fragment administration, or genetic delivery with an RNA or DNA sequence or vector encoding the antibody or antibody fragment. In some aspects, the methods reduce the viral load in the patient. In some aspects, the methods further comprise administering at least a second antibody that binds to a distinct epitope relative to the antibody or antibody fragment of any one of the present embodiments.

In some aspects, provided herein are methods of detecting a coronavirus infection in a patient, the method comprising: (a) contacting a sample obtained from the patient with an antibody or antibody fragment of any one of the present embodiments; and (b) detecting the coronavirus in the sample by detecting binding of the antibody or antibody fragment to a coronavirus antigen in the sample. In some aspects, the sample is a body fluid. In some aspects, the sample is blood, sputum, tears, saliva, mucous or serum, semen, cervical or vaginal secretions, amniotic fluid, placental tissues, urine, exudate, transudate, tissue scrapings or feces. In some aspects, detecting comprises an ELISA, RIA, lateral flow assay or Western blot. In some aspects, the methods further comprise performing steps (a) and (b) a second time and determining a change in coronavirus antigen levels as compared to the first assay. In some aspects, the coronavirus is an emerging coronavirus. In some aspects, the coronavirus has not yet been identified at the time the method is performed.

In some aspects, provided herein are methods of detecting a coronavirus spike protein in an in vitro sample, the method comprising contacting the in vitro sample with an antibody or antibody fragment of any one of the present embodiments and detecting the binding of the antibody or antibody fragment to the sample. In some aspects, the detecting is by flow cytometry, mass spectrometry, western blot, immunohistochemistry, ELISA, biolayer interferometry (BLI), or RIA.

In one embodiment, provided herein are antibodies or antibody fragments or pharmaceutical formulations of any of the present embodiments, for use in treating or preventing a coronavirus infection in a patient. In one embodiment, provided herein are uses of an antibody or antibody fragment or a pharmaceutical formulation of the present embodiments, in the manufacture of a medicament for treating or preventing a coronavirus infection in a patient.

In one embodiment, provided herein are methods of identifying an antibody that binds to an antigen of interest, the method comprising: (a) obtaining antibody VH chain sequence data for at least one antibody that binds to the antigen of interest; and (b) identifying productive V_{L} pairs for the identified VH chain by pairing the V_{H} chain with a V_{L} repertoire and testing for binding to the antigen of interest. An antigen of interest may be any antigen for which it is desirable to having a binding antibody, such as tumor associated antigens, infectious disease associated antigens, and the like. In some aspects, the light chain repertoire is a repertoire of public light chains. In some aspects, the public light chains are light chains that have been identified as showing high frequency of productive pairing with a diverse set of V_{H} sequences. In some aspects, the public light chains each comprise a V-gene sequence that is prevalent in homeostatic repertoires. In some aspects, the public light chains each further comprise a J-gene sequence is the most commonly associated with the respective V-gene. In some aspects, the public light chains each have a germline sequence. In some aspects, the antibody is a human antibody, and the public light chains have CDR sequences derived from any of SEQ ID NOS: 2, 4, 12, 15, 19, 23, 26, and 29. In some aspects, the antibody is a human antibody, and the public light chains have sequences according to SEQ ID NOS: 2, 4, 12, 15, 19, 23, 26, and 29. In some aspects, the antibody is a murine antibody, and the public light chains have CDR sequences derived from any of SEQ ID NOS: 299-309. In some aspects, the antibody is a murine antibody, and the public light chains have sequences according to SEQ ID NOS: 299-309. In some aspects, testing for binding in step (b) comprises performing ELISA. In some aspects, step (a) comprises: (i) obtaining antibody sequence data from PBMCs isolated from a subject previously exposed to the antigen; (ii) obtaining mass spectra of CDR3 peptides derived from antibody V_{H} chains of the subject following enrichment for binding to the antigen of interest; and (iii) using the sequence information and the mass spectra to identify the amino acid sequences of the antibody V_{H} chains enriched in step (iii), thereby obtaining antibody V_{H} chain sequence data. In some aspects, the antibody sequence data are IgG and IgM antibody sequence data. In some aspects, the antigen of interest is a coronavirus spike protein. In some aspects, the enrichment in step (ii) is enrichment for binding to the SARS-COV-2 spike protein extracellular domain. In some aspects, the antigen of interest is derived from an emerging infectious disease. In some aspects, the methods further comprise subjecting the identified antibody to affinity maturation and/or directed evolution. In some aspects, provided herein are antibodies generated by these method.

TABLE A
Murine public light chains.

| | V- | J- | | | | Full-length |
|---|---|---|---|---|---|---|
| Name | Gene | Gene | Light CDR1 | Light CDR2 | Light CDR3 | Light |
| mPLC1 | IGKV10- | IGKJ1 | QDISNY | YTS | QQGNTLPRT | SEQ ID NO: |
| | 96 | | (SEQ ID NO: | (SEQ ID NO: | (SEQ ID NO: 328) | 299 |
| | | | 310) | 321) | | |
| mPLC2 | IGKV12- | IGKJ1 | ENIYGA | GAT | QNVLSTPPWT | SEQ ID NO: |
| | 89 | | (SEQ ID NO: | (SEQ ID NO: | (SEQ ID NO: 329) | 300 |
| | | | 311) | 322) | | |
| mPLC3 | IGKV12- | IGKJ2 | ENIYSY | NAK | QHHYGTPPYT | SEQ ID NO: |
| | 44 | | (SEQ ID NO: | (SEQ ID NO: | (SEQ ID NO: 330) | 301 |
| | | | 312) | 323) | | |
| mPLC4 | IGKV12- | IGKJ1 | ENIYSN | AAT | QHFWGTPPWT | SEQ ID NO: |
| | 46 | | (SEQ ID NO: | (SEQ ID NO: | (SEQ ID NO: 331) | 302 |
| | | | 313) | 324) | | |
| mPLC5 | IGKV19- | IGKJ1 | QDINKY | YTS | LQYDNLLPWT | SEQ ID NO: |
| | 93 | | (SEQ ID NO: | (SEQ ID NO: | (SEQ ID NO: 332) | 303 |
| | | | 314) | 321) | | |
| mPLC6 | IGKV6- | IGKJ5 | QDVGTA | WAS | QQYSSYPLLT | SEQ ID NO: |
| | 23 | | (SEQ ID NO: | (SEQ ID NO: | (SEQ ID NO: 333) | 304 |
| | | | 315) | 110) | | |
| mPLC7 | IGKV6- | IGKJ5 | QNVGTN | SAS | QQYNSYPLLT | SEQ ID NO: |
| | 15 | | (SEQ ID NO: | (SEQ ID NO: | (SEQ ID NO: 334) | 305 |
| | | | 316) | 325) | | |
| mPLC8 | IGKV8- | IGKJ1 | QSVLYSSNQ | WAS | HQYLSSWT | SEQ ID NO: |
| | 27 | | KNY | (SEQ ID NO: | (SEQ ID NO: 335) | 306 |
| | | | (SEQ ID NO: | 110) | | |
| | | | 317) | | | |
| mPLC9 | IGKV1- | IGKJ1 | QSIVHSNGN | KVS | FQGSHVPPWT | SEQ ID NO: |
| | 117 | | TY | (SEQ ID NO: | (SEQ ID NO: 336) | 307 |
| | | | (SEQ ID NO: | 326) | | |
| | | | 318) | | | |
| mPLC10 | IGKV1- | IGKJ1 | QSLLDSDGK | LVS | WQGTHFPQWT | SEQ ID NO: |
| | 135 | | TY | (SEQ ID NO: | (SEQ ID NO: 337) | 308 |
| | | | (SEQ ID NO: | 327) | | |
| | | | 319) | | | |
| mPLC11 | IGKV8- | IGKJ2 | QSLLYSSNQ | WAS | QQYYSYPPYT | SEQ ID NO: |
| | 30 | | KNY | (SEQ ID NO: | (SEQ ID NO: 338) | 309 |
| | | | (SEQ ID NO: | 110) | | |
| | | | 320) | | | |

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Overview of complementary strategies for discovering convalescent patient-derived anti-SARS-COV-2 neutralizing antibodies. Serum antibodies and B-cells are isolated from patient blood post infection. (FIG. 1A) IgG is antigen-enriched, digested, and analyzed with tandem mass spectrometry. Searching against a database of donor B cell receptor sequences yields antigen-specific heavy chain candidates. (FIG. 1B) To find productive VH-VL pairs, the proteomically identified VH are randomly combined with donor VL, generating a large library of Fab amplicons for selection in yeast. (FIG. 1C) Additional productive VH-VL are recovered by matching each proteomic VH candidate against a fixed set of nine “public” light chains significantly overrepresented in prior repertoire studies. All combinations are screened for binding. (FIG. 1D) Yeast surface display provides an independent method to recover VH or VH-VL candidates. VH and VL sequences are amplified from donor B-cells, and pairs are randomly assembled into large Fab libraries and selected using fluorescent cell sorting. Yeast pools are then sequenced after each selection to track individual clone dynamics round over round. Promising candidates are expressed as IgG for further characterization.

FIGS. 2A-2B. Public light chain screening and neutralization. (FIG. 2A) Public light chain and YSD-IgSeq abundance and screening. Nine PLCs emerge from analysis of 100,450 previously published paired VH-VL sequences. (FIG. 2B) Neutralization data against SARS-COV-2 WA1 live virus for the VH-VL pairs of Table 9.

FIGS. 3A-3J. Non-cognate paired antibodies neutralize SARS-COV-2 live virus. (FIG. 3A) SARS-COV-2 WA1 live virus neutralization assays. The graphs depict the neutralizers binned by their target spike subdomain. (FIG. 3B) VH gene usage of the neutralizing antibodies as a fraction of the whole. (FIG. 3C) Stacked bar chart showing neutralizer VH gene usage broken down by discovery method. (FIGS. 3D-3E) Same analysis described in FIGS. 3B-3C but by VL gene. (FIG. 3F) VH gene usage and discovery method of the neutralizing antibodies. (FIG. 3G) Same analysis for VL. (FIG. 3H) Neutralization IC50 values by VH CDR3 sequence, VH-VL discovery method, and epitope. SARS-COV-2 WA1 live virus neutralization assays for IgG using (FIG. 3I) heavy chain A7 and (FIG. 3J) heavy chain N3.

FIGS. 4A-4E. NTD-directed mAb A7V3 reveals a common binding mode. (FIG. 4A) Cryo-EM structure of A7V3 bound to SARS-COV-2 S at a global resolution of 3.0 Å. Side view and top-down views of the complex are shown in the upper and the lower panel, respectively. Each protomer is depicted in steel blue, royal blue, and sky blue. The heavy chain of A7V3 is colored firebrick, and the light chain is colored coral. (FIG. 4B) Focused map of A7V3 bound to NTD reveals a major binding site on the N3 and N5 loops. (FIG. 4C) The tip of the N5 loop is surrounded by the hydrophobic residues from L-CDR2 and H-CDR3. Arg246 and Tyr248 contact Glu31 and Asp101 via polar interactions. (FIG. 4D) Pro97 and Phe98 in H-CDR3 insert into a groove walled by the N3 and N5 loops. Conserved Phe51 in H-CDR2 forms a pi-cation interaction with Lys147. (FIG. 4E) Superimposed structure of 4A8-NTD complex (PDB ID: 7C2L) with A7V3-bound NTD. The molecular surface of the N3 and N5 loops is shown in blue. Unlike 4A8, the relatively short H-CDR3 from A7V3 barely contacts the N3 loop.

FIGS. 5A-5E. RBD-directed mAb N3-1 exhibits a unique binding mode by recognizing two distinct epitopes. (FIG. 5A) Cryo-EM structure of N3-1-bound SARS-CoV-2 spike at a global resolution of 2.8 Å. Side view and top-down views of the complex are shown in the upper and the lower panel, respectively. Each protomer is depicted in steel blue, royal blue and sky blue. The heavy chain of N3-1 is colored firebrick, and the light chain is colored coral. (FIG. 5B) Focused map of N3-1 bound to RBDs in the up and down conformations with five CDRs involved in the binding interface. (FIG. 5C) One face of H-CDR3 contacts a conserved hydrophobic pocket (transparent royal blue surface) on RBD-up. Arg408 forms multiple polar interactions with Tyr49 and Glu55. The other face of H-CDR3 contacts the ACE2-binding site on RBD-down. H-CDR1 contacts the epitopes on RBD-up, but it is omitted for clarity. (FIG. 5D) The epitope on RBD-down is centered on Phe486, which fits into a hydrophobic surface formed by Trp96, Tyr58, Tyr52, Arg 100e, and Val100g (clockwise). Arg100e also forms a cation-pi interaction with Phe486 and a hydrogen bond with Tyr489. (FIG. 5E) Superimposed crystal structure of RBD-ACE2 complex (PDB ID: 6M0J) with N3-1 bound RBDs. The molecular surface of ACE2 is shown in transparent pale green. The light chain of N3-1 heavily clashes with ACE2. The ACE2-binding site on RBD-down is completely blocked by H-CDR2, H-CDR3 and L-CDR3 (FIG. 5D).

FIGS. 6A-6L. RBD-directed mAb N3-1 neutralizes all variants of concern. (FIG. 6A) Model of N3-1 bound to RBD-up and RBD-down. Mutation sites in B.1.1.7, B.1.1.248, and B.1.351 are highlighted in green. (FIG. 6B) The B.1.1.7, B1.1.248, and B.1.351 spike variants were tested with A7V3, N3-1, and chimeric human ACE2-Fc using mammalian display of spike variants and compared to SARS-COV-2 Wuhan-Hu-1 S-HexaPro-D614G. (FIG. 6C) Spike variants were assayed against VOCs and Regeneron REGN10987 and/or REGN10933 escape mutants using mammalian surface display. HEK293T cells transiently expressing full length spike protein were stained with anti-spike antibodies and analyzed by flow cytometry. The median fluorescence intensity of the stained cells was normalized to the HexaPro-D614G spike. The SARS-COV2 RBD subunit and SARS-COV-2 spike with a deleted RBD (ARBD) were included as controls. *p<0.05; ****p<0.0001; using one sample t test. (FIG. 6D) Normalized binding of N3-1 to HexaPro and the Delta and Kappa variants of lineage B.1.617, assayed as in (FIG. 6C) but with multiple IgG concentrations to obtain dose response curves. (FIGS. 6E-6F) SARS-COV-2-S binding was assessed by surface plasmon resonance for Fab N3-1 binding to Wuhan-Hu-1 (FIG. 6E) and IgG N3-1 binding to Wuhan-Hu-1, Alpha and Beta (FIG. 6F). (FIGS. 6G-6H) Independent SARS-COV-2 live virus neutralization assays for N3-1 IgG. (FIG. 61) V_{H}3-30 CDR3s of length 13 to 15. The sequences shown are, from top to bottom, SEQ ID NOs: 120, 397, 397, 391, 168, 114, 452, 453, 280, 393, 415, 294, 292, 409, 282, 454, 455, and 281. (FIG. 6J) Binding of S2-directed antibodies to various SARS-COV-2 variants. (FIG. 6K) Neutralization by S2-directed antibodies to various SARS-COV-2 variants. (FIG. 6L) ELISAs of a subset of N3-1 variants. These included rationally designed heavy chain variants, which were designed by structure overlayed with an Omicron structure, in combination with PLC1, PLC3, and PLC7. The XN3 variants have an AA change at R100e and were made in response to the 371-373 mutations found in Omicron. The N3X mutants were made at position T56 in response to the Q493R mutation in omicron.

FIG. 7. Somatic hypermutation as a function of gene family calculated using Geneious Biologics.

FIG. 8. Representative YSD cell sorting lineage plots. Donor repertoires were cloned as Fab libraries and displayed in yeast. Yeast were labeled for expression (y-axis, anti-FLAG-FITC) and antigen binding (x-axis, biotinylated ECD, Streptavidin-Alexa Flour 488; human-Fc RBD, anti-human Alexa Fluor 488). Each library was subjected to selection in the presence of either RBD or spike ECD. Each population was subjected to sorting into up to two gates at a time, such that after several rounds of selection, populations with various expression and binding characteristics were enriched (see Rd 3). Combinatorial libraries consisted of randomly paired VHs and VLs cloned from donor cDNA.

FIGS. 9A-9B. Definition of candidate V_{H}-V_{L} sequences from error-prone MinION sequencing.

FIGS. 10A-10D. Representative YSD single clone flow cytometry. The x-axis is binding, and the y-axis is expression. Each of the four columns (FIGS. 10A-10D, respectively) represents a single clone assayed against Ecto (top row) and RBD (bottom row). Sequencing confirmed clones c and d are identical. Clones were initially prioritized on expression normalized binding and lack of RBD binding.

FIGS. 11A-11C. Yeast surface display of a combinatorially assembled donor repertoire. (FIG. 11A) Representative example of YSD cell sorting showing donor 3 Fab selection with the ECD antigen. The x-axis (AF-647) shows antigen binding and the y-axis (FITC) shows Fab expression level. Each round was sorted using one or two gates to enrich populations with different phenotypes. (FIG. 11B) Histogram showing enrichment of binders from round one (purple) to round 3 (light green) in the highlighted lineage. (FIG. 11C) Area chart showing HCDR3 enrichment throughout selection. Each unique HCDR3 is represented as a fraction of all HCDR3s in that population. Significant bias exists in the initial library, but by the end of the selection top variants represent over 10% of the total population.

FIGS. 12A-12C. Somatic hypermutation by gene family for YSD selected VHs from donors 1-3 (FIGS. 12A-12C, respectively) after three rounds of selection, as calculated by Geneious Biologics.

FIG. 13. Cryo-EM data processing workflow for A7V3 bound SARS-CoV-2 S.

FIG. 14. Cryo-EM data processing workflow for N3-1 bound SARS-COV-2 S.

FIG. 15A-15B. Avidity of mAb N3-1 likely achieved by a single IgG binding to a trimeric spike. (FIG. 15A) Representative 2D class averages of IgG N3-1 complexed with SARS-COV-2 S by negative stain electron microscopy (nsEM). Although the density of Fc is not well-resolved, two clear densities of Fabs are clearly visible per trimeric spike. (FIG. 15B) A schematic model generated by Gaussian-smoothened cryoEM map of N3-1 bound to SARS-COV-2 S. The Fab density is highlighted in brick red, and the spike is shown in light blue. The unobserved Fc is shown as orange ovals.

FIGS. 16A-16D. mAb N3-1 exhibits cross-reactivity and avidity to CoV spikes. (FIGS. 16A-16C) Binding of Fab N3-1 to SARS-COV-2 S WuHan-Hu-1 (FIG. 16A), its variants B.1.1.7 (FIG. 16B) and B.1.351 (FIG. 16C) were assessed by surface plasmon resonance (SPR) using an NTA sensor chip. (FIG. 16D) Binding of IgG N3-1 to SARS-COV-1 S was also assessed by SPR. Binding data are shown as black lines. For FIGS. 16A-16C, the best fit to a heterogeneous binding model is shown as red lines. For FIG. 16D, the best fit was achieved using a 1:1 binding model and shown as red lines.

FIGS. 17A-17F. Cryo-EM data validation. (FIGS. 17A-17B) FSC curves (top) and the viewing direction distribution plots (bottom) for global reconstruction of A7V3 bound to SARS-COV-2 S (FIG. 17A) and focused reconstruction of A7V3 bound to S-NTD (FIG. 17B). (FIG. 17C) Cryo-EM density map of A7V3 bound to SARS-COV-2 S (left) and S-NTD (right), respectively. The local resolution is depicted by a spectrum of rainbow color as a scale bar. (FIG. 17D-17E) FSC curves (top) and the viewing direction distribution plots (bottom) for global reconstruction of N3-1 bound to SARS-COV-2 S (FIG. 17D) and focused reconstruction of N3-1 bound to S-RBDs (FIG. 17E). (FIG. 17F) cryo-EM density map of N3-1 bound to SARS-COV-2 S (left) and S-RBDs (right), respectively. The local resolution is depicted by a spectrum of rainbow color as a scale bar.

FIG. 18. N3-1 binding to Regeneron escape mutants and S477N, as measured using the mammalian surface display assay. HEK293T cells transiently expressing full length spike protein were stained with anti-spike antibodies and analyzed by flow cytometry. The median fluorescence intensity of the stained cells was normalized to the HexaPro-D614G spike. Spike variants with single RBD mutations shown to reduce REGN10987 and/or REGN10933 binding were tested with N3-1. The SARS-COV2 RBD subunit and SARS-COV-2 spike with a deleted RBD (ARBD) were included as controls.

FIG. 19. Yeast Surface Display platform. (FIG. 19A) Donor heavy and light chain cDNAs were combinatorially assembled into a GFP/RFP dropout Golden Gate vector. (FIG. 19B) Libraries were integrated into humanized yeast strains optimized for surface display. Yeast libraries were sorted via FACS and subject to short and long read sequencing. (FIG. 19C) Enriched antibodies determined by next-gen sequencing were synthesized as e-blocks (Integrated DNA Technologies) or PCRed directly from yeast genomes. The sequence upstream of the vH insert is SEQ ID NO: 456. The sequence downstream of the vH insert is SEQ ID NO: 457. The sequence upstream of the VL Kappa insert is SEQ ID NO: 458. The sequence downstream of the VL Kappa insert is SEQ ID NO: 459. The sequence upstream of the VL Lambda insert is SEQ ID NO: 458. The sequence downstream of the VL Lambda insert is SEQ ID NO: 460. (FIG. 19D) VH or VL inserts were cloned into mammalian GFP/RFP dropout Golden Gate vectors. The sequence upstream of the RFP DO in the heavy chain vector is SEQ ID NO: 461. The sequence downstream of the RFP DO in the heavy chain vector is SEQ ID NO: 462. The sequence upstream of the GFP DO in the kappa vector is SEQ ID NO: 463. The sequence downstream of the GFP DO in the kappa vector is SEQ ID NO: 464. The sequence upstream of the GFP DO in the lambda vector is SEQ ID NO: 463. The sequence downstream of the GFP DO in the lambda vector is SEQ ID NO: 465. (FIG. 19E) Cloned mAbs were drop-plated for automated colony picking in a QPix 420 (Molecular Devices). Verified mAbs were transfected, purified, and subject to a high-throughput ELISA assay programmed on a Tecan Fluent 1080.

### DETAILED DESCRIPTION

The ongoing evolution of SARS-COV-2 into more easily transmissible and infectious variants has sparked concern over the continued effectiveness of existing therapeutic antibodies and vaccines. Hence, together with increased genomic surveillance, methods to rapidly develop and assess effective interventions are critically needed. Provided herein are SARS-COV-2 spike protein neutralizing antibodies isolated from first disease wave COVID-19 patients using a high-throughput platform. Antibodies were identified from unpaired donor B-cell and serum repertoires using yeast surface display, proteomics, and public light chain screening. Cryo-EM and functional characterization of the antibodies identified N3-1, an antibody with a quaternary binding mode that binds avidly (K_{d,app}=68 pM) to the receptor binding domain (RBD) of the spike protein and robustly neutralizes the virus in vitro. This antibody likely binds all three RBDs of the trimeric spike protein with a single IgG. Importantly, N3-1 equivalently binds spike proteins from emerging SARS-COV-2 variants of concern and binds SARS-COV-1 spike with low nanomolar affinity, which suggests promising cross-reactive therapeutic potential. Taken together, the platform described herein will prove broadly applicable in interrogating adaptive immunity and developing rapid response biological countermeasures to emerging pathogens.

### I. Aspects of the Present Disclosure

SARS-COV-2 and its evolved progeny represent a moving target for therapeutic development. While the rapid response of the global research community has led to the discovery of many neutralizing mAbs (Xiaojie et al., 2021; Chi et al., 2020; Liu et al., 2020), the emergence and widespread dissemination of concern, such as the U.K. variant B.1.1.7, Brazilian variant B.1.1.248, and South African variant B.1.351, stresses the need for continued efforts to rapidly identify and develop antibodies that can bind to diverse epitopes. By combining proteomic analyses of antibody repertoires from patient samples and yeast-display with public light chain pairing, the inventors developed a high-throughput antibody discovery strategy that identified 49 neutralizing antibodies that target multiple regions of the SARS-COV-2 spike protein, including the RBD, NTD, and S2 domains, and that broadly bind multiple viral variants.

To speed the discovery of neutralizing antibodies, Ig-Seq proteomic analyses (see, e.g., U.S. Pat. No. 9,146,241) were performed on patient samples. However, because Ig-Seq does not provide information on relative affinities, epitope targeting, and light chain pairings, two strategies were adopted for light chain discovery: a rapid screening method utilizing public light chains (PLCs) and a YSD light chain selection. The rapid PLC screening method appears particularly useful not only for expediting functional testing of VHs, but also to generate high-affinity antibodies. Ultimately, both methods independently yielded multiple neutralizing antibodies, with several of the most potently neutralizing mAbs converging in two or more of the approaches.

The neutralizing antibodies identified relied on seven IGHV genes, including IGHV3-30 and IGHV1-2, which is consistent with studies that show these genes are overrepresented in SARS-COV-2 neutralizing antibodies (Xiaojie et al., 2021). In addition, IGHV1-24 was previously found to contribute to a number of NTD-directed neutralizing antibodies (Chi et al., 2020; Voss et al., 2020) and was observed in four of the neutralizing antibodies herein. Representative neutralizers were found from each of the discovery strategies, although each method yielded one or more unique IGVH or IGVL genes, validating the high coverage afforded by this multi-pronged approach.

The rapid PLC screening method appears particularly useful not only for expediting functional testing of Ig-Seq-derived heavy chains (each PLC was remarkably represented at least once in a neutralizing antibody), but also to generate high-affinity antibodies. For instance, N3-1 exhibits a sub-nanomolar IC₅₀ (252 pM) and binding affinity (68 pM), while A7V3 has an IC₅₀ of 950 pM. A7V3 is a particularly interesting demonstration of the utility of the strategy, because its heavy chain was first identified from one donor, while the light chain was mapped to another donor. Initial PLC screening indicated a nearly identical IGLC VJ gene (PLC8) as a productive partner, which further confirmed the utility of PLC screening for productive antibody VH-VL pairings.

High-resolution cryo-EM structures of these two newly discovered neutralizing antibodies were resolved, and N3-1 was found to target the RBD, whereas A7V3 was found to target the NTD. Structural analysis of N3-1 revealed that the L-CDR2 and L-CDR3s were both engaged in binding, which suggests that the bioinformatically derived light chains may directly contribute to neutralization.

A7V3 bound to a common neutralization site on the NTD (Voss et al., 2020), and ultimately, PLC8 was found to productively pair with four different neutralizing antibodies that were most commonly associated with NTD-binding heavy chains. This work not only establishes the first empirical demonstration that public light chains can form functional pairings across heavy chains, but also debuts their utility as a prolific method for high affinity antibody discovery, potentially improving rapid-response antibody discovery for neo-antigens in future epidemics. These results further suggest that naturally paired repertoires may be unnecessary for efficient antibody discovery, in contrast to a recently published work where cognate pairing was deemed necessary for effective neutralization (Banach et al., 2020).

The cryo-EM structure of N3-1 is of particular importance because it reveals a novel binding mechanism at a quaternary epitope of the spike trimer, thus allowing this antibody to bind the RBD in both the ‘up’ and ‘down’ conformations. This greatly enhances the binding affinity of the full mAb as it enables binding to all three RBD subunits in the spike protein trimer. As a consequence of this unique binding mode, N3-1 is minimally perturbed by spike mutations in all important variants of concern, including variants B.1.1.7, B.1.1.248, and B.1.351, where it was shown to still have low picomolar affinities and to neutralize the variants with similar titers to WA1. N3-1 is also capable of binding the distantly related SARS-COV-1 spike protein with low nanomolar affinity (28 nM). Given the unique mode of binding and the fact that the N3-1 binding epitope for the RBD-down conformation corresponds to the ACE2-binding site, these data offer a potential mechanism of protection afforded by antibodies elicited from both natural infection and vaccination based on the prefusion stabilized spike of WA1.

The strategy described herein will aid in the rapid identification and development of monoclonal antibodies to extant SARS-COV-2 variants, and can be readily utilized for identifying antibody therapeutics for variants yet to be discovered and for other emerging zoonotic pathogens. Candidate mAbs may be identified, produced, and validated in less than 10 days. Furthermore, these approaches identify mAbs with desirable characteristics—presence in immune responses, public light chain robustness, validation of expression via yeast display—that may enable production at scale. The ability to detect multiple functional VLs for a VH also provides the benefit of increasing the pool of manufacturable candidates. Because of the modular nature of the synthetic biology-like discovery process, the overall strategy could also be readily extended to affinity maturation or directed evolution of tighter binding mAbs to distinct antigens or epitopes. In the long term, the ability to store, combine, and continually reassess repertoires should prove broadly useful in not only discovering novel antibodies with potential therapeutic applications, but also for studying the evolution of adaptive immunity.

### II. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

Other terms used in the fields of recombinant nucleic acid technology, microbiology, immunology, antibody engineering, and molecular and cell biology as used herein will be generally understood by one of ordinary skill in the applicable arts.

### III. Antibodies and Modifications of Antibodies

Provided herein are human monoclonal antibodies having clone-paired variable regions as illustrated in Table 1 as well as clone-paired CDRs from the heavy and light chains as illustrated in Table 2. Such antibodies may be produced using methods described herein.

TABLE 1
Amino acid sequences of the full-
length antibody variable regions.

| | | | SEQ ID |
|---|---|---|---|
| Clone | Chain | Variable Sequence | NO: |
| N3-1 | Heavy | QVQLQESGPGLVNPSETLSLTCSVSGGSFATENYYWSWIR | 1 |
| | | QHPGEGLEWIGNIYFSGNTYYNPSLNNRFTISFDISKNHL | |
| | | SLKLPSVTAADTAVYYCARGTIYFDRSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| 12C8 | Heavy | QVQLVQSGAEVKKPGASVKVSCKVSGYTLTELSMHWVRQA | 3 |
| | | PGKGLEWMGGFDPEDGETIYAQKFQGRVTMTEDTSTDTAY | |
| | | MELSSLRSEDTAVYYCATGPAVRRGSWFDPWGQGTLVTVS | |
| | | S | |
| | Light | QSVLTQPPSVSAAPGQKVTISCSGSSSNIGNNYVSWYQQL | 4 |
| | | PGTAPKLLIYDNNKRPSGIPDRFSGSKSGTSATLGITGLQ | |
| | | TGDEADYYCGTWDSSLSAVVFGGGTKLTVL | |
| A7V3 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVVTQPPSVSAAPGQKVTISCSGSSSNIGNNYVSWYQQL | 6 |
| | | PGTAPKLLIYDNNKRPSGIPDRFSGSKSGTSATLGITGLQ | |
| | | TGDEADYYCGTWDSSLSAVVFGGGTKLTVL | |
| 7-6 | Heavy | QVQLVESGAEVKKPGASVKVSCKVSGYTLTELSMHWVRQA | 7 |
| | | PGKGLEWMGGFDPEDGETIYAQKFQGRVTMTEDTSTDTAY | |
| | | MELSSLRSEDTAVYYCATRFAVYGDYLIDYWGQGTLVTVS | |
| | | S | |
| | Light | SSELSQEPAVSVALGQTVRITCQGDSLRIYYASWYQQKPR | 8 |
| | | QAPVLVIYGKHNRPSGIPDRFSGSSSGNTASLTITGAQAE | |
| | | DEADYYCNSRDSSGDLVVFGGGTKVTVL | |
| 4C7 | Heavy | QVQLVESGAEVQKPGASVKVSCKVSGYTLPELSMHWVRQA | 9 |
| | | PGKGLEWMGGFDPEDGETFYAQKFQGRVTMTEDSSTDTAY | |
| | | MELSSLGSEDTAVYYCATAAAVRGRGTIDYWGQGTLVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| 7A8 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVLTQPPSVSAAPGQKVTISCSGSSSNIGNNYVSWYQQL | 4 |
| | | PGTAPKLLIYDNNKRPSGIPDRFSGSKSGTSATLGITGLQ | |
| | | TGDEADYYCGTWDSSLSAVVFGGGTKLTVL | |
| 3-26 | Heavy | QVQLVQSGGGVVQPGRSLRLSCAASGFTFSSYAMHWVRQA | 13 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELTQLPSASGTPGQRVTISCSGGSSNIGSNYVYWYQQL | 14 |
| | | PETAPKLLIYRNNKRPSGVPDRFSGSKSGTSASLAISGLR | |
| | | SEDEADYYCAAWDDSLSGPVFGTGTQLTVL | |
| N3-3 | Heavy | QVQLQESGPGLVNPSETLSLTCSVSGGSFATENYYWSWIR | 1 |
| | | QHPGEGLEWIGNIYFSGNTYYNPSLNNRFTISFDISKNHL | |
| | | SLKLPSVTAADTAVYYCARGTIYFDRSGYRRVDPFHIWGQ | |
| | | GTMVIVSS | |
| | Light | EIVLTQSPATLSLSPGERATLSCRASQSVSSYLAWYQQKP | 15 |
| | | GQAPRLLIYDASNRATGIPARFSGSGSGTDFTLTISSLEP | |
| | | EDFAVYYCQQRSNWPPLTFGGGTKVEIK | |
| P4B3 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | NFMLTQPHSVSESPGKAVTISCTRNSGSIASNFVQWFQQR | 17 |
| | | PGSYPTTIMYENDNRPSGVPDRFSGSKSGTSATLGITGLQ | |
| | | AEDEADYFCQSSDDSNQLVFGGGTKVTVL | |
| 1D4 | Heavy | QVTLKESGPALVKPTQTLTLTCTFSGFSLSTSGMCVTWVR | 18 |
| | | QPPGKALEWLARIDWDDDKYYSTSLKTRLTISKDTSKNQV | |
| | | VLKMTNMDPVDTATYYCARIPIATHLGSDYWGQGTLVTVS | |
| | | S | |
| | Light | EIVMTQSPATLSVSPGERATLSCRASQSVSSNLAWYQQKP | 19 |
| | | GQAPRLLIYGASTRATGIPARFSGSGSGTEFTLTISSLQS | |
| | | EDFAVYYCQQYNNWPPWTFGQGTKVEIK | |
| N3-7 | Heavy | QVQLQESGPGLVNPSETLSLTCSVSGGSFATENYYWSWIR | 1 |
| | | QHPGEGLEWIGNIYFSGNTYYNPSLNNRFTISFDTSKNHL | |
| | | SLKLPSVTAADTAVYYCARGTIYFDRSGYRRVDPFHIWGQ | |
| | | GTMVIVSS | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| 6-3A | Heavy | QVQLQESGPGLVNPSETLSLTCSVSGGSFATENYYWSWIR | 1 |
| | | QHPGEGLEWIGNIYFSGNTYYNPSLNNRFTISFDTSKNHL | |
| | | SLKLPSVTAADTAVYYCARGTIYFDRSGYRRVDPFHIWGQ | |
| | | GTMVIVSS | |
| | Light | SSELSQEPSFSVSPGGTVTLTCGLSSGSVSTNYYSSWYQQ | 21 |
| | | TPGQAPRTLIYSTNTRSSGVPDRFSGSILGNKAALTITGA | |
| | | QADDESDYYCTLYMGGGLLVFGGGTQLTVL | |
| 8B5 | Heavy | QVQLQESGAEVKKPGASVKVSCKASGYTFTGYYMHWVRQA | 22 |
| | | PGQGLEWMGWINPNSGGTNYAQKFQGRVTMTADRSTNTVY | |
| | | MELSSLKSEDTAVYYCARELPPGRMVVPATYWHFDLWGRG | |
| | | TLVTVSL | |
| | Light | EIVLTQSPGTLSLSPGERATLSCRASQSVSSSYLAWYQQK | 23 |
| | | PGQAPRLLIYGASSRATGIPDRFSGSGSGTDFTLTISRLE | |
| | | PEDFAVYYCQQYGSSPPWTFGQGTKVEIK | |
| 4A5 | Heavy | QVQLVESGGGVVQPGRSLRLSCAASGFTFSSYAMHWVRQA | 24 |
| | | PGKGLEWVAVISYDGSNKYYADSVKGRFTISRDNSKNTLY | |
| | | LQMDSLRPEDTAVYYCAKASQLFWLGQFTRDGFDIWGQGT | |
| | | MVTVSS | |
| | Light | EIVLTQSPGTLSLSPGERATLSCRASQSVSSSYLAWYQQK | 23 |
| | | PGQAPRLLIYGASSRATGIPDRFSGSGSGTDFTLTISRLE | |
| | | PEDFAVYYCQQYGSSPPWTFGQGTKVEIK | |
| 3B9 | Heavy | QVQLVQSGGGVVQPGRSLRLSCAASGFTFSSYAMHWVRQA | 25 |
| | | PGKGLEWVAVISYDGSNKYYADSVKGRFTISRDNSKNTLY | |
| | | LQMNSLRAEDTAVYYCARDGGGYVSYWGQGTLVTVSS | |
| | Light | SYELTQPPSVSVSPGQTASITCSGDKLGDKYACWYQQKPG | 26 |
| | | QSPVLVIYQDSKRPSGIPERFSGSNSGNTATLTISGTQAM | |
| | | DEADYYCQAWDSSTVVFGGGTKLTVL | |
| 6-3B | Heavy | QVQLQESGPGLVNPSETLSLTCSVSGGSFATENYYWSWIR | 1 |
| | | QHPGEGLEWIGNIYFSGNTYYNPSLNNRFTISFDTSKNHL | |
| | | SLKLPSVTAADTAVYYCARGTIYFDRSGYRRVDPFHIWGQ | |
| | | GTMVIVSS | |
| | Light | QSVLTQPPSVSGAPGQRVTISCTGSRSNIGVGYDVHWYQQ | 27 |
| | | LPGRAPKLVIYSNNNRPSGVSDRFSGSRSGSSASLAITGL | |
| | | QAEDEADYYCQSYDGSLNDDVIFGGGTQLTVL | |
| B3.1 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| 1D1 | Heavy | QVTLKESGPALVKPTQTLTLTCTFSGFSLSTSGMCVTWVR | 18 |
| | | QPPGKALEWLARIDWDDDKYYSTSLKTRLTISKDTSKNQV | |
| | | VLKMTNMDPVDTATYYCARIPIATHLGSDYWGQGTLVTVS | |
| | | S | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| 1D9 | Heavy | QVTLKESGPALVKPTQTLTLTCTFSGFSLSTSGMCVTWVR | 18 |
| | | QPPGKALEWLARIDWDDDKYYSTSLKTRLTISKDTSKNQV | |
| | | VLKMTNMDPVDTATYYCARIPIATHLGSDYWGQGTLVTVS | |
| | | S | |
| | Light | SYELTQPPSVSVSPGQTASITCSGDKLGDKYACWYQQKPG | 26 |
| | | QSPVLVIYQDSKRPSGIPERFSGSNSGNTATLTISGTQAM | |
| | | DEADYYCQAWDSSTVVFGGGTKLTVL | |
| N6-2 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | DIVMTQSPLSLPVTPGEPASISCRSSQSLLHSNGYNYLDW | 29 |
| | | YLQKPGQSPQLLIYLGSNRASGVPDRFSGSGSGTDFTLKI | |
| | | SRVEAEDVGVYYCMQALQTPPYTFGQGTKLEIK | |
| P4D3 | Heavy | QVQLVESGGGVVQPGRSLRLSCAASGFIFNNYGMHWVRQA | 30 |
| | | PGKGLEWVAVISYDGSNKYYADSVKGRFTISRDNSKNTLY | |
| | | LQMNSLRAEDTAVYYCAKAPGQWLRFHYYGMDVWGQGTTV | |
| | | TVSS | |
| | Light | QSVVTQPPSVSGAPGQRVTISCTGSSSNIGAHYDVHWYQQ | 31 |
| | | VPGTAPKLLIYGDNNRPSGVPARFSGAKSGTSASLAITGL | |
| | | QADDEAHYYCQSYGNNQGVFGGGTKVTVL | |
| P4A3 | Heavy | QVQLVESGGGVVQPGRSLRLSCAASGFTFSSYAMHWVRQA | 32 |
| | | PGKGLEWVAVISYDGSNKYYADSVKGRFAISRDISKNTLY | |
| | | LQMNSLRAEDTAVYYCARDDTGRVGSGWYCPLYWGQGTLV | |
| | | TVSS | |
| | Light | DIRVTQSPSSLSASVGDRVTITCRASQSISSYLNWYQQKL | 33 |
| | | GKAPKLLIYAASSLQSGVPSRFSGSGSGTDFTLTISSLQP | |
| | | EDFATYYCQQSYSTPLSFGGGTKVEIK | |
| 3-18 | Heavy | QVQLVQSGGGVVQPGRSLRLSCAASGFIFSSYGMHWVRQA | 34 |
| | | PGKGLEWVAVISYDGSNKYYADSVKGRFTISRDNSKNTLY | |
| | | LQMNSLRAEDTAVYYCAKQAGAYCSGGSCYSSSEADYWGQ | |
| | | GTLVTVSS | |
| | Light | QSVVTQPPSVSGAPGQRVTISCTGSSSNIGAHYDVHWYQQ | 35 |
| | | VPGTAPKLLIYGDNNRPSGVPARFSGAKSGTSASLAITGL | |
| | | QADDEAHYYCQSYGNNQGMFGGGTQLTVL | |
| P3B10 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELMQLPSVSVSPGQTASITCSGDKLGDKYACWYQQKPG | 36 |
| | | QSPVLVIYQDSKRPSGIPERFSGSNSGNTATLTISGTQAM | |
| | | DEADYYCQAWDSSTFFGTGTKVTVL | |
| 6-3C | Heavy | QVQLQESGPGLVNPSETLSLTCSVSGGSFATENYYWSWIR | 1 |
| | | QHPGEGLEWIGNIYFSGNTYYNPSLNNRFTISFDTSKNHL | |
| | | SLKLPSVTAADTAVYYCARGTIYFDRSGYRRVDPFHIWGQ | |
| | | GTMVIVSS | |
| | Light | SYELTQPLSVSVALGQTARITCGGNKLGDKYACWYQQKPG | 37 |
| | | QSPVLVIYQDSKRPSGIPERFSGSNSGNTATLTISGTQAM | |
| | | DEADYYCQAWDSSTVVFGGGTKLTVL | |
| 4A7 | Heavy | QVQLVESGGGVVQPGRSLRLSCAASGFTFSSYAMHWVRQA | 24 |
| | | PGKGLEWVAVISYDGSNKYYADSVKGRFTISRDNSKNTLY | |
| | | LQMDSLRPEDTAVYYCAKASQLFWLGQFTRDGFDIWGQGT | |
| | | MVTVSS | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| 1D5 | Heavy | QVTLKESGPALVKPTQTLTLTCTFSGFSLSTSGMCVTWVR | 18 |
| | | QPPGKALEWLARIDWDDDKYYSTSLKTRLTISKDTSKNQV | |
| | | VLKMTNMDPVDTATYYCARIPIATHLGSDYWGQGTLVTVS | |
| | | S | |
| | Light | EIVLTQSPGTLSLSPGERATLSCRASQSVSSSYLAWYQQK | 23 |
| | | PGQAPRLLIYGASSRATGIPDRFSGSGSGTDFTLTISRLE | |
| | | PEDFAVYYCQQYGSSPPWTFGQGTKVEIK | |
| P3C6 | Heavy | QVQLVESGGGLVQPGGSLTLSCAASGFTFSSYAMTWVRQA | 38 |
| | | PGKGLEWVSSISTRGNTYYADSVKGRFTISRDNSKNTLYL | |
| | | QMNSLRGEDTAVYYCAPGRSLYWGQGTLVTVSS | |
| | Light | QSVLTQPPSVSGAPGQTVTISCTGSTSNIGADYDVQWYQQ | 39 |
| | | LPGRAPKLLIYGNTKRPSGVADRFSASKSGTSASLAITGL | |
| | | QAGDEGDYHCQSYDSGLSGSIFGGGTKLTVL | |
| H6 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQLTQSPDSLAVSLGERATINCKSSQSVLYSSNNKNYLA | 41 |
| | | WYQQKPGQPPKLLIYWASTRESGVPDRFSGSGSGTDFTLT | |
| | | ISSLQAEDVAVYYCQQYYSTPPYTFGQGTKVEIK | |
| H4 | Heavy | QVQLQESGPGLVNPSETLSLTCSVSGGSFATENYYWSWIR | 1 |
| | | QHPGEGLEWIGNIYFSGNTYYNPSLNNRFTISFDISKNHL | |
| | | SLKLPSVTAADTAVYYCARGTIYFDRSGYRRVDPFHIWGQ | |
| | | GTMVIVSS | |
| | Light | EIVLTQSPATLSLSPGERATLSCRASQSVSSYLAWYQQKP | 15 |
| | | GQAPRLLIYDASNRATGIPARFSGSGSGTDFTLTISSLEP | |
| | | EDFAVYYCQQRSNWPPLTFGGGTKVEIK | |
| G3 | Heavy | EVQLVESGGDLVQPGGSLRLSCAASGFTFSNYAMSWVRQA | 42 |
| | | PGKGLEWVSAISNSGDKTYYADSVKGRFTISRDNSKNTLF | |
| | | LQMNRLRTEDTAVYYCARVRGYSVNWRYYYHYGMDVWGQG | |
| | | TLVTVSS | |
| | Light | QPGLTQPHSVSESPGKAVTISCTRNSGSITSNFVQWFQQR | 43 |
| | | PGSYPTTIMYENDKRPTGVPHRFSGSIDSSSNSASLSISG | |
| | | LRAEDEADYYCQSSDDSNQLVFGGGTKLTVL | |
| F6 | Heavy | QVQLVESGGGVVQPGGSLRLSCAASGFTFSSYGMHWVRQA | 20 |
| | | PGKGLEWVAFIRYDGSNKYYADSVKGRFTISRDNSKNTLY | |
| | | LQMNSLRAEDTAVYYCAKDTHSSSWRKYYYYYYGMDVWGQ | |
| | | GTTVTVSS | |
| | Light | EIVLTQSPGTLSLSPGERATLSCRASQSVSSSYLAWYQQK | 23 |
| | | PGQAPRLLIYGASSRATGIPDRFSGSGSGTDFTLTISRLE | |
| | | PEDFAVYYCQQYGSSPPWTFGQGTKVEIK | |
| F2 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELMQLPSVSVSPGQTASITCSGDKLGDKYACWYQQKPG | 36 |
| | | QSPVLVIYQDSKRPSGIPERFSGSNSGNTATLTISGTQAM | |
| | | DEADYYCQAWDSSTFFGTGTKVTVL | |
| E4 | Heavy | QVQLVESGGGVVQPGGSLRLSCAASGFTFSSYGMHWVRQA | 11 |
| | | PGKGLEWVAFIRYDGSNKYYADSVKGRFTISRDNSKNTLY | |
| | | LQMNSLRAEDTALYYCAKATRANYYYYYGMDVWGQGTTVT | |
| | | VSS | |
| | Light | ETTLTQSPLSLPVTPGEPASISCRSSQSLLHSNGYNYLDW | 44 |
| | | YLQKPGQSPQLLIYLGSNRASGVPDRFSGSGSGTDFTLKI | |
| | | SRVEAEDVGVYYCMQALQTPPYTFGQGTKVEIK | |
| E3 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELIQPPSVSVSPGQTASITCSGDKLGDKYACWYQQKPG | 45 |
| | | QSPVLVIYQDSKRPSGIPERFSGSNSGNTATLTISGTQAM | |
| | | DEADYYCQAWDSSTGVFGGGTKVTVL | |
| E2 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELMQLPSVSVSPGQTASITCSGDKLGDKYACWYQQKPG | 36 |
| | | QSPVLVIYQDSKRPSGIPERFSGSNSGNTATLTISGTQAM | |
| | | DEADYYCQAWDSSTFFGTGTKVTVL | |
| D6 | Heavy | QVQLVQSGGGLVQPGGSLRLSCAASGFTFSNYAMNWVRQA | 46 |
| | | PGKGLEWVSGISGSGGSTYYADSVKGRFTISRDNSKNTLY | |
| | | LQMNSLRAEDTAVYYCARDRFQSYTYYYYGMDVWGQGTTV | |
| | | TVSS | |
| | Light | EIVLTQSPGTLSLSPGERATLSCRASQSVSSSYLAWYQQK | 23 |
| | | PGQAPRLLIYGASSRATGIPDRFSGSGSGTDFTLTISRLE | |
| | | PEDFAVYYCQQYGSSPPWTFGQGTKVEIK | |
| C6 | Heavy | QITLKESGPVLVKPTETLTLTCTVSGFSLNNRMGVSWLRQ | 47 |
| | | PPGKALEWLAHTFSSGEPSYSTSLKNRVTISKDTSKGQVV | |
| | | LTMANVDPLDTATYYCARVMTSMRLWYWGYWEDPWGQGIR | |
| | | VTVSS | |
| | Light | QSVLIQPPSVSGAPGQRVTISCTGSRSNIGVGYDVHWYQQ | 48 |
| | | LPGRAPKLVIYSNNNRPSGVSDRFSGSRSGSSASLAITGL | |
| | | QAEDEADYYCQSYDGSLNDDVIFGGGTKLTVL | |
| C4 | Heavy | QVQLVESGGGLVQPGGSLTLSCAASGFTFSSYAMTWVRQA | 38 |
| | | PGKGLEWVSSISTRGNTYYADSVKGRFTISRDNSKNTLYL | |
| | | QMNSLRGEDTAVYYCAPGRSLYWGQGTLVTVSS | |
| | Light | QSVVTQPPSVSGAPGQTVTISCTGSTSNIGADYDVQWYQQ | 49 |
| | | LPGRAPKLLIYGNTKRPSGVADRFSASKSGTSASLAITGL | |
| | | QAGDEGDYHCQSYDSGLSGSIFGGGTQLTVL | |
| C3 | Heavy | EVQLVESGGGVVQPGRSLRLSCAASGFTFSSYAMHWVRQA | 50 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SSELSQPPSASGTPGQRVTISCSGGSSNIGSNYVYWYQQL | 51 |
| | | PETAPKLLIYRNNKRPSGVPDRFSGSKSGTSASLAISGLR | |
| | | SEDEADYYCAAWDDSLSGPVFGTGTKLTVL | |
| B4 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELMQLPSVSVSPGQTASITCSGDKLGDKYACWYQQKPG | 36 |
| | | QSPVLVIYQDSKRPSGIPERFSGSNSGNTATLTISGTQAM | |
| | | DEADYYCQAWDSSTFFGTGTKVTVL | |
| B3 | Heavy | QVQLQESGPGLVNPSETLSLTCSVSGGSFATENYYWSWIR | 1 |
| | | QHPGEGLEWIGNIYFSGNTYYNPSLNNRFTISFDISKNHL | |
| | | SLKLPSVTAADTAVYYCARGTIYFDRSGYRRVDPFHIWGQ | |
| | | GTMVIVSS | |
| | Light | QSVLTQPPSVSAAPGQKVTISCSGSSSNIGNNYVSWYQQL | 4 |
| | | PGTAPKLLIYDNNKRPSGIPDRFSGSKSGTSATLGITGLQ | |
| | | TGDEADYYCGTWDSSLSAVVFGGGTKLTVL | |
| A6 | Heavy | QVQLVQSGGGLVQPGGSLRLSCAASGFTFSNYAMNWVRQA | 46 |
| | | PGKGLEWVSGISGSGGSTYYADSVKGRFTISRDNSKNTLY | |
| | | LQMNSLRAEDTAVYYCARDRFQSYTYYYYGMDVWGQGTTV | |
| | | TVSS | |
| | Light | QSVLTQPPSVSGAPGQRVTISCTGSTSNIGATYDVHWYQQ | 52 |
| | | RPGAAPKLLIYGNNNRPSGVPDRFSGSKSGTSASLAITGL | |
| | | QADDEAHYYCQSYGNNQGVFGGGTKLTVL | |
| A5 | Heavy | QVTLKESGAEVKKPGASVKVSCKASGYTFTGYYLHWVRQA | 53 |
| | | PGQGLEWMGWINPNSGGTNYAQKFQGRVTMTRDTSISTAY | |
| | | MELSRLRSDDTAVYYCARLRGYSYGHYYGMDVWGQGTTVT | |
| | | VSS | |
| | Light | DIVLTQSPATLSVSPGERATLSCRASQSVSSNLAWYQQKP | 54 |
| | | GQAPRLLIYGASTRATGIPARFSGSGSGTEFTLTISSLQS | |
| | | EDFAVYYCQQYNNWPPWTFGQGTKVDIK | |
| A4 | Heavy | QVQLVESGGGVVQPGRSLRLSCAASGFTFSSYAMHWVRQA | 55 |
| | | PGKGLEWVAVISYDGSNKYYADSVKGRFTISRDNSKNTLY | |
| | | LQMNSLRAEDTAVYYCARDARRFKYYYYYGMDVWGQGTTV | |
| | | TVSS | |
| | Light | QSVLTQPPSVSGAPGQRVTISCTGGSSNLGAAYDVHWYQQ | 56 |
| | | YPGRAPKLLIYNNNNRPSGVPDRFSGSKSDTSASLVIAGL | |
| | | QAEDEAEYYCQSYDISLSGLYVFGTGTKVTVL | |
| A3 | Heavy | QVQLVESGGGVVQPGGSLRLSCAASGFTFSSYGMHWVRQA | 20 |
| | | PGKGLEWVAFIRYDGSNKYYADSVKGRFTISRDNSKNTLY | |
| | | LQMNSLRAEDTAVYYCAKDTHSSSWRKYYYYYYGMDVWGQ | |
| | | GTTVTVSS | |
| | Light | QPVLTQPPSASGTPGQRVTISCSGSSSNIGSNTVNWYQQL | 57 |
| | | PGTAPKLLIYSNNQRPSGVPDRFSGSKSGTSASLAISGLQ | |
| | | SEDEADYYCAAWDDSLNGPVVFGGGTKLTVL | |
| A2 | Heavy | QVQLVQSGGGLVQPGGSLRLSCAASGFTFGGYPMHWVRLA | 58 |
| | | PGKGLEWLAVISYDGSSAYYIDSVKGRFTVSRDNSKNTLY | |
| | | LEMSSLGTEDTAVYYCARPRSYRNHRFDYWGQGTLVTVSS | |
| | Light | EIVLTQSPATLSLSPGERATLSCRASQSVSSYLAWYQQKP | 15 |
| | | GQAPRLLIYDASNRATGIPARFSGSGSGTDFTLTISSLEP | |
| | | EDFAVYYCQQRSNWPPLTFGGGTKVEIK | |
| 96 | Heavy | QVQLVQSGGGVVQPGRSLRLSCAASGFIFNNYGMHWVRQA | 59 |
| | | PGKGLEWVAVISYDGSNKYYADSVKGRFTISRDNSKNTLY | |
| | | LQMNSLRAEDTAVYYCAKAPGQWLRFHYYGMDVWGQGTTV | |
| | | TVSS | |
| | Light | SSELTQDPAVSVALGQTVKITCQGDSSNIGAHYDVHWYQQ | 60 |
| | | VPGTAPKLLIYGKNNRPSGVPARFSGAKSGTSASLAITGL | |
| | | QADDEAHYYCNSRDINSNHVLFGGGTKVTVL | |
| 100 | Heavy | QVQLVESGRRGPAWGSLRLSCAASGFTFSSYGMHWVRQAP | 61 |
| | | GKGLEWVAFIRYDGSNKYYADSVKGRFTISRDNSKNTLYL | |
| | | QMNSLRAEDTAVYYCAKDRRYYDFWSGYVGSPYYYYYYGM | |
| | | DVWGQGTTVTVSS | |
| | Light | SYELMHTSVSVSPGQTASITCSGDKLGDKYACWYQQKPGQ | 62 |
| | | SPVLVIYSNNKRPSGIPERFLSGSNSGNTATDHQRTQAMD | |
| | | EADYYCQSYGNNQGVFGGGTKVTVL | |
| 118 | Heavy | QVQLVQSGGGVVQPGRSLRLSCAASGFTFSDYGMHWVRQA | 63 |
| | | PGKGLEWVAVVSYDGSNKYYADSVKGRFTISRDNSKNTVY | |
| | | LQMNSLRAEDTAVYYCASPTVTMGHGKLVTVSS | |
| | Light | DIQMTQSPSSLSASVGDRVTITCRASQSISTYLNWYQQKP | 64 |
| | | GKAPKLLIYAASRLQSEVPSRFSGSGSGTDFTLTISSLEP | |
| | | EDFATYSCQQSYSSSWTFGQGTKVEIK | |
| 97 | Heavy | QVQLVESGGGVVQPGRSLRLSCAASGFTFSSYAMHWVRQA | 65 |
| | | PGKGLEWVAVISYDGSNKYYADSVKGRFTISRDNSKNTLY | |
| | | LQMNSLRAEDTAVYYCARGNWGSYYYGMDVWGQGTTVTVS | |
| | | S | |
| | Light | SYELMQLPSVSVSPGQTASITCSGDNIGSKSVHWYQQKPG | 66 |
| | | QAPVLVVYDDSKRPSGIPERFSGSNSGNTATLTISGTQAM | |
| | | DEADYYCQSSDDSNQLVFGGGTKVTVL | |
| 3 | Heavy | QVQLVQSGGGLVQPGGSLRLSCAASGFTVSSNYMSWVRQA | 67 |
| | | PGKGLEWVSVIYSGGSTYYADSVKGRFTISRDNSKNTLYL | |
| | | QMNSLRAEDTAVYYCARGGVVDTYYYYGMDVWGQGTTVTV | |
| | | SS | |
| | Light | QAVVTQEPSLTVSPGGTVTLTCGSSTGPVTTGHWPYWLQQ | 68 |
| | | KTGQAPRTLIYDTVNKHSWTPARFSGSLLGGKAALTLSGA | |
| | | QPEDEADHYCLLSQSGAWVFGGGTKLTVL | |
| 114 | Heavy | QVQLVQSGAEVKKPGASVKVSCKVSGYTLTELSMHWVRQA | 3 |
| | | PGKGLEWMGGFDPEDGETIYAQKFQGRVTMTEDTSTDTAY | |
| | | MELSSLRSEDTAVYYCATGPAVRRGSWFDPWGQGTLVTVS | |
| | | S | |
| | Light | QSVLTQPPSVSAAPGQKVTISCSGSSSNIGNNYVSWYQQL | 69 |
| | | PGTAPKLLIYDNNKRPSGIPDRFSGSKSGTSATLGITGLQ | |
| | | TGDEADYYCGTWDSSLSGYVFGTGTKVTVL | |
| 144 | Heavy | QVQLVQSGGGVVQPGRSLRLSCAASGFIFSSYGMHWVRQA | 34 |
| | | PGKGLEWVAVISYDGSNKYYADSVKGRFTISRDNSKNTLY | |
| | | LQMNSLRAEDTAVYYCAKQAGAYCSGGSCYSSSEADYWGQ | |
| | | GTLVTVSS | |
| | Light | QSVLIQPPSVSGAPGQRVTISCTGSSSNIGAHYDVHWYQQ | 70 |
| | | VPGTAPKLLIYGDNNRPSGVPARFSGAKSGTSASLAITGL | |
| | | QADDEAHYYCQSYGNNQGVFGGGTKVTVL | |
| 32 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVLTQPPSASGSPGQSVTISCTGTSSDVGGYNYVSWYQQ | 71 |
| | | HPGKAPKLMIYEVSKRPSGVPDRFSGSKSGNTASLTVSGL | |
| | | QAEDEADYYCSSYAGSNNLAFGGGTKLTVL | |
| 131 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVVTQPPSVSAAPGQKVTISCSGSRSNIGNNYVSWYQQL | 72 |
| | | PGTAPKLLIYDYDKRPSGIPDRFSGSKSGTSATLGITGLQ | |
| | | TGDEADYYCGTWDNSLSAGVFGGGTRLTVL | |
| 130 | Heavy | QVQLQESGGGVVQPGRSLRLSCAASGFTFSSYAMHWVRQA | 73 |
| | | PGKGLEWVAVISYDGSNKYYADSVKGRFTISRDNSKNTLY | |
| | | LQMNSLRAEDTAVYYCATGPAVRRGSWFDPWGQGTLVTVS | |
| | | S | |
| | Light | AIQLTQSPSSLSASVGDRVTITCRASQSISSYLNWYQQKP | 74 |
| | | GKAPKLLIYAASSMQSGVPSRFSGSGSGTDFTLTISSLQP | |
| | | EDFATYYCQQSYSTPRTFGQGTKVEIK | |
| 132 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVLTQPPSVSAAPGQKVTISCSGSSSNIGNNYVSWYQQL | 75 |
| | | PGTAPKLLIYDNNKRPSGIPDRFSGSKSGTSATLGITGLQ | |
| | | TGDEADYYCGTWDSSLSAGVFGGGTKLTVL | |
| 19 | Heavy | QVQLQQSGPGLVNPSETLSLTCSVSGGSFATENYYWSWIR | 76 |
| | | QHPGEGLEWIGNIYFSGNTYYNPSLNNRFTISFDTSKNHL | |
| | | SLKLPSVTAADTAVYYCARGTIYFDRSGYRRVDPFHIWGQ | |
| | | GTMVIVSS | |
| | Light | AIQLTQSPSSLSASVGDRVTITCRASQSISSYLNWYQQKP | 77 |
| | | GKAPKLLIYAASSLQSGVPSRFSGSGSGTDFTLTISSLQP | |
| | | EDFATYYCQQSYSTRPLTFGGGTKVEIK | |
| 31 | Heavy | QVQLVQSGAEVKKPGASVKVSCKVSGYTLTELSMHWVRQA | 5 |
| | | PGKGLEWMGGFDPEDGETIYAQKFQGRVTMTEDTSTDTAY | |
| | | MELSSLRSEDTAVYYCATGSPFDRTQNWFDPWGQGTLVTV | |
| | | SS | |
| | Light | QSVLTQPPSVSAAPGQKVTISCSGSSSNIGKNYVSWYQQF | 78 |
| | | PGTAPKLLIYSNNQRPSGVPDRFFGSKSGTSASLAISGLQ | |
| | | SEDEADYYCAAWDDGLSAVVFGGGTKLTVL | |
| 42 | Heavy | QVQLVQSGGGVVQPGRSLRLSCAASGFTFSSYAMHWVRQA | 79 |
| | | PGKGLEWVAVISYDGSNKYYADSVKGRFTISRDNSKNTLY | |
| | | LQMNSLRAEDTAIYYCARDYGRGGVWGQGTTVTVSS | |
| | Light | AIQLTQSPSSLSASVGDRVTITCRASQSISSYLNWYQQKP | 77 |
| | | GKAPKLLIYAASSLQSGVPSRFSGSGSGTDFTLTISSLQP | |
| | | EDFATYYCQQSYSTRPLTFGGGTKVEIK | |
| 36 | Heavy | EVQLLESGGGLVKPGGSLRLSCAASGFTFSNAWMSWVRQA | 80 |
| | | PGKGLEWVGRIKSKTDGGTTDYAAPVKGRFTISRDDSKNT | |
| | | LYLQMNSLKTEDTAVYYCTTDRTYDYVWGSYRYEDYWGQG | |
| | | TLVTVSS | |
| | Light | DIQMTQSPSSLSASVGDRVTITCRASQSISSYLNWYQQKP | 81 |
| | | GKAPKLLIYAASSLQSGVPSRFSGSGSGTDFTLTISSLQP | |
| | | EDFATYYCQQSYSTPEFGQGTKVEIK | |
| 46 | Heavy | QVQLVESGGGVVQPGRSLRLSCVVSGFTFNNFGMHWVRQA | 82 |
| | | PGKGLEWVAYISYEGSIKYYAQSLKGRFTISRDSSKNTLY | |
| | | LQMNSLRADDTAMYYCAKDRAIFYLNPRYYLDYWGQGILV | |
| | | TVSS | |
| | Light | SYVLTQPPSVSVAPGKTARITCGGNKLGDKYACWYQQKPG | 83 |
| | | QSPVLVIYQDSKRPSGIPERFSGSNSGNTATLTISGTQAM | |
| | | DEADYYCQAWDSSTAVVFGGGTKLTVL | |
| IgG-S2-14 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | QSVLTQPPSVSGAPGQRVTISCTGGSSNLGAAYDVHWYQQ | 340 |
| | | YPGRAPKLLIYNNNNRPSGVPDRFSGSKSDTSASLVIAGL | |
| | | QAEDEAGYYCQSYDISLSGLYVFGTGTKLTVL | |
| IgG-S2-15 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | QSVLTQPPSVSGAPGQRVTISCTGSSSNIGAGYDVHWYQQ | 341 |
| | | VPGTATKLLIYGNNNRPPGVPDRFSGSKSGTSASLTITGL | |
| | | RAEDEADFFCQSYDRSVSGWVFGGGTKLTVL | |
| IgG-S2-21 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | NFMLTQPHSVSESPGKAVTISCTRNSGSIASNFVQWFQQR | 342 |
| | | PGSYPTTIMYENDKRPTGVPHRFSGSIDSSSNSASLSISG | |
| | | LRAEDEADYYCQSSDDSNQLVFGGGTKVTVL | |
| IgG-S2-03 | Heavy | QVQLQQSGGGVVQPGRSLRLSCAASGFTFSSYAMHWVRQA | 383 |
| | | PGKGLEWVAVISYDGDRVYYADSVRGRFNISRDNSKNTLY | |
| | | LQMNSLRPEDTAVYYCAKSRSGSYYYGMDVWGQGTTVTVS | |
| | | S | |
| | Light | SYELIQPPSVSVAPGQTARITCGNNIGSKTVHWYQQKPGQ | 343 |
| | | APVLVVYDDSDRPSGIPERFSGSNSGNTATLTISRVEAGD | |
| | | EADYYCQVWESSGDHYWVFGRGTKVTVL | |
| IgG-S2-16 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | SSELSQDPAVSVALGQTVRITCQGDSLRSYYASWYQQRPG | 344 |
| | | RAPVLVIYGKNNRPSGIPDRFSGSSSGNTASLTITGAQAE | |
| | | DEADYYCDSRDSSGKRRVFGGGTKLTVL | |
| IgG-S2-19 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | QSALIQPPSVSGAPGQRVTISCTGSSSNIGADYDVHWCQL | 345 |
| | | LPGIAPKLLIFANSPRPSGVPDRFSGSKSATSASLAITGL | |
| | | LAEDEASYYCQSYDSILNDSAVGGGTKLTVL | |
| IgG-S2-18 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | QSALIQPPSVSGAPGQRVTISCTGSSSNIGADYDVHWCQL | 346 |
| | | LPGIAPKLLIFANSPRPSGVPDRFSGSKSATSASLAITGL | |
| | | LAEDEASYYCQSYDSILNDSGVVGGGTKLTVL | |
| IgG-S2-17 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | QSALIQPPSVSGAPGQRVTISCTGSSSNIGADYDVHWCQL | 347 |
| | | LPGIAPKLLIFANSPRPSGVPDRFSGSKSATSASLAITGL | |
| | | LAEDEASYYCQSYDSILNDSVVGGGTKLTVL | |
| IgG-S2-09 | Heavy | QVQLVQSGGGVVQPGRSLRLSCAASGFTFSSYAMHWVRQA | 387 |
| | | PGKGLEWVAVISYDGSNKYYADSVKGRFTISRDNSKNTLY | |
| | | LQMNSLRAEDTAVYYCARAYGGGYLTPFDYWGQGTLVTVS | |
| | | S | |
| | Light | QSVLTQPPSVSGAPGQRVTISCTGGSSNIGAGYDVHWYQQ | 348 |
| | | VPGTATKLLIYGNNNRPSGVPDRFSGSKSGTSASLTITGL | |
| | | RAEDEADFFCQSYDRSVSGWVFGGGTQLTVL | |
| IgG-S2-10 | Heavy | QVQLVESGGGVVQPGGSLRLSCAASGFTFSSYAMHWVRQA | 388 |
| | | PGKGLEWVAVISYDGSNKYYADSVKGRFTISRDNSKNTLY | |
| | | LQMNSLRAEDTAVYYCARDGSGGYSNPDYWGQGTLVTVSS | |
| | Light | SSELTQEPAVSVALGQTVRITCQGDSLRIYYASWYQQKPR | 349 |
| | | QAPVLVIYGKHNRPSGIPDRFSGSSSGNTASLTITGAQAE | |
| | | DEADYYCNSRDSSGDLVVFGGGTKVTVL | |
| IgG-S2-08 | Heavy | QVQLVESGGGVVQPGRSLRLSCAASGFTFSSYAMHWVRQA | 390 |
| | | PGKGLEWVAVISYDGSNKYYADSVKGRFTISRDNSKNTLY | |
| | | LQMNSLRAEDTAVYYCARASHGGYSSAFDPWGQGTLVTVS | |
| | | S | |
| | Light | SSELSQDPAVSVALGQTVRITCQGDSLRIYYASWYQQKPR | 350 |
| | | QAPVLVIYGKHNRPSGIPDRFSGSSSGNTASLTITGAQAE | |
| | | DEADYYCNSRDSSGDLVVFGGGTKLTVL | |
| IgG-S2-01 | Heavy | QVQLVQSGGGVVQPGRSLRLSCAASGFTFSSYGMHWVRLA | 351 |
| | | PGKGLEWVAVISYDGSNKYYADSVKGRFTISRDNSKNTLY | |
| | | LQMNSLRAEDTAVYYCAKPQGGYNGAFDIWGQGTMVTVSS | |
| | Light | SSELTQDPAVSVALGQTVRITCQGDSLRIYYASWYQQKPR | 355 |
| | | QAPVLVIYGKHNRPSGIPDRFSGSSSGNTASLTITGAQAE | |
| | | DEADYYCNSRDSSGDLVVFGGGTKLTVL | |
| IgG-S2-02 | Heavy | QVQLVQSGGGVVQPGRSLRLSCAASGFTFSSYGMHWVRLA | 351 |
| | | PGKGLEWVAVISYDGSNKYYADSVKGRFTISRDNSKNTLY | |
| | | LQMNSLRAEDTAVYYCAKPQGGYNGAFDIWGQGTMVTVSS | |
| | Light | SSELTQEPAVSVALGQTVRITCQGDSLRIYYASWYQQKPR | 352 |
| | | QAPVLVIYGKHNRPSGIPDRFSGSSSGNTASLTITGAQAE | |
| | | DEADYYCNSRDSSGDLVVFGGGTKLTVL | |
| IgG-S2-04 | Heavy | QVQLVQSGGGVVQPGRSLRLSCAASGVSITSYHWSWIRQS | 353 |
| | | PGKGLEWVAFMYYISGQTSSNPSLKGRVIMSLDTSKNQVS | |
| | | LKLMSVTAADTAIYFCARADSCNGGICYYQGMDVWGQGTA | |
| | | VTVSS | |
| | Light | SYELIQPPSVSVAPGQTARITCGGNKLGDKYACWYQQKPG | 354 |
| | | QSPVLVIYQDSKRPSGIPERFSGSNSGNTATLTISGTQAM | |
| | | DEADYYCQAWDSSTVVFGGGTKLTVL | |
| IgG-S2-06 | Heavy | QVQLQQSGGGVVQPGRSLRLSCAASGFTFSSYAMHWVRQA | 356 |
| | | PGKGLEWVAVISYDGSNKYYADSVKGRFTISRDNSKNTLY | |
| | | LQMNSLRAEDTAVYYCARAGGGGYQGPFDYWGQGTLVTVS | |
| | | S | |
| | Light | SYELTQEPAVSVALGQTVRITCQGDSLRIYYASWYQQKPR | 364 |
| | | QAPVLVIYGKHNRPSGIPDRFSGSSSGNTASLTITGAQAE | |
| | | DEADYYCNSRDSSGDLVVFGGGTKLTVL | |
| IgG-S2-07 | Heavy | QVQLQQSGGGVVQPGRSLRLSCAASGFTFSSYAMHWVRQA | 356 |
| | | PGKGLEWVAVISYDGSNKYYADSVKGRFTISRDNSKNTLY | |
| | | LQMNSLRAEDTAVYYCARAGGGGYQGPFDYWGQGTLVTVS | |
| | | S | |
| | Light | SSELTQDPAVSVALGQTVRITCQGDSLRIYYASWYQQKPR | 355 |
| | | QAPVLVIYGKHNRPSGIPDRFSGSSSGNTASLTITGAQAE | |
| | | DEADYYCNSRDSSGDLVVFGGGTKLTVL | |
| IgG-S2-05 | Heavy | QVQLQQSGGGVVQPGRSLRLSCAASGFTFSSYAMHWVRQA | 356 |
| | | PGKGLEWVAVISYDGSNKYYADSVKGRFTISRDNSKNTLY | |
| | | LQMNSLRAEDTAVYYCARAGGGGYQGPFDYWGQGTLVTVS | |
| | | S | |
| | Light | SYELTQEPAVSVALGQTVRITCQGDSLRIYYASWYQQKPR | 357 |
| | | QAPVLVIYGKHNRPSGIPERFSGSNSGNTATLTISGTQAM | |
| | | DEADYYCQAWDSSTAWNVFGTGTKVTVL | |
| IgG-S2-11 | Heavy | QVQLVQSGGGVVQPGRSLRLSCAASGFTFSSYAMHWVRQA | 358 |
| | | PGKGLEWVAVISYDGSNKYYADSVKGRFTISRDNSKNTLY | |
| | | LQMNSLRAEDTAVYYCARDLRGSYSGLLDYWGQGTLVTVS | |
| | | S | |
| | Light | QSVLTQPPSVSGAPGQRVTISCTGSSSNIGAGYDVHWYQQ | 359 |
| | | VPGTATKLLIYGNNNRPSWVPDRFSGSKSGTSASLTITGL | |
| | | RAEDEADFFCQSYDRSVSGWVFGGGTKLTVL | |
| IgG-S2-22 | Heavy | EVQLVQSGGGLVQPGGSLRLSCAASGFTFSNYAMNWVRQA | 360 |
| | | PGKGLEWVSGISGSGGSTYYADSVKGRFTISRDNSKNTLY | |
| | | LQMNSLRAEDTAVYYCARDRFQSYTYYYYGMDVWGQGTTV | |
| | | TVSS | |
| | Light | QSVLTQPPSVSGAPGQRVTISCTGSTSNIGATYDVHWYQQ | 52 |
| | | RPGAAPKLLIYGNNNRPSGVPDRFSGSKSGTSASLAITGL | |
| | | QADDEAHYYCQSYGNNQGVFGGGTKLTVL | |
| IgG-S2-12 | Heavy | AVQLVQSGGGLVQPGGSLRLSCAASGFTFSSYDMHWVRQA | 361 |
| | | TGKGLEWVSAIGTAGDTYYPGSVKGRFTISRENAKNSLYL | |
| | | QMNSLRAGDTAVYYCARGSHPRKRSGLSLDAFDIWGQGTM | |
| | | VTVSS | |
| | Light | QSVVTQPPSMSAAPGQRVTISCSGSNSNIGSNYLSWYQQL | 362 |
| | | PGAAPKLLIYENNKRPSGIPDRFSGSKSGTSATLGITGLQ | |
| | | TGDEADYYCATWDSSLSAVVFGGGTKLTVL | |
| IgG-S2-13 | Heavy | QVQLQESGGGVVQPGRSLRLSCAASGFTFSSYAMHWVRQA | 28 |
| | | PGKGLEWVAVISYDGSNKYYADSVKGRFTISRDNSKNTLY | |
| | | LQMNSLRAEDTAVYYCARPYSGSYWGYFDYWGQGTLVTVS | |
| | | S | |
| | Light | SYELMQLPSVSVSPGQTASITCSGDKLGDKYACWYQQKPG | 36 |
| | | QSPVLVIYQDSKRPSGIPERFSGSNSGNTATLTISGTQAM | |
| | | DEADYYCQAWDSSTFFGTGTKVTVL | |
| IgG-S2-20 | Heavy | QVQLVQSGAEVKKPGASVKVSCKASGYTFTNYGLHWVRQA | 363 |
| | | PGQRLEWMGWISTGSGNTQHSQKFQGRVTITRDTSASTAY | |
| | | MELSSLRSEDTAVYYCVKGAGGYFDSWGQGTLVTVSS | |
| | Light | SYELTQEPAVSVALGQTVRITCQGDSLRIYYASWYQQKPR | 364 |
| | | QAPVLVIYGKHNRPSGIPDRFSGSSSGNTASLTITGAQAE | |
| | | DEADYYCNSRDSSGDLVVFGGGTKLTVL | |
| IgG-S2-30 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-31 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSLSASVGDRVTITCRASQSVSSYLNWYQQKP | 365 |
| | | GKAPKLLIYAASGLQSGVPSRFSGRGSGTDFTLTINSLQP | |
| | | EDFATYFCQQSSSIPRTFGQGTKVDIK | |
| IgG-S2-32 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSLSASVGDRVTITCRASQSVSSYLNWYQQNQ | 366 |
| | | ESPKLLIYAASGLQSGVPSRFSGRGSGTDFTLTINSLQPE | |
| | | DFATYFCQQSSSIPRTFGQGTKVDIK | |
| IgG-S2-33 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSLSASVGDRVTITCRASQSVSSYLNWYQQNQ | 367 |
| | | GKPKLLIYAASGLQSGVPSRFSGRGSGTDFTLTINSLQPE | |
| | | DFATYFCQQSSSIPRTFGQGTKVDIK | |
| IgG-S2-34 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-35 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | SYELTQPPSVSVAPGQTARVTCGGNNIGSKSVHWYQQKPG | 368 |
| | | QAPVLVVYDDSDRPSGIPERFSGSNSGNTATLTISGTQAM | |
| | | DEADYYCQAWDSSVFGGGTKLTVL | |
| IgG-S2-36 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | SYELTQPPSVSVAPGQTARVTCGGNNIGSKSVHWYQQKPG | 369 |
| | | QAPVLVVYDDSDRPSGIPERFSGSNSGNTATLTISGTQAM | |
| | | DEADYYCQAWDSVVFGGGTKLTVL | |
| IgG-S2-37 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | SYELTQPPSVSVAPGQTARVTCGGNNIGSKSVHWYQQKPG | 370 |
| | | QAPVLVVYDDSDRPSGIPERFSGSNSGNTATLTISGTQAM | |
| | | DEADYYCQAWDSSVEPGGGTKLTVL | |
| IgG-S2-38 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-39 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | QSVLTQPPSASGTPGQRVTISCSGSSSNIGSNYVYWYQQL | 375 |
| | | PGTAPKLLIYRNNQRPSGVPDRFSGSKSGTSASLAISGLR | |
| | | SEDEADYYCAAWDDSLSGWVFGGGTKLTVL | |
| IgG-S2-40 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSTLSASVGDRVTITCRASQTIYSWLAWYQQKP | 376 |
| | | GKAPKFLIHKASSLESGVPSRFSGSGSGTEFTLTISSLQP | |
| | | DDFATYYCQQYSSYPRTFGQGTKVEIK | |
| IgG-S2-41 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIVMTQSPDSLAVSLGERATINCKSSQSVLYSSNNKNYLA | 12 |
| | | WYQQKPGQPPKLLIYWASTRESGVPDRFSGSGSGTDFTLT | |
| | | ISSLQAEDVAVYYCQQYYSTPPYTFGQGTKLEIK | |
| IgG-S2-42 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSMSASVGDRVIITCRASQSISSSLNWYQQKP | 377 |
| | | GKAPNLLIYSASTLQSEVPSRFSGSGSGTDFTLTISSLQP | |
| | | EDFATYYCQQSYSTPPAFGQGTKVEFK | |
| IgG-S2-43 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSAMSASVGDRVTITCRASQGISNYLAWFQQKP | 379 |
| | | GKVPKRLIYAASSLQSGVPSRFCGSGSGTDFTLTIASLQP | |
| | | EDFATYYCQQIYSFPITFGQGTRLETK | |
| IgG-S2-44 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSMSASVGDRVIITCRASESLITWLAWYQQRP | 380 |
| | | GEAPKVLIYKGSTLVRGVPSRFSGRGSGTEFTLTITSLQP | |
| | | DDFATYYCQQYSGFPITFGQGTKVEIK | |
| IgG-S2-45 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | QSVLTQPPSVSAAPGQKVTISCTGSSSNIGAGYNVHWYQQ | 371 |
| | | LPGTAPKLLIYGNSNRPSGVPDRFSGSKSGTSASLAITGL | |
| | | QAEDEADYYCQSYDSSLSGSGVFGGGTKLTVL | |
| IgG-S2-46 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | SYELTQPPSVSVAPGKTARITCGGNNIGSKSVHWYQQKPG | 372 |
| | | QAPVLVVYDDSDRPSGIPERFSGSNSGNTATLTISRVEAG | |
| | | DEADYYCQVWDSSSDHWVFGGGTKLTVL | |
| IgG-S2-47 | Heavy | QVQLVQSGGGVVQPGRSLRLSCAASGFTFSSYAMHWVRQA | 339 |
| | | PGKGLEWVAVISYDGSNKYYADSVKGRFTISRDNSKNTLY | |
| | | LQMNSLRAEDTAVYYCARSTYGSYYYGMDVWGQGTTVTVS | |
| | | S | |
| | Light | DIQMTQSPSSLSASVGDRVTITCRASQSISSYLNWYQQKP | 373 |
| | | GKAPKLLIYAASSLQSGVPSRFSGSGSGTDFTLTISSLQP | |
| | | EDFATYYCQQSYSTPWTFGQGTKLEIK | |
| IgG-S2-48 | Heavy | EVQLVESGGGVVQPGKSLRLSCAASGFTENNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-49 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-50 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-51 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | QSVLTQPPSASGTPGQRVTISCSGSSSNIGSNYVYWYQQL | 375 |
| | | PGTAPKLLIYRNNQRPSGVPDRFSGSKSGTSASLAISGLR | |
| | | SEDEADYYCAAWDDSLSGWVFGGGTKLTVL | |
| IgG-S2-52 | Heavy | EVQLVESGGGVVQPGKSLRLSCAASGFTENNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPSTLSASVGDRVTITCRASQTIYSWLAWYQQKP | 376 |
| | | GKAPKFLIHKASSLESGVPSRFSGSGSGTEFTLTISSLQP | |
| | | DDFATYYCQQYSSYPRTFGQGTKVEIK | |
| IgG-S2-53 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIVMTQSPDSLAVSLGERATINCKSSQSVLYSSNNKNYLA | 12 |
| | | WYQQKPGQPPKLLIYWASTRESGVPDRFSGSGSGTDFTLT | |
| | | ISSLQAEDVAVYYCQQYYSTPPYTFGQGTKLEIK | |
| IgG-S2-54 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPSSMSASVGDRVIITCRASQSISSSLNWYQQKP | 377 |
| | | GKAPNLLIYSASTLQSEVPSRFSGSGSGTDFTLTISSLQP | |
| | | EDFATYYCQQSYSTPPAFGQGTKVEFK | |
| IgG-S2-55 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPDSLAVSLGERATINCKSGQSVLYSSNNKNYLA | 378 |
| | | WYQQKPGQPPKLLIYWASTRESGVPDRFSGSGSGTDFTLT | |
| | | ITSLQAEDVAVYYCQQYYSLYTFGQGTKLEIK | |
| IgG-S2-56 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPSAMSASVGDRVTITCRASQGISNYLAWFQQKP | 379 |
| | | GKVPKRLIYAASSLQSGVPSRFCGSGSGTDFTLTIASLQP | |
| | | EDFATYYCQQIYSFPITFGQGTRLETK | |
| IgG-S2-57 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPSSMSASVGDRVIITCRASESLTTWLAWYQQRP | 380 |
| | | GEAPKVLIYKGSTLVRGVPSRFSGRGSGTEFTLTITSLQP | |
| | | DDFATYYCQQYSGFPITFGQGTKVEIK | |
| IgG-S2-58 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIQMTQSPDSLAVSLGERATINCKSSQSVLYSSNNKNYLA | 382 |
| | | WYQHKPGQPPKLLIYWASTRESGVPDRFSGSGSGTDFTLT | |
| | | ISSLQAEDVAVYYCQQCYSSPPTFGQGTKLEIK | |
| IgG-S2-59 | Heavy | EVQLVESGGGVVQPGKSLRLSCAASGFTFNNYGMHWVRQA | 40 |
| | | PGKGLEWVALISYDGSNKYYADSVKGRFTISRDNSKNTLY | |
| | | LQMNSLRAEDTAVYYCARDRSGSYYRGWFDPWGQGTLVTV | |
| | | SS | |
| | Light | DIVMTQSPDSLAVSLGERATINCKSSQSVLYSSNNKNYLA | 374 |
| | | WYQQKPGQPPKLLIYWASTRESGVPDRFSGSGSGTDFTLT | |
| | | ISSLQAEDVAVYYCQQYSTPPYTFGQGTKLEIK | |
| IgG-S2-60 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-61 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-62 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-63 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | QSVLTQPPSASGTPGQRVTISCSGSSSNIGSNYVYWYQQL | 375 |
| | | PGTAPKLLIYRNNQRPSGVPDRFSGSKSGTSASLAISGLR | |
| | | SEDEADYYCAAWDDSLSGWVFGGGTKLTVL | |
| IgG-S2-64 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPSTLSASVGDRVTITCRASQTIYSWLAWYQQKP | 376 |
| | | GKAPKFLIHKASSLESGVPSRFSGSGSGTEFTLTISSLQP | |
| | | DDFATYYCQQYSSYPRTFGQGTKVEIK | |
| IgG-S2-65 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIVMTQSPDSLAVSLGERATINCKSSQSVLYSSNNKNYLA | 12 |
| | | WYQQKPGQPPKLLIYWASTRESGVPDRFSGSGSGTDFTLT | |
| | | ISSLQAEDVAVYYCQQYYSTPPYTFGQGTKLEIK | |
| IgG-S2-66 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPSSMSASVGDRVIITCRASQSISSSLNWYQQKP | 377 |
| | | GKAPNLLIYSASTLQSEVPSRFSGSGSGTDFTLTISSLQP | |
| | | EDFATYYCQQSYSTPPAFGQGTKVEFK | |
| IgG-S2-67 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPDSLAVSLGERATINCKSGQSVLYSSNNKNYLA | 378 |
| | | WYQQKPGQPPKLLIYWASTRESGVPDRFSGSGSGTDFTLT | |
| | | ITSLQAEDVAVYYCQQYYSLYTFGQGTKLEIK | |
| IgG-S2-68 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPSAMSASVGDRVTITCRASQGISNYLAWFQQKP | 379 |
| | | GKVPKRLIYAASSLQSGVPSRFCGSGSGTDFTLTIASLQP | |
| | | EDFATYYCQQIYSFPITFGQGTRLETK | |
| IgG-S2-69 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPSSMSASVGDRVIITCRASESLTTWLAWYQQRP | 380 |
| | | GEAPKVLIYKGSTLVRGVPSRFSGRGSGTEFTLTITSLQP | |
| | | DDFATYYCQQYSGFPITFGQGTKVEIK | |
| IgG-S2-70 | Heavy | QVQLQQSGGGLVKPGGSLRLSCAASGFTFSSYIMKWVRQA | 381 |
| | | PGKGLEWVAVISYDGSNKYYADSVKGRFTISRDNSKNTLY | |
| | | LQMNSLRAEDTAVYYCARVSYSGYGPPGYWGQGTLVTVSS | |
| | Light | DIQMTQSPDSLAVSLGERATINCKSSQSVLYSSNNKNYLA | 382 |
| | | WYQHKPGQPPKLLIYWASTRESGVPDRFSGSGSGTDFTLT | |
| | | ISSLQAEDVAVYYCQQCYSSPPTFGQGTKLEIK | |
| IgG-S2-71 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-72 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-73 | Heavy | QVQLVQSGGGVVQPGRSLRLSCAASGFTFSSYAMHWVRQA | 16 |
| | | PGKGLEWVAVISYDGSNKYYADSVKGRFTISRDNSKNTLY | |
| | | LQMNSLRAEDTAVYYCARARGGSYYYGMDVWGQGTTVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-74 | Heavy | QVQLQQSGGGVVQPGRSLRLSCAASGFTFSSYAMHWVRQA | 383 |
| | | PGKGLEWVAVISYDGDRVYYADSVRGRFNISRDNSKNTLY | |
| | | LQMNSLRPEDTAVYYCAKSRSGSYYYGMDVWGQGTTVTVS | |
| | | S | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-75 | Heavy | QVQLQQSGGGVVQPGRSLRLSCAASGFTFSSYAMHWVRQA | 383 |
| | | PGKGLEWVAVISYDGDRVYYADSVRGRFNISRDNSKNTLY | |
| | | LQMNSLRPEDTAVYYCAKSRSGSYYYGMDVWGQGTTVTVS | |
| | | S | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-76 | Heavy | QVQLQQSGGGVVQPGRSLRLSCAASGFTFSSYAMHWVRQA | 383 |
| | | PGKGLEWVAVISYDGDRVYYADSVRGRFNISRDNSKNTLY | |
| | | LQMNSLRPEDTAVYYCAKSRSGSYYYGMDVWGQGTTVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-77 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | DIQMTQSPSSLSASVGDRVTITCRASQSVSSYLNWYQQNQ | 384 |
| | | GKAPKLLIYAASGLQSGVPSRFSGRGSGTDFTLTINSLQP | |
| | | EDFATYFCQQSSSIPRTFGQGTKVDIK | |
| IgG-S2-78 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | SYELTQPPSVSVAPGQTARVTCGGNNIGSKSVHWYQQKPG | 385 |
| | | QAPVLVVYDDSDRPSGIPERFSGSNSGNTATLTISGTQAM | |
| | | DEADYYCQAWDSSVVFGGGTKLTVL | |
| IgG-S2-79 | Heavy | QVQLVQSGGGVVQPGRSLRLSCAASGFTFSSYAMHWVRQA | 386 |
| | | PGKGLEWVAVISYDGSNKYYADSVKGRFTISRDNSKNTLY | |
| | | LQMNSLRAEDTAVYYCASGGGSYYSPFDYWGQGTLVTVSS | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-80 | Heavy | QVQLVQSGGGVVQPGRSLRLSCAASGFTFSSYAMHWVRQA | 387 |
| | | PGKGLEWVAVISYDGSNKYYADSVKGRFTISRDNSKNTLY | |
| | | LQMNSLRAEDTAVYYCARAYGGGYLTPFDYWGQGTLVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| IgG-S2-81 | Heavy | QVQLVESGGGVVQPGGSLRLSCAASGFTFSSYAMHWVRQA | 388 |
| | | PGKGLEWVAVISYDGSNKYYADSVKGRFTISRDNSKNTLY | |
| | | LQMNSLRAEDTAVYYCARDGSGGYSNPDYWGQGTLVTVSS | |
| | Light | DIQMTQSPSSLSASVGDRVTITCRASKHVSNYLNWYQQKP | 389 |
| | | GKAPKLLIHAASSLQSGVPSRFSGSGSGTDFTLTISSLQP | |
| | | EDFAIYYCQQSFSTLPYTFGQGTKLKIK | |
| IgG-S2-82 | Heavy | QVQLVESGGGVVQPGRSLRLSCAASGFTFSSYAMHWVRQA | 390 |
| | | PGKGLEWVAVISYDGSNKYYADSVKGRFTISRDNSKNTLY | |
| | | LQMNSLRAEDTAVYYCARASHGGYSSAFDPWGQGTLVTVS | |
| | | S | |
| | Light | QSVLTQPPSASGTPGQRVTISCSGSSSNIGSNTVNWYQQL | 10 |
| | | PGTAPKLLIYSNNQRPSGVPDRFSGSKSGTSASLAISGLQ | |
| | | SEDEADYYCAAWDDSLNGPVVFGGGTKLTVL | |
| AN3-1 | Heavy | QVQLQESGPGLVNPSETLSLTCSVSGGSFATENYYWSWIR | 471 |
| | | QHPGEGLEWIGNIYFSGNTYYNPSLNNRFTISFDTSKNHL | |
| | | SLKLPSVTAADTAVYYCARGTIYFDASGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| DN3-1 | Heavy | QVQLQESGPGLVNPSETLSLTCSVSGGSFATENYYWSWIR | 472 |
| | | QHPGEGLEWIGNIYFSGNTYYNPSLNNRFTISFDTSKNHL | |
| | | SLKLPSVTAADTAVYYCARGTIYFDDSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| EN3-1 | Heavy | QVQLQESGPGLVNPSETLSLTCSVSGGSFATENYYWSWIR | 473 |
| | | QHPGEGLEWIGNIYFSGNTYYNPSLNNRFTISFDTSKNHL | |
| | | SLKLPSVTAADTAVYYCARGTIYFDESGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| FN3-1 | Heavy | QVQLQESGPGLVNPSETLSLTCSVSGGSFATENYYWSWIR | 474 |
| | | QHPGEGLEWIGNIYFSGNTYYNPSLNNRFTISFDTSKNHL | |
| | | SLKLPSVTAADTAVYYCARGTIYFDFSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| GN3-1 | Heavy | QVQLQESGPGLVNPSETLSLTCSVSGGSFATENYYWSWIR | 475 |
| | | QHPGEGLEWIGNIYFSGNTYYNPSLNNRFTISFDTSKNHL | |
| | | SLKLPSVTAADTAVYYCARGTIYFDGSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| HN3-1 | Heavy | QVQLQESGPGLVNPSETLSLTCSVSGGSFATENYYWSWIR | 476 |
| | | QHPGEGLEWIGNIYFSGNTYYNPSLNNRFTISFDTSKNHL | |
| | | SLKLPSVTAADTAVYYCARGTIYFDHSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| IN3-1 | Heavy | QVQLQESGPGLVNPSETLSLTCSVSGGSFATENYYWSWIR | 477 |
| | | QHPGEGLEWIGNIYFSGNTYYNPSLNNRFTISFDTSKNHL | |
| | | SLKLPSVTAADTAVYYCARGTIYFDISGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| KN3-1 | Heavy | QVQLQESGPGLVNPSETLSLTCSVSGGSFATENYYWSWIR | 478 |
| | | QHPGEGLEWIGNIYFSGNTYYNPSLNNRFTISFDTSKNHL | |
| | | SLKLPSVTAADTAVYYCARGTIYFDKSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| LN3-1 | Heavy | QVQLQESGPGLVNPSETLSLTCSVSGGSFATENYYWSWIR | 479 |
| | | QHPGEGLEWIGNIYFSGNTYYNPSLNNRFTISFDTSKNHL | |
| | | SLKLPSVTAADTAVYYCARGTIYFDLSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| MN3-1 | Heavy | QVQLQESGPGLVNPSETLSLTCSVSGGSFATENYYWSWIR | 480 |
| | | QHPGEGLEWIGNIYFSGNTYYNPSLNNRFTISFDTSKNHL | |
| | | SLKLPSVTAADTAVYYCARGTIYFDMSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| NN3-1 | Heavy | QVQLQESGPGLVNPSETLSLTCSVSGGSFATENYYWSWIR | 481 |
| | | QHPGEGLEWIGNIYFSGNTYYNPSLNNRFTISEDTSKNHL | |
| | | SLKLPSVTAADTAVYYCARGTIYFDNSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| PN3-1 | Heavy | QVQLQESGPGLVNPSETLSLTCSVSGGSFATENYYWSWIR | 482 |
| | | QHPGEGLEWIGNIYFSGNTYYNPSLNNRFTISFDTSKNHL | |
| | | SLKLPSVTAADTAVYYCARGTIYFDPSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| QN3-1 | Heavy | QVQLQESGPGLVNPSETLSLTCSVSGGSFATENYYWSWIR | 483 |
| | | QHPGEGLEWIGNIYFSGNTYYNPSLNNRFTISFDTSKNHL | |
| | | SLKLPSVTAADTAVYYCARGTIYFDQSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| SN3-1 | Heavy | QVQLQESGPGLVNPSETLSLTCSVSGGSFATENYYWSWIR | 484 |
| | | QHPGEGLEWIGNIYFSGNTYYNPSLNNRFTISFDTSKNHL | |
| | | SLKLPSVTAADTAVYYCARGTIYFDSSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| TN3-1 | Heavy | QVQLQESGPGLVNPSETLSLTCSVSGGSFATENYYWSWIR | 485 |
| | | QHPGEGLEWIGNIYFSGNTYYNPSLNNRFTISFDTSKNHL | |
| | | SLKLPSVTAADTAVYYCARGTIYFDTSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| VN3-1 | Heavy | QVQLQESGPGLVNPSETLSLTCSVSGGSFATENYYWSWIR | 486 |
| | | QHPGEGLEWIGNIYFSGNTYYNPSLNNRFTISFDTSKNHL | |
| | | SLKLPSVTAADTAVYYCARGTIYFDVSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| WN3-1 | Heavy | QVQLQESGPGLVNPSETLSLTCSVSGGSFATENYYWSWIR | 487 |
| | | QHPGEGLEWIGNIYFSGNTYYNPSLNNRFTISFDTSKNHL | |
| | | SLKLPSVTAADTAVYYCARGTIYFDWSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| YN3-1 | Heavy | QVQLQESGPGLVNPSETLSLTCSVSGGSFATENYYWSWIR | 488 |
| | | QHPGEGLEWIGNIYFSGNTYYNPSLNNRFTISFDTSKNHL | |
| | | SLKLPSVTAADTAVYYCARGTIYFDYSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| N3D-1 | Heavy | QVQLQESGPGLVNPSETLSLTCSVSGGSFATENYYWSWIR | 489 |
| | | QHPGEGLEWIGNIYFSGNDYYNPSLNNRFTISFDTSKNHL | |
| | | SLKLPSVTAADTAVYYCARGTIYFDRSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| N3E-1 | Heavy | QVQLQESGPGLVNPSETLSLTCSVSGGSFATENYYWSWIR | 490 |
| | | QHPGEGLEWIGNIYFSGNEYYNPSLNNRFTISFDTSKNHL | |
| | | SLKLPSVTAADTAVYYCARGTIYFDRSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |
| N3G-1 | Heavy | QVQLQESGPGLVNPSETLSLTCSVSGGSFATENYYWSWIR | 491 |
| | | QHPGEGLEWIGNIYFSGNGYYNPSLNNRFTISFDTSKNHL | |
| | | SLKLPSVTAADTAVYYCARGTIYFDRSGYRRVDPFHIWGQ | |
| | | GTMVIVSS | |
| | Light | DIQMTQSPSTLSASVGDRVTITCRASQSISSWLAWYQQKP | 2 |
| | | GKAPKLLIYDASSLESGVPSRFSGSGSGTEFTLTISSLQP | |
| | | DDFATYYCQQYNSYSPWTFGQGTKVEIK | |

TABLE 2
CDRs of heavy and light chain variable sequences
of the antibodies as predicted by IMGT/DomainGapAlign
(Ehrenmann et al., 2010; Ehrenmann & Lefranc, 2011).

| | | CDR1 | CDR2 | CDR3 |
|---|---|---|---|---|
| Clone | Chain | (SEQ ID NO:) | (SEQ ID NO:) | (SEQ ID NO:) |
| N3-1 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| 12C8 | Heavy | GYTLTELS | FDPEDGET | ATGPAVRRGSWFDP |
| | | (SEQ ID NO: | (SEQ ID NO: | (SEQ ID NO: 92) |
| | | 90) | 91) | |
| | Light | SSNIGNNY | DNN | GTWDSSLSAVV |
| | | (SEQ ID NO: | (SEQ ID NO: | (SEQ ID NO: 95) |
| | | 93) | 94) | |
| A7V3 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | SSNIGNNY | DNN | GTWDSSLSAVV |
| | | (SEQ ID NO: | (SEQ ID NO: | (SEQ ID NO: 95) |
| | | 93) | 94) | |
| 7-6 | Heavy | GYTLTELS | FDPEDGET | ATRFAVYGDYLIDY |
| | | (SEQ ID NO: | (SEQ ID NO: | (SEQ ID NO: 97) |
| | | 90) | 91) | |
| | Light | SLRIYY | GKH | NSRDSSGDLVV |
| | | (SEQ ID NO: | (SEQ ID NO: | (SEQ ID NO: 100) |
| | | 98) | 99) | |
| 4C7 | Heavy | GYTLPELS | FDPEDGET | ATAAAVRGRGTIDY |
| | | (SEQ ID NO: | (SEQ ID NO: | (SEQ ID NO: 102) |
| | | 101) | 91) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| 7A8 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | SSNIGNNY | DNN | GTWDSSLSAVV |
| | | (SEQ ID NO: | (SEQ ID NO: | (SEQ ID NO: 95) |
| | | 93) | 94) | |
| 3-26 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | SSNIGSNY | RNN | AAWDDSLSGPV |
| | | (SEQ ID NO: | (SEQ ID NO: | (SEQ ID NO: 117) |
| | | 115) | 116) | |
| N3-3 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | QSVSSY | DAS | QQRSNWPPLT |
| | | (SEQ ID NO: | (SEQ ID NO: | (SEQ ID NO: 119) |
| | | 118) | 88) | |
| P4B3 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | SGSIASNF | END | QSSDDSNQLV |
| | | (SEQ ID NO: | (SEQ ID NO: | (SEQ ID NO: 123) |
| | | 121) | 122) | |
| 1D4 | Heavy | GFSLSTSGMC | IDWDDDK | ARIPIATHLGSDY |
| | | (SEQ ID NO: | (SEQ ID NO: | (SEQ ID NO: 126) |
| | | 124) | 125) | |
| | Light | QSVSSN | GAS | QQYNNWPPWT |
| | | (SEQ ID NO: | (SEQ ID NO: | (SEQ ID NO: 129) |
| | | 127) | 128) | |
| N3-7 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| 6-3A | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | SGSVSTNYY | STN | TLYMGGGLLV |
| | | (SEQ ID NO: | (SEQ ID NO: | (SEQ ID NO: 133) |
| | | 131) | 132) | |
| 8B5 | Heavy | GYTFTGYY | INPNSGGT | ARELPPGRMVVPATYWHFDL |
| | | (SEQ ID NO: | (SEQ ID NO: | (SEQ ID NO: 136) |
| | | 134) | 135) | |
| | Light | QSVSSSY | GAS | QQYGSSPPWT |
| | | (SEQ ID NO: | (SEQ ID NO: | (SEQ ID NO: 138) |
| | | 137) | 128) | |
| 4A5 | Heavy | GFTFSSYA | ISYDGSNK | AKASQLFWLGQFTRDGFDI |
| | | (SEQ ID NO: | (SEQ ID NO: | (SEQ ID NO: 139) |
| | | 112) | 113) | |
| | Light | QSVSSSY | GAS | QQYGSSPPWT |
| | | (SEQ ID NO: | (SEQ ID NO: | (SEQ ID NO: 138) |
| | | 137) | 128) | |
| 3B9 | Heavy | GFTFSSYA | ISYDGSNK | ARDGGGYVSY |
| | | (SEQ ID NO: | (SEQ ID NO: | (SEQ ID NO: 140) |
| | | 112) | 113) | |
| | Light | KLGDKY | QDS | QAWDSSTVV |
| | | (SEQ ID NO: | (SEQ ID NO: | (SEQ ID NO: 143) |
| | | 141) | 142) | |
| 6-3B | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | RSNIGVGYD | SNN | QSYDGSLNDDVI |
| | | (SEQ ID NO: | (SEQ ID NO: | (SEQ ID NO: 145) |
| | | 144) | 104) | |
| B3.1 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | QSISSW | DAS | QQYNSYSPWT |
| | | (SEQ ID NO: | (SEQ ID NO: | (SEQ ID NO: 89) |
| | | 87) | 88) | |
| 1D1 | Heavy | GFSLSTSGMC | IDWDDDK | ARIPIATHLGSDY |
| | | (SEQ ID NO: | (SEQ ID NO: | (SEQ ID NO: 126) |
| | | 124) | 125) | |
| | Light | QSISSW | DAS | QQYNSYSPWT |
| | | (SEQ ID NO: | (SEQ ID NO: | (SEQ ID NO: 89) |
| | | 87) | 88) | |
| 1D9 | Heavy | GFSLSTSGMC | IDWDDDK | ARIPIATHLGSDY |
| | | (SEQ ID NO: | (SEQ ID NO: | (SEQ ID NO: 126) |
| | | 124) | 125) | |
| | Light | KLGDKY | QDS | QAWDSSTVV |
| | | (SEQ ID NO: | (SEQ ID NO: | (SEQ ID NO: 143) |
| | | 141) | 142) | |
| N6-2 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | QSLLHSNGYNY | LGS | MQALQTPPYT |
| | | (SEQ ID NO: | (SEQ ID NO: | (SEQ ID NO: 148) |
| | | 146) | 147) | |
| P4D3 | Heavy | GFIFNNYG | ISYDGSNK | AKAP GQWLRFHYYGMDV |
| | | (SEQ ID NO: | (SEQ ID NO: | (SEQ ID NO: 150) |
| | | 149) | 113) | |
| | Light | SSNIGAHYD | GDN | QSYGNNQGV |
| | | (SEQ ID NO: | (SEQ ID NO: | (SEQ ID NO: 153) |
| | | 151) | 152) | |
| P4A3 | Heavy | GFTFSSYA | ISYDGSNK | ARDDTGRVGSGWYCPLY |
| | | (SEQ ID NO: | (SEQ ID NO: | (SEQ ID NO: 154) |
| | | 112) | 113) | |
| | Light | QSISSY | AAS | QQSYSTPLS |
| | | (SEQ ID NO: | (SEQ ID NO: | (SEQ ID NO: 157) |
| | | 155) | 156) | |
| 3-18 | Heavy | GFIFSSYG | ISYDGSNK | AKQAGAYCSGGSCYSSSEADY |
| | | (SEQ ID NO: | (SEQ ID NO: | (SEQ ID NO: 159) |
| | | 158) | 113) | |
| | Light | SSNIGAHYD | GDN | QSYGNNQGM |
| | | (SEQ ID NO: | (SEQ ID NO: | (SEQ ID NO: 160) |
| | | 151) | 152) | |
| P3B10 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTF |
| | | (SEQ ID NO: | (SEQ ID NO: | (SEQ ID NO: 161) |
| | | 141) | 142) | |
| 6-3C | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | KLGDKY | QDS | QAWDSSTVV |
| | | (SEQ ID NO: | (SEQ ID NO: | (SEQ ID NO: 143) |
| | | 141) | 142) | |
| 4A7 | Heavy | GFTFSSYA | ISYDGSNK | AKASQLFWLGQFTRDGFDI |
| | | (SEQ ID NO: | (SEQ ID NO: | (SEQ ID NO: 139) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| 1D5 | Heavy | GFSLSTSGMC | IDWDDDK | ARIPIATHLGSDY |
| | | (SEQ ID NO: | (SEQ ID NO: | (SEQ ID NO: 126) |
| | | 124) | 125) | |
| | Light | QSVSSSY | GAS | QQYGSSPPWT |
| | | (SEQ ID NO: | (SEQ ID NO: | (SEQ ID NO: 138) |
| | | 137) | 128) | |
| P3C6 | Heavy | GFTFSSYA | ISTRGNT | APGRSLY |
| | | (SEQ ID NO: | (SEQ ID NO: | (SEQ ID NO: 163) |
| | | 112) | 162) | |
| | Light | TSNIGADYD | GNT | QSYDSGLSGSI |
| | | (SEQ ID NO: | (SEQ ID NO: | (SEQ ID NO: 166) |
| | | 164) | 165) | |
| H6 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVLYSSNNKNY | WAS | QQYYSTPPYT |
| | | (SEQ ID NO: | (SEQ ID NO: | (SEQ ID NO: 111) |
| | | 109) | 110) | |
| H4 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | QSVSSY | DAS | QQRSNWPPLT |
| | | (SEQ ID NO: | (SEQ ID NO: | (SEQ ID NO: 119) |
| | | 118) | 88) | |
| G3 | Heavy | GFTFSNYA | ISNSGDKT | ARVRGYSVNWRYYYHYGMDV |
| | | (SEQ ID NO: | (SEQ ID NO: | (SEQ ID NO: 171) |
| | | 169) | 170) | |
| | Light | SGSITSNF | END | QSSDDSNQLV |
| | | (SEQ ID NO: | (SEQ ID NO: | (SEQ ID NO: 123) |
| | | 172) | 122) | |
| F6 | Heavy | GFTFSSYG | IRYDGSNK | AKDTHSSSWRKYYYYYYGMDV |
| | | (SEQ ID NO: | (SEQ ID NO: | (SEQ ID NO: 130) |
| | | 106) | 107) | |
| | Light | QSVSSSY | GAS | QQYGSSPPWT |
| | | (SEQ ID NO: | (SEQ ID NO: | (SEQ ID NO: 138) |
| | | 137) | 128) | |
| F2 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTF |
| | | (SEQ ID NO: | (SEQ ID NO: | (SEQ ID NO: 161) |
| | | 141) | 142) | |
| E4 | Heavy | GFTFSSYG | IRYDGSNK | AKATRANYYYYYGMDV |
| | | (SEQ ID NO: | (SEQ ID NO: | (SEQ ID NO: 108) |
| | | 106) | 107) | |
| | Light | QSLLHSNGYNY | LGS | MOALOTPPYT |
| | | (SEQ ID NO: | (SEQ ID NO: | (SEQ ID NO: 148) |
| | | 146) | 147) | |
| E3 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTGV |
| | | (SEQ ID NO: | (SEQ ID NO: | (SEQ ID NO: 173) |
| | | 141) | 142) | |
| E2 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTF |
| | | (SEQ ID NO: | (SEQ ID NO: | (SEQ ID NO: 161) |
| | | 141) | 142) | |
| D6 | Heavy | GFTFSNYA | ISGSGGST | ARDRFQSYTYYYYGMDV |
| | | (SEQ ID NO: | (SEQ ID NO: | (SEQ ID NO: 175) |
| | | 169) | 174) | |
| | Light | QSVSSSY | GAS | QQYGSSPPWT |
| | | (SEQ ID NO: | (SEQ ID NO: | (SEQ ID NO: 138) |
| | | 137) | 128) | |
| C6 | Heavy | GFSLNNRMG | TFSSGEP | ARVMTSMRLWYWGYWFDP |
| | | (SEQ ID NO: | (SEQ ID NO: | (SEQ ID NO: 178) |
| | | 176) | 177) | |
| | Light | RSNIGVGYD | SNN | QSYDGSLNDDVI |
| | | (SEQ ID NO: | (SEQ ID NO: | (SEQ ID NO: 145) |
| | | 144) | 104) | |
| C4 | Heavy | GFTFSSYA | ISTRGNT | APGRSLY |
| | | (SEQ ID NO: | (SEQ ID NO: | (SEQ ID NO: 163) |
| | | 112) | 162) | |
| | Light | TSNIGADYD | GNT | QSYDSGLSGSI |
| | | (SEQ ID NO: | (SEQ ID NO: | (SEQ ID NO: 166) |
| | | 164) | 165) | |
| C3 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | SSNIGSNY | RNN | AAWDDSLSGPV |
| | | (SEQ ID NO: | (SEQ ID NO: | (SEQ ID NO: 117) |
| | | 115) | 116) | |
| B4 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTF |
| | | (SEQ ID NO: | (SEQ ID NO: | (SEQ ID NO: 161) |
| | | 141) | 142) | |
| B3 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | SSNIGNNY | DNN | GTWDSSLSAVV |
| | | (SEQ ID NO: | (SEQ ID NO: | (SEQ ID NO: 95) |
| | | 93) | 94) | |
| A6 | Heavy | GFTFSNYA | ISGSGGST | ARDRFQSYTYYYYGMDV |
| | | (SEQ ID NO: | (SEQ ID NO: | (SEQ ID NO: 175) |
| | | 169) | 174) | |
| | Light | TSNIGATYD | GNN | QSYGNNQGV |
| | | (SEQ ID NO: | (SEQ ID NO: | (SEQ ID NO: 153) |
| | | 179) | 180) | |
| A5 | Heavy | GYTFTGYY | INPNSGGT | ARLRGYSYGHYYGMDV |
| | | (SEQ ID NO: | (SEQ ID NO: | (SEQ ID NO: 181) |
| | | 134) | 135) | |
| | Light | QSVSSN | GAS | QQYNNWPPWT |
| | | (SEQ ID NO: | (SEQ ID NO: | (SEQ ID NO: 129) |
| | | 127) | 128) | |
| A4 | Heavy | GFTFSSYA | ISYDGSNK | ARDARRFKYYYYYGMDV |
| | | (SEQ ID NO: | (SEQ ID NO: | (SEQ ID NO: 182) |
| | | 112) | 113) | |
| | Light | SSNLGAAYD | NNN | QSYDISLSGLYV |
| | | (SEQ ID NO: | (SEQ ID NO: | (SEQ ID NO: 185) |
| | | 183) | 184) | |
| A3 | Heavy | GFTFSSYG | IRYDGSNK | AKDTHSSSWRKYYYYYYGMDV |
| | | (SEQ ID NO: | (SEQ ID NO: | (SEQ ID NO: 130) |
| | | 106) | 107) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| A2 | Heavy | GFTFGGYP | ISYDGSSA | ARPRSYRNHRFDY |
| | | (SEQ ID NO: | (SEQ ID NO: | (SEQ ID NO: 188) |
| | | 186) | 187) | |
| | Light | QSVSSY | DAS | QQRSNWPPLT |
| | | (SEQ ID NO: | (SEQ ID NO: | (SEQ ID NO: 119) |
| | | 118) | 88) | |
| 96 | Heavy | GFIFNNYG | ISYDGSNK | AKAPGQWLRFHYYGMDV |
| | | (SEQ ID NO: | (SEQ ID NO: | (SEQ ID NO: 150) |
| | | 149) | 113) | |
| | Light | SSNIGAHYD | GKN | NSRDINSNHVL |
| | | (SEQ ID NO: | (SEQ ID NO: | (SEQ ID NO: 190) |
| | | 151) | 189) | |
| 100 | Heavy | GFTFSSYG | IRYDGSNK | AKDRRYYDFWSGYVGSPYYYY |
| | | (SEQ ID NO: | (SEQ ID NO: | YYGMDV |
| | | 106) | 107) | (SEQ ID NO: 191) |
| | Light | KLGDKY | SNN | QSYGNNQGV |
| | | (SEQ ID NO: | (SEQ ID NO: | (SEQ ID NO: 153) |
| | | 141) | 104) | |
| 118 | Heavy | GFTFSDYG | VSYDGSNK | ASPTVT |
| | | (SEQ ID NO: | (SEQ ID NO: | (SEQ ID NO: 194) |
| | | 192) | 193) | |
| | Light | QSISTY | AAS | QQSYSSSWT |
| | | (SEQ ID NO: | (SEQ ID NO: | (SEQ ID NO: 196) |
| | | 195) | 156) | |
| 97 | Heavy | GFTFSSYA | ISYDGSNK | ARGNWGSYYYGMDV |
| | | (SEQ ID NO: | (SEQ ID NO: | (SEQ ID NO: 197) |
| | | 112) | 113) | |
| | Light | NIGSKS | DDS | QSSDDSNQLV |
| | | (SEQ ID NO: | (SEQ ID NO: | (SEQ ID NO: 123) |
| | | 198) | 199) | |
| 3 | Heavy | GFTVSSNY | IYSGGST | ARGGVVDTYYYYGMDV |
| | | (SEQ ID NO: | (SEQ ID NO: | (SEQ ID NO: 202) |
| | | 200) | 201) | |
| | Light | TGPVTTGHW | DTV | LLSQSGAWV |
| | | (SEQ ID NO: | (SEQ ID NO: | (SEQ ID NO: 205) |
| | | 203) | 204) | |
| 114 | Heavy | GYTLTELS | FDPEDGET | ATGPAVRRGSWFDP |
| | | (SEQ ID NO: | (SEQ ID NO: | (SEQ ID NO: 92) |
| | | 90) | 91) | |
| | Light | SSNIGNNY | DNN | GTWDSSLSGYV |
| | | (SEQ ID NO: | (SEQ ID NO: | (SEQ ID NO: 206) |
| | | 93) | 94) | |
| 144 | Heavy | GFIFSSYG | ISYDGSNK | AKQAGAYCSGGSCYSSSEADY |
| | | (SEQ ID NO: | (SEQ ID NO: | (SEQ ID NO: 159) |
| | | 158) | 113) | |
| | Light | SSNIGAHYD | GDN | QSYGNNQGV |
| | | (SEQ ID NO: | (SEQ ID NO: | (SEQ ID NO: 153) |
| | | 151) | 152) | |
| 32 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | SSDVGGYNY | EVS | SSYAGSNNLA |
| | | (SEQ ID NO: | (SEQ ID NO: | (SEQ ID NO: 209) |
| | | 207) | 208) | |
| 131 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | RSNIGNNY | DYD | GTWDNSLSAGV |
| | | (SEQ ID NO: | (SEQ ID NO: | (SEQ ID NO: 212) |
| | | 210) | 211) | |
| 130 | Heavy | GFTFSSYA | ISYDGSNK | ATGPAVRRGSWFDP |
| | | (SEQ ID NO: | (SEQ ID NO: | (SEQ ID NO: 92) |
| | | 112) | 113) | |
| | Light | QSISSY | AAS | QQSYSTPRT |
| | | (SEQ ID NO: | (SEQ ID NO: | (SEQ ID NO: 213) |
| | | 155) | 156) | |
| 132 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | SSNIGNNY | DNN | GTWDSSLSAGV |
| | | (SEQ ID NO: | (SEQ ID NO: | (SEQ ID NO: 214) |
| | | 93) | 94) | |
| 19 | Heavy | GGSFATENYY | IYFSGNT | ARGTIYFDRSGYRRVDPFHI |
| | | (SEQ ID NO: | (SEQ ID NO: | (SEQ ID NO: 86) |
| | | 84) | 85) | |
| | Light | QSISSY | AAS | QQSYSTRPLT |
| | | (SEQ ID NO: | (SEQ ID NO: | (SEQ ID NO: 215) |
| | | 155) | 156) | |
| 31 | Heavy | GYTLTELS | FDPEDGET | ATGSPFDRTQNWFDP |
| | | (SEQ ID NO: | (SEQ ID NO: | (SEQ ID NO: 96) |
| | | 90) | 91) | |
| | Light | SSNIGKNY | SNN | AAWDDGLSAVV |
| | | (SEQ ID NO: | (SEQ ID NO: | (SEQ ID NO: 217) |
| | | 216) | 104) | |
| 42 | Heavy | GFTFSSYA | ISYDGSNK | ARDYGRGGV |
| | | (SEQ ID NO: | (SEQ ID NO: | (SEQ ID NO: 218) |
| | | 112) | 113) | |
| | Light | QSISSY | AAS | QQSYSTRPLT |
| | | (SEQ ID NO: | (SEQ ID NO: | (SEQ ID NO: 215) |
| | | 155) | 156) | |
| 36 | Heavy | GFTFSNAW | IKSKTDGGTT | TTDRSYDYVWGSYRYPDY |
| | | (SEQ ID NO: | (SEQ ID NO: | (SEQ ID NO: 221) |
| | | 219) | 220) | |
| | Light | QSISSY | AAS | QQSYSTPE |
| | | (SEQ ID NO: | (SEQ ID NO: | (SEQ ID NO: 222) |
| | | 155) | 156) | |
| 46 | Heavy | GFTFNNFG | ISYEGSIK | AKDRAIFYLNPRYYLDY |
| | | (SEQ ID NO: | (SEQ ID NO: | (SEQ ID NO: 225) |
| | | 223) | 224) | |
| | Light | KLGDKY | QDS | QAWDSSTAVV |
| | | (SEQ ID NO: | (SEQ ID NO: | (SEQ ID NO: 226) |
| | | 141) | 142) | |
| IgG-S2-14 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | SSNLGAAYD | NNN | QSYDISLSGLYV |
| | | (SEQ ID NO: | (SEQ ID NO: | (SEQ ID NO: 185) |
| | | 183) | 184) | |
| IgG-S2-15 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | SSNIGAGYD | GNN | QSYDRSVSGWV |
| | | (SEQ ID NO: | (SEQ ID NO: | (SEQ ID NO: 395) |
| | | 394) | 180) | |
| IgG-S2-21 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | SGSIASNF | END | QSSDDSNQLV |
| | | (SEQ ID NO: | (SEQ ID NO: | (SEQ ID NO: 123) |
| | | 121) | 122) | |
| IgG-S2-03 | Heavy | GFTFSSYA | ISYDGDRV | AKSRSGSYYYGMDV |
| | | (SEQ ID NO: | (SEQ ID NO: | (SEQ ID NO: 397) |
| | | 112) | 396) | |
| | Light | NIGSKS | DDS | QVWESSGDHYWV |
| | | (SEQ ID NO: | (SEQ ID NO: | (SEQ ID NO: 398) |
| | | 198) | 199) | |
| IgG-S2-16 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | SLRSYY | GKN | DSRDSSGKRRV |
| | | (SEQ ID NO: | (SEQ ID NO: | (SEQ ID NO: 401) |
| | | 400) | 189) | |
| IgG-S2-19 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | SSNIGADYD | ANS | QSYDSILNDSA |
| | | (SEQ ID NO: | (SEQ ID NO: | (SEQ ID NO: 404) |
| | | 402) | 403) | |
| IgG-S2-18 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | SSNIGADYD | ANS | QSYDSILNDSGV |
| | | (SEQ ID NO: | (SEQ ID NO: | (SEQ ID NO: 405) |
| | | 402) | 403) | |
| IgG-S2-17 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | SSNIGADYD | ANS | QSYDSILNDSV |
| | | (SEQ ID NO: | (SEQ ID NO: | (SEQ ID NO: 406) |
| | | 402) | 403) | |
| IgG-S2-09 | Heavy | GFTFSSYA | ISYDGSNK | ARAYGGGYLTPFDY |
| | | (SEQ ID NO: | (SEQ ID NO: | (SEQ ID NO: 407) |
| | | 112) | 113) | |
| | Light | SSNIGAGYD | GNN | QSYDRSVSGWV |
| | | (SEQ ID NO: | (SEQ ID NO: | (SEQ ID NO: 395) |
| | | 394) | 180) | |
| IgG-S2-10 | Heavy | GFTFSSYA | ISYDGSNK | ARDGSGGYSNPDY |
| | | (SEQ ID NO: | (SEQ ID NO: | (SEQ ID NO: 408) |
| | | 112) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-08 | Heavy | GFTFSSYA | ISYDGSNK | ARASHGGYSSAFDP |
| | | (SEQ ID NO: | (SEQ ID NO: | (SEQ ID NO: 292) |
| | | 112) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-01 | Heavy | GFTFSSYG | ISYDGSNK | AKPQGGYNGAFDI |
| | | (SEQ ID NO: | (SEQ ID NO: | (SEQ ID NO: 409) |
| | | 106) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-02 | Heavy | GFTFSSYG | ISYDGSNK | AKPQGGYNGAFDI |
| | | (SEQ ID NO: | (SEQ ID NO: | (SEQ ID NO: 409) |
| | | 106) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-04 | Heavy | GVSITSYH | MYYISGQT | ARADSCNGGICYYQGMDV |
| | | (SEQ ID NO: | (SEQ ID NO: | (SEQ ID NO: 412) |
| | | 410) | 411) | |
| | Light | KLGDKY | QDS | QAWDSSTVV |
| | | (SEQ ID NO: | (SEQ ID NO: | (SEQ ID NO: 143) |
| | | 141) | 142) | |
| IgG-S2-06 | Heavy | GFTFSSYA | ISYDGSNK | ARAGGGGYQGPFDY |
| | | (SEQ ID NO: | (SEQ ID NO: | (SEQ ID NO: 413) |
| | | 112) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-07 | Heavy | GFTFSSYA | ISYDGSNK | ARAGGGGYQGPFDY |
| | | (SEQ ID NO: | (SEQ ID NO: | (SEQ ID NO: 413) |
| | | 112) | 113) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-05 | Heavy | GFTFSSYA | ISYDGSNK | ARAGGGGYQGPFDY |
| | | (SEQ ID NO: | (SEQ ID NO: | (SEQ ID NO: 413) |
| | | 112) | 113) | |
| | Light | SLRIYY | GKH | QAWDSSTAWNV |
| | | (SEQ ID NO: | (SEQ ID NO: | (SEQ ID NO: 414) |
| | | 98) | 99) | |
| IgG-S2-11 | Heavy | GFTFSSYA | ISYDGSNK | ARDLRGSYSGLLDY |
| | | (SEQ ID NO: | (SEQ ID NO: | (SEQ ID NO: 415) |
| | | 112) | 113) | |
| | Light | SSNIGAGYD | GNN | QSYDRSVSGWV |
| | | (SEQ ID NO: | (SEQ ID NO: | (SEQ ID NO: 395) |
| | | 394) | 180) | |
| IgG-S2-22 | Heavy | GFTFSNYA | ISGSGGST | ARDRFQSYTYYYYGMDV |
| | | (SEQ ID NO: | (SEQ ID NO: | (SEQ ID NO: 175) |
| | | 169) | 174) | |
| | Light | TSNIGATYD | GNN | QSYGNNQGV |
| | | (SEQ ID NO: | (SEQ ID NO: | (SEQ ID NO: 153) |
| | | 179) | 180) | |
| IgG-S2-12 | Heavy | GFTFSSYD | IGTAGDT | ARGSHPRKRSGLSLDAFDI |
| | | (SEQ ID NO: | (SEQ ID NO: | (SEQ ID NO: 418) |
| | | 416) | 417) | |
| | Light | NSNIGSNY | ENN | ATWDSSLSAVV |
| | | (SEQ ID NO: | (SEQ ID NO: | (SEQ ID NO: 421) |
| | | 419) | 420) | |
| IgG-S2-13 | Heavy | GFTFSSYA | ISYDGSNK | ARPYSGSYWGYFDY |
| | | (SEQ ID NO: | (SEQ ID NO: | (SEQ ID NO: 114) |
| | | 112) | 113) | |
| | Light | KLGDKY | QDS | QAWDSSTF |
| | | (SEQ ID NO: | (SEQ ID NO: | (SEQ ID NO: 161) |
| | | 141) | 142) | |
| IgG-S2-20 | Heavy | GYTFTNYG | ISTGSGNT | VKGAGGYFDS |
| | | (SEQ ID NO: | (SEQ ID NO: | (SEQ ID NO: 424) |
| | | 422) | 423) | |
| | Light | SLRIYY | GKH | NSRDSSGDLVV |
| | | (SEQ ID NO: | (SEQ ID NO: | (SEQ ID NO: 100) |
| | | 98) | 99) | |
| IgG-S2-30 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-31 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSVSSY | AAS | QQSSSIPRT |
| | | (SEQ ID NO: | (SEQ ID NO: | |
| | | 118) | 156) | (SEQ ID NO: 425) |
| IgG-S2-32 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-33 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-34 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-35 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | NIGSKS | DDS | QAWDSSV |
| | | (SEQ ID NO: | (SEQ ID NO: | (SEQ ID NO: 427) |
| | | 198) | 199) | |
| IgG-S2-36 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | NIGSKS | DDS | QAWDSSVV |
| | | (SEQ ID NO: | (SEQ ID NO: | (SEQ ID NO: 428) |
| | | 198) | 199) | |
| IgG-S2-37 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | NIGSKS | DDS | QAWDSSVE |
| | | (SEQ ID NO: | (SEQ ID NO: | (SEQ ID NO: 429) |
| | | 198) | 199) | |
| IgG-S2-38 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-39 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | SSNIGSNY | RNN | AAWDDSLSGWV |
| | | (SEQ ID NO: | (SEQ ID NO: | (SEQ ID NO: 430) |
| | | 115) | 116) | |
| IgG-S2-40 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QTIYSW | KAS | QQYSSYPRT |
| | | (SEQ ID NO: | (SEQ ID NO: | (SEQ ID NO: 433) |
| | | 431) | 432) | |
| IgG-S2-41 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSVLYSSNNKNY | WAS | QQYYSTPPYT |
| | | (SEQ ID NO: | (SEQ ID NO: | (SEQ ID NO: 111) |
| | | 109) | 110) | |
| IgG-S2-42 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSISSS | SAS | QQSYSTPPA |
| | | (SEQ ID NO: | (SEQ ID NO: | (SEQ ID NO: 436) |
| | | 434) | 435) | |
| IgG-S2-43 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QGISNY | AAS | QQIYSFPIT |
| | | (SEQ ID NO: | (SEQ ID NO: | (SEQ ID NO: 438) |
| | | 437) | 156) | |
| IgG-S2-44 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | ESLTTW | KGS | QQYSGFPIT |
| | | (SEQ ID NO: | (SEQ ID NO: | (SEQ ID NO: 441) |
| | | 439) | 440) | |
| IgG-S2-45 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | SSNIGAGYN | GNS | QSYDSSLSGSGV |
| | | (SEQ ID NO: | (SEQ ID NO: | (SEQ ID NO: 444) |
| | | 442) | 443) | |
| IgG-S2-46 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | NIGSKS | DDS | QVWDSSSDHWV |
| | | (SEQ ID NO: | (SEQ ID NO: | (SEQ ID NO: 445) |
| | | 198) | 199) | |
| IgG-S2-47 | Heavy | GFTFSSYA | ISYDGSNK | ARSTYGSYYYGMDV |
| | | (SEQ ID NO: | (SEQ ID NO: | (SEQ ID NO: 391) |
| | | 112) | 113) | |
| | Light | QSISSY | AAS | QQSYSTPWT |
| | | (SEQ ID NO: | (SEQ ID NO: | (SEQ ID NO: 446) |
| | | 155) | 156) | |
| IgG-S2-48 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-49 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-50 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-51 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | SSNIGSNY | RNN | AAWDDSLSGWV |
| | | (SEQ ID NO: | (SEQ ID NO: | (SEQ ID NO: 430) |
| | | 115) | 116) | |
| IgG-S2-52 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QTIYSW | KAS | QQYSSYPRT |
| | | (SEQ ID NO: | (SEQ ID NO: | (SEQ ID NO: 433) |
| | | 431) | 432) | |
| IgG-S2-53 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVLYSSNNKNY | WAS | QQYYSTPPYT |
| | | (SEQ ID NO: | (SEQ ID NO: | (SEQ ID NO: 111) |
| | | 109) | 110) | |
| IgG-S2-54 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSISSS | SAS | QQSYSTPPA |
| | | (SEQ ID NO: | (SEQ ID NO: | (SEQ ID NO: 436) |
| | | 434) | 435) | |
| IgG-S2-55 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVLYSSNNKNY | WAS | QQYYSLYT |
| | | (SEQ ID NO: | (SEQ ID NO: | (SEQ ID NO: 447) |
| | | 109) | 110) | |
| IgG-S2-56 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QGISNY | AAS | QQIYSFPIT |
| | | (SEQ ID NO: | (SEQ ID NO: | |
| | | 437) | 156) | (SEQ ID NO: 438) |
| IgG-S2-57 | Heavy | GFTENNYG | ISYDGSNK | ARDRSGSYYRGWFDP |
| | | (SEQ ID NO: | (SEQ ID NO: | (SEQ ID NO: 168) |
| | | 167) | 113) | |
| | Light | ESLTTW | KGS | QQYSGFPIT |
| | | (SEQ ID NO: | (SEQ ID NO: | (SEQ ID NO: 441) |
| | | 439) | 440) | |
| IgG-S2-58 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVLYSSNNKNY | WAS | QQCYSSPPT |
| | | (SEQ ID NO: | (SEQ ID NO: | (SEQ ID NO: 448) |
| | | 109) | 110) | |
| IgG-S2-59 | Heavy | GFTFNNYG | ISYDGSNK | ARDRSGSYYRGWFDP |
| | | (SEQ ID NO: | (SEQ ID NO: | (SEQ ID NO: 168) |
| | | 167) | 113) | |
| | Light | QSVLYSSNNKNY | WAS | QQYSTPPYT |
| | | (SEQ ID NO: | (SEQ ID NO: | (SEQ ID NO: 449) |
| | | 109) | 110) | |
| IgG-S2-60 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-61 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-62 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-63 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | SSNIGSNY | RNN | AAWDDSLSGWV |
| | | (SEQ ID NO: | (SEQ ID NO: | (SEQ ID NO: 430) |
| | | 115) | 116) | |
| IgG-S2-64 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QTIYSW | KAS | QQYSSYPRT |
| | | (SEQ ID NO: | (SEQ ID NO: | (SEQ ID NO: 433) |
| | | 431) | 432) | |
| IgG-S2-65 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QSVLYSSNNKNY | WAS | QQYYSTPPYT |
| | | (SEQ ID NO: | (SEQ ID NO: | (SEQ ID NO: 111) |
| | | 109) | 110) | |
| IgG-S2-66 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QSISSS | SAS | QQSYSTPPA |
| | | (SEQ ID NO: | (SEQ ID NO: | (SEQ ID NO: 436) |
| | | 434) | 435) | |
| IgG-S2-67 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QSVLYSSNNKNY | WAS | QQYYSLYT |
| | | (SEQ ID NO: | (SEQ ID NO: | (SEQ ID NO: 447) |
| | | 109) | 110) | |
| IgG-S2-68 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QGISNY | AAS | QQIYSFPIT |
| | | (SEQ ID NO: | (SEQ ID NO: | (SEQ ID NO: 438) |
| | | 437) | 156) | |
| IgG-S2-69 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | ESLTTW | KGS | QQYSGFPIT |
| | | (SEQ ID NO: | (SEQ ID NO: | (SEQ ID NO: 441) |
| | | 439) | 440) | |
| IgG-S2-70 | Heavy | GFTFSSYI | ISYDGSNK | ARVSYSGYGPPGY |
| | | (SEQ ID NO: | (SEQ ID NO: | (SEQ ID NO: 393) |
| | | 392) | 113) | |
| | Light | QSVLYSSNNKNY | WAS | QQCYSSPPT |
| | | (SEQ ID NO: | (SEQ ID NO: | (SEQ ID NO: 448) |
| | | 109) | 110) | |
| IgG-S2-71 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-72 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-73 | Heavy | GFTFSSYA | ISYDGSNK | ARARGGSYYYGMDV |
| | | (SEQ ID NO: | (SEQ ID NO: | (SEQ ID NO: 120) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-74 | Heavy | GFTFSSYA | ISYDGDRV | AKSRSGSYYYGMDV |
| | | (SEQ ID NO: | (SEQ ID NO: | (SEQ ID NO: 397) |
| | | 112) | 396) | |
| | Light | QSVSSY | AAS | QQSSSIPRT |
| | | (SEQ ID NO: | (SEQ ID NO: | (SEQ ID NO: 425) |
| | | 118) | 156) | |
| IgG-S2-75 | Heavy | GFTFSSYA | ISYDGDRV | AKSRSGSYYYGMDV |
| | | (SEQ ID NO: | (SEQ ID NO: | (SEQ ID NO: 397) |
| | | 112) | 396) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-76 | Heavy | GFTFSSYA | ISYDGDRV | AKSRSGSYYYGMDV |
| | | (SEQ ID NO: | (SEQ ID NO: | (SEQ ID NO: 397) |
| | | 112) | 396) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-77 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | QSVSSY | AAS | QQSSSIPRI |
| | | (SEQ ID NO: | (SEQ ID NO: | (SEQ ID NO: 425) |
| | | 118) | 156) | |
| IgG-S2-78 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | NIGSKS | DDS | QAWDSSVV |
| | | (SEQ ID NO: | (SEQ ID NO: | (SEQ ID NO: 426) |
| | | 198) | 199) | |
| IgG-S2-79 | Heavy | GFTFSSYA | ISYDGSNK | ASGGGSYYSPFDY |
| | | (SEQ ID NO: | (SEQ ID NO: | (SEQ ID NO: 399) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-80 | Heavy | GFTFSSYA | ISYDGSNK | ARAYGGGYLTPFDY |
| | | (SEQ ID NO: | (SEQ ID NO: | (SEQ ID NO: 407) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| IgG-S2-81 | Heavy | GFTFSSYA | ISYDGSNK | ARDGSGGYSNPDY |
| | | (SEQ ID NO: | (SEQ ID NO: | (SEQ ID NO: 408) |
| | | 112) | 113) | |
| | Light | KHVSNY | AAS | QQSFSTLPYT |
| | | (SEQ ID NO: | (SEQ ID NO: | (SEQ ID NO: 451) |
| | | 450) | 156) | |
| IgG-S2-82 | Heavy | GFTFSSYA | ISYDGSNK | ARASHGGYSSAFDP |
| | | (SEQ ID NO: | (SEQ ID NO: | (SEQ ID NO: 292) |
| | | 112) | 113) | |
| | Light | SSNIGSNT | SNN | AAWDDSLNGPVV |
| | | (SEQ ID NO: | (SEQ ID NO: | (SEQ ID NO: 105) |
| | | 103) | 104) | |
| AN3-1 | Heavy | GGSFATENYY | IYFSGNT | ARGTIYFDASGYRRVDPFHI |
| | | (SEQ ID NO: | (SEQ ID NO: | (SEQ ID NO: 492) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| DN3-1 | Heavy | GGSFATENYY | IYFSGNT | ARGTIYFDDSGYRRVDPFHI |
| | | (SEQ ID NO: | (SEQ ID NO: | (SEQ ID NO: 493) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| EN3-1 | Heavy | GGSFATENYY | IYFSGNT | ARGTIYFDESGYRRVDPFHI |
| | | (SEQ ID NO: | (SEQ ID NO: | (SEQ ID NO: 494) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| FN3-1 | Heavy | GGSFATENYY | IYFSGNT | ARGTIYFDFSGYRRVDPFHI |
| | | (SEQ ID NO: | (SEQ ID NO: | (SEQ ID NO: 495) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| GN3-1 | Heavy | GGSFATENYY | IYFSGNT | ARGTIYFDGSGYRRVDPFHI |
| | | (SEQ ID NO: | (SEQ ID NO: | (SEQ ID NO: 496) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| HN3-1 | Heavy | GGSFATENYY | IYFSGNT | ARGTIYFDHSGYRRVDPFHI |
| | | (SEQ ID NO: | (SEQ ID NO: | (SEQ ID NO: 497) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| IN3-1 | Heavy | GGSFATENYY | IYFSGNT | ARGTIYFDISGYRRVDPFHI |
| | | (SEQ ID NO: | (SEQ ID NO: | (SEQ ID NO: 498) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| KN3-1 | Heavy | GGSFATENYY | IYFSGNT | ARGTIYFDKSGYRRVDPFHI |
| | | (SEQ ID NO: | (SEQ ID NO: | (SEQ ID NO: 499) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| LN3-1 | Heavy | GGSFATENYY | IYFSGNT | ARGTIYFDLSGYRRVDPFHI |
| | | (SEQ ID NO: | (SEQ ID NO: | (SEQ ID NO: 500) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| MN3-1 | Heavy | GGSFATENYY | IYFSGNT | ARGTIYFDMSGYRRVDPFHI |
| | | (SEQ ID NO: | (SEQ ID NO: | (SEQ ID NO: 501) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| NN3-1 | Heavy | GGSFATENYY | IYFSGNT | ARGTIYFDNSGYRRVDPFHI |
| | | (SEQ ID NO: | (SEQ ID NO: | (SEQ ID NO: 502) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| PN3-1 | Heavy | GGSFATENYY | IYFSGNT | ARGTIYFDP SGYRRVDPFHI |
| | | (SEQ ID NO: | (SEQ ID NO: | (SEQ ID NO: 503) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| QN3-1 | Heavy | GGSFATENYY | IYFSGNT | ARGTIYFDQSGYRRVDPFHI |
| | | (SEQ ID NO: | (SEQ ID NO: | (SEQ ID NO: 504) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| SN3-1 | Heavy | GGSFATENYY | IYFSGNT | ARGTIYFDSSGYRRVDPFHI |
| | | (SEQ ID NO: | (SEQ ID NO: | (SEQ ID NO: 505) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| TN3-1 | Heavy | GGSFATENYY | IYFSGNT | ARGTIYFDTSGYRRVDPFHI |
| | | (SEQ ID NO: | (SEQ ID NO: | (SEQ ID NO: 506) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| VN3-1 | Heavy | GGSFATENYY | IYFSGNT | ARGTIYFDVSGYRRVDPFHI |
| | | (SEQ ID NO: | (SEQ ID NO: | (SEQ ID NO: 507) |
| | | 84) | 85) | |
| | Light | QSISSW | DAS | QQYNSYSPWT |
| | | | (SEQ ID NO: | |
| | | (SEQ ID NO: | 88) | (SEQ ID NO: 89) |
| | | 87) | | |
| WN3-1 | Heavy | GGSFATENYY | IYFSGNT | ARGTIYFDWSGYRRVDPFHI |
| | | (SEQ ID NO: | (SEQ ID NO: | (SEQ ID NO: 508) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| YN3-1 | Heavy | GGSFATENYY | IYFSGNT | ARGTIYFDYSGYRRVDPFHI |
| | | (SEQ ID NO: | (SEQ ID NO: | (SEQ ID NO: 509) |
| | | 84) | 85) | |
| | Light | QSISSW | DAS | QQYNSYSPWT |
| | | (SEQ ID NO: | (SEQ ID NO: | (SEQ ID NO: 89) |
| | | 87) | 88) | |
| N3D-1 | Heavy | GGSFATENYY | IYFSGND | ARGTIYFDRSGYRRVDPFHI |
| | | (SEQ ID NO: | (SEQ ID NO: | (SEQ ID NO: 86) |
| | | 84) | 510) | |
| | Light | QSISSW | DAS | QQYNSYSPWT |
| | | (SEQ ID NO: | (SEQ ID NO: | (SEQ ID NO: 89) |
| | | 87) | 88) | |
| N3E-1 | Heavy | GGSFATENYY | IYFSGNE | ARGTIYFDRSGYRRVDPFHI |
| | | (SEQ ID NO: | (SEQ ID NO: | (SEQ ID NO: 86) |
| | | 84) | 511) | |
| | Light | QSISSW | DAS | QQYNSYSPWT |
| | | (SEQ ID NO: | (SEQ ID NO: | (SEQ ID NO: 89) |
| | | 87) | 88) | |
| N3G-1 | Heavy | GGSFATENYY | IYFSGNG | ARGTIYFDRSGYRRVDPFHI |
| | | (SEQ ID NO: | (SEQ ID NO: | (SEQ ID NO: 86) |
| | | 84) | 512) | |
| | Light | QSISSW | DAS | QQYNSYSPWT |
| | | (SEQ ID NO: | (SEQ ID NO: | (SEQ ID NO: 89) |
| | | 87) | 88) | |

The monoclonal antibodies of the present invention have several applications, including the production of diagnostic kits for use in detecting and diagnosing coronavirus infections, as well as for treating or preventing coronavirus infections in patients. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (C_{H}). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

An IgM antibody, for example, consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (V_{L}) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (CL) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H1} domains; (iv) the Fd′ fragment having V_{H} and C_{H1} domains and one or more cysteine residues at the C-terminus of the C_{H1} domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}—C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

### B. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The monoclonal antibodies may also be isolated from phage antibody libraries using the techniques described in Clackson et al., Nature, 352:624-628 (1991) and Marks et al., J. Mol. Biol., 222:581-597 (1991), for example.

Methods for producing monoclonal antibodies of various types, including chimeric and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024, each incorporated herein by reference.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

### C. Single Chain Antibodies

A single chain variable fragment (scFv) is a fusion of the variable regions of the heavy and light chains of immunoglobulins, linked together with a short linker. This chimeric molecule retains the specificity of the original immunoglobulin, despite removal of the constant regions and the introduction of a linker peptide. This modification usually leaves the specificity unaltered. scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. For example, the linker may have a proline residue two residues after the V_{H} C terminus and an abundance of arginines and prolines at other positions.

A single-chain antibody may also be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

For example, SMPT is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site. The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Flexible linkers may also be used.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### D. Bispecific and Multispecific Antibodies

Antibodies may be bispecific or multispecific. “Bispecific antibodies” are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). Taki et al. (2015) describes a bispecific anti-HSP70/anti-CD3 antibody.

Methods for making bispecific antibodies are known in the art. Traditional production of full-length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

The bispecific antibodies may be composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175:217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148 (5): 1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

A bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400). Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579:3264; Wong and Scott, Nat. Rev. Mol. Cell Biol. 2004; 5:959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147:60, 1991; Xu et al., Science, 358 (6359): 85-90, 2017). The antibodies may also involve sequences or moieties that permit dimerization or multimerization of the receptors. Such sequences include those derived from IgA, which permit formation of multimers in conjunction with the J-chain. Another multimerization domain is the Gal4 dimerization domain.

A multivalent antibody may be internalized (and/or catabolized) faster than a bivalent antibody by a cell expressing an antigen to which the antibody binds. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. Multivalent antibodies may comprise (or consist of) three to about eight, for example four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2) n-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

### E. BiTES

A bi-specific T-cell engagers (BiTE®) is an artificial bispecific monoclonal antibody that directs a host's immune system, more specifically the T cells' cytotoxic activity, to target diseased cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on target cells by producing proteins like perforin and granzymes, independently of the presence of MHC I or co-stimulatory molecules. These proteins enter the target cells and initiate apoptosis. This action mimics physiological processes observed during T cell attacks against infected cells.

### F. Antibody Conjugates

Antibodies of the present disclosure may be linked to at least one agent to form an antibody conjugate. The conjugate can be, for example, an antibody conjugated to another proteinaceous, carbohydrate, lipid, or mixed moiety molecule(s). Such antibody conjugates include, but are not limited to, modifications that include linking the antibody to one or more polymers. For example, an antibody may be linked to one or more water-soluble polymers. Linkage to a water-soluble polymer reduces the likelihood that the antibody will precipitate in an aqueous environment, such as a physiological environment. One skilled in the art can select a suitable water-soluble polymer based on considerations including, but not limited to, whether the polymer/antibody conjugate will be used in the treatment of a patient and, if so, the pharmacological profile of the antibody (e.g., half-life, dosage, activity, antigenicity, and/or other factors).

In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, an enzyme (e.g., that catalyzes a colorimetric or fluorometric reaction), a substrate, a solid matrix, such as biotin. An antibody may comprise one, two, or more of any of these labels.

Antibody conjugates may be used to deliver cytotoxic agents to target cells. Cytotoxic agents of this type may improve antibody-mediated cytotoxicity, and include such moieties as cytokines that directly or indirectly stimulate cell death, radioisotopes, chemotherapeutic drugs (including prodrugs), bacterial toxins (e.g., pseudomonas exotoxin, diphtheria toxin, etc.), plant toxins (e.g., ricin, gelonin, etc.), chemical conjugates (e.g., maytansinoid toxins, auristatins, α-amanitin, anthracyclines, calechaemicin, etc.), radioconjugates, enzyme conjugates (e.g., RNase conjugates, granzyme antibody-directed enzyme/prodrug therapy), and the like.

Antibody conjugates are also used as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

The paramagnetic ions contemplated for use as conjugates include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and bismuth (III).

The radioactive isotopes contemplated for use as conjugated include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred. Technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

The fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

Another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Derivatization of immunoglobulins by selectively introducing sulfhydryl groups in the Fc region of an immunoglobulin, using reaction conditions that do not alter the antibody combining site are also contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### G. Antibody Drug Conjugates

Antibody drug conjugates, or ADCs, are a class of highly potent biopharmaceutical drugs designed as a targeted therapy for the treatment of people with disease. ADCs are complex molecules composed of an antibody (a whole mAb or an antibody fragment, such as a scFv) linked, via a stable chemical linker with labile bonds, to a biological active cytotoxic/anti-viral payload or drug. Antibody drug conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on diseased cells). Antibodies track these proteins down in the body and attach themselves to the surface of the diseased cells. The biochemical reaction between the antibody and the target protein (antigen) triggers a signal in the targeted cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (e.g., anti-cancer) drug. For example, a non-cleavable linker keeps the drug within the cell. As a result, the entire antibody, linker, and cytotoxic agent enter the targeted cell where the antibody is degraded to the level of amino acids. The resulting complex-amino acid, linker and cytotoxic agent-now becomes the active drug. In contrast, cleavable linkers are catalyzed by enzymes in the host cell, thereby releasing the cytotoxic agent.

Another type of cleavable linker adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation. Future direction in the development of ADCs also include the development of site-specific conjugation (TDCs) to further improve stability and therapeutic index and α-emitting immunoconjugates and antibody-conjugated nanoparticles.

### H. Intrabodies

In a particular embodiment, the antibody is a recombinant antibody that is suitable for action inside of a cell-such antibodies are known as “intrabodies.” These antibodies may interfere with target function by a variety of mechanisms, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required. An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

The two major issues impacting the implementation of intrabody therapeutics are delivery, including cell/tissue targeting, and stability. With respect to delivery, a variety of approaches have been employed, such as tissue-directed delivery, use of cell-type specific promoters, viral-based delivery, use of cell-permeability/membrane translocating peptides, and delivery using exosomes. One means of delivery comprises the use of lipid-based nanoparticles, or exosomes, as taught in U.S. Pat. Appln. Publn. 2018/0177727, which is incorporated by reference here in its entirety. With respect to stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

### I. Production and Purification of Antibodies

The methods for generating monoclonal antibodies generally begin along the same lines as those for preparing polyclonal antibodies. The first step for both of these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59, and combinations of immunostimulatory molecules (“Adjuvant Systems”, such as AS01 or AS03). Additional experimental forms of inoculation to induce antigen-specific B cells are possible, including nanoparticle vaccines, or gene-encoded antigens delivered as DNA or RNA genes in a physical delivery system (such as lipid nanoparticle or on a gold biolistic bead), and delivered with needle, gene gun, or transcutaneous electroporation device. The antigen gene also can be carried as encoded by a replication competent or defective viral vector such as adenovirus, adeno-associated virus, poxvirus, herpesvirus, or alphavirus replicon, or alternatively a virus-like particle.

Methods for generating hybrids of antibody-producing spleen or lymph node cells and myeloma cells usually comprise mixing somatic cells with myeloma cells in a 2:1 proportion, though the proportion may vary from about 20:1 to about 1:1, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus or polyethylene glycol (PEG) are also known. The use of electrically induced fusion methods is also appropriate. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide monoclonal antibodies. The cell lines may be exploited for monoclonal antibody production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide monoclonal antibodies in high concentration. The individual cell lines could also be cultured in vitro, where the monoclonal antibodies are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Alternatively, a molecular cloning approach may be used to generate monoclonal antibodies. Single B cells labeled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

Monoclonal antibodies produced by any means may be purified, if desired, using filtration, centrifugation, and various chromatographic methods, such as FPLC or affinity chromatography. Fragments of the monoclonal antibodies of the disclosure can be obtained from the purified monoclonal antibodies by methods that include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

The antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

### J. Modification of Antibodies

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing: 1) Unpaired Cys residues, 2) N-linked glycosylation, 3) Asn deamidation, 4) Asp isomerization, 5) SYE truncation, 6) Met oxidation, 7) Trp oxidation, 8) N-terminal glutamate, 9) Integrin binding, 10) CD11c/CD18 binding, or 11) Fragmentation. Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### K. Characterization of Antibodies

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody binds may consist of a single contiguous sequence of 3 or more (e.g. 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248:443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267:252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce monoclonal antibodies having the desired characteristics. MAP may be used to sort the antibodies of the disclosure into groups of antibodies binding different epitopes.

The present disclosure includes antibodies that may bind to the same epitope, or a portion of the same epitope. One can easily determine whether an antibody binds to the same epitope as, or competes for binding with, a reference antibody by using routine methods known in the art. For example, to determine if a test antibody binds to the same epitope as a reference antibody, the reference antibody is allowed to bind to the target molecule under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if a test antibody competes for binding with a disclosed antibody, the above-described binding methodology is performed in two orientations: In a first orientation, the disclosed antibody is allowed to bind to an coronavirus spike protein under saturating conditions followed by assessment of binding of the test antibody to the coronavirus spike protein. In a second orientation, the test antibody is allowed to bind to a coronavirus spike protein under saturating conditions followed by assessment of binding of the disclosed antibody to the coronavirus spike protein. If, in both orientations, only the first (saturating) antibody is capable of binding to the coronavirus spike protein, then it is concluded that the test antibody and the disclosed antibody compete for binding to the coronavirus spike protein. As will be appreciated by a person of ordinary skill in the art, a test antibody that competes for binding with a disclosed antibody may not necessarily bind to the identical epitope as the disclosed antibody, but may sterically block binding of the disclosed antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90%, or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

In another aspect, the antibodies may be defined by their variable sequence, which include additional “framework” regions. These are provided in Table 1 that represent full variable regions. Furthermore, the antibodies sequences may vary from these sequences, optionally using methods discussed in greater detail below. For example, amino acid sequences, and nucleic acid sequences encoding those amino acid sequences, may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions. Each of the foregoing applies to the amino acid sequences of Tables 1 and 2.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the antibodies provided herein and their antigen-binding fragments. A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art.

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

One can determine the biophysical properties of antibodies. One can use elevated temperature to unfold antibodies to determine relative stability, using average apparent melting temperatures. Differential Scanning calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cutoff. The most straightforward method is induction of amorphous precipitation, which measures protein solubility using a method involving protein precipitation using ammonium sulfate (Trevino et al., J Mol Biol, 366:449-460, 2007). Ammonium sulfate precipitation 15 gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection; however, it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

### IV. Chimeric Antigen Receptors

Chimeric antigen receptor (CAR) molecules are recombinant fusion protein and are distinguished by their ability to both bind antigen and transduce activation signals via immunoreceptor activation motifs (ITAMs) present in their cytoplasmic tails in order to activate genetically modified immune effector cells for killing, proliferation, and cytokine production. Receptor constructs utilizing an antigen-binding moiety (for example, generated from single chain antibodies (scFv)) afford the additional advantage of being “universal” in that they bind native antigen on the target cell surface in an HLA-independent fashion.

Embodiments of the CARs described herein include nucleic acids encoding an antigen-specific CAR polypeptide comprising an intracellular signaling domain, a transmembrane domain, and an extracellular domain comprising an antigen-binding domain. A CAR may recognize an epitope comprised of the shared space between one or more antigens. Optionally, a CAR can comprise a hinge domain positioned between the transmembrane domain and the antigen binding domain. A CAR may further comprise a signal peptide that directs expression of the CAR to the cell surface. For example, a CAR may comprise a signal peptide from GM-CSF. A CAR may also be co-expressed with a membrane-bound cytokine to improve persistence. For example, a CAR may be co-expressed with membrane-bound IL-15.

Depending on the arrangement of the domains of the CAR and the specific sequences used in the domains, immune effector cells expressing the CAR may have different levels activity against target cells. Different CAR sequences may be introduced into immune effector cells to generate engineered cells, the engineered cells selected for elevated SRC, and the selected cells tested for activity to identify the CAR constructs predicted to have the greatest therapeutic efficacy.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

The chimeric construct may be introduced into immune effector cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune effector cells. Suitable vectors for use in accordance with the method of the present invention are non-replicating in the immune effector cells. A large number of vectors are known that are based on viruses, where the copy number of the virus maintained in the cell is low enough to maintain the viability of the cell, such as, for example, vectors based on HIV, SV40, EBV, HSV, or BPV.

### A. Antigen Binding Domains

An antigen binding domain may comprise complementary determining regions of a monoclonal antibody, variable regions of a monoclonal antibody, and/or antigen binding fragments thereof. The antigen binding regions or domains may comprise a fragment of the VH and VL chains of a single-chain variable fragment (scFv) derived from a particular mouse, humanized, or human monoclonal antibody. The fragment can also be any number of different antigen binding domains of an antigen-specific antibody. The fragment may be an antigen-specific scFv encoded by a sequence that is optimized for human codon usage for expression in human cells. In certain aspects, VH and VL domains of a CAR are separated by a linker sequence, such as a Whitlow linker.

The prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to coronavirus spike protein. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., pre-fusion conformations of Spike protein).

Alternatively, a CAR may be engineered using VH and VL chains derived from different mAbs to generate a panel of CAR+ immune effector cells. The antigen binding domain of a CAR may contain any combination of the LCDR1-3 sequences of a first antibody and the HCDR1-3 sequences of a second antibody.

### B. Hinge Domains

A CAR polypeptide may include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-modified immune effector cells. The hinge domain may comprise a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor.

A CAR hinge domain may be derived from human immunoglobulin (Ig) constant region or a portion thereof including the Ig hinge, or from human CD8 α transmembrane domain and CD8a-hinge region. A CAR hinge domain may comprise a hinge-CH₂-CH₃ region of antibody isotype IgG4. The hinge domain (and/or the CAR) may not comprise a wild type human IgG4 CH2 and CH3 sequence. Point mutations may be introduced in antibody heavy chain CH₂ domain to reduce glycosylation and non-specific Fc gamma receptor binding of CAR-modified immune effector cells.

A CAR hinge domain may comprise an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. A CAR hinge domain may comprise an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. A CAR hinge domain may comprise an IgG4-Fc domain having an amino acid substitution at position L235 for an amino acid that is hydrophilic, such as R, H, K, D, E, S, T, N or Q, or that has similar properties to an “E,” such as D. A CAR hinge domain may comprise an IgG4-Fc domain having an amino acid substitution at position N297 for an amino acid that has similar properties to a “Q,” such as S or T.

The hinge domain may comprise a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain, or an engineered hinge domain.

### C. Transmembrane Domains

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3ζ domain, a cysteine mutated human CD3ζ domain, or other transmembrane domains from other human transmembrane signaling proteins, such as CD16, CD8, and erythropoietin receptor. For example, the transmembrane domain may comprise a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

### D. Intracellular Signaling Domains

The intracellular signaling domain of a CAR is responsible for activation of at least one of the normal effector functions of the immune cell engineered to express the CAR. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. The intracellular signaling domain may be derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB/CD137, ICOS/CD278, IL-2RB/CD122, IL-2Rα/CD132, DAP10, DAP12, CD40, OX40/CD134, and combinations thereof, as well as other similar molecules and fragments. Intracellular signaling portions of other members of the families of activating proteins can be used.

While the entire intracellular signaling domain may be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. One or multiple cytoplasmic domains may be employed, as so-called third generation CARs have at least two or three signaling domains fused together for additive or synergistic effect, for example the CD28 and 4-1BB can be combined in a CAR construct. In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

### E. Immune Effector Cells

Immune effectors cells may be T cells (e.g., regulatory T cells, CD4+ T cells, CD8+ T cells, or gamma-delta T cells), natural killer (NK) cells, invariant NK cells, or NKT cells. Also provided herein are methods of producing and engineering the immune effector cells as well as methods of using and administering the cells for adoptive cell therapy, in which case the cells may be autologous or allogeneic. Thus, the immune effector cells may be used as immunotherapy, such as to target cancer cells.

The immune effector cells may be isolated from subjects, particularly human subjects. The immune effector cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. Immune effector cells can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

Tissues/organs from which the immune effector cells are enriched, isolated, and/or purified may be isolated from both living and non-living subjects, wherein the non-living subjects are organ donors. Immune effector cells isolated from cord blood may have enhanced immunomodulation capacity, such as measured by CD4- or CD8-positive T cell suppression. The immune effector cells may be isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune effector cell activity. Thus, the cells will be autologous to the subject in need of therapy. Alternatively, the population of immune effector cells can be obtained from a donor, preferably an allogeneic donor. Allogeneic donor cells may or may not be human-leukocyte-antigen (HLA)-compatible. To be rendered subject-compatible, allogeneic cells can be treated to reduce immunogenicity.

### 2. T Cells

The immune effector cells may be T cells. The T cells may be derived from the blood, bone marrow, lymph, umbilical cord, or lymphoid organs. The T cells may be human T cells. The T cells typically are primary cells, such as those isolated directly from a subject and/or isolated from a subject and frozen. The cells may include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. For off-the-shelf technologies, the cells may be derived from pluripotent and/or multipotent cells, such as stem cells, such as induced pluripotent stem cells (iPSCs).

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

One or more of the T cell populations may be enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

T cells may be separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

CD8⁺ T cells may be further enriched for or depleted of naive, central memory, effector memory, and/or central memory stem cells, such as by positive or negative selection based on surface antigens associated with the respective subpopulation. Enrichment for central memory T (T_{CM}) cells may be carried out to increase efficacy, such as to improve long-term survival, expansion, and/or engraftment following administration, which in some aspects is particularly robust in such sub-populations.

The T cells may be autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., MOLECULAR CLONING: A LABORATORY MANUAL, 3^{rd} ed., Cold Spring Harbor Press, Cold Spring Harbor, N.Y. 2001; and Ausubel et al., CURRENT PROTOCOLS IN MOLECULAR BIOLOGY, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

### 3. NK Cells

The immune effector cells may be natural killer (NK) cells. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells constitute about 10% of the lymphocytes in human peripheral blood. When lymphocytes are cultured in the presence of interleukin 2 (IL-2), strong cytotoxic reactivity develops. NK cells are effector cells known as large granular lymphocytes because of their larger size and the presence of characteristic azurophilic granules in their cytoplasm. NK cells differentiate and mature in the bone marrow, lymph nodes, spleen, tonsils, and thymus. NK cells can be detected by specific surface markers, such as CD16, CD56, and CD8 in humans. NK cells do not express T-cell antigen receptors, the pan T marker CD3, or surface immunoglobulin B cell receptors.

Stimulation of NK cells is achieved through a cross-talk of signals derived from cell surface activating and inhibitory receptors. The activation status of NK cells is regulated by a balance of intracellular signals received from an array of germ-line-encoded activating and inhibitory receptors. When NK cells encounter an abnormal cell (e.g., tumor or virus-infected cell) and activating signals predominate, the NK cells can rapidly induce apoptosis of the target cell through directed secretion of cytolytic granules containing perforin and granzymes or engagement of death domain-containing receptors. Activated NK cells can also secrete type I cytokines, such as interferon-γ, tumor necrosis factor-α and granulocyte-macrophage colony-stimulating factor (GM-CSF), which activate both innate and adaptive immune cells as well as other cytokines and. Production of these soluble factors by NK cells in early innate immune responses significantly influences the recruitment and function of other hematopoietic cells. Also, through physical contacts and production of cytokines, NK cells are central players in a regulatory crosstalk network with dendritic cells and neutrophils to promote or restrain immune responses.

NK cells may be derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. In certain aspects, the NK cells are isolated and expanded ex vivo. For example, CB mononuclear cells may be isolated by ficoll density gradient centrifugation and cultured in a bioreactor with IL-2 and artificial antigen presenting cells (aAPCs). After 7 days, the cell culture may be depleted of any cells expressing CD3 and re-cultured for an additional 7 days. The cells may be again CD3-depleted and characterized to determine the percentage of CD56⁺/CD3⁻ cells or NK cells. In other methods, umbilical CB may be used to derive NK cells by the isolation of CD34⁺ cells and differentiation into CD56⁺/CD3⁻ cells by culturing in medium contain SCF, IL-7, IL-15, and IL-2.

### F. Engineering of Immune Effector Cells

The immune effectors cells (e.g., autologous or allogeneic T cells (e.g., regulatory T cells, CD4⁺ T cells, CD8⁺ T cells, or gamma-delta T cells), NK cells, invariant NK cells, or NKT cells) may be genetically engineered to express antigen receptors such as chimeric antigen receptors (CARs). For example, the host cells (e.g., autologous or allogeneic T-cells) may be modified to express a CAR having antigenic specificity for coronavirus spike protein. In particular embodiments, NK cells are engineered to express a CAR. Multiple CARs, such as to different antigens, may be added to a single cell type, such as T cells or NK cells.

The cells may comprise one or more nucleic acids introduced via genetic engineering that encode one or more antigen receptors, and genetically engineered products of such nucleic acids. The nucleic acids may be heterologous, i.e., normally not present in a cell or sample obtained from the cell, such as one obtained from another organism or cell, which for example, is not ordinarily found in the cell being engineered and/or an organism from which such cell is derived. The nucleic acids may not be naturally occurring, such as a nucleic acid not found in nature (e.g., chimeric).

### V. Pharmaceutical Formulations

The present disclosure provides pharmaceutical compositions comprising antibodies that selectively target coronavirus spike protein. Such compositions comprise a prophylactically or therapeutically effective amount of an antibody or a fragment thereof and a pharmaceutically acceptable carrier. Also provided herein are pharmaceutical compositions and formulations comprising immune cells (e.g., T cells or NK cells) expressing a CAR and a pharmaceutically acceptable carrier.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Water is a particular carrier when the pharmaceutical composition is administered intravenously. Saline solutions and aqueous dextrose and glycerol solutions can also be employed as liquid carriers, particularly for injectable solutions. The pH and exact concentration of the various components in a pharmaceutical composition are adjusted according to well-known parameters.

The active ingredients can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in Remington's Pharmaceutical Sciences. Such compositions will contain a prophylactically or therapeutically effective amount of the antibody or fragment thereof, preferably in purified form, together with a suitable amount of carrier so as to provide the form for proper administration to the patient.

Passive transfer of antibodies generally will involve the use of intravenous or intramuscular injections. The forms of antibody can be as monoclonal antibodies. Such immunity generally lasts for only a short period of time, and there is also a potential risk for hypersensitivity reactions, and serum sickness, especially from gamma globulin of non-human origin. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, pharmaceutical compositions may comprise, for example, at least about 0.1% of an active ingredient. In other embodiments, an active ingredient may comprise between about 2% to about 75% of the weight of the unit, or between about 25% to about 60%, for example, and any range derivable therein.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

### VI. Methods of Treatment

Certain aspects of the present embodiments can be used to prevent or treat a disease or disorder associated with a coronavirus infection, such as a SARS-COV-2 infection or COVID-19. In certain embodiments, the compositions and methods of the present embodiments involve administering an antibody or an antibody fragment against coronavirus spike protein, optionally in combination with a second or additional therapy.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of at least one antibody that targets coronavirus spike protein, either alone or in combination with other therapies.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of a coronavirus infection may involve, for example, a reduction in viral load. Treatment of coronavirus may also refer to increasing the likely hood of survival of a subject with a severe coronavirus infection.

In addition to being used as a monotherapy, the antibodies of the present invention may also find use in combination therapies. Effective combination therapy may be achieved with a single composition or pharmacological formulation that includes both agents, or with two distinct compositions or formulations, administered at the same time, wherein one composition includes at least one antibody of this invention, and the other includes the second agent(s). Alternatively, the antibody therapy may precede or follow the other agent treatment by intervals ranging from minutes to months.

Various combinations may be employed, such as when an antibody of the present invention is “A” and “B” represents a secondary agent, non-limiting examples of which are described below:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is contemplated that other therapeutic agents may be used in conjunction with the treatments of the current invention. In some embodiments, the present invention contemplates the use of one or more other therapies for the treatment of COVID-19 include the use of a coronavirus protease inhibitor, anti-platelet drugs, an anti-coagulation agent, a human type I interferon, a corticosteroid, or remdesivir.

In some embodiments, the anti-platelet drug is aspirin, an ADP receptor antagonist (e.g., ticlopidine, clopidogrel, cangrelor, prasugrel, ticagrelor, thienopyridine), or a glycoprotein IIb/IIIa receptor inhibitor (e.g., abciximab, eptifibatide, ticofiban). In some embodiment, the anti-coagulation agent is rivaroxaban, apixaban, dipyridamole, cilostazol, atromentin, edoxaban, fondaprinux, betrixaban, letaxaban, eribaxaban, hirudin, a thrombin inhibitor (e.g., lepirudin, desirudin, dabigatran, bivalirudin, ximelagatran), argatroban, batroxobin, hementin, low molecular weight heparin, unfractionated heparin, vitamin E, or a vitamin K antagonist (e.g., warfarin (Coumadin), acenocoumarol, phenprocoumon, phenindione).

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™ analogues, Betaseron™, Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly (I)-poly (C), imiquimod, cridanimod, bropirimine.

It is contemplated that other agents may be used in combination with certain aspects of the present invention to improve the therapeutic efficacy of treatment. These additional agents include anti-virals, corticosteroids (e.g., dexamethasone), chloroquine, hydroxychloroquine, remdesivir, favipiravir, lopinavir, and ritonavir.

### VII. Methods of Detection

In some aspects, the present disclosure concerns immunodetection methods for detecting the presence of a coronavirus spike protein. A wide variety of assay formats are contemplated for detecting protein products, including immunohistochemistry, enzyme linked immunosorbent assay (ELISA), radioimmunoassay (RIA), immunoradiometric assay, fluoroimmunoassay, chemiluminescent assay, bioluminescent assay, dot blotting, FACS analyses, and Western blot to mention a few. The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, blood, serum, plasma, cerebral spinal fluid, urine, stool, saliva, circulating tumor cells, exosomes, or aspirates and bodily secretions, such as sweat. In some aspects, the biological sample contains cell-free DNA.

### VIII. Kits

In various aspects of the embodiments, a kit is envisioned containing therapeutic agents and/or other therapeutic and delivery agents. In some embodiments, a kit is provided for preparing and/or administering a therapy of the embodiments. The kit may comprise one or more sealed vials containing any of the pharmaceutical compositions of the present embodiments. The kit may include, for example, at least one coronavirus spike protein-specific antibody or coronavirus spike protein-specific CAR construct, as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

In still further embodiments, the present disclosure concerns immunodetection kits for use with the immunodetection methods described above. As the antibodies may be used to detect a coronavirus or a coronavirus antigen, the antibodies may be included in the kit. The immunodetection kits will thus comprise, in suitable container means, a first antibody that binds to a coronavirus or a coronavirus antigen, and optionally an immunodetection reagent.

In certain embodiments, the coronavirus spike protein-specific antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the coronavirus or the coronavirus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Strains and media. E. coli strain DH10B was used for all routine cloning of yeast and mammalian constructs. Yeast strain EBY100 (MATa AGA1::GAL1-AGA1::URA3 ura3-52 trp1 leu2-delta200 his3-delta200 pep4::HIS3 prbd1.6R can1 GAL) was acquired from ATCC (cat. no. MYA-4941) and used for antibody surface display and selection. To improve antibody surface expression the human chaperone BIP (binding immunoglobulin protein) and yeast PDI (protein disulfide isomerase) were genomically integrated as an expression cassette in the HO locus. Yeast were grown in rich medium (YPD; Takara, cat. no. 630409) or in selective medium for leucine prototrophs after library transformation (Takara cat. no. 630310). YEP-galactose was used for expression of displayed antibody libraries (1% yeast extract, 1% bacto-peptone, 0.5% NaCl, 2% galactose, 0.2% glucose).

Antigens and antibodies. Stabilized spike ECD antigen S-2P was biotinylated using the EZ-link kit (Thermo Scientific, cat. no. 21435) and labeled with streptavidin-AF647 (Invitrogen, cat. no. S32357). RBD was labeled with a mouse anti-human Fc-AF647 (Southern Biotech, cat. no. 9042-31). Fab library light chains were labeled with anti-FLAG M2-FITC (Sigma, cat. no. F4049).

Donors. The acquisition of blood specimens from convalescent individuals was approved by the University of Texas at Austin Institutional Review Board (protocol 2020-03-085; Breadth of serum antibody immune responses prior to, or following, patient recovery in asymptomatic and non-severe COVID-19). Informed consent was obtained from all participants. Blood was collected from 3 PCR-confirmed, symptomatic patients. Donors 1 and 2 were collected at day 12 post-onset of symptoms. Donor 3 was collected on day 11. None of the donors were hospitalized or experienced severe disease. PBMCs and plasma were both collected by density gradient centrifugation using Histopaque-1077 (Sigma-Aldrich).

Preparation of serum antibodies for Ig-Seq proteomic analysis. Serum samples were prepared for Ig-Seq analysis as previously described (Voss et al., 2020). Briefly, total IgG was isolated from plasma using Pierce Protein G Plus Agarose (Pierce Thermo Fisher Scientific) and cleaved into F(ab′)2 fragments with IdeS protease. Antigen-specific F(ab′)2 was enriched by affinity chromatography against recombinant SARS-COV-2 S-2P or RBD protein cross-linked to NHS-activated agarose resin (Thermo Fisher Scientific). Eluted F(ab′)2 fractions were concentrated by vacuum centrifugation and prepared for mass spectrometry-based proteomic analysis as previously described (Lavinder et al., 2014).

LC-MS/MS analysis of antigen-enriched antibodies. Liquid chromatography-tandem mass spectrometry analysis was carried out on a Dionex Ultimate 3000 RSLCnano system coupled to an Orbitrap Fusion Lumos Mass Spectrometer (Thermo Scientific). Samples were loaded onto an Acclaim PepMap 100 trap column (75 μm×2 cm; Thermo Scientific) and separated on an Acclaim PepMap RSLC C18 column (75 μm×25 cm; Thermo Scientific) with a 3%-40% acetonitrile gradient over 60 min at a flow-rate of 300 nL/min. Peptides were eluted directly into the Lumos mass spectrometer using a nano-electrospray source. Mass spectra were acquired in data-dependent mode with a 3 sec cycle time. Full (MS1) scans were collected by FTMS at 120,000 resolution (375-1600 m/z, AGC target=5E5). Parent ions with positive charge state of 2-6 and minimum intensity of 3.4E4 were isolated by quadrupole (1 m/z isolation window) and fragmented by HCD (stepped collision energy=30+/−3%). Fragmentation (MS2) scans collected by ITMS (rapid scan rate, AGC target=1E4). Selected ions and related isotopes were dynamically excluded for 20 sec (mass tolerance=+/−10 ppm).

Antibody variable chain sequencing. Peripheral blood mononuclear cells (PBMCs) from processed donor samples were provided in Trizol as a kind gift from Dr. Gregory C. Ippolito (Voss et al., 2020). IgG and IgM V_{H} and V_{L} cDNA libraries were separately amplified from PBMC RNA of three donors and sequenced to create donor specific reference databases, from which the complete amino acid sequences of serum IgG proteins could subsequently be determined based on their mass spectral identifications. Tables 5 & 6 provide the distribution and diversity of clonotypes and V-gene usage for the variable heavy chain repertoires of donors 1 and 2.

Ig-Seq MS data analysis. Mass spectra were analyzed using Proteome Discoverer 2.2 software (Thermo Scientific). Precursor masses were first recalibrated with the Spectrum File RC node using a consensus human reference proteome database (UniProt) with common contaminants (MaxQuant) and precursor mass tolerance of 20 ppm. Recalibrated mass spectra were searched against a custom database for each donor consisting of donor-derived V_{H} sequences, V_{L} sequences, and the human and contaminant sequences using the Sequest HT node. Mass tolerances of 5 ppm (precursor) and 0.6 Da (fragment) were used. Static carbamidomethylation of cysteine (+57.021 Da) and dynamic oxidation of methionine (+15.995 Da) were considered. False discovery rates for peptide-spectrum matches (PSMs) were estimated by decoy-based error modelling through the Percolator node. Label-free quantitation (LFQ) abundances were calculated from precursor areas using the Minora Feature Detector and Precursor Ions Quantifier nodes.

Resulting PSMs were filtered according to methods described (Boutz et al., 2014). Briefly, peptide sequences differing only by isoleucine/leucine substitution were considered equivalent and combined into a single PSM. PSMs were re-ranked by posterior error probability, q-value, and Xscore. Only top-ranked, high-confidence PSMs (FDR<1%) were retained for each scan. If two or more PSMs had identical top-ranked scores, they were considered ambiguous and removed. PSMs for the same peptide sequence were summed and the average mass deviation (AMD) was calculated for each peptide. Peptides with AMD greater than 2 ppm were filtered out. Peptides mapping to VH sequences from a single clono-group were considered clono-specific. Clono-specific peptides overlapping the CDR3 sequence by four amino acids or more were considered CDR3-informative.

For each clono-group, PSMs and LFQ abundances of clono-specific CDR3-informative peptides were summed. Ratios of elution: flow-through PSMs and LFQ abundances were calculated; only clono-groups with both ratios >5 were considered elution-specific.

Public light chain screening. A previously published dataset of VH-VL pairs was analyzed and the top 6 most abundant kappa and top 3 most abundant lambda V-genes chosen (FIG. 2A). We then constructed germline versions of these V-genes with their most frequently observed J gene as published on the iRepertoire website (Table 18) and obtained full-length light chains for each PLC. SARS-COV-2 specific Ig-Seq or YSD heavy chains were then expressed with each PLC as a full-length IgG1 and screened for binding through ELISA using recombinant S2P.

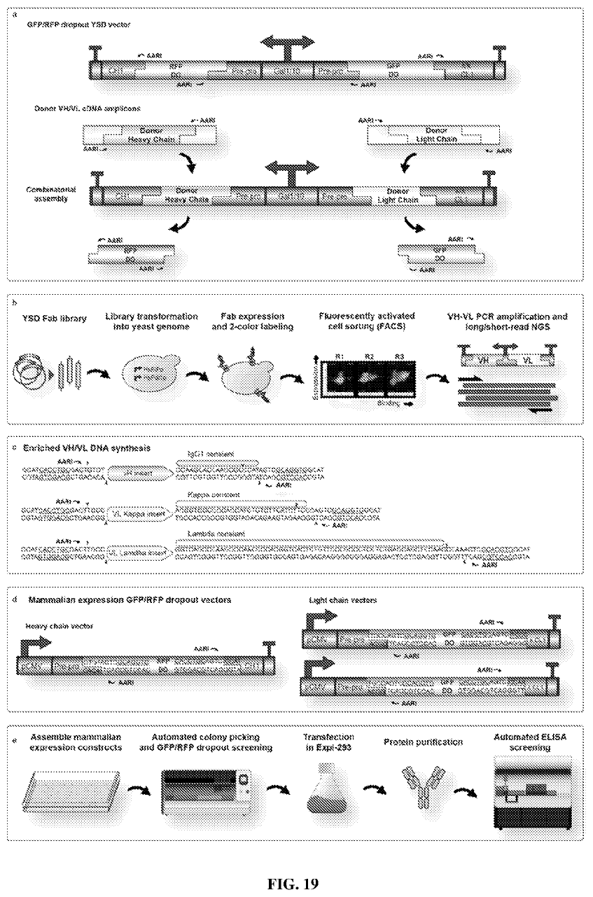

Library assembly and bacterial transformation. Donor B-cell V_{H} and V_{L} amplicons were amplified via PCR to include adapters for cloning into yeast expression vectors (FIG. 19A). Assembly into the yeast kappa and lambda expression vectors was done via Golden Gate assembly. Library assemblies were prepared in 20 μL reactions as follows: 2 AL 10×AARI buffer (ThermoFisher Scientific, cat. no. B27), 0.4 μL 50× oligo buffer (ThermoFisher Scientific, cat. no. ER1582), 0.2 μL 100 mM ATP (ThermoFisher Scientific, cat. no. R0441), 20 fmol backbone DNA, 40 fmol VH and VL amplicons, 0.5 μL (2 U/μL) AARI endonuclease (ThermoFisher Scientific, cat. no. ER1582), and 0.5 μL (3000 U/μL) T7 ligase (NEB, cat. no. M0318). Each assembly was scaled up to 16 total reactions in 8-well strips. Thermocycling consisted of the following protocol: 37° C., 15 minutes; 37° C., 2 minutes, 16° C., 1 minute; go to step 2, x74; 37° C., 60 minutes; 80° C., 15 minutes; hold at 4° C. Assemblies were consolidated and column purified using Promega Binding Solution (Promega, cat. no. A9303) to bind DNA to a Zymo-spin II column (Zymo Research, cat. no. C1008). The column was washed twice with DNA Wash Buffer (Zymo Research, cat. no. D4003) and eluted in 30 μL nuclease-free water. For library transformations, DH10B cells were diluted 1:100 from confluent culture into 50 mL Superior broth (AthenaES, cat. no. 0105). When cells reached an OD₆₀₀ of 0.4-0.6, they were washed 3× with cold 10% glycerol and resuspended to a final volume of 600 μL. The purified library was added to cells and electroporated at 1.8 kV in an E. coli Pulser electroporator (Bio-Rad) using Genepulser 0.2 cm cuvettes (Bio-Rad, cat. no. 1652086) at 200 μL per transformation.

Library transformation into yeast and protein expression (FIG. 19B). Purified libraries were linearized for integration into the yeast genome via homologous recombination at the Leu2 locus. For each 1 μg library plasmid, 0.5 μL NotI (10 units/μL) (NEB, cat. no. R0189) was used with the supplied Buffer 3.1 in 10 μL. Reactions were incubated at 37° C. overnight and heat inactivated at 80° C. for 20 minutes. Digests were pooled and column purified as described in previous sections and eluted in 25 μL nuclease-free water. The strain was electroporated as described elsewhere (Zhou et al., 2020). 10 μg of linearized DNA was sufficient for library sizes of 10⁶, and that library sizes could reach >10⁷ with 20 μg DNA. Transformed yeast were recovered in SD-Leu medium (see “strains and media” section). Libraries were passaged once at 1:100 before protein expression to reduce contamination from untransformed cells. To express Fab libraries, yeast were washed in YEP-galactose (see “strains and media” section) and diluted 1:10 into 10 mL final volume. Cells were induced for 48 hours at 20° C. with shaking.

Fab library labeling and selection (FIG. 19B). Expressed yeast libraries were harvested at 100 μL (representing approximately 10⁷ cells) and washed with PBSA buffer (1×PBS, 2 mM EDTA, 0.1% Tween-20, 1% BSA, pH 7.4). Antigen was incubated with cells in 1 mL in PBSA at 200 nMat RT for one hour, washed with PBSA at 4° C., and labeled with secondary antibodies (mouse anti-human FITC, 1:100; streptavidin-AF647, 1:100; mouse anti-human Fc-AF647, 1:50). Cells were washed 2× and resuspended in 2 mL in cold PBSA for sorting. Cell sorting was performed using a Sony SH800 fluorescent cell sorter. For first round libraries, 10⁷ events were sorted into 2 mL SD-Leu medium supplemented with penicillin/streptomycin (Gibco, cat. no. 15140122). Cells were recovered by shaking incubation for 1-2 days for further rounds of selection or plated directly for phenotyping clones.

Phenotyping assays. Sorted clones from rounds two or three of selection were picked into microplates as described previously. After antibody expression, 10 μL of cells were transferred to a fresh 2-mL microplate and washed 2× with 200 μL cold PBSA buffer. Cells were labeled with 200 nM spike or RBD antigen in 50-100 μL PBSA at RT for 1 h with shaking at 1000 rpm, 3 mm orbit. Labeled cells were washed 2×, and secondary labels were applied as previously described. Cells were resuspended in 200 μL ice-cold PBSA just before analysis. Samples were analyzed on a Sony SA3800 Spectral Cell Analyzer.

Next generation sequencing. Genome extraction was performed on yeast cultures of libraries and sorted rounds underwent genome extraction using a commercial kit (Promega, cat. no. A1120) with zymolyase (Zymo Research, cat. no. E1004). 100 ng genomic template was used to amplify the heavy and light chains separately or as one amplicon for short or long-read sequencing, respectively. For amplification of heavy chain genes only, primers JG.VHVLK.F and JG.VH.R were used. For amplification of light chain genes only, primers JG.VL.F and JG.VHVK.R or JG.VHVL.R were used for kappa and lambda vectors, respectively. For amplification of paired genes, primers JG.VHVLK.F and JG.VHVK.R or JG.VHVL.R were used. Amplicons were column purified and deep sequenced with an iSeq. In parallel, ˜1.8 kb sequences spanning the entire VH and VL were obtained using MinION nanopore sequencing (Oxford Nanopore Technologies Ltd., MinION R10.3).

Colony PCR and Sanger sequencing. Sorted yeast populations were plated on SD-Leu and 8-32 colonies per plate were picked into 2 mL microplates either by hand or using a QPIX 420 (Molecular Devices) automatic colony picker. Cultures were grown at 1000 rpm at 3 mm orbit at 30° C. overnight. Cells (20 μL) were transferred to a fresh microplate and washed with 1 mL TE buffer (10 mM Tris, 1 mM EDTA). Cells were incubated with 20 μL zymolyase solution (5 mg/mL zymolyase, 100T in TE) at 37° C. for 1 hour. Cells (5 μL) were then used in colony PCR to amplify the paired heavy and light chains. Amplicons were column purified with the Wizard SV 96 PCR Clean-Up System (Promega, cat. no. A9342) and yields were quantified with a Nanodrop spectrophotometer or the Quant-it Broad-Range dsDNA kit (Invitrogen, cat. no. Q33130). Approximately 10 ng (2.5-5 μL) of purified PCR products were then subjected to Sanger sequencing.

Long-read sequencing (donors 1 & 2). Sequencing libraries were prepared from 18 amplicon samples using the Native Barcoding Kit (Oxford Nanopore Technologies; cat. no. EXP-NBD103) paired with the Ligation Sequencing Kit (Oxford Nanopore Technologies; cat. no. SQK-LSK109) according to the manufacturer's directions. Between four and eight sequencing libraries per flowcell were pooled for sequencing on three MinION flow cells (Oxford Nanopore Technologies; R9.4.1) for 72 hours on an Oxford Nanopore Technologies MinION Mk1B device (Oxford Nanopore Technologies). Raw data was basecalled using the high accuracy model in Guppy (v.3.5.2).

Short-read sequencing (phenotyping plates). Sequencing libraries were prepared from 308 amplicon samples using the Nextera DNA Flex Library Preparation kit (Illumina; cat. no. 20018705) according to the manufacturer's directions. Sequencing libraries were pooled and sequenced (2×151 bp) on an iSeq 100 (Illumina; California, USA) using iSeq 100 i1 Reagents v.1 (Illumina; cat. no. 20021533).

Long-read sequencing (donor 3). Sequencing libraries were prepared from 32 amplicon samples using the Native Barcoding Kit (Oxford Nanopore Technologies; cat. no. EXP-NBD104) paired with the Ligation Sequencing Kit (Oxford Nanopore Technologies; cat. no. SQK-LSK109) according to the manufacturer's directions. Between five and eight sequencing libraries per flow cell were pooled for sequencing on five GridION flow cells (Oxford Nanopore Technologies; R9.4.1) for 72 hours on a GridION Mk1 device (Oxford Nanopore Technologies; Oxford, England, UK). Raw data was live basecalled using the high accuracy model in Guppy (v.3.2.10).

Short-read sequencing (donor 3). Sequencing libraries were prepared from 32 samples using the Nextera DNA Flex Library Preparation kit (Illumina; cat. no. 20018705) according to the manufacturer's directions. Sequencing libraries were pooled and sequenced (2×151 bp) on an iSeq 100 (Illumina; California, USA) using the iSeq 100 i1 Reagents v2 (Illumina; cat. no. 2009584).

Long-read sequencing (YSD-IgSeq). Sequencing libraries were prepared from 16 amplicon samples using the Native Barcoding Kit (Oxford Nanopore Technologies; Cat. No. EXP-NBD104) paired with the Ligation Sequencing Kit (Oxford Nanopore Technologies; Cat. No. SQK-LSK109) according to the manufacturer's directions. Four sequencing libraries were pooled per flow cell and sequenced on four GridION flow cells (Oxford Nanopore Technologies; R9.4.1) for 72 hours on a GridION Mk1 device (Oxford Nanopore Technologies; Oxford, England, UK). Raw data was live basecalled using the high accuracy model in Guppy (v.4.0.11).

Short-read sequencing (YSD-IgSeq). Sequencing libraries were prepared from 16 samples using the Nextera DNA Flex Library Preparation kit (Illumina; cat. no. 20018705) according to the manufacturer's directions. Sequencing libraries were pooled and sequenced (2×150 bp) on an iSeq 100 (Illumina; California, USA) using the iSeq 100 i1 Reagents v2 (Illumina; Cat. No. 2009584).

Sequence processing and consolidation into VHVL clones. Individual reads after Guppy base calling typically average more than 10% error per base, and numerous tools exist to align and reduce reads into consensus sequences with substantially improved accuracy (Wick et al., 2019). However, such tools were not designed for antibody library sequencing and its huge populations of subtly different sequences which, even assuming successful alignment, group into a myriad of very short, disconnected assemblies. Therefore, a bioinformatic pipeline was implemented to obtain accurate VHVL sequences from the MinION and iSeq data and to estimate, within each YSD round, the relative abundance of individual VHVL pairs.

These methods proceed through antibody V (D) J annotation of raw MinION reads using MiXCR (v3.0.13) (Bolotin et al., 2015); iteratively growing and shrinking sequence clusters based on annotated features from each read; sequence error correction and consolidation within each cluster, optionally including high quality Illumina reads; and finally, VHVL clone definition within each sample and quantitation by number of reads mapped to each clone. For enumeration, only counts for reads with a length of 1700 to 2100 base pairs were included. This points to a secondary advantage of MinION sequencing, as shorter reads proliferate during PCR and inflate apparent abundance of particular species. Without length-filtering, relative VHVL abundance calculated from both MinION and iSeq reads are strongly correlated.

Tissue culture and transient transfection of ECD and RBD. Spike ECD protein and RBD proteins were expressed in Expi293F cells using the manufacturer provided guidelines with slight modifications. In short, a 1 ml frozen working cell bank of Expi293F cells at 1×10⁷ viable cells/mL were thawed in a bead bath at 37° C. for 2-3 mins. The vial was sprayed with 70% isopropyl alcohol and transferred into a biosafety cabinet. The thawed cells were transferred to a 125 mL non-baffled vented shake flask containing 29 mL of fresh pre-warmed ExpiExpression medium at 37° C. Cells were incubated in at 37° C. with ≥80% humidity and 8% CO₂ on an orbital shaker at 120 rpm and grown until they reached a cell density of 3×10⁶ viable cells/mL. Fresh pre-warmed ExpiExpression medium was added to 1 L non-baffled vented shake flask and the 30 mL cell suspension was carefully introduced, making the seeding density of 0.4×10⁶ viable cells/mL in a final culture volume of 225 mL. After the cell density reached 3×10⁶ viable cells/mL, the culture was expanded to a final volume of 2.25 L in two 2.8 L Thomson Optimum Growth flasks with a seeding density of 0.3×10⁶ viable cells/mL. After the cell density reached 3×10⁶ viable cells/mL, 2 L of the culture was split into four Thompson flasks with each flask containing 500 mL of culture medium and 500 mL of fresh pre-warmed ExpiExpression medium. The final culture volume in each of the four flasks was 1 L. The cells were incubated at 37° C. with ≥80% humidity and 8% CO₂ on an orbital shaker at 100 rpm. When the cells reached a density of 3×10⁶ viable cells/mL, the culture was transferred to two 500 mL sterile centrifuge bottles and the cells were spun down at 100×g for 10 min. The supernatant was removed, and the cells were resuspended in 4 L of fresh, prewarmed medium. The cells were allowed to re-stabilize in the incubator for 24 h and were transfected at 3×10⁶ viable cells/mL in 4 L.

Transfection was performed by diluting 4 mg of plasmid DNA (pDNA) in 240 mL of OptiMEM medium in a sterile bottle and gently inverting 3-4 times before incubating at room temperature (RT) for 5 min. ExpiFectamine 293 reagent (13 mL) was then diluted in 225 mL OptiMEM in a sterile bottle and inverted 3-4 times before incubating for 5 min at RT. The diluted pDNA and ExpiFectamine reagent were carefully mixed and incubated at RT for 15 min. One-fourth of the combined complex was then slowly transferred to each of the flasks while gently swirling the cells during addition. The cultures were again placed in the incubator with shaking at 100 rpm for 18 h. Post-transfection, ExpiFectamine 293 Transfection Enhancer 1 (6 mL) and ExpiFectamine 293 Transfection Enhancer 2 (60 mL) were added to each flask. The cell viability was monitored every 24 h and the cells were harvested when the viability dropped below 70% or after approximately 3 d. Harvesting was done by centrifugation at 15,900×g for 45 min at 4° C. and the supernatant was transferred into sterile bottles.

Antigen purification. The feed was prepared adding 0.2 M NaCl and 10 mM imidazole to the supernatant while mixing. The feed material was filtered using a 0.45 μm PES filter membrane pre-wetted with PBS before loading on a prepared Ni-IMAC column. A metal affinity column was prepared by packing IMAC FF beads (Cytiva) into an AxiChrom 70 column housing to a bed height of 9.5 cm and then charging with 50% column volume (CV) of a 0.2 M nickel sulfate solution, washing with water and then 50% CV of 100% “B” buffer (50 mM sodium phosphate buffer containing 300 mM NaCl and 250 mM imidazole, pH 7.8) to remove weakly bound nickel ions. The column was then washed with 100% “A” buffer (50 mM sodium phosphate buffer containing 300 mM NaCl and 20 mM imidazole, pH 7.2) prior to sample loading. The prepared (4.8 L) feed was then loaded onto the column at a linear flow rate of 90 cm/h. After loading, the column was washed with two CVs of 100% A buffer and two CVs of a 13% B buffer (containing 50 mM imidazole) before eluting the protein using 100% B (250 mM imidazole) for 3 CVs. All steps except for the loading step were done at 150 cm/h.

Fractions from the elution peak were pooled and then concentrated 4- to 8-fold by ultrafiltration (UF) using a 115 cm² hollow fiber cartridge with either a 50 kDa (S-2P) or a 10 kDa (RBD) molecular weight cutoff membrane (Repligen) and then diafiltered after concentration by exchanging with 5 volumes of PBS.

The concentration of diafiltered protein was determined by measuring the absorbance at 280 nm versus a PBS blank. The protein concentration in mg/mL was obtained using a divisor of 1.03 mL/mg-cm for S-2P and 1.19 mL/mg-cm for RBD. A qualitative assessment of protein quality was made using SDS-PAGE with SYPRO Ruby staining (BioRad) for reduced and non-reduced samples. Only those preparations showing predominantly full-length S-2P (160 kDa subunits) or RBD (70 kDa) were used in ELISAs for assessing antibody binding.

Monoclonal antibody expression and purification. VHVL candidates were cloned into custom Golden Gate compatible pCDNA3.4 vectors for IgG1 expression. For transfections, V_{L} was mixed 3:1 with a corresponding V_{H}. Plasmids were transfected into Expi293F (Invitrogen) cells using the recommended protocol. Monoclonal antibodies were harvested at 5-7 days post-transfection. Expi293F cells were centrifuged at 300×g for 5 min, supernatants were collected and centrifuged at 3000×g for 20 min at 4° C. and diluted to 1×PBS final concentration. Each supernatant was passed through a Protein G or A agarose affinity column (Thermo Scientific). Flow through was collected and passed through the column three times. Columns were washed with 10 CV of PBS and antibodies were eluted with 5 mL 100 mM glycine, pH 2.7 directly in neutralization buffer containing 500 μL 1 M Tris-HCl, pH 8.0.

Analysis of mammalian cell surface displayed spike proteins with flow cytometry. The assay has been described in detail in the recent paper by Javanmardi et al. Briefly, plasmids expressing full-length SARS-COV-2 spike proteins (Spike-Linker-3×FLAG-TM), WT (HexaPro-D614G) and variants, were transfected into HEK293T cells using Lipofectamine 2000 according to manufacturer's instructions. After 48 h, cells were collected and resuspended in PBS-BSA and incubated with anti-FLAG (mouse) and anti-spike (human) mAbs for 1 h, shaking at RT. Cells were washed 3× with PBS-BSA, resuspended in one mL and incubated with Alexa Fluor 488 (anti-mouse) and Alexa Fluor 647 (anti-human) antibodies for 30 min, shaking at 4° C. Cells were washed again and resuspended in PBS-BSA prior to flow cytometry analysis (SA3900 Spectral Analyzer, Sony Biotechnology). All data was analyzed with FlowJo (BD Bioscience).

ELISA. Antigen ELISA plates were made using high-binding plates (Corning, cat. no. 3366) with antigen diluted in PBS to a final concentration of 2 μg/mL. Antigen solution (50 μL) was added to microplates and incubated overnight at 4° C. with shaking at 100 rpm, 3 mm orbit. Plates were blocked with PBSM (2% milk in PBS) at RT for 1 h. Plates were washed 3× with 300 μL PBS-T (0.1% Tween-20). Purified antibodies were prepared to 10 μg/mL in PBSM and serially diluted. Antibodies were incubated for 1 h at RT. Plates were washed 3× with PBS-T, and secondary goat anti-human Fab-HRP (Sigma-Aldrich, cat. no. A0293) was applied at 1:5000 in PBSM in 50 μL and incubated at RT for 45 min. HRP substrate (50 μL) was added to wells and the reaction proceeded for 5-15 min until quenched with 50 μL 4M H₂SO₄ and analyzed for absorbance at 450 nm in a plate reader.

Live virus neutralization assay. Assays were performed by two independent groups. The first SARS-COV-2 microneutralization assay was adapted from an assay used to study Ebola virus (Brown et al., 2018). This assay also used SARS-COV-2 strain WA1. Antibodies were diluted in cell culture medium in triplicate. A SARS-COV-2 monoclonal antibody was used as a positive control. An antibody that does not bind SARS-CoV-2A was used as negative control. Diluted antibodies were mixed with the SARS-COV-2 WA1 strain, incubated at 37° C. for 1 h, then added to Vero-E6 cells at target MOI of 0.4. Unbound virus was removed after 1 h incubation at 37° C., and culture medium was added. Cells were fixed 24 h post-infection, and the number of infected cells was determined using SARS-COV-S specific mAb (Sino Biological, cat. no. 401430-R001) and fluorescently labeled secondary antibody. The percent of infected cells was determined with an Operetta high-content imaging system (PerkinElmer) and Harmonia software. Percent neutralization for each monoclonal antibody at each dilution was determined relative to untreated, virus-only control wells.

The second set of live virus neutralization (VN) assays for variants of concern and WA1 was performed in an orthogonal assay. The ability of the monoclonal antibodies to neutralize SARS-COV-2 was determined with a traditional VN assay using SARS-COV-2 strain USA-WA1/2020 (NR-52281-BEI resources). All experiments with SARS-COV-2 were performed in the Eva J Pell BSL-3 laboratory at Penn State and were approved by the Penn State Institutional Biosafety Committee (IBC #48625). For each mAb a series of 12 two-fold serial dilutions were assessed. Triplicate wells were used for each antibody dilution. 100 tissue culture infective dose 50 (TCID50) units of SARS-COV-2 were added to 2-fold dilutions of the diluted mAb. After incubating for 1 hour at 37° C., the virus and mAb mixture was then added to Vero E6 cells (ATCC CRL-1586) in a 96-well microtiter plate and incubated at 37° C. After 3 days, the cells were stained for 1 hour with crystal violet-formaldehyde stain (0.013% crystal violet, 2.5% ethanol, and 10% formaldehyde in 0.01 M PBS). The endpoint of the microneutralization assay was determined as the highest mAb dilution, at which all 3, or 2 of 3, wells are not protected from virus infection. Percent neutralization ability of each dilution of the mAb was calculated based on the number of wells protected, 3, 2, 1, 0 of 3 wells protected was expressed as 100%, 66.6%, 33.3%, or 0%.

Surface plasmon resonance. To investigate the binding kinetics of mAb N3-1 binding to the spikes, purified His-tagged spike variants (SARS-COV Tor2 S-2P, SARS-COV-2 Wuhan-Hu-1 S-HexaPro, SARS-COV-2 B.1.1.7 S-Hexapro and SARS-COV-2 B.1.351 S-HexaPro) were immobilized on a Ni-NTA sensor chip (GE Healthcare) using a Biacore X100 (GE Healthcare). For Fab binding experiments, spike proteins were immobilized to a level of ˜450 response units (RUs). Serial dilutions of purified Fab N3-1 were injected at concentrations ranging from 400 to 6.25 nM over spike-immobilized flow cell and the control flow cell in a running buffer composed of 10 mM HEPES pH 8.0, 150 mM NaCl and 0.05% Tween 20 (HBS-T). Between each cycle, the sensor chip was regenerated with 0.35 M EDTA, 50 mM NaOH and followed by 0.5 mM NiCl₂. For IgG binding experiments, spike immobilization of 200 RUs was used instead to avoid mass transport effect. Serial dilutions of purified IgG N3-1 were injected at concentrations ranging from 25 to 1.56 nM over a spike-immobilized flow cell and the control flow cell. For the SARS-COV Tor2 S-2P binding experiments, IgG N3-1 concentrations ranging from 100 to 6.25 nM were used. Response curves were double-reference subtracted and fit to a 1:1 binding model or heterogeneous ligand binding model using Biacore X100 Evaluation Software (GE Healthcare).

Negative stain EM for spike-IgG complexes. To investigate mAb N3-1 binding to spike proteins, purified SARS-COV-2 Wuhan-Hu-1 S-HexaPro was incubated with 1.2-fold molar excess of IgG N3-1 in 2 mM Tris pH 8.0, 200 mM NaCl and 0.02% NaN₃ on ice for 10 min. The spike-IgG complexes were at a concentration of 0.05 mg/mL in 2 mM Tris pH 8.0, 200 mM NaCl and 0.02% NaN₃ prior to deposition on a CF-400-CU grid (Electron Microscopy Sciences) that was plasma cleaned for 30 sec in a Solarus 950 plasma cleaner (Gatan) with a 4:1 ratio of O2/H2 and stained using methylamine tungstate (Nanoprobes). Grids were imaged at a magnification of 92,000× (corresponding to a calibrated pixel size of 1.63 Å/pix) in a Talos F200C T_{EM} microscope equipped with a Ceta 16M detector. The CTF-estimation, particle picking and 2D classification were all performed in cisTEM.

Cryo-EM sample preparation and data collection. Purified SARS-CoV-2 S (HexaPro variant) at 0.2 mg/mL was incubated with 5-fold molar excess of Fab N3-1 in 2 mM Tris pH 8.0, 200 mM NaCl and 0.02% NaN₃ at RT for 30 min. The sample was then deposited on plasma-cleaned UltrAuFoil 1.2/1.3 grids before being blotted for 4 sec with −3 force in a Vitrobot Mark IV and plunge-frozen into liquid ethane. Purified SARS-COV-2 S with three RBDs covalently trapped in the down conformations (HexaPro-RBD-down variant, S383C/D985C (Xiong et al., 2020; Henderson et al., 2020; McCallum et al., 2020) at 0.2 mg/mL, complexed with 2-fold molar excess of Fab A7V3, was deposited on plasma-cleaned UltrAuFoil 1.2/1.3 grids before being blotted for 3 sec with −4 force in a Vitrobot Mark IV and plunge-frozen into liquid ethane. For the HexaPro-N3-1 sample, 3,203 micrographs were collected from a single grid. For the HexaPro-RBD-down-A7V3 sample, 3,636 micrographs were collected from a single grid. FEI Titan Krios equipped with a K3 direct electron detector (Gatan) was used for imaging. Data were collected at a magnification of 22,500×, corresponding to a calibrated pixel size of 1.07 Å/pix. A full description of the data collection parameters can be found in Tables 3-4.

TABLE 3
N3-1 CryoEM Statistics.
EM data collection

| Microscope | FEI Titan Krios |
|---|---|
| Voltage (kV) | 300 |
| Detector | Gatan K3 |
| Magnification (nominal) | 22500 |
| Pixel size (Å/pix) | 1.1 |
| Flux (e⁻/pix/sec) | 8 |
| Frames per exposure | 80 |
| Exposure (e⁻/Å²) | 80 |
| Defocus range (μm) | 1.0-2.5 |
| Micrographs collected | 3203 |
| Sample | SARS-CoV-2 S + N3-1 Fab |

3D reconstruction statistics

| | | |
|---|---|---|
| | Overall | RBDs-N3-1 subcomplex |
| Particles | 269,553 | 269,553 |
| Symmetry | C1 | C1 |
| Map sharpening B-factor | −123.6 | −106.8 |
| Unmasked resolution at | 3.90 | 4.50 |
| 0.143 FSC (Å) | | |
| Masked resolution at | 2.78 | 3.16 |
| 0.143 FSC (Å) | | |

Model refinement and validation statistics

| Composition | |
|---|---|
| Amino acids | 579 |
| RMSD bonds (Å) | 0.003 |
| RMSD angles (°) | 0.59 |
| Average B-factors | |
| Amino acids | 102.3 |
| Ramachandran | |
| Favored (%) | 95.8 |
| Allowed (%) | 4.2 |
| Outliers (%) | 0 |
| Rotamer outliers (%) | 0 |
| Clash score | 4.0 |
| C-beta outliers (%) | 0 |
| CaBLAM outliers (%) | 1.6 |
| CC (mask) | 0.83 |
| MolProbity score | 1.48 |
| EMRinger score | 4.95 |

TABLE 4
A7V3 CryoEM Statistics.
EM data collection

| Microscope | FEI Titan Krios |
|---|---|
| Voltage (kV) | 300 |
| Detector | Gatan K3 |
| Magnification (nominal) | 22500 |
| Pixel size (Å/pix) | 1.1 |
| Flux (e⁻/pix/sec) | 8 |
| Frames per exposure | 80 |
| Exposure (e⁻/Å²) | 80 |
| Defocus range (μm) | 1.0-2.5 |
| Micrographs collected | 3636 |
| Sample | SARS-CoV-2 S + A7V3 Fab |

3D reconstruction statistics

| | | |
|---|---|---|
| | Overall | RBDs-N3-1 subcomplex |
| Particles | 715,398 | 216,070 |
| Symmetry | C1 | C1 |
| Map sharpening B-factor | −151.7 | −94.1 |
| Unmasked resolution at | 4.00 | 8.70 |
| 0.143 FSC (Å) | | |
| Masked resolution at | 2.99 | 3.36 |
| 0.143 FSC (Å) | | |

Model refinement and validation statistics

| Composition | |
|---|---|
| Amino acids | 452 |
| RMSD bonds (Å) | 0.003 |
| RMSD angles (°) | 0.63 |
| Average B-factors | |
| Amino acids | 112.2 |
| Ramachandran | |
| Favored (%) | 94.0 |
| Allowed (%) | 6.0 |
| Outliers (%) | 0 |
| Rotamer outliers (%) | 0 |
| Clash score | 4.1 |
| C-beta outliers (%) | 0 |
| CaBLAM outliers (%) | 3.1 |
| CC (mask) | 0.79 |
| MolProbity score | 1.59 |
| EMRinger score | 3.48 |

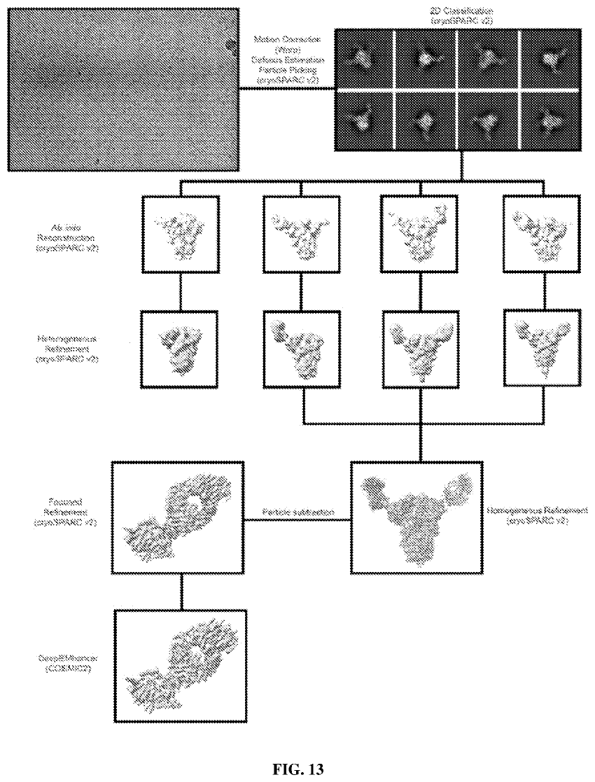

Cryo-EM data processing. Gain reference- and motion-corrected micrographs processed by Warp (Tegunov & Cramer, 2019) were imported into cryoSPARC v2.15.0 (Punjani et al., 2017), which was used to perform CTF correction, micrograph curation, particle picking, and particle curation via iterative rounds of 2D classification. The final global reconstructions were then obtained via ab initio reconstruction, iterative rounds of heterogeneous refinement, and subsequently non-uniform homogeneous refinement of final classes by applying C1 symmetry. For the HexaPro-RBD-down-A7V3 sample, C3 symmetry was attempted in the initial refinement process. Given the low occupancy of the Fabs on a trimeric spike, C1 symmetry was used for the final runs of heterogeneous and homogeneous refinement. To better resolve the Fab-spike interfaces, both datasets were subjected to particle subtraction and focused refinement. Finally, both global and focused maps were sharpened using DeepEMhancer (Sanchez-Garcia et al., 2020). For A7V3-NTD model building, a NTD from 4A8 complexed spike structure (PDB ID: 7C2L; Chi et al., 2020) and a homologous Fab structure (PDB ID: 6IEK) were used as an initial model to build into map density using UCSF ChimeraX (Goddard et al., 2018). For N3-1-RBDs model building, one RBD-up and one RBD-down from S-HexaPro (PDB ID: 6XKL; Hsieh et al., 2020) and two homologous Fab structures (PDB ID: 5BV7 and 5ITB) were used as an initial model to build into map density via UCSF ChimeraX. Both models were built further and iteratively refined using a combination of Coot (Emsley & Cowtan, 2004), Phenix (Adams et al., 2002), and ISOLDE (Croll, 2018). The detailed workflows of cryo-EM data processing and data validation can be found in FIGS. 13,14,17A-17F and Tables 3-4.

### Example 2—Ig-Seq Analysis of the Serological Repertoire Reveals Candidate Heavy Chains

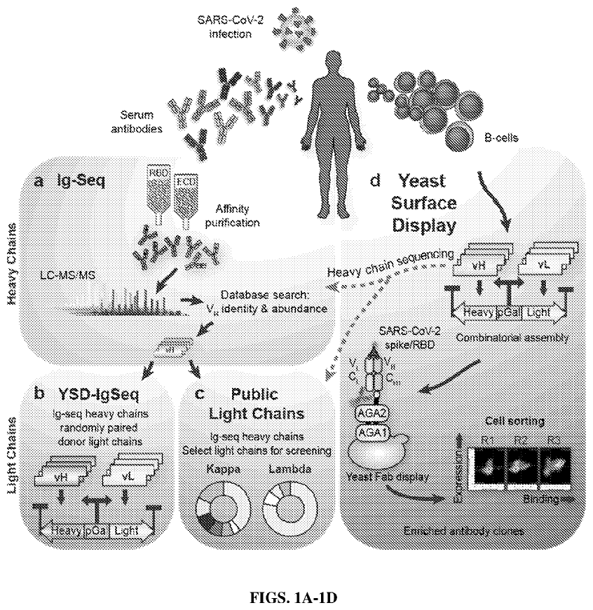

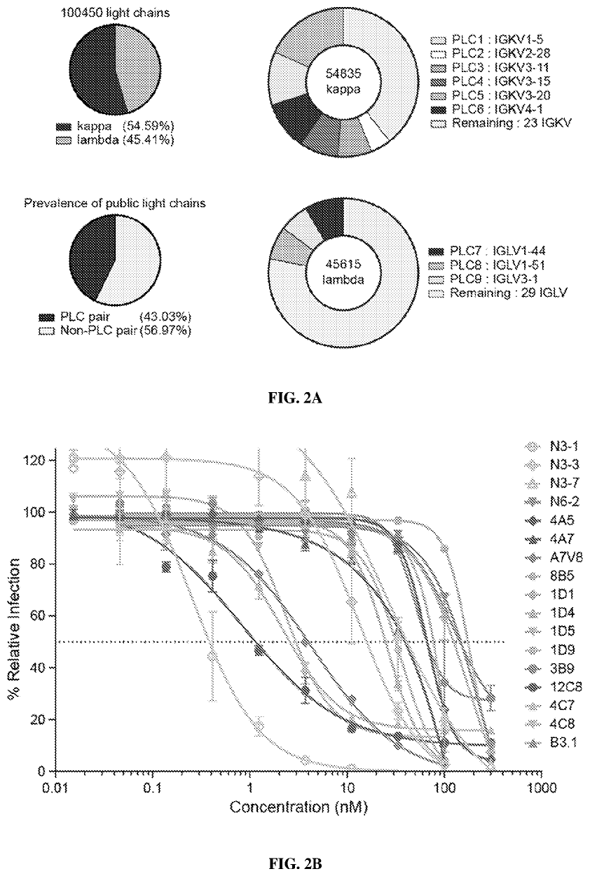

Ig-Seq provides a proteomic snapshot of the serum antibody repertoire by using mass spectrometry to identify heavy chain complementarity-determining region 3 (CDR3) peptides of antigen-enriched antibodies (FIG. 1) (DeKoskey et al., 2015). To identify relevant SARS-COV-2 antibodies present in the serological repertoire, antibodies were first isolated from the serum of two donors by antigen enrichment chromatography using immobilized SARS-COV-2 RBD and ectodomain (ECD) fragments. Then, Ig-Seq mass spectrometry was used to identify abundant IgG clonotypes. A total of 15 and 21 unique clonotypes were identified with high confidence from donors 1 and 2, respectively (Tables 5-7). This is notably lower than what has been observed in previous Ig-Seq studies (DeKoskey et al., 2015; Immune Repertoire Sequencing available at irweb.irepertoire.com/nir/showsample?sampleid=21746 #; Immune Repertoire Sequencing available at irweb.irepertoire.com/nir/showsample?sampleid=21745; Immune Repertoire Sequencing available at irweb.irepertoire.com/nir/showsample?sampleid=21744), perhaps because the previous studies involved donors after boost vaccination which is likely to elicit a more robust and diverse response than the primary response to natural SARS-COV-2 infection observed here. Regardless, the clonotypes identified provided a sufficient set of VH candidates to express and characterize as recombinant anti-SARS-COV-2 monoclonal antibodies (mAbs) in combination with suitable light chains.

TABLE 5
Summary of IgG VH-only sequencing of donor 1. Libraries generated from
these sequences were used for IgSeq proteomics and as a starting point
for YSD selections. Values represent the number of sequences.

| | IGHJ1 | IGHJ2 | IGHJ3 | IGHJ4 | IGHJ5 | IGHJ6 | ! Not Found |
|---|---|---|---|---|---|---|---|
| ! Not Found | 0 | 0 | 0 | 0 | 0 | 0 | 486 |
| IGHV1-2 | 689.17 | 278.5 | 1542.5 | 8289.25 | 1000.92 | 7030.67 | 0 |
| IGHV1-3 | 1 | 10 | 302.5 | 1082 | 499 | 515 | 0 |
| IGHV1-8 | 12 | 876 | 100.5 | 2089.33 | 2213 | 2909 | 0 |
| IGHV1-18 | 126.2 | 336.7 | 3802 | 23522.62 | 2668.62 | 18115.03 | 0 |
| IGHV1-24 | 10.7 | 1034.53 | 982.5 | 6680.53 | 7195.37 | 2356.7 | 0 |
| IGHV1-45 | 0 | 1 | 0 | 0 | 0 | 0 | 0 |
| IGHV1-46 | 30.5 | 77 | 2259.5 | 8560.08 | 1638.58 | 9163.83 | 0 |
| IGHV1-58 | 0 | 1 | 271 | 6 | 1 | 6 | 0 |
| IGHV1-69 | 20 | 54.5 | 974 | 16502.08 | 803.25 | 3699.5 | 0 |
| IGHV1-60D | 0 | 0 | 0 | 0 | 0 | 0 | 0 |
| IGHV1-69D | 0 | 0 | 0 | 45.5 | 0.67 | 1.17 | 0 |
| IGHV1/OR15-1 | 0 | 0 | 0 | 0 | 0 | 2 | 0 |
| IGHV2-5 | 2 | 2 | 85 | 2764.5 | 240.5 | 24 | 0 |
| IGHV2-26 | 1 | 0 | 1 | 69 | 17 | 152 | 0 |
| IGHV2-70 | 43.5 | 1 | 323.5 | 4122.25 | 6468.25 | 132 | 0 |
| IGHV2-70D | 0.5 | 0 | 2 | 39.75 | 96.25 | 0 | 0 |
| IGHV3-7 | 333.83 | 620 | 718.33 | 4924.5 | 1271.67 | 2913.5 | 0 |
| IGHV3-9 | 21.67 | 29.17 | 2208.75 | 6509.17 | 654.17 | 1127.17 | 0 |
| IGHV3-11 | 360.83 | 118 | 534.03 | 4249.5 | 1958 | 8348.33 | 1 |
| IGHV3-13 | 2 | 67 | 64 | 820.5 | 163.5 | 442 | 0 |
| IGHV3-15 | 43 | 20.5 | 162.5 | 3198 | 698.5 | 735.5 | 0 |
| IGHV3-20 | 3 | 1 | 17 | 66.5 | 14.5 | 468 | 0 |
| IGHV3-21 | 50.2 | 134 | 1022.07 | 6604.28 | 3812.62 | 4366.37 | 0 |
| IGHV3-23 | 977.17 | 3697.5 | 1825.83 | 9721.33 | 5682 | 3950.67 | 0 |
| IGHV3-23D | 24.5 | 63.5 | 48 | 241.83 | 98.5 | 91.33 | 0 |
| IGHV3-30 | 370.47 | 589.6 | 48347.77 | 20210.6 | 1909.52 | 10164.79 | 0 |
| IGHV3-30-3 | 102.54 | 915.42 | 6111.08 | 18120.08 | 2307.42 | 6901.46 | 0 |
| IGHV3-30-5 | 293.93 | 94.27 | 45901.93 | 9214.06 | 724.56 | 5360.17 | 0 |
| IGHV3-33 | 85.95 | 270.37 | 2016.57 | 29608.11 | 948.19 | 5157.98 | 0 |
| IGHV3-38-3 | 0 | 0 | 0 | 0.5 | 0.5 | 0 | 0 |
| IGHV3-43 | 0 | 0 | 19 | 212 | 59.5 | 186 | 0 |
| IGHV3-48 | 164.5 | 45 | 647.87 | 4076.58 | 787.42 | 2965.17 | 0 |
| IGHV3-49 | 0.2 | 3.2 | 95 | 322.7 | 5.2 | 414.2 | 0 |
| IGHV3-53 | 4.33 | 298.5 | 108.83 | 663.58 | 85.08 | 495.5 | 0 |
| IGHV3-64 | 0 | 11 | 76.7 | 234.5 | 65.5 | 130 | 0 |
| IGHV3-64D | 6 | 3 | 14.5 | 450.33 | 6 | 53 | 0 |
| IGHV3-66 | 60.33 | 190 | 738.83 | 2621.08 | 218.58 | 1972 | 0 |
| IGHV3-72 | 4 | 3 | 279 | 133.5 | 48.5 | 20.5 | 0 |
| IGHV3-73 | 1 | 5 | 9 | 221 | 51 | 22 | 0 |
| IGHV3-74 | 26 | 16 | 132.5 | 1448.5 | 774 | 590 | 0 |
| IGHV3/OR16-9 | 0 | 0 | 0 | 0 | 0 | 0 | 0 |
| IGHV3/OR16-10 | 0 | 0 | 0 | 0 | 0 | 0 | 0 |
| IGHV3/OR16-13 | 0 | 0 | 0 | 0 | 0 | 0 | 0 |
| IGHV3-NL1 | 1 | 11 | 28.28 | 44.5 | 35.5 | 21.33 | 0 |
| IGHV4-4 | 29.2 | 1683.7 | 1087.03 | 10193.95 | 1579.03 | 5819 | 0 |
| IGHV4-28 | 0 | 0 | 5 | 79.33 | 4 | 10.17 | 0 |
| IGHV4-30-2 | 7 | 228 | 21 | 511.5 | 459.17 | 105.25 | 0 |
| IGHV4-30-4 | 37.17 | 5450.42 | 3075.25 | 8768.75 | 1037.92 | 3763.75 | 1 |
| IGHV4-31 | 7.33 | 159.67 | 507.17 | 4632.58 | 501.58 | 1150.33 | 0 |
| IGHV4-34 | 1466 | 2259.33 | 1837.5 | 1170315.25 | 8529.08 | 16395.08 | 1 |
| IGHV4-38-2 | 0 | 0 | 0 | 9.25 | 2 | 9.58 | 0 |
| IGHV4-39 | 74 | 548.17 | 357.17 | 5487.75 | 3203 | 2286.25 | 0 |
| IGHV4-59 | 563.75 | 1556.33 | 5303 | 22174.42 | 5231.83 | 71387.5 | 0 |
| IGHV4-61 | 9.33 | 31.5 | 59.67 | 365.17 | 47.67 | 846.25 | 0 |
| IGHV4/OR15-8 | 0 | 0 | 2 | 18.75 | 3 | 5.17 | 0 |
| IGHV5-10-1 | 1092.6 | 37 | 4460.5 | 3841.1 | 6065.1 | 5284.8 | 0 |
| IGHV5-51 | 123.87 | 300.37 | 80036 | 17000 | 6464.4 | 14071 | 1 |
| IGHV6-1 | 7.33 | 9 | 12 | 654.83 | 330.83 | 778 | 0 |

TABLE 6
Summary of IgG VH-only sequencing of donor 2. Libraries generated from
these sequences were used for IgSeq proteomics and as a starting point
for YSD selections. Values represent the number of sequences.

| | IGHJ1 | IGHJ2 | IGHJ3 | IGHJ4 | IGHJ5 | IGHJ6 | ! Not Found |
|---|---|---|---|---|---|---|---|
| ! Not Found | 0 | 0 | 0 | 0 | 0 | 0 | 155 |
| IGHV1-2 | 57.33 | 1227.5 | 3458.5 | 19007 | 16592 | 28596 | 1 |
| IGHV1-3 | 0 | 3 | 54 | 17 | 3 | 147.5 | 0 |
| IGHV1-8 | 91.83 | 995 | 1416 | 4302.2 | 1359.3 | 4323 | 0 |
| IGHV1-18 | 20 | 393.5 | 1025 | 6924.3 | 2019.7 | 7911.3 | 0 |
| IGHV1-24 | 30.75 | 619.5 | 2159.3 | 11910 | 20044 | 6137.8 | 0 |
| IGHV1-45 | 0 | 14 | 3814.5 | 24.5 | 11.5 | 24 | 0 |
| IGHV1-46 | 293 | 1034.5 | 1460 | 8983.2 | 4676.3 | 20695 | 0 |
| IGHV1-58 | 0 | 288 | 26 | 132 | 11 | 95 | 0 |
| IGHV1-69 | 53.7 | 54464 | 1341.3 | 11794 | 4957.9 | 4948.2 | 0 |
| IGHV1-69D | 0.5 | 13.5 | 202.5 | 23.83 | 12.5 | 19.5 | 0 |
| IGHV1/OR15-1 | 0 | 1 | 0 | 1 | 0.5 | 2 | 0 |
| IGHV2-5 | 23 | 24.5 | 85 | 1496 | 7888.5 | 88 | 0 |
| IGHV2-26 | 0 | 4 | 11 | 416.17 | 7395 | 78.5 | 0 |
| IGHV2-70 | 10 | 12 | 1111.5 | 14093 | 1674.5 | 2137.5 | 0 |
| IGHV2-70D | 0 | 0 | 4 | 126.17 | 28.5 | 23 | 0 |
| IGHV3-7 | 50 | 116.33 | 900 | 6944.1 | 1168.7 | 2385.6 | 0 |
| IGHV3-9 | 118.83 | 246 | 3983.5 | 4532.8 | 1451.1 | 1795.3 | 0 |
| IGHV3-11 | 75 | 27.83 | 304.5 | 2348.2 | 156 | 885 | 0 |
| IGHV3-13 | 6 | 85 | 189 | 246.5 | 57.5 | 742 | 0 |
| IGHV3-15 | 17 | 13 | 159.83 | 2302.5 | 192.5 | 557.5 | 0 |
| IGHV3-20 | 7.33 | 32 | 76.5 | 907.33 | 131.83 | 255 | 0 |
| IGHV3-21 | 331.5 | 451.83 | 621.33 | 7024.8 | 1441.4 | 4789 | 0 |
| IGHV3-23 | 101.03 | 1005.5 | 573.87 | 13230 | 1069.5 | 3264.3 | 0 |
| IGHV3-23D | 2.5 | 26.5 | 11 | 255.5 | 22 | 62.5 | 0 |
| IGHV3-30 | 516.92 | 1371.7 | 14130 | 42894 | 11677 | 20746 | 0 |
| IGHV3-30-3 | 342.08 | 944.83 | 5553.7 | 24857 | 4072.5 | 19721 | 0 |
| IGHV3-30-5 | 189.25 | 318.5 | 11695 | 26273 | 7976.6 | 7550.6 | 0 |
| IGHV3-33 | 16.42 | 33.5 | 109.08 | 1043.7 | 191.33 | 422.58 | 0 |
| IGHV3-38-3 | 0 | 0 | 0 | 1 | 0 | 0 | 0 |
| IGHV3-43 | 1 | 9 | 1039 | 246.25 | 58.25 | 141 | 0 |
| IGHV3-48 | 176.5 | 260.5 | 1420.8 | 6461.6 | 925 | 4593.7 | 0 |
| IGHV3-49 | 4 | 7 | 95 | 298.5 | 53.5 | 1870 | 0 |
| IGHV3-53 | 7.5 | 893.5 | 144.5 | 839.42 | 131.67 | 1195.8 | 0 |
| IGHV3-64 | 6 | 14 | 160 | 2175.5 | 929 | 283.5 | 0 |
| IGHV3-64D | 0 | 0 | 0 | 1.5 | 1 | 0 | 0 |
| IGHV3-66 | 12 | 330 | 1016.5 | 3322.8 | 461 | 2092.8 | 0 |
| IGHV3-72 | 2 | 87 | 222 | 681.5 | 142 | 121.5 | 0 |
| IGHV3-73 | 13.2 | 10.2 | 39 | 1670.2 | 92.2 | 702.2 | 0 |
| IGHV3-74 | 56 | 64.5 | 172.5 | 1643 | 319.5 | 771 | 0 |
| IGHV3/OR16-9 | 0 | 0 | 0 | 1 | 0 | 0 | 0 |
| IGHV3/OR16-10 | 0 | 0 | 0 | 0 | 0 | 4 | 0 |
| IGHV3/OR16-13 | 0 | 0 | 0 | 1 | 0 | 0 | 0 |
| IGHV3-NL1 | 4 | 8 | 14 | 120.12 | 14.79 | 63 | 0 |
| IGHV4-4 | 237.75 | 1529.9 | 670.33 | 15879 | 6826.1 | 8480.7 | 0 |
| IGHV4-28 | 0 | 10.5 | 1 | 19 | 0 | 0 | 0 |
| IGHV4-30-2 | 24 | 156.17 | 539.5 | 2481.6 | 508.75 | 482.58 | 0 |
| IGHV4-30-4 | 31.58 | 671.25 | 272.5 | 4150.9 | 1137.3 | 2601.3 | 0 |
| IGHV4-31 | 25 | 578.17 | 1806.5 | 9777.2 | 2484.7 | 3506.9 | 0 |
| IGHV4-34 | 347.12 | 70963 | 4819 | 23585 | 9575.7 | 34140 | 1 |
| IGHV4-38-2 | 0 | 11.53 | 4 | 197.33 | 66 | 25.83 | 0 |
| IGHV4-39 | 471 | 895 | 1294.5 | 15740 | 7251.5 | 3844.6 | 0 |
| IGHV4-59 | 403 | 12207 | 6947.3 | 484719 | 26660 | 43651 | 1 |
| IGHV4-61 | 7.5 | 148.33 | 103.5 | 2888.8 | 358.92 | 543.58 | 0 |
| IGHV4/OR15-8 | 0 | 1.37 | 0 | 3.83 | 0.5 | 0.67 | 0 |
| IGHV5-10-1 | 0 | 0 | 0 | 1 | 0 | 2 | 0 |
| IGHV5-51 | 295.5 | 670.5 | 3864.3 | 11803 | 3453.7 | 2380.3 | 0 |
| IGHV6-1 | 3175.4 | 79 | 55 | 1193.4 | 103.42 | 611.75 | 0 |

TABLE 7
Anti-SARS-CoV-2 ECD and RBD antibody clonotypes identified in the serum of
patients 1 and 2 (P1, P2) by Ig-Seq proteomic analysis. Values represent the Log₁₀ of the
relative abundances of unique clonotypes calculated as the sum of XIC peak areas of CDR3-
peptides observed by LC-MS/MS. ND, not detected.

| | | SEQ ID | ECD | RBD |
|---|---|---|---|---|
| Donor | Representative CDR3 | NO: | Log₁₀ (XIC) | Log₁₀ (XIC) |
| 1 | AKASQLFWLGQFTRDGFDIW | 243 | 8.74 | 8.46 |
| 1 | ARDVFDPTAVSPIGGFDYW | 244 | 8.36 | ND |
| 1 | ARGVGAMQLWLRGYYFDSW | 245 | 8.21 | ND |
| 1 | ARGGRPITILGAVLAGGRNYFDPW | 246 | 7.90 | 7.71 |
| 1 | ARDTFDPIDSKEGGWFDPW | 247 | 7.84 | ND |
| 1 | ATGSPFDRTQNWEDPW | 248 | 7.77 | ND |
| 1 | ARRESPPGYW | 249 | 6.86 | 7.57 |
| 1 | ARHLGSGRYLRYLQHW | 250 | 6.91 | 7.36 |
| 1 | ARDAHCIKGVCTHHGMDVW | 251 | 7.32 | ND |
| 1 | AHRTHALPPSVAGFDYW | 252 | 7.25 | ND |
| 1 | AKGRKGAALDYW | 253 | 5.55 | 7.06 |
| 1 | AKDYTRTDWYGGEVIDYW | 254 | 6.09 | ND |
| 1 | ARTCKGSPGGRDGYNCLDYW | 255 | ND | 5.78 |
| 1 | ARAPRGIAGDYW | 256 | 5.40 | 5.72 |
| 1 | ARASEGRDIGGGYW | 257 | 5.42 | 5.56 |
| 2 | AREGFGVVIDYW | 258 | 8.86 | 8.54 |
| 2 | AHRVPRMELWLRGGWEDPW | 259 | 8.68 | ND |
| 2 | TKNRGGPSDHW | 260 | 8.39 | 8.61 |
| 2 | ATAAGVRGRGTIDYW | 261 | 8.54 | 5.21 |
| 2 | ARADSCNGGICYYRGMDVW | 262 | 8.25 | ND |
| 2 | ARHMGLQLWLRAHDAFDLW | 263 | 8.15 | ND |
| 2 | ARLRGVEDIVVVPAARTYNWFDPW | 264 | ND | 7.81 |
| 2 | ATDGYCPGGVCHRLGSFDFW | 265 | 7.77 | ND |
| 2 | ARGTIYFDRSGYRRVDPFHIW | 266 | 7.70 | 6.55 |
| 2 | ARHGPALYNYGSGAYLDYW | 267 | 7.64 | 6.08 |
| 2 | ARIPIATHLGSDYW | 268 | ND | 7.64 |
| 2 | ARRQWTFDYW | 269 | 5.92 | 7.63 |
| 2 | VRGRRGGYNSPDYW | 270 | ND | 7.48 |
| 2 | ASPEASYYYGDRGHFFPARRIDFDFW | 271 | 7.48 | 7.25 |
| 2 | ARQNVVLRFLERASRFDPW | 272 | ND | 7.34 |
| 2 | ASPTVTM | 273 | 7.28 | ND |
| 2 | ARVGTTVTANYYYGMDVW | 274 | 5.60 | 7.28 |
| 2 | ATRLEYNTPEKKPFDFW | 275 | 7.17 | 7.25 |
| 2 | ARDYGRGGVW | 276 | 7.10 | 7.12 |
| 2 | ARRSGSGWTWGYFDYW | 277 | ND | 6.42 |
| 2 | ARIPRDGYNFIDFW | 278 | 5.89 | 6.17 |

### Example 3—Parallel Selections Via Yeast Surface Display Yield Functionally Diverse Antibodies

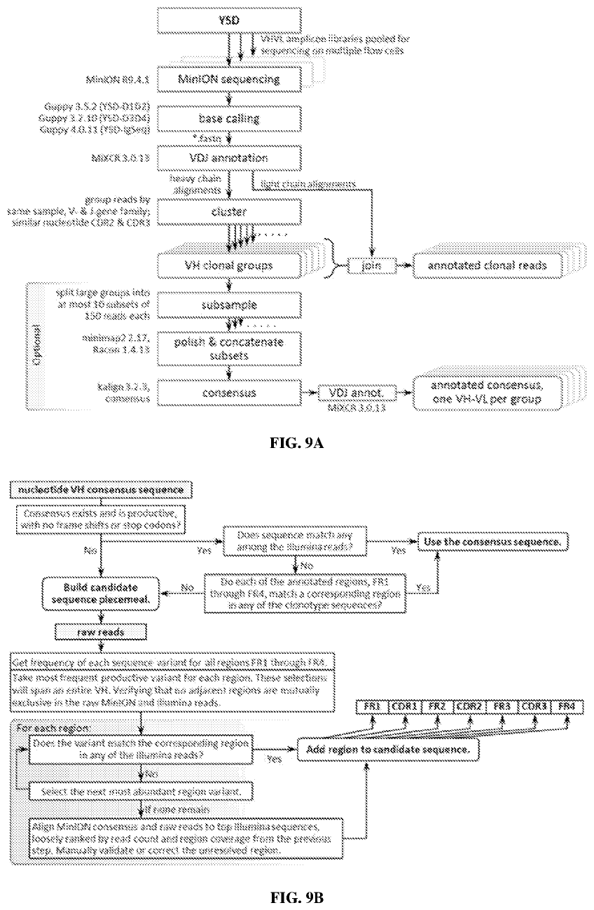

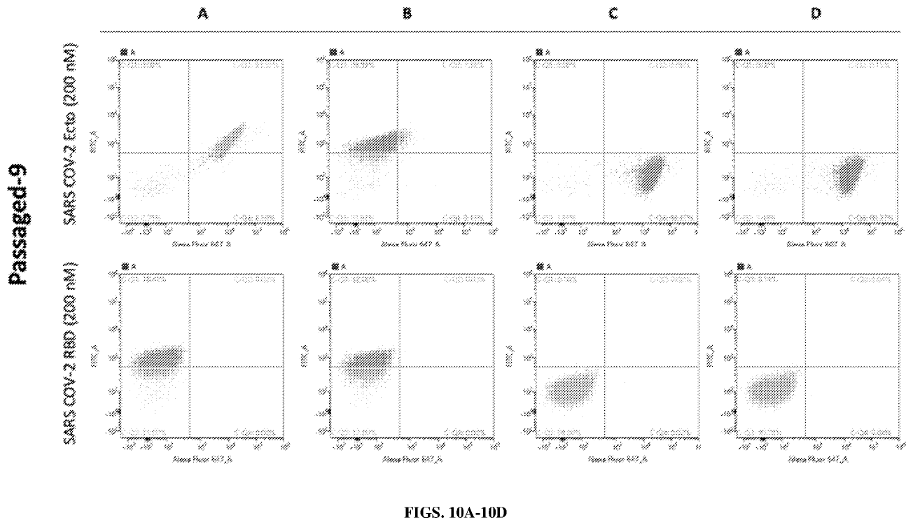

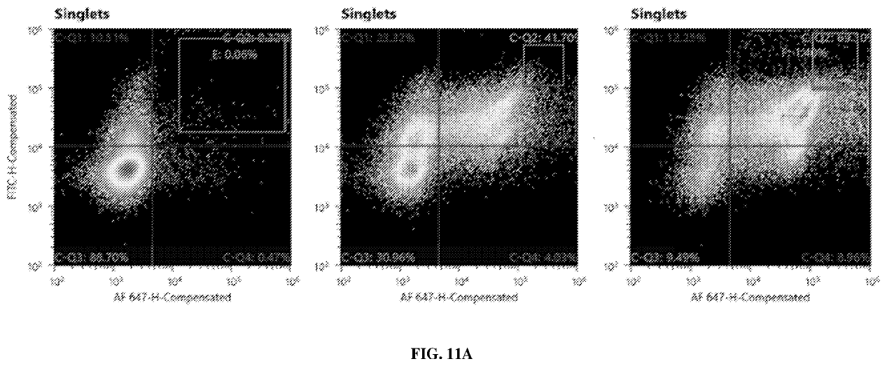

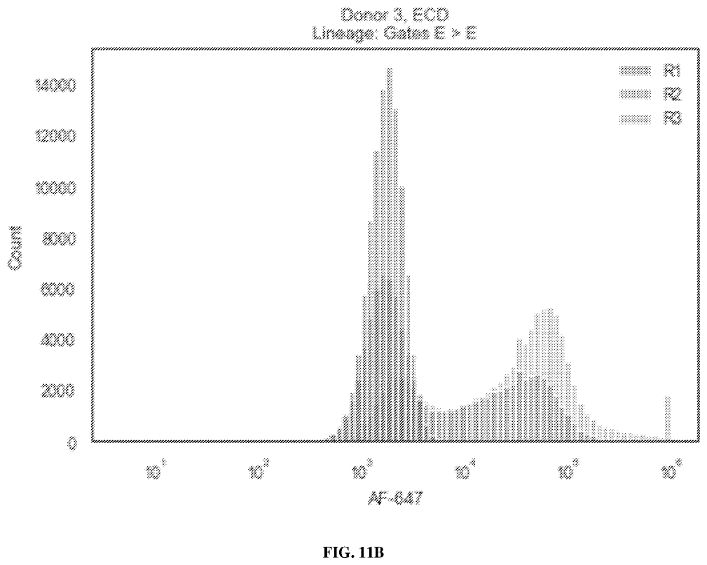

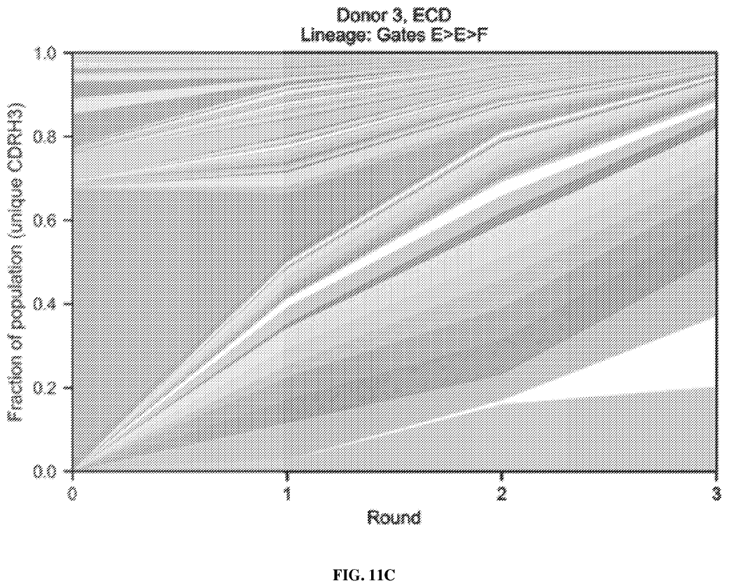

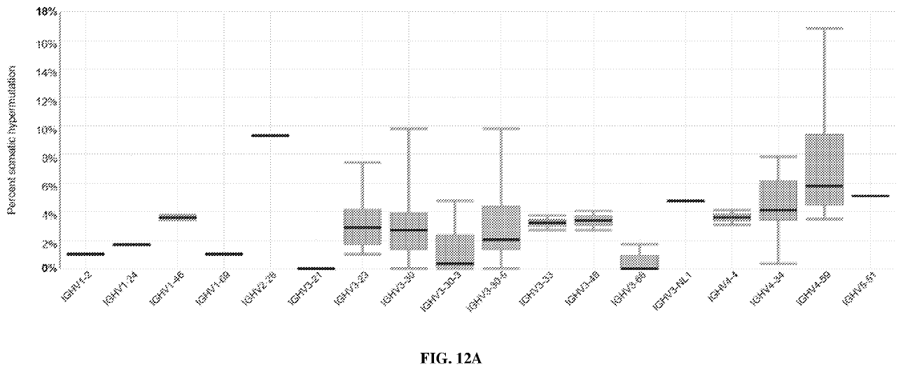

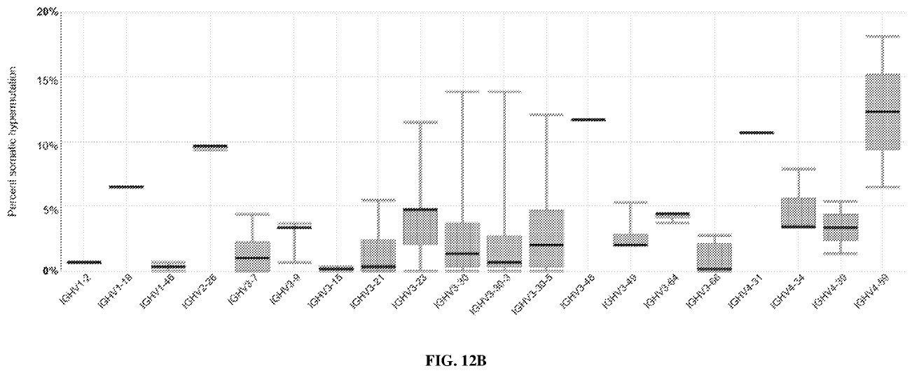

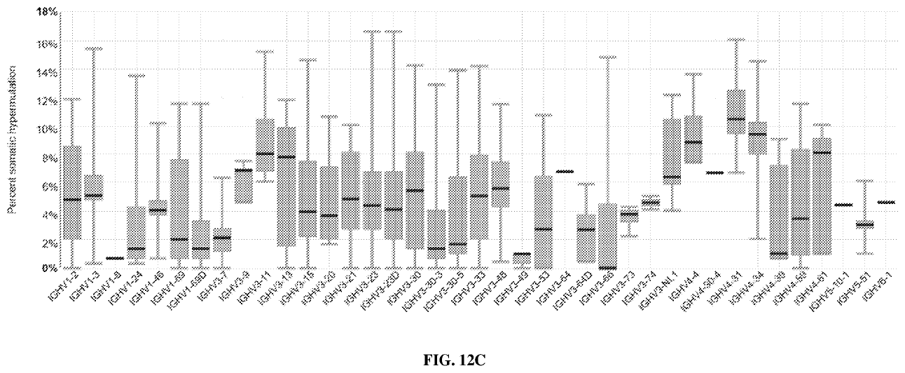

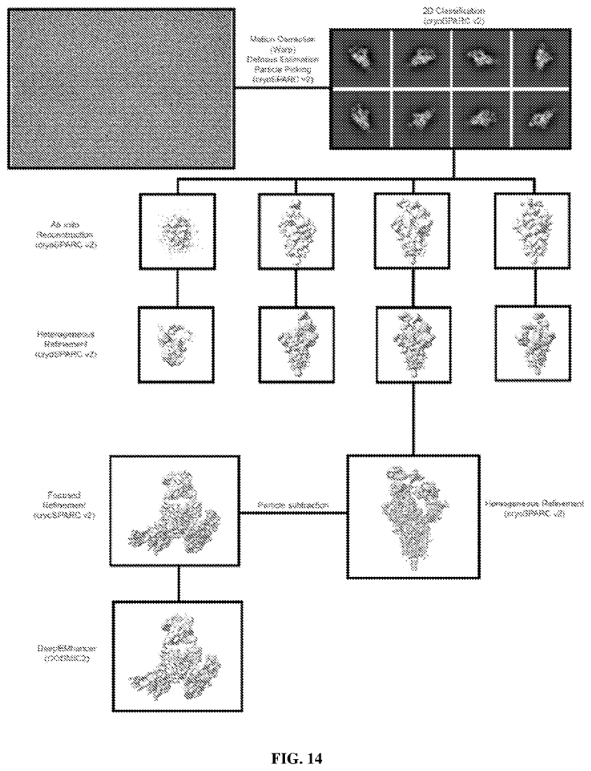

Much of the early stage primary antibody response is inaccessible to Ig-Seq discovery, as detection is limited to secreted antibody proteins with sufficient abundance in serum (Lavinder et al., 2014; U.S. Pat. No. 9,146,241, which is incorporated by reference herein in its entirety). To broaden the search for neutralizing antibodies, a humanized YSD platform (FIG. 19) was used to directly mine the cellular repertoire for high affinity B-cell receptor sequences by combinatorially assembling donor VH-VL pairs (FIGS. 1D, 13, 14, and Table 16). Large VH-VL libraries (>10⁷) created by combinatorial pairing of donor IgG heavy and light chain amplicons were expressed and displayed. To maximize the functional diversity of selected antibodies, three rounds of selection were performed on the initial libraries, using various gating strategies to ensure phenotypic diversity (i.e., a variety of antibody expression and antigen binding strengths). Ultimately, the parallelized selection strategy yielded populations with distinct binding characteristics as confirmed by cytometric phenotyping of single clones (FIGS. 9A,9B,10). Progressive enrichment of specific antibodies was observed with the dominant clone from each course of selections generally approaching 20% of the total population after round three (FIGS. 11A-11C; Table 17). Individual clones from enriched populations were selected as candidates for further characterization either at random or based on their abundance from the bioinformatic analysis. Importantly, YSD detected enrichment of VH sequences that were not observed by Ig-Seq proteomics, demonstrating the increased sensitivity of this multi-pronged approach to antibody discovery.

TABLE 17
Table showing the top HCDR3s as ranked by enrichment in this lineage. Relative
ranking by raw readcount is also compared. The bolded HCDR3 represents
neutralizing mAb 7-6.

| | | | | Rank by | |
|---|---|---|---|---|---|
| | | SEQ ID | Enrichment | read | |
| Rank | CDRH3 | NO: | factor | count | Change |
| 1 | ATASPHCSSTSCSINWFDP | 279 | 9988 | 2 | 1 |
| 2 | ASGGGSYYSPFDY | 280 | 2413 | 7 | 5 |
| 3 | ARAGGGGYQGPFDY | 281 | 1991 | 1 | −2 |
| 4 | ARAYGGGYLTPFDY | 282 | 1391 | 4 | 0 |
| 5 | AKPQGGGYNGAFDI | 283 | 720 | 6 | 1 |
| 6 | ATVSPFWSGYYNNWFDP | 284 | 407 | 3 | −3 |
| 7 | ARPVTGGYYYYMDV | 285 | 380 | 8 | 1 |
| 8 | TTERYYDYIWGSYRYSDY | 286 | 342 | 10 | 2 |
| 9 | ARDLRGSYSGLLDY | 287 | 307 | 11 | 2 |
| 10 | ATRFAVYGDYLIDY | 97 | 286 | 9 | −1 |
| 11 | AKVVRHLYSSGYSNWFDP | 288 | 233 | 20 | 9 |
| 12 | ANQVTYYDYIWGSYRPGAFDI | 289 | 150 | 13 | 1 |
| 13 | ARDKGRGILFTHRDAFDI | 290 | 140 | 29 | 16 |
| 14 | ARRRVYPSGFDY | 291 | 138 | 18 | 4 |
| 15 | ARASHGGYSSAFDP | 292 | 131 | 15 | 0 |
| 16 | VRSGHCNGGVCYGAEYFQH | 293 | 112 | 31 | 15 |
| 17 | ARDGSGGYSNPDY | 294 | 111 | 12 | −5 |
| 18 | VKGAGGYFDS | 295 | 110 | 32 | 14 |
| 19 | ARAPAVVVGNWFDP | 296 | 94 | 34 | 15 |
| 20 | ARGLDIIVVPAALDH | 297 | 92 | 5 | −15 |

### Example 4—Public Light Chains Recover Productive VH-VL Pairs

While Ig-Seq provides valuable information for antibody heavy chain discovery, it does not identify light chain partners, which usually requires additional laborious techniques. Further, the combinatorial pairing of VH-VL in YSD may not resolve optimal antibody candidates due to limitations in transformation of libraries. To expedite light chain discovery, a published dataset of natively-paired memory B-cell sequences (DeKosky et al., 2015) from three healthy donors was analyzed and a panel of nine light chains that show high frequencies of productive pairings with a diverse set of VH sequences was bioinformatically derived (FIGS. 1C and 2A).

At the time of blood draw, the donors in the dataset were healthy and asymptomatic. This lack of polarization and relative homeostasis potentially affords broader insight into common VH-VL pairings, independent of antigen specificity. Indeed, many of the observed light chains paired promiscuously with a diverse set of VH genes. The V-gene usage of undiscerning light chains was remarkably consistent among the three donors and therefore were termed “public light chains” (PLCs). From a secondary analysis of this dataset, six kappa V genes were identified that accounted for 61% of the kappa VH-VL pairs and three lambda V genes representing 22% of lambda pairs (FIG. 2A). Together, these nine public light chain (PLC) V genes accounted for 43% of observed VH-VL pairs in the analyzed repertoires.

This abundance could indicate an enhanced ability to form productive VH-VL pairs independent of VH sequence and antigen specificity. To test this hypothesis, germline versions of these light chains were constructed with their most frequently observed J gene as published on the iRepertoire website (Immune Repertoire Sequencing available at irweb.irepertoire.com/nir/showsample?sampleid=21746 #; Immune Repertoire Sequencing available at irweb.irepertoire.com/nir/showsample?sampleid=21745; Immune Repertoire Sequencing available at irweb.irepertoire.com/nir/showsample?sampleid=21744; Immune Repertoire Sequencing available at irweb.irepertoire.com/nir/showsample?sampleid=21743) (Tables 8 and 18) and full-length light chains were obtained for each PLC. SARS-COV-2 specific Ig-Seq heavy chains were then expressed with each PLC as a full-length IgG1. EC₅₀ determination via ELISA using recombinant spike protein demonstrated that of the 22 VHs tested, all paired productively with one or more of the PLCs, resulting in several highly potent and specific antibodies (FIG. 2A and Table 9).

Through this simple and rapid screening method, a panel of functional antibody candidates were identified for SARS-COV-2 neutralization testing. Seventeen neutralizing VH-PLC pairs, composed of 10 unique VHs (FIG. 2B and Tables 8 and 9), were recovered. Interestingly, NTD-directed VHs showed strong preference for lambda PLCs, most commonly PLC-8 (IGLV1-51). Four VHs produced neutralizing antibodies with more than one PLC. The VH N3, for example, revealed itself to be a highly potent RBD-directed binder, which showed the greatest neutralization potential with PLC-1 (IC50=0.25 nM), and formed additional neutralizing pairs with PLC-3 (IC50=13.1 nM) and PLC-7 (IC50=27.7 nM). Structural analysis of N3-1 in complex with the spike protein, revealed that PLC-1 not only serves to stabilize N3 but actively takes part in binding to a quaternary epitope.

TABLE 8
List of human public light chains screened in this study. Light chain V-genes were
derived from a previously published dataset of homeostatic repertoires from three donors.
For each of these V-genes, its most commonly associated germline J-gene was chosen.

| | V- | J- | | | | Full-length |
|---|---|---|---|---|---|---|
| Name | Gene | Gene | Light CDR1 | Light CDR2 | Light CDR3 | Light |
| PLC1 | IGKV1- | IGKJ1 | QSISSW | DAS | QQYNSYSPWT | SEQ ID NO: |
| | 5 | | (SEQ ID NO: | (SEQ ID NO: | (SEQ ID NO: 89) | 2 |
| | | | 87) | 88) | | |
| PLC2 | IGKV2- | IGKJ2 | QSLLHSNGY | LGS | MQALQTPPYT | SEQ ID NO: |
| | 28 | | NY | (SEQ ID NO: | (SEQ ID NO: 148) | 29 |
| | | | (SEQ ID NO: | 147) | | |
| | | | 146) | | | |
| PLC3 | IGKV3- | IGKJ4 | QSVSSY | DAS | QQRSNWPPLT | SEQ ID NO: |
| | 11 | | (SEQ ID NO: | (SEQ ID NO: | (SEQ ID NO: 119) | 15 |
| | | | 118) | 88) | | |
| PLC4 | IGKV3- | IGKJ1 | QSVSSN | GAS | QQYNNWPPWT | SEQ ID NO: |
| | 15 | | (SEQ ID NO: | (SEQ ID NO: | (SEQ ID NO: 129) | 19 |
| | | | 127) | 128) | | |
| PLC5 | IGKV3- | IGKJ1 | QSVSSSY | GAS | QQYGSSPPWT | SEQ ID NO: |
| | 20 | | (SEQ ID NO: | (SEQ ID NO: | (SEQ ID NO: 138) | 23 |
| | | | 137) | 128) | | |
| PLC6 | IGKV4- | IGKJ2 | QSVLYSSNN | WAS | QQYYSTPPYT | SEQ ID NO: |
| | 1 | | KNY | (SEQ ID NO: | (SEQ ID NO: 111) | 12 |
| | | | (SEQ ID NO: | 110) | | |
| | | | 109) | | | |
| PLC7 | IGLV1- | IGLJ3 | SSNIGSNT | SNN | AAWDDSLNGPV | SEQ ID NO: |
| | 44 | | (SEQ ID NO: | (SEQ ID NO: | V | 10 |
| | | | 103) | 104) | (SEQ ID NO: 105) | |
| PLC8 | IGLV1- | IGLJ3 | SSNIGNNY | DNN | GTWDSSLSAVV | SEQ ID NO: |
| | 51 | | (SEQ ID NO: | (SEQ ID NO: | (SEQ ID NO: 95) | 4 |
| | | | 93) | 94) | | |
| PLC9 | IGLV3- | IGLJ3 | KLGDKY | QDS | QAWDSSTVV | SEQ ID NO: |
| | 1 | | (SEQ ID NO: | (SEQ ID NO: | (SEQ ID NO: 143) | 26 |
| | | | 141) | 142) | | |

TABLE 9
Screening with VHs (H-CDR3 depicted) identified by Ig-Seq and YSD against the panel of nine PLCs to determine productive VH-VL
pairings. IgG mAbs ELISA EC₅₀s (nM) revealed that partnering VHs with PLCs can create low nM affinity binders. ND, no binding was
detected. †, binding detected, EC₅₀ incalculable. Bold values/symbols indicate pairs that neutralize SARS-CoV-2 WA1 live virus.

| | SEQ | | | | | | | | | | |
|---|---|---|---|---|---|---|---|---|---|---|---|
| | ID | | | | | | | | | | |
| VH CDR3 | NO: | label | PLC1 | PLC2 | PLC3 | PLC4 | PLC5 | PLC6 | PLC7 | PLC8 | PLC9 |
| ARDRSGSYYRGWFDP | 168 | N1 | >250 | 208.3 | ND | ND | >250 | 64.84 | ND | ND | >250 |
| ARGTIYFDRSGYRRVDPFHI | 86 | N3 | 0.1863 | 186.6 | 1.649 | ND | 99.63 | ND | 1.225 | ND | 76.34 |
| AKDTHSSSWRKYYYYYYGMDV | 130 | N4 | ND | ND | 21.99 | ND | 35.51 | ND | ND | ND | ND |
| APGRSLY | 163 | N5 | ND | 54.98 | ND | ND | 47.02 | 32.63 | ND | ND | ND |
| ARPYSGSYWGYFDY | 114 | N6 | >250 | 0.5606 | >250 | >250 | >250 | >250 | >250 | >250 | >250 |
| AKATRANYYYYYGMDV | 108 | N7 | >250 | 13.68 | >250 | >250 | 8.744 | ND | >250 | >250 | >250 |
| ARDRFQSYTYYYYGMDV | 175 | N10 | >250 | >250 | ND | ND | ND | ND | 71.93 | 69.02 | >250 |
| AKASQLFWLGQFTRDGFDI | 139 | 4A | ND | ND | ND | >250 | 31.66 | >250 | † | >250 | ND |
| ATGSPFDRTQNWFDP | 96 | 7A | ND | ND | ND | ND | ND | ND | >250 | 33.41 | >250 |
| ARELPPGRMVVPATYWHFDL | 136 | 8B | >250 | ND | >250 | >250 | † | >250 | >250 | >250 | ND |
| ARIPIATHLGSDY | 126 | 1D | † | >250 | >250 | † | † | ND | † | >250 | 8.111 |
| ARDWRVGATTQDVGY | 227 | 4B | 28.31 | >250 | ND | ND | ND | 72.35 | >250 | >250 | ND |
| AKDRAIFYLNPRYYLDY | 225 | 10B | † | ND | 64.66 | >250 | † | ND | >250 | † | † |
| AKATQLFWLGQFTRDGFDI | 228 | 1A | >250 | 123.8 | >250 | 187.4 | 145.1 | >250 | >250 | >250 | >250 |
| AKANGFNWKTPENGLDV | 229 | 2C | ND | >250 | >250 | ND | ND | ND | >250 | † | >250 |
| ARDGGGYVSY | 140 | 3B | ND | ND | ND | ND | ND | ND | ND | >250 | 1.987 |
| ARGVGAMQLWLRGYYFDS | 230 | 5A | >250 | 30.12 | >250 | >250 | >250 | ND | ND | ND | 145.6 |
| ATGPAVRRGSWFDP | 92 | 12C | ND | ND | ND | ND | >250 | ND | >250 | 11.25 | ND |
| ASPTVT | 194 | 9C | >250 | >250 | >250 | ND | >250 | ND | >250 | >250 | ND |
| ARRPPQCRNGICQHHLYGALDV | 231 | 5C | ND | >250 | ND | >250 | ND | ND | 152.5 | ND | ND |
| ATAAAVRGRGTIDY | 102 | 4C | ND | ND | ND | ND | ND | >250 | 6.031 | 1.616 | ND |
| ARARGGSYYYGMDV | 120 | B3 | 1.079 | >250 | 1.123 | 1.976 | 1.837 | 4.661 | >250 | >250 | >250 |

### Example 5—Yeast Surface Display-IgSeq Identifies Additional Candidate Antibodies

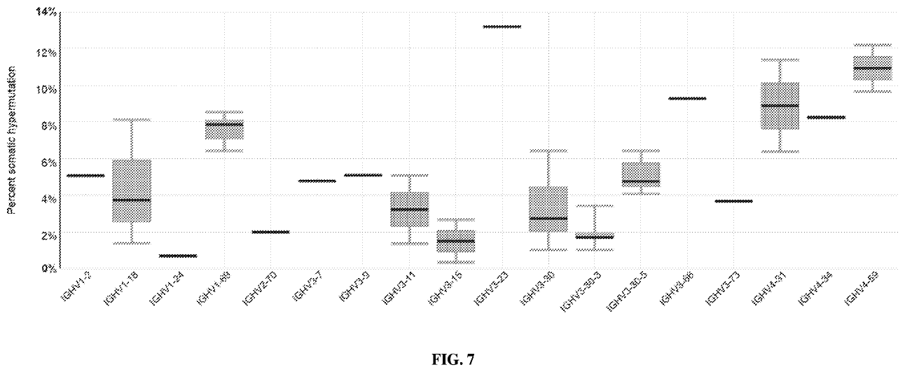

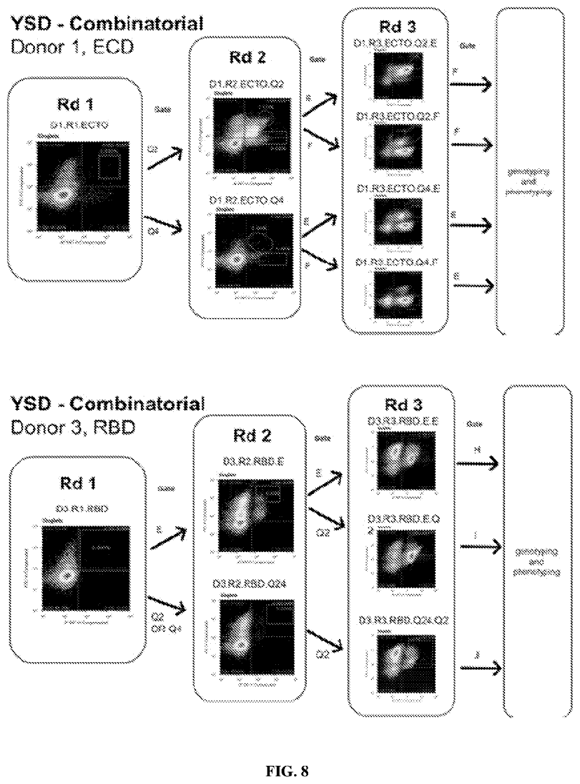

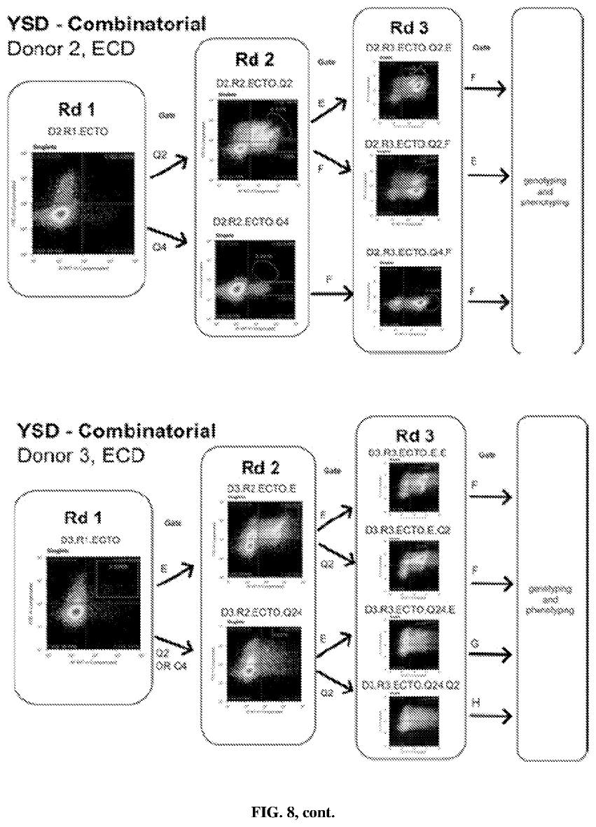

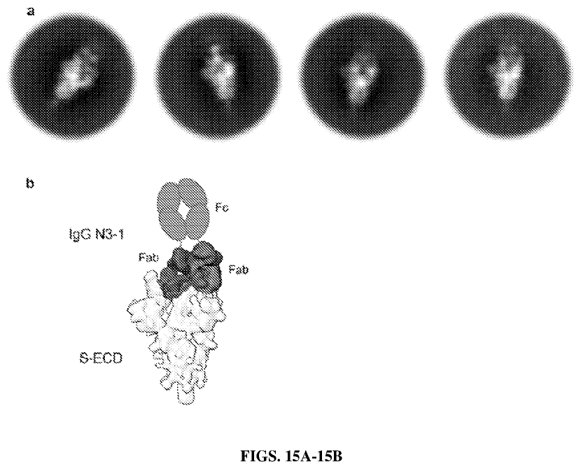

The PLC screening strategy provides a method to rapidly generate functionally paired antibodies, but the decreased sequence space of the nine PLCs may limit the ability to efficiently identify potent binders. Thus, to search for additional high-affinity antibodies, 24 Ig-Seq heavy chains were randomly paired with donor-derived light chain libraries for yeast surface display (YSD) selections (FIG. 1B), which allowed us to efficiently screen Fabs for each heavy chain/light chain library combination (FIGS. 8 and 11). In YSD, heavy/light chain pairs were displayed as Fab fragments on the surface of a humanized yeast strain that expresses human protein chaperones to improve antibody expression. Then, antigen-binding Fab fragments were selected using two-color fluorescence cell sorting to maximize Fab expression and binding to either SARS-COV-2 spike (S-2P) or RBD (FIGS. 7-8; Tables 11-12). As Fab enrichment over successive rounds of selection indicates antigen specificity, three rounds were performed and productive VH-VL pairs were identified and quantified based on MinION nanopore sequencing of Fab amplicons with sequence error correction and complementary Illumina reads (Tables 10 & 16; FIGS. 9A-9B and 15A-15B). When light chain preference was compared, extensive correspondence was observed between YSD selection and PLC screening.

IgSeq VHs successfully paired with donor VLs to produce 17 IgSeq-YSD neutralizing VH-VL pairs, including our most potent neutralizer A7V32 with an IC50 of 18 PM and three additional binders with subnanomolar IC50 values. A7V3 was of particular interest due to the different donor origins of its VH and VL. The A7 VH (IGHV1-24) was discovered in both IgSeq and YSD from donor 1. However, the VL (IGLV1-51) was mapped to donor 2 and is also related to PLC8. The VH of A7V3 showed a strong preference to PLC8 in ELISA screening, and related light chains appeared in six other neutralizing VH-VL pairs, commonly pairing with IGHV1-24 VHs.

TABLE 10
VH CDR3 read counts and relative abundance from the YSD-IgSeq experiment shown for MinION reads of 1.7 kb to 2.1 kb. Values
are CDR3 frequencies, or read counts normalized within each respective sample.

| | SEQ | | RBD | | | | | | | | antigen |
|---|---|---|---|---|---|---|---|---|---|---|---|
| | ID | Total | R1 | | R2 | | R3 | | | | round |
| VH CDR3 | NO: | reads | 1 | 2 | 5 | 6 | 9 | 10 | 11 | 12 | selection |
| AKTSGYNLPDY | 232 | 2467936 | 4746 | 21913 | 610618 | 933228 | 305045 | 257272 | 302117 | 26015 | |
| ARGTIYFDRSGYRRVDPFHI | 86 | 682539 | 62 | 62 | 215 | 265 | 115 | 0 | 0 | 0 | |
| ATGPAVRRGSWFDP | 92 | 189383 | 23492 | 54280 | 15688 | 2913 | 8677 | 19861 | 889 | 0 | |
| ARRQWTFDY | 233 | 170557 | 600 | 44 | 38 | 25 | 0 | 0 | 0 | 0 | |
| ATGSPFDRTQNWFDP | 96 | 150096 | 23502 | 60082 | 3732 | 2905 | 790 | 2480 | 945 | 0 | |
| ARDGGGYVSY | 140 | 137057 | 14203 | 40397 | 1176 | 1308 | 191 | 639 | 0 | 0 | |
| ARGVGAMQLWLRGYYFDS | 230 | 101311 | 1868 | 4754 | 30704 | 8968 | 18857 | 13973 | 1662 | 538 | |
| TTDRTYDYVWGSYRYEDY | 234 | 18251 | 1546 | 5486 | 172 | 130 | 0 | 0 | 0 | 0 | |
| ASPEASYYYGDRGHFFPARRIDFDF | 235 | 14123 | 3538 | 3459 | 58 | 159 | 0 | 0 | 160 | 0 | |
| TTDRSYDYVWGSYRYPDY | 221 | 13957 | 1166 | 7639 | 437 | 357 | 172 | 83 | 0 | 0 | |
| ARIPIATHLGSDY | 126 | 13507 | 2001 | 5807 | 145 | 264 | 0 | 0 | 0 | 5 | |
| ATAAAVRGRGTIDY | 102 | 11135 | 1377 | 5252 | 126 | 0 | 72 | 0 | 0 | 0 | |
| ARDYGRGGV | 218 | 9303 | 307 | 1418 | 152 | 177 | 0 | 50 | 0 | 0 | |
| AKDRAIFYLNPRYYLDY | 225 | 9245 | 288 | 171 | 0 | 0 | 0 | 0 | 0 | 0 | |
| ASPTVT | 194 | 8847 | 947 | 3482 | 122 | 245 | 0 | 0 | 741 | 15 | |
| ARELPPGRMVVPATYWHFDL | 136 | 7591 | 782 | 2891 | 21 | 446 | 0 | 0 | 458 | 30 | |
| TSPRADYGSNSVGNY | 236 | 5736 | 110 | 118 | 0 | 0 | 0 | 0 | 0 | 0 | |
| ARDGITNSGVVTHFGMDV | 237 | 4214 | 278 | 2188 | 46 | 274 | 0 | 234 | 101 | 0 | |
| ARHMGMQLWLRAHDAFDL | 238 | 908 | 84 | 44 | 22 | 7 | 0 | 0 | 19 | 0 | |
| ARLGTGYP | 239 | 655 | 127 | 0 | 0 | 0 | 0 | 0 | 0 | 0 | |
| ACSRARGFMFFDY | 240 | 631 | 76 | 139 | 14 | 23 | 9 | 0 | 2 | 0 | |
| AKATQLFWLGQFTRDGFDI | 228 | 366 | 0 | 0 | 0 | 0 | 0 | 0 | 0 | 0 | |
| ARADSCNGGICYYQGMDV | 241 | 215 | 25 | 26 | 6 | 3 | 0 | 0 | 6 | 0 | |
| AREQPPGRMVVPATYWHFDL | 242 | 70 | 0 | 0 | 0 | 0 | 0 | 0 | 0 | 0 | |
| | SEQ | | ECD | | | | | | | | antigen |
| | ID | Total | R1 | | R2 | | R3 | | | | round |
| VH CDR3 | NO: | reads | 3 | 4 | 7 | 8 | 13 | 14 | 15 | 16 | selection |
| AKTSGYNLPDY | 232 | 2467936 | 2257 | 2166 | 1002 | 1044 | 118 | 277 | 118 | 0 | |
| ARGTIYFDRSGYRRVDPFHI | 86 | 682539 | 4245 | 14770 | 31572 | 579414 | 2098 | 13607 | 31359 | 4755 | |
| ATGPAVRRGSWFDP | 92 | 189383 | 16256 | 10212 | 19927 | 8611 | 483 | 8039 | 51 | 4 | |
| ARRQWTFDY | 233 | 170557 | 18238 | 101 | 8176 | 130388 | 768 | 3086 | 7479 | 1614 | |
| ATGSPFDRTQNWFDP | 96 | 150096 | 14828 | 11766 | 20334 | 171 | 1077 | 7484 | 0 | 0 | |
| ARDGGGYVSY | 140 | 137057 | 26399 | 26912 | 9267 | 10889 | 856 | 4673 | 101 | 46 | |
| ARGVGAMQLWLRGYYFDS | 230 | 101311 | 5663 | 11064 | 2054 | 438 | 43 | 725 | 0 | 0 | |
| TTDRTYDYVWGSYRYEDY | 234 | 18251 | 3707 | 3514 | 1472 | 286 | 0 | 1938 | 0 | 0 | |
| ASPEASYYYGDRGHFFPARRIDFDF | 235 | 14123 | 2699 | 2484 | 654 | 557 | 123 | 214 | 0 | 18 | |
| TTDRSYDYVWGSYRYPDY | 221 | 13957 | 1191 | 648 | 1340 | 151 | 0 | 773 | 0 | 0 | |
| ARIPIATHLGSDY | 126 | 13507 | 1893 | 2152 | 382 | 51 | 16 | 791 | 0 | 0 | |
| ATAAAVRGRGTIDY | 102 | 11135 | 2241 | 1321 | 579 | 167 | 0 | 0 | 0 | 0 | |
| ARDYGRGGV | 218 | 9303 | 1792 | 1284 | 911 | 2965 | 49 | 114 | 11 | 73 | |
| AKDRAIFYLNPRYYLDY | 225 | 9245 | 3467 | 2871 | 892 | 485 | 64 | 1007 | 0 | 0 | |
| ASPTVT | 194 | 8847 | 1776 | 673 | 459 | 378 | 9 | 0 | 0 | 0 | |
| ARELPPGRMVVPATYWHFDL | 136 | 7591 | 448 | 1950 | 247 | 241 | 58 | 19 | 0 | 0 | |
| TSPRADYGSNSVGNY | 236 | 5736 | 1409 | 810 | 1209 | 1048 | 66 | 966 | 0 | 0 | |
| ARDGITNSGVVTHFGMDV | 237 | 4214 | 813 | 179 | 101 | 0 | 0 | 0 | 0 | 0 | |
| ARHMGMQLWLRAHDAFDL | 238 | 908 | 110 | 329 | 111 | 157 | 2 | 23 | 0 | 0 | |
| ARLGTGYP | 239 | 655 | 410 | 0 | 43 | 75 | 0 | 0 | 0 | 0 | |
| ACSRARGFMFFDY | 240 | 631 | 77 | 103 | 65 | 64 | 5 | 54 | 0 | 0 | |
| AKATQLFWLGQFTRDGFDI | 228 | 366 | 344 | 0 | 22 | 0 | 0 | 0 | 0 | 0 | |
| ARADSCNGGICYYQGMDV | 241 | 215 | 27 | 39 | 37 | 22 | 5 | 19 | 0 | 0 | |
| AREQPPGRMVVPATYWHFDL | 242 | 70 | 0 | 0 | 57 | 13 | 0 | 0 | 0 | 0 | |

TABLE 11
IgSeq VH gene usage.

| | IGHJ2 | IGHJ3 | IGHJ4 | IGHJ5 | IGHJ6 |
|---|---|---|---|---|---|
| IGHV1-2 | 1 | 0 | 0 | 0 | 0 |
| IGHV1-18 | 0 | 0 | 3 | 0 | 0 |
| IGHV1-24 | 0 | 0 | 0 | 2 | 0 |
| IGHV1-69 | 2 | 0 | 2 | 1 | 0 |
| IGHV2-70 | 0 | 0 | 1 | 0 | 0 |
| IGHV3-7 | 0 | 0 | 1 | 0 | 0 |
| IGHV3-9 | 0 | 1 | 0 | 0 | 0 |
| IGHV3-11 | 0 | 0 | 1 | 0 | 1 |
| IGHV3-15 | 0 | 0 | 2 | 0 | 0 |
| IGHV3-23 | 0 | 1 | 0 | 0 | 0 |
| IGHV3-30 | 0 | 1.5 | 5.5 | 0.5 | 0.5 |
| IGHV3-30-3 | 0 | 1 | 1.5 | 0.5 | 0.5 |
| IGHV3-30-5 | 0 | 1.5 | 1 | 0 | 0 |
| IGHV3-66 | 0 | 0 | 0 | 0 | 1 |
| IGHV3-73 | 0 | 0 | 1 | 0 | 0 |
| IGHV4-31 | 0 | 1 | 1 | 0 | 0 |
| IGHV4-34 | 0 | 1 | 0 | 0 | 0 |
| IGHV4-59 | 0 | 0 | 1 | 0 | 1 |

TABLE 12
YSD-IgSeq VL gene usage.

| | IGKJ1 | IGKJ2 | IGKJ3 | IGKJ4 | IGKJ5 | IGLJ1 | IGLJ2 | IGLJ3 |
|---|---|---|---|---|---|---|---|---|
| IGKV1-5 | 2 | 0 | 0 | 0 | 0 | 0 | 0 | 0 |
| IGKV1-8 | 0 | 0 | 0 | 0 | 2 | 0 | 0 | 0 |
| IGKV1-39 | 3 | 0.5 | 0 | 2 | 0 | 0 | 0 | 0 |
| IGKV1D-39 | 3 | 0.5 | 0 | 2 | 0 | 0 | 0 | 0 |
| IGKV4-1 | 0 | 2 | 1 | 0 | 0 | 0 | 0 | 0 |
| IGLV1-40 | 0 | 0 | 0 | 0 | 0 | 0 | 0 | 3 |
| IGLV1-50 | 0 | 0 | 0 | 0 | 0 | 0 | 0 | 1 |
| IGLV1-51 | 0 | 0 | 0 | 0 | 0 | 0 | 0.5 | 2.5 |
| IGLV2-8 | 0 | 0 | 0 | 0 | 0 | 1 | 0 | 0 |
| IGLV3-1 | 0 | 0 | 0 | 0 | 0 | 0 | 0.5 | 0.5 |
| IGLV3-21 | 0 | 0 | 0 | 0 | 0 | 0 | 0 | 1 |

TABLE 13
YSD selected VHs from donor 1 after three rounds of selection.

| | IGHJ2 | IGHJ3 | IGHJ4 | IGHJ5 | IGHJ6 |
|---|---|---|---|---|---|
| IGHV1-2 | 0 | 0 | 1 | 0 | 0 |
| IGHV1-24 | 0 | 0 | 0 | 1 | 0 |
| IGHV1-46 | 0 | 0 | 0 | 0 | 2 |
| IGHV1-69 | 0 | 0 | 0 | 0 | 1 |
| IGHV2-26 | 0 | 0 | 0 | 1 | 0 |
| IGHV3-21 | 0 | 0 | 0 | 0 | 1 |
| IGHV3-23 | 1 | 0 | 6 | 0 | 9 |
| IGHV3-30 | 0 | 0 | 7.5 | 0 | 10 |
| IGHV3-30-3 | 0 | 0 | 3.5 | 0 | 4.5 |
| IGHV3-30-5 | 0 | 0 | 7 | 0 | 5.5 |
| IGHV3-33 | 0 | 0 | 0 | 0 | 2 |
| IGHV3-48 | 0 | 0 | 0 | 0 | 2 |
| IGHV3-66 | 0 | 0 | 0 | 0 | 5 |
| IGHV3-NL1 | 0 | 0 | 0 | 0 | 1 |
| IGHV4-4 | 0 | 0 | 2 | 0 | 0 |
| IGHV4-34 | 0 | 0 | 14 | 0 | 3 |
| IGHV4-59 | 0 | 0 | 2 | 0 | 5 |
| IGHV5-51 | 0 | 1 | 0 | 0 | 0 |

TABLE 14
YSD selected VHs from donor 2 after three rounds of selection.

| | IGHJ2 | IGHJ3 | IGHJ4 | IGHJ5 | IGHJ6 |
|---|---|---|---|---|---|
| IGHV1-2 | 0 | 0 | 0 | 0 | 4 |
| IGHV1-18 | 0 | 0 | 1 | 0 | 0 |
| IGHV1-46 | 0 | 0 | 2 | 0 | 2 |
| IGHV2-26 | 0 | 0 | 0 | 3 | 0 |
| IGHV3-7 | 0 | 0 | 2 | 0 | 4 |
| IGHV3-9 | 0 | 0 | 0 | 0 | 5 |
| IGHV3-15 | 0 | 0 | 0 | 0 | 2 |
| IGHV3-21 | 0 | 0 | 2 | 0 | 18 |
| IGHV3-23 | 0 | 0 | 4 | 0 | 9 |
| IGHV3-30 | 0 | 0 | 14.25 | 7.25 | 34.67 |
| IGHV3-30-3 | 0 | 1 | 23.25 | 3.25 | 40.17 |
| IGHV3-30-5 | 0 | 0 | 4 | 0 | 16.17 |
| IGHV3-48 | 0 | 0 | 0 | 0 | 4 |
| IGHV3-49 | 0 | 0 | 4 | 0 | 0 |
| IGHV3-64 | 0 | 0 | 1 | 0 | 2 |
| IGHV3-66 | 0 | 0 | 1 | 0 | 5 |
| IGHV4-31 | 0 | 2 | 0 | 0 | 0 |
| IGHV4-34 | 1 | 0 | 0 | 0 | 2 |
| IGHV4-39 | 0 | 0 | 1 | 0 | 1 |
| IGHV4-59 | 0 | 0 | 0 | 2 | 0 |

TABLE 15
YSD selected VHs from donor 3 after three rounds of selection.

| | IGHJ1 | IGHJ2 | IGHJ3 | IGHJ4 | IGHJ5 | IGHJ6 |
|---|---|---|---|---|---|---|
| IGHV1-2 | 0 | 0 | 2 | 19.5 | 3.5 | 7 |
| IGHV1-3 | 0 | 0 | 0 | 21 | 1 | 1 |
| IGHV1-8 | 0 | 0 | 0 | 0 | 0 | 1 |
| IGHV1-24 | 0 | 0 | 3 | 40 | 47 | 12 |
| IGHV1-46 | 0 | 0 | 1 | 2 | 1 | 5 |
| IGHV1-69 | 0 | 1 | 18.5 | 8 | 2.5 | 30 |
| IGHV1-69D | 0 | 0 | 1.5 | 0 | 0.5 | 5 |
| IGHV3-7 | 0 | 0 | 0 | 2 | 0 | 14 |
| IGHV3-9 | 0 | 0 | 0 | 5 | 0 | 0 |
| IGHV3-11 | 12 | 0 | 11 | 12.5 | 0 | 0.5 |
| IGHV3-13 | 0 | 0 | 3 | 0 | 4 | 0 |
| IGHV3-15 | 2 | 0 | 5 | 21 | 2 | 11 |
| IGHV3-20 | 0 | 0 | 0 | 15 | 5 | 4 |
| IGHV3-21 | 0 | 0 | 0 | 8.5 | 0 | 16 |
| IGHV3-23 | 0.5 | 2 | 16 | 58.25 | 28 | 23.5 |
| IGHV3-23D | 0.5 | 0 | 0 | 13.25 | 7 | 13.5 |
| IGHV3-30 | 4 | 7.17 | 14.17 | 89.5 | 3.58 | 19.33 |
| IGHV3-30-3 | 2 | 8 | 19 | 154.58 | 27.5 | 38 |
| IGHV3-30-5 | 2 | 7.17 | 11.17 | 27.25 | 0.83 | 7.83 |
| IGHV3-33 | 0 | 7.67 | 3.67 | 31.42 | 1.08 | 21.83 |
| IGHV3-48 | 0 | 1 | 0 | 3 | 0 | 3.5 |
| IGHV3-49 | 0 | 0 | 0 | 5 | 0 | 0 |
| IGHV3-53 | 0 | 0 | 1 | 9 | 0 | 0 |
| IGHV3-64 | 0 | 0 | 0 | 0.25 | 0 | 0 |
| IGHV3-64D | 4 | 0 | 0 | 1 | 0 | 0 |
| IGHV3-66 | 0 | 0 | 5 | 2 | 0 | 0 |
| IGHV3-73 | 0 | 0 | 2 | 2 | 0 | 0 |
| IGHV3-74 | 0 | 0 | 0 | 2 | 0 | 0 |
| IGHV3-NL1 | 0 | 0 | 0 | 4.5 | 1 | 3 |
| IGHV4-4 | 0 | 0 | 0 | 1 | 0 | 5 |
| IGHV4-30-4 | 0 | 0 | 0 | 0 | 0 | 0.5 |
| IGHV4-31 | 0 | 0 | 7 | 4 | 0 | 1.5 |
| IGHV4-34 | 0 | 0 | 0 | 16 | 0 | 14 |
| IGHV4-39 | 0 | 4 | 1 | 2 | 0 | 0 |
| IGHV4-59 | 0 | 0 | 0 | 3 | 4 | 4 |
| IGHV4-61 | 0 | 0 | 4 | 0 | 3 | 2 |
| IGHV5-10-1 | 0 | 0 | 0 | 0 | 1 | 0 |
| IGHV5-51 | 0 | 1 | 4 | 0 | 0 | 1 |
| IGHV6-1 | 0 | 0 | 0 | 0 | 0 | 1 |

TABLE 16
VH CDR3 read counts and relative abundance from the YSD-IgSeq experiment, shown for MinION reads of 1.7 kb to 2.1 kb, all
MinION reads, and all iSeq reads. Values are CDR3 frequencies, or read counts normalized within each respective sample. Length requirements
improve VHVL abundance estimation by, in particular, removing the inflated counts of short artifactual reads due to unbalanced PCR
amplification. Comparison with and without filtering shows certain CDR3 are disproportionately affected. The iSeq read counts for a VH CDR3
include only reads with identical amino acid CDR3 annotation. The respective MinION counts also include reads diversified through sequencing
error, recovered through our clustering methods. High MinION error rates create enormous sequence diversity. Full-length VH diversity is
bounded only by the total read count. (center, right panels) There is strong correspondence between the unfiltered MinION and iSeq CDR3
frequencies. This indicates that the independent MinION read consolidation techniques successfully recapitulate CDR3 patterns observed from
the much more accurate Illumina sequencing platform.
MinION, 1.7-21 kb-RBD

| | SEQ | | RBD | | | | | | | | antigen |
|---|---|---|---|---|---|---|---|---|---|---|---|
| | ID | Total | R1 | | R2 | | R3 | | | | round |
| VH CDR3 | NO: | reads | 1 | 2 | 5 | 6 | 9 | 10 | 11 | 12 | selection |
| AKTSGYNLPDY | 232 | 2467936 | 4746 | 21913 | 610618 | 933228 | 305045 | 257272 | 302117 | 26015 | |
| ARGTIYFDRSGYRRVDPFHI | 86 | 682539 | 62 | 62 | 215 | 265 | 115 | 0 | 0 | 0 | |
| ATGPAVRRGSWFDP | 92 | 189383 | 23492 | 54280 | 15688 | 2913 | 8677 | 19861 | 889 | 0 | |
| ARRQWTFDY | 233 | 170557 | 600 | 44 | 38 | 25 | 0 | 0 | 0 | 0 | |
| ATGSPFDRTQNWFDP | 96 | 150096 | 23502 | 60082 | 3732 | 2905 | 790 | 2480 | 945 | 0 | |
| ARDGGGYVSY | 140 | 137057 | 14203 | 40397 | 1176 | 1308 | 191 | 639 | 0 | 0 | |
| ARGVGAMQLWLRGYYFDS | 230 | 101311 | 1868 | 4754 | 30704 | 8968 | 18857 | 13973 | 1662 | 538 | |
| TTDRTYDYVWGSYRYEDY | 234 | 18251 | 1546 | 5486 | 172 | 130 | 0 | 0 | 0 | 0 | |
| ASPEASYYYGDRGHFFPARRIDFDF | 235 | 14123 | 3538 | 3459 | 58 | 159 | 0 | 0 | 160 | 0 | |
| TTDRSYDYVWGSYRYPDY | 221 | 13957 | 1166 | 7639 | 437 | 357 | 172 | 83 | 0 | 0 | |
| ARIPIATHLGSDY | 126 | 13507 | 2001 | 5807 | 145 | 264 | 0 | 0 | 0 | 5 | |
| ATAAAVRGRGTIDY | 102 | 11135 | 1377 | 5252 | 126 | 0 | 72 | 0 | 0 | 0 | |
| ARDYGRGGV | 218 | 9303 | 307 | 1418 | 152 | 177 | 0 | 50 | 0 | 0 | |
| AKDRAIFYLNPRYYLDY | 225 | 9245 | 288 | 171 | 0 | 0 | 0 | 0 | 0 | 0 | |
| ASPTVT | 194 | 8847 | 947 | 3482 | 122 | 245 | 0 | 0 | 741 | 15 | |
| ARELPPGRMVVPATYWHFDL | 136 | 7591 | 782 | 2891 | 21 | 446 | 0 | 0 | 458 | 30 | |
| TSPRADYGSNSVGNY | 236 | 5736 | 110 | 118 | 0 | 0 | 0 | 0 | 0 | 0 | |
| ARDGITNSGVVTHFGMDV | 237 | 4214 | 278 | 2188 | 46 | 274 | 0 | 234 | 101 | 0 | |
| ARHMGMQLWLRAHDAFDL | 238 | 908 | 84 | 44 | 22 | 7 | 0 | 0 | 19 | 0 | |
| ARLGTGYP | 239 | 655 | 127 | 0 | 0 | 0 | 0 | 0 | 0 | 0 | |
| ACSRARGFMFFDY | 240 | 631 | 76 | 139 | 14 | 23 | 9 | 0 | 2 | 0 | |
| AKATQLFWLGQFTRDGFDI | 228 | 366 | 0 | 0 | 0 | 0 | 0 | 0 | 0 | 0 | |
| ARADSCNGGICYYQGMDV | 241 | 215 | 25 | 26 | 6 | 3 | 0 | 0 | 6 | 0 | |
| AREQPPGRMVVPATYWHFDL | 242 | 70 | 0 | 0 | 0 | 0 | 0 | 0 | 0 | 0 | |

MinION, 1.7-21 kb-ECD

| | SEQ | | ECD | | | | | | | | antigen |
|---|---|---|---|---|---|---|---|---|---|---|---|
| | ID | Total | R1 | | R2 | | R3 | | | | round |
| VH CDR3 | NO: | reads | 3 | 4 | 7 | 8 | 13 | 14 | 15 | 16 | selection |
| AKTSGYNLPDY | 232 | 2467936 | 2257 | 2166 | 1002 | 1044 | 118 | 277 | 118 | 0 | |
| ARGTIYFDRSGYRRVDPFHI | 86 | 682539 | 4245 | 14770 | 31572 | 579414 | 2098 | 13607 | 31359 | 4755 | |
| ATGPAVRRGSWFDP | 92 | 189383 | 16256 | 10212 | 19927 | 8611 | 483 | 8039 | 51 | 4 | |
| ARRQWTFDY | 233 | 170557 | 18238 | 101 | 8176 | 130388 | 768 | 3086 | 7479 | 1614 | |
| ATGSPFDRTQNWFDP | 96 | 150096 | 14828 | 11766 | 20334 | 171 | 1077 | 7484 | 0 | 0 | |
| ARDGGGYVSY | 140 | 137057 | 26399 | 26912 | 9267 | 10889 | 856 | 4673 | 101 | 46 | |
| ARGVGAMQLWLRGYYFDS | 230 | 101311 | 5663 | 11064 | 2054 | 438 | 43 | 725 | 0 | 0 | |
| TTDRTYDYVWGSYRYEDY | 234 | 18251 | 3707 | 3514 | 1472 | 286 | 0 | 1938 | 0 | 0 | |
| ASPEASYYYGDRGHFFPARRIDFDF | 235 | 14123 | 2699 | 2484 | 654 | 557 | 123 | 214 | 0 | 18 | |
| TTDRSYDYVWGSYRYPDY | 221 | 13957 | 1191 | 648 | 1340 | 151 | 0 | 773 | 0 | 0 | |
| ARIPIATHLGSDY | 126 | 13507 | 1893 | 2152 | 382 | 51 | 16 | 791 | 0 | 0 | |
| ATAAAVRGRGTIDY | 102 | 11135 | 2241 | 1321 | 579 | 167 | 0 | 0 | 0 | 0 | |
| ARDYGRGGV | 218 | 9303 | 1792 | 1284 | 911 | 2965 | 49 | 114 | 11 | 73 | |
| AKDRAIFYLNPRYYLDY | 225 | 9245 | 3467 | 2871 | 892 | 485 | 64 | 1007 | 0 | 0 | |
| ASPTVT | 194 | 8847 | 1776 | 673 | 459 | 378 | 9 | 0 | 0 | 0 | |
| ARELPPGRMVVPATYWHFDL | 136 | 7591 | 448 | 1950 | 247 | 241 | 58 | 19 | 0 | 0 | |
| TSPRADYGSNSVGNY | 236 | 5736 | 1409 | 810 | 1209 | 1048 | 66 | 966 | 0 | 0 | |
| ARDGITNSGVVTHFGMDV | 237 | 4214 | 813 | 179 | 101 | 0 | 0 | 0 | 0 | 0 | |
| ARHMGMQLWLRAHDAFDL | 238 | 908 | 110 | 329 | 111 | 157 | 2 | 23 | 0 | 0 | |
| ARLGTGYP | 239 | 655 | 410 | 0 | 43 | 75 | 0 | 0 | 0 | 0 | |
| ACSRARGFMFFDY | 240 | 631 | 77 | 103 | 65 | 64 | 5 | 54 | 0 | 0 | |
| AKATQLFWLGQFTRDGFDI | 228 | 366 | 344 | 0 | 22 | 0 | 0 | 0 | 0 | 0 | |
| ARADSCNGGICYYQGMDV | 241 | 215 | 27 | 39 | 37 | 22 | 5 | 19 | 0 | 0 | |
| AREQPPGRMVVPATYWHFDL | 242 | 70 | 0 | 0 | 57 | 13 | 0 | 0 | 0 | 0 | |

MinION, all-RBD

| | SEQ | | RBD | | | | | | | | antigen |
|---|---|---|---|---|---|---|---|---|---|---|---|
| | ID | Total | R1 | | R2 | | R3 | | | | round |
| VH CDR3 | NO: | reads | 1 | 2 | 5 | 6 | 9 | 10 | 11 | 12 | selection |
| AKTSGYNLPDY | 232 | 3218688 | 6664 | 26643 | 683949 | 1118570 | 327024 | 275797 | 419902 | 351072 | |
| ARGTIYFDRSGYRRVDPFHI | 86 | 1153588 | 83798 | 113214 | 17975 | 35948 | 242 | 113 | 12 | 0 | |
| ATGPAVRRGSWFDP | 92 | 250994 | 31358 | 68340 | 20775 | 3663 | 10183 | 23208 | 1273 | 174 | |
| ARRQWTFDY | 233 | 198948 | 847 | 64 | 53 | 41 | 0 | 0 | 0 | 0 | |
| ATGSPFDRTQNWFDP | 96 | 195000 | 30348 | 76332 | 4946 | 3572 | 887 | 2924 | 1188 | 0 | |
| ARDGGGYVSY | 140 | 184361 | 22115 | 53919 | 1443 | 1524 | 220 | 706 | 0 | 0 | |
| ARGVGAMQLWLRGYYFDS | 230 | 136418 | 5159 | 6806 | 36585 | 10653 | 20616 | 15181 | 2060 | 565 | |
| TTDRTYDYVWGSYRYEDY | 234 | 26192 | 2920 | 6840 | 232 | 145 | 0 | 0 | 0 | 0 | |
| ASPEASYYYGDRGHFFPARRIDFDF | 235 | 19357 | 4519 | 4441 | 70 | 183 | 0 | 0 | 191 | 0 | |
| TTDRSYDYVWGSYRYPDY | 221 | 20327 | 3862 | 9682 | 607 | 434 | 187 | 91 | 0 | 0 | |
| ARIPIATHLGSDY | 126 | 20181 | 2791 | 8218 | 183 | 407 | 0 | 0 | 0 | 248 | |
| ATAAAVRGRGTIDY | 102 | 17518 | 4120 | 6919 | 391 | 0 | 76 | 0 | 0 | 0 | |
| ARDYGRGGV | 218 | 12940 | 427 | 1853 | 175 | 216 | 0 | 64 | 0 | 0 | |
| AKDRAIFYLNPRYYLDY | 225 | 12123 | 401 | 245 | 0 | 0 | 0 | 0 | 0 | 0 | |
| ASPTVT | 194 | 11590 | 1489 | 4323 | 142 | 271 | 0 | 0 | 888 | 17 | |
| ARELPPGRMVVPATYWHFDL | 136 | 11084 | 1019 | 4107 | 32 | 529 | 0 | 0 | 548 | 34 | |
| TSPRADYGSNSVGNY | 236 | 7607 | 147 | 171 | 0 | 0 | 0 | 0 | 0 | 0 | |
| ARDGITNSGVVTHFGMDV | 237 | 5713 | 525 | 2806 | 64 | 393 | 0 | 263 | 126 | 0 | |
| ARHMGMQLWLRAHDAFDL | 238 | 1137433 | 244342 | 126324 | 28952 | 16160 | 2292 | 567 | 26794 | 0 | |
| ARLGTGYP | 239 | 1077 | 348 | 0 | 0 | 0 | 0 | 0 | 0 | 0 | |
| ACSRARGFMFFDY | 240 | 1850171 | 383401 | 298072 | 76867 | 84338 | 11758 | 539 | 5518 | 12367 | |
| AKATQLFWLGQFTRDGFDI | 228 | 2110 | 1328 | 0 | 0 | 0 | 0 | 0 | 0 | 0 | |
| ARADSCNGGICYYQGMDV | 241 | 602990 | 117789 | 119416 | 17015 | 7319 | 1338 | 46 | 15680 | 0 | |
| AREQPPGRMVVPATYWHFDL | 242 | 8668 | 0 | 4107 | 0 | 529 | 0 | 0 | 0 | 0 | |

MinION, all-ECD

| | SEQ | | ECD | | | | | | | | antigen |
|---|---|---|---|---|---|---|---|---|---|---|---|
| | ID | Total | R1 | | R2 | | R3 | | | | round |
| VH CDR3 | NO: | reads | 3 | 4 | 7 | 8 | 13 | 14 | 15 | 16 | selection |
| AKTSGYNLPDY | 232 | 3218688 | 2994 | 2858 | 1356 | 1255 | 130 | 291 | 183 | 0 | |
| ARGTIYFDRSGYRRVDPFHI | 86 | 1153588 | 48395 | 64390 | 65571 | 646501 | 4739 | 33287 | 33785 | 5618 | |
| ATGPAVRRGSWFDP | 92 | 250994 | 23473 | 14360 | 28393 | 14557 | 691 | 9944 | 590 | 12 | |
| ARRQWTFDY | 233 | 198948 | 22481 | 153 | 10507 | 150524 | 863 | 3258 | 8135 | 2022 | |
| ATGSPFDRTQNWFDP | 96 | 195000 | 20115 | 16676 | 28331 | 219 | 1278 | 8184 | 0 | 0 | |
| ARDGGGYVSY | 140 | 184361 | 34699 | 37412 | 12661 | 13458 | 1002 | 5035 | 106 | 61 | |
| ARGVGAMQLWLRGYYFDS | 230 | 136418 | 8805 | 23836 | 4193 | 654 | 51 | 1254 | 0 | 0 | |
| TTDRTYDYVWGSYRYEDY | 234 | 26192 | 4939 | 6592 | 2101 | 364 | 0 | 2059 | 0 | 0 | |
| ASPEASYYYGDRGHFFPARRIDFDF | 235 | 19357 | 3917 | 3953 | 973 | 717 | 139 | 232 | 0 | 22 | |
| TTDRSYDYVWGSYRYPDY | 221 | 20327 | 1668 | 967 | 1814 | 189 | 0 | 826 | 0 | 0 | |
| ARIPIATHLGSDY | 126 | 20181 | 2960 | 3683 | 698 | 75 | 20 | 898 | 0 | 0 | |
| ATAAAVRGRGTIDY | 102 | 17518 | 3054 | 1914 | 822 | 222 | 0 | 0 | 0 | 0 | |
| ARDYGRGGV | 218 | 12940 | 2384 | 2299 | 1327 | 3652 | 302 | 125 | 21 | 95 | |
| AKDRAIFYLNPRYYLDY | 225 | 12123 | 4568 | 3974 | 1213 | 569 | 98 | 1055 | 0 | 0 | |
| ASPTVT | 194 | 11590 | 2370 | 959 | 637 | 481 | 13 | 0 | 0 | 0 | |
| ARELPPGRMVVPATYWHFDL | 136 | 11084 | 654 | 3281 | 400 | 389 | 70 | 21 | 0 | 0 | |
| TSPRADYGSNSVGNY | 236 | 7607 | 1896 | 1203 | 1693 | 1332 | 81 | 1084 | 0 | 0 | |
| ARDGITNSGVVTHFGMDV | 237 | 5713 | 1139 | 258 | 139 | 0 | 0 | 0 | 0 | 0 | |
| ARHMGMQLWLRAHDAFDL | 238 | 1137433 | 286366 | 121926 | 98414 | 114284 | 18517 | 52495 | 0 | 0 | |
| ARLGTGYP | 239 | 1077 | 582 | 0 | 55 | 92 | 0 | 0 | 0 | 0 | |
| ACSRARGFMFFDY | 240 | 1850171 | 410151 | 214435 | 144263 | 86504 | 19750 | 102177 | 31 | 0 | |
| AKATQLFWLGQFTRDGFDI | 228 | 2110 | 692 | 0 | 90 | 0 | 0 | 0 | 0 | 0 | |
| ARADSCNGGICYYQGMDV | 241 | 602990 | 129185 | 53080 | 70045 | 10077 | 14848 | 47152 | 0 | 0 | |
| AREQPPGRMVVPATYWHFDL | 242 | 8668 | 654 | 3281 | 84 | 13 | 0 | 0 | 0 | 0 | |

iSeq-RBD

| | SEQ | | RBD | | | | | | | | antigen |
|---|---|---|---|---|---|---|---|---|---|---|---|
| | ID | Total | R1 | | R2 | | R3 | | | | round |
| VH CDR3 | NO: | reads | 1 | 2 | 5 | 6 | 9 | 10 | 11 | 12 | selection |
| AKTSGYNLPDY | 232 | 82347 | 256 | 815 | 14954 | 18767 | 11036 | 8856 | 13409 | 13884 | |
| ARGTIYFDRSGYRRVDPFHI | 86 | 41256 | 3085 | 4441 | 1471 | 1442 | 35 | 8 | 4 | 0 | |
| ATGPAVRRGSWFDP | 92 | 14595 | 1313 | 3152 | 1394 | 156 | 830 | 1732 | 137 | 23 | |
| ARRQWTFDY | 233 | 5961 | 25 | 4 | 5 | 0 | 0 | 0 | 0 | 0 | |
| ATGSPFDRTQNWFDP | 96 | 9471 | 1087 | 3124 | 370 | 152 | 103 | 229 | 122 | 0 | |
| ARDGGGYVSY | 140 | 7760 | 683 | 2084 | 65 | 53 | 42 | 39 | 0 | 0 | |
| ARGVGAMQLWLRGYYFDS | 230 | 10220 | 225 | 515 | 2630 | 562 | 1521 | 1037 | 291 | 218 | |
| TTDRTYDYVWGSYRYEDY | 234 | 1497 | 118 | 332 | 19 | 8 | 0 | 0 | 0 | 0 | |
| ASPEASYYYGDRGHFFPARRIDFDF | 235 | 1356 | 235 | 291 | 6 | 10 | 0 | 1 | 27 | 0 | |
| TTDRSYDYVWGSYRYPDY | 221 | 1046 | 149 | 456 | 48 | 22 | 14 | 6 | 0 | 0 | |
| ARIPIATHLGSDY | 126 | 1058 | 100 | 403 | 13 | 14 | 0 | 0 | 0 | 46 | |
| ATAAAVRGRGTIDY | 102 | 924 | 186 | 309 | 46 | 0 | 13 | 1 | 0 | 0 | |
| ARDYGRGGV | 218 | 631 | 24 | 64 | 20 | 7 | 0 | 8 | 0 | 0 | |
| AKDRAIFYLNPRYYLDY | 225 | 881 | 36 | 24 | 0 | 0 | 0 | 0 | 0 | 0 | |
| ASPTVT | 194 | 436 | 46 | 134 | 7 | 11 | 0 | 1 | 37 | 5 | |
| ARELPPGRMVVPATYWHFDL | 136 | 928 | 54 | 206 | 7 | 38 | 0 | 0 | 123 | 30 | |
| TSPRADYGSNSVGNY | 236 | 725 | 13 | 15 | 0 | 0 | 0 | 0 | 0 | 0 | |
| ARDGITNSGVVTHFGMDV | 237 | 320 | 21 | 114 | 4 | 27 | 0 | 35 | 16 | 0 | |
| ARHMGMQLWLRAHDAFDL | 238 | 46761 | 6549 | 5147 | 1905 | 367 | 189 | 44 | 1831 | 0 | |
| ARLGTGYP | 239 | 32 | 7 | 0 | 0 | 0 | 0 | 0 | 0 | 0 | |
| ACSRARGFMFFDY | 240 | 71835 | 8968 | 10943 | 4860 | 2819 | 870 | 32 | 411 | 3085 | |
| AKATQLFWLGQFTRDGFDI | 228 | 128 | 69 | 0 | 0 | 1 | 0 | 0 | 0 | 0 | |
| ARADSCNGGICYYQGMDV | 241 | 27489 | 3810 | 5320 | 1319 | 359 | 139 | 4 | 1261 | 0 | |
| AREQPPGRMVVPATYWHFDL | 242 | 222 | 12 | 100 | 0 | 2 | 0 | 0 | 0 | 0 | |

iSeq-ECD

| | SEQ | | ECD | | | | | | | | antigen |
|---|---|---|---|---|---|---|---|---|---|---|---|
| | ID | Total | R1 | | R2 | | R3 | | | | round |
| VH CDR3 | NO: | reads | 3 | 4 | 7 | 8 | 13 | 14 | 15 | 16 | selection |
| AKTSGYNLPDY | 232 | 82347 | 119 | 133 | 76 | 4 | 15 | 6 | 17 | 0 | |
| ARGTIYFDRSGYRRVDPFHI | 86 | 41256 | 2122 | 3982 | 5295 | 14007 | 1356 | 558 | 2954 | 496 | |
| ATGPAVRRGSWFDP | 92 | 14595 | 1051 | 985 | 2253 | 717 | 532 | 91 | 225 | 4 | |
| ARRQWTFDY | 233 | 5961 | 565 | 9 | 509 | 3869 | 89 | 60 | 699 | 127 | |
| ATGSPFDRTQNWFDP | 96 | 9471 | 811 | 1002 | 1963 | 10 | 349 | 149 | 0 | 0 | |
| ARDGGGYVSY | 140 | 7760 | 1082 | 2066 | 827 | 514 | 174 | 108 | 16 | 7 | |
| ARGVGAMQLWLRGYYFDS | 230 | 10220 | 609 | 1887 | 554 | 65 | 94 | 8 | 4 | 0 | |
| TTDRTYDYVWGSYRYEDY | 234 | 1497 | 241 | 458 | 217 | 17 | 86 | 0 | 0 | 1 | |
| ASPEASYYYGDRGHFFPARRIDFDF | 235 | 1356 | 233 | 303 | 134 | 62 | 21 | 19 | 7 | 7 | |
| TTDRSYDYVWGSYRYPDY | 221 | 1046 | 92 | 81 | 133 | 14 | 25 | 6 | 0 | 0 | |
| ARIPIATHLGSDY | 126 | 1058 | 136 | 221 | 62 | 2 | 58 | 3 | 0 | 0 | |
| ATAAAVRGRGTIDY | 102 | 924 | 139 | 132 | 71 | 27 | 0 | 0 | 0 | 0 | |
| ARDYGRGGV | 218 | 631 | 88 | 134 | 90 | 171 | 5 | 6 | 3 | 11 | |
| AKDRAIFYLNPRYYLDY | 225 | 881 | 233 | 323 | 145 | 43 | 57 | 20 | 0 | 0 | |
| ASPTVT | 194 | 436 | 63 | 55 | 50 | 23 | 0 | 4 | 0 | 0 | |
| ARELPPGRMVVPATYWHFDL | 136 | 928 | 30 | 288 | 91 | 32 | 6 | 23 | 0 | 0 | |
| TSPRADYGSNSVGNY | 236 | 725 | 118 | 123 | 221 | 104 | 109 | 22 | 0 | 0 | |
| ARDGITNSGVVTHFGMDV | 237 | 320 | 58 | 29 | 16 | 0 | 0 | 0 | 0 | 0 | |
| ARHMGMQLWLRAHDAFDL | 238 | 46761 | 9670 | 6359 | 6587 | 5079 | 1654 | 1380 | 0 | 0 | |
| ARLGTGYP | 239 | 32 | 19 | 0 | 5 | 1 | 0 | 0 | 0 | 0 | |
| ACSRARGFMFFDY | 240 | 71835 | 11763 | 10470 | 9082 | 4015 | 3249 | 1267 | 1 | 0 | |
| AKATQLFWLGQFTRDGFDI | 228 | 128 | 51 | 0 | 7 | 0 | 0 | 0 | 0 | 0 | |
| ARADSCNGGICYYQGMDV | 241 | 27489 | 4675 | 2473 | 4540 | 700 | 1723 | 1166 | 0 | 0 | |
| AREQPPGRMVVPATYWHFDL | 242 | 222 | 20 | 74 | 12 | 2 | 0 | 0 | 0 | 0 | |

### Example 6—Non-Cognate V_{H}-V_{L} Pairs are Potent Neutralizing Antibodies to SARS-CoV-2

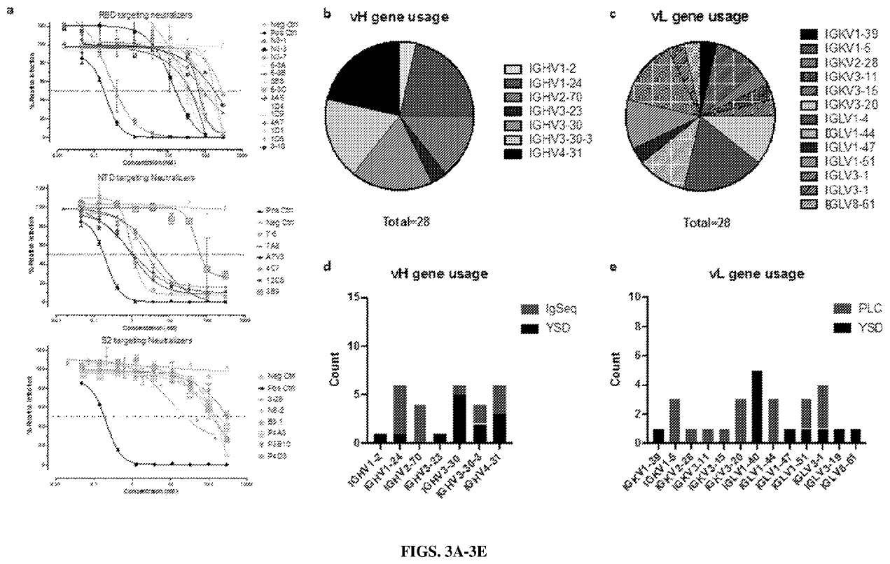

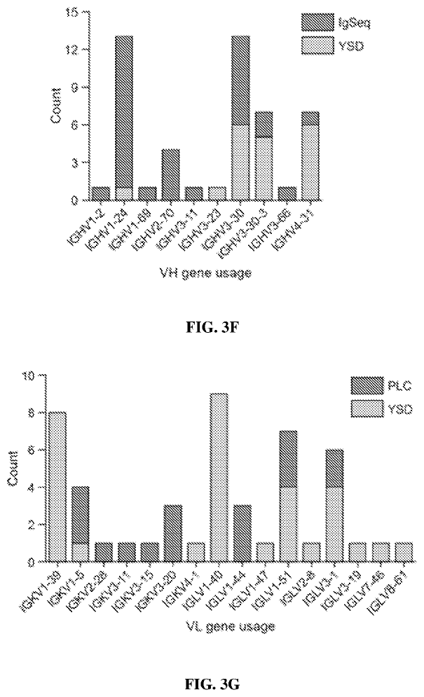

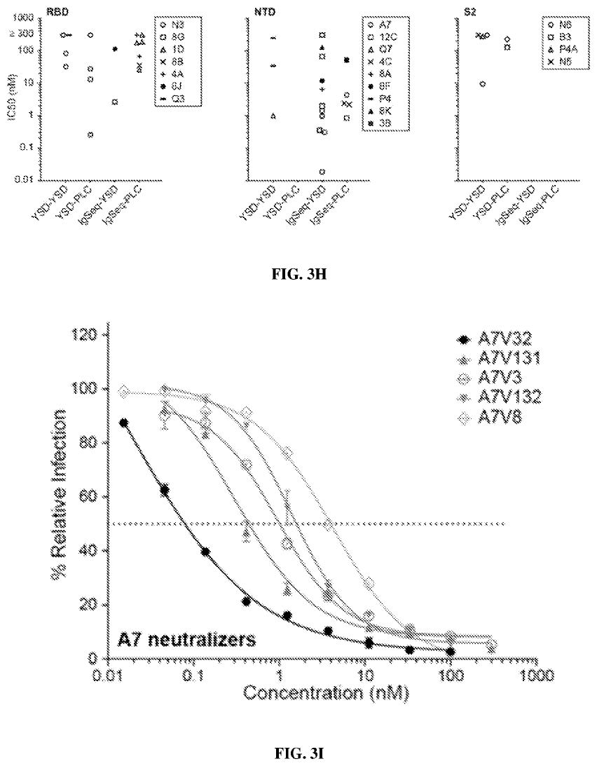

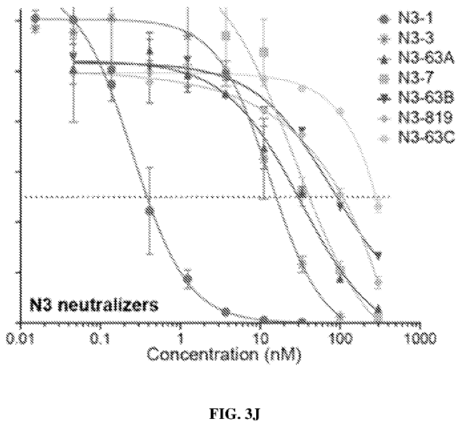

Next, the top candidates from each of the discovery strategies was tested for in vitro live virus neutralization of SARS-COV-2. Initially, isolated IgG was screened against the ECD, NTD, and RBD of the spike protein using ELISA to identify the most promising candidates. Non-RBD antibodies were prioritized in the screens in order to gain insights into alternative neutralization mechanisms. From binding data of the full-length IgGs, 120 candidate VH-VL pairs were selected for live virus neutralization, and 49 pairs successfully neutralized authentic SARS-COV-2/WA1 (FIGS. 3A-3J, Tables 8 and 18), representing 26 distinct VHs. Neutralizing VH usage encompassed 10 IGHV genes, with preferences for IGHV1-24, IGHV3-30 and IGHV3-30-3 consistent with other published SARS-COV-2 neutralizing antibodies (FIG. 3F) (Zhou et al., 2021).

VL partners from 16 IGLV germline genes were identified, and all PLCs except for PLC-6 participated in at least one neutralizing VH-VL pair. YSD discovered an additional 8 IGLV genes, yielding a total of 32 antibodies with YSD-derived VH or VLs. Several VHs successfully paired with both PLC-derived and YSD-selected VLs. Interestingly, the VLs for a given VH were often derived from the same germline genes. For example, 12C pairs with PLC8 (12C8) as well as a YSD-selected VL (12C114). Both VLs utilize the same V-gene (IGLV1-51) and the corresponding mAbs exhibited nearly equivalent IC50 values of 0.84 nM and 0.35 nM, respectively.

High affinity mAbs targeting the NTD (A7V32, A7V131, 8-114, 12C8, A7V3, 7-6, 4C7, 4C8, A7V8) showed strong preference for IGHV1-24 gene usage. The A7 heavy chain is a highly potent NTD-directed neutralizer when paired with any one of five different light chains, both YSD and PLC-derived (V_{H}-V_{L} names: A7V32, A7V3, A7V8, A7V131 and A7V132, FIG. 61). The most effective pairing for neutralization involved IGLV2-8 (A7V32, IC50=18 pM), while all other functional pairs utilized IGLV1-51.

Curve fitting calculated IC₅₀ values as low as 251.9 pM, with four antibodies showing sub-nanomolar values (N3-1, 251.9 PM; 12C8, 835.5 pM; A7V3, 954.7 pM; and 7-6, 977.0 pM). Nineteen neutralizing antibodies had IC₅₀ values less than 100 nM (FIG. 3). The high affinity NTD binders (12C8, A7V3, 7-6, 4C7, 7A8) showed strong preference for IGHV1-24 gene usage. PLC screening successfully identified VL partners for both YSD and Ig-Seq derived VHs (FIGS. 3B-3E). Seven of the mAbs were shown to be directed to the S2 domain. N3 proved to be a highly potent RBD directed IGHV4-31 and displayed strong neutralization activity with several PLCs as well as YSD VLs (Table 18). Seven VLs were found that successfully paired with N3 to neutralize SARS-COV-2 (FIG. 3J). The pairs for N3 are more diverse in VL gene usage and display a broad range of IC50 values (251.9 pM to >300 nM). In total, seven antibodies from four distinct VH clonotypes have sub-nanomolar IC50 values (A7V32, 18 pM; N3-1, 251.9 pM; A7V131, 300 pM; 8-114, 350 pM; 12C8, 836 pM; A7V3, 955 pM; and 7-6, 977 pM), and 28 neutralizing antibodies composed of 10 VH clonotypes have IC50 values below 100 nM (Table 8).

TABLE 18
Summary of neutralizing antibodies.

| | VH | IC50 | | | | | Epi- | HC | LC |
|---|---|---|---|---|---|---|---|---|---|
| mAb | label | (nM) | VH gene | H-CDR3 | VL gene | L-CDR3 | tope | source | source |
| A7V32 | A7 | 0.01806 | IGHV1-24 | ATGSPFDRTQNWFDP | IGLV2-8 | SSYAGSNNLA | NTD | IgSeq | YSD |
| | | | | (SEQ ID NO: 96) | | (SEQ ID NO: 209) | | | |
| N3-1 | N3 | 0.25 | IGHV4-31 | ARGTIYFDRSGYRRVDPF | IGKV1-5 | QQYNSYSPWT | RBD | YSD | PLC |
| | | | | HI | | (SEQ ID NO: 89) | | | |
| | | | | (SEQ ID NO: 86) | | | | | |
| A7V131 | A7 | 0.3045 | IGHV1-24 | ATGSPFDRTQNWFDP | IGLV1-51 | GTWDNSLSAGV | NTD | IgSeq | YSD |
| | | | | (SEQ ID NO: 96) | | (SEQ ID NO: 212) | | | |
| 12C114 | 12C | 0.3516 | IGHV1-24 | ATGPAVRRGSWFDP | IGLV1-51 | GTWDSSLSGYV | NTD | IgSeq | YSD |
| | | | | (SEQ ID NO: 92) | | (SEQ ID NO: 206) | | | |
| 12C8 | 12C | 0.84 | IGHV1-24 | ATGPAVRRGSWFDP | IGLV1-51 | GTWDSSLSAVV | NTD | IgSeq | PLC |
| | | | | (SEQ ID NO: 92) | | (SEQ ID NO: 95) | | | |
| A7V3 | A7 | 0.95 | IGHV1-24 | ATGSPFDRTQNWFDP | IGLV1-51 | GTWDSSLSAVV | NTD | IgSeq | YSD |
| | | | | (SEQ ID NO: 96) | | (SEQ ID NO: 95) | | | |
| Q76 | Q7 | 0.98 | IGHV1-24 | ATRFAVYGDYLIDY | IGLV3-19 | NSRDSSGDLVV | NTD | YSD | YSD |
| | | | | (SEQ ID NO: 97) | | (SEQ ID NO: 100) | | | |
| A7V132 | A7 | 1.391 | IGHV1-24 | ATGSPFDRTQNWFDP | IGLV1-51 | GTWDSSLSAGV | NTD | IgSeq | YSD |
| | | | | (SEQ ID NO: 96) | | (SEQ ID NO: 214) | | | |
| 12C115 | 12C | 1.929 | IGHV1-24 | ATGPAVRRGSWFDP | IGLV1-40 | QSYDSSLSGWV | NTD | IgSeq | YSD |
| | | | | (SEQ ID NO: 92) | | (SEQ ID NO: 466) | | | |
| 4C7 | 4C | 2.15 | IGHV1-24 | ATAAAVRGRGTIDY | IGLV1-44 | AAWDDSLNGPVV | NTD | IgSeq | PLC |
| | | | | (SEQ ID NO: 102) | | (SEQ ID NO: 105) | | | |
| 4C8 | 4C | 2.353 | IGHV1-24 | ATAAAVRGRGTIDY | IGLV1-51 | GTWDSSLSAVV | NTD | IgSeq | PLC |
| | | | | (SEQ ID NO: 102) | | (SEQ ID NO: 95) | | | |
| 8G91 | 8G | 2.586 | IGHV1-69 | AREQPPGRMVVPATYWH | IGKV1-39 | QQSYSTPYT | RBD | IgSeq | YSD |
| | | | | FDL | | (SEQ ID NO: 467) | | | |
| | | | | (SEQ ID NO: 242) | | | | | |
| A7V8 | A7 | 4.24 | IGHV1-24 | ATGSPFDRTQNWFDP | IGLV1-51 | GTWDSSLSAVV | NTD | IgSeq | PLC |
| | | | | (SEQ ID NO: 96) | | (SEQ ID NO: 95) | | | |
| 8H97 | 8H | 4.955 | IGHV3- | ARGNWGSYYYGMDV | IGLV3-1 | QSSDDSNQLV | ECD | YSD | YSD |
| | | | 30-3 | (SEQ ID NO: 197) | | (SEQ ID NO: 123) | | | |
| 8A3 | 8A | 6.422 | IGHV3-66 | ARGGVVDTYYYYGMDV | IGLV7-46 | LLSQSGAWV | NTD | IgSeq | YSD |
| | | | | (SEQ ID NO: 202) | | (SEQ ID NO: 205) | | | |
| N6-326 | N6 | 9.42 | IGHV3- | ARPYSGSYWGYFDY | IGLV1-47 | AAWDDSLSGPV | S2 | YSD | YSD |
| | | | 30-3 | (SEQ ID NO: 114) | | (SEQ ID NO: 117) | | | |
| 8F90 | 8F | 11.56 | IGHV3-11 | ARDGITNSGVVTHFGMDV | IGKV4-1 | QQYNSYSRVT | NTD | IgSeq | YSD |
| | | | | (SEQ ID NO: 237) | | (SEQ ID NO: 468) | | | |
| N3-3 | N3 | 13.1 | IGHV4-31 | ARGTIYFDRSGYRRVDP | IGKV3-11 | QQRSNWPPLT | RBD | YSD | PLC |
| | | | | FHI | | (SEQ ID NO: 119) | | | |
| | | | | (SEQ ID NO: 86) | | | | | |
| 8C100 | 8C | 20.45 | IGHV3-30 | AKDRRYYDFWSGYVGSP | IGLV3-1 | QSYGNNQGV | ECD | YSD | YSD |
| | | | | YYYYYYGMDV | | (SEQ ID NO: 153) | | | |
| | | | | (SEQ ID NO: 191) | | | | | |
| 1D4 | 1D | 25.5 | IGHV2-70 | ARIPIATHLGSDY | IGKV3-15 | QQYNNWPPWT | RBD | IgSeq | PLC |
| | | | | (SEQ ID NO: 126) | | (SEQ ID NO: 129) | | | |
| N3-7 | N3 | 27.7 | IGHV4-31 | ARGTIYFDRSGYRRVDP | IGLV1-44 | AAWDDSLNGPVV | RBD | YSD | PLC |
| | | | | FHI | | (SEQ ID NO: 105) | | | |
| | | | | (SEQ ID NO: 86) | | | | | |
| N3-63A | N3 | 32.0 | IGHV4-31 | ARGTIYFDRSGYRRVDP | IGLV8-61 | TLYMGGGLLV | RBD | YSD | YSD |
| | | | | FHI | | (SEQ ID NO: 133) | | | |
| | | | | (SEQ ID NO: 86) | | | | | |
| P4D96 | P4 | 33.54 | IGHV3-30 | AKAPGQWLRFHYYGMDV | IGLV1-40 | NSRDINSNHVL | NTD | YSD | YSD |
| | | | | (SEQ ID NO: 150) | | (SEQ ID NO: 190) | | | |
| 8B5 | 8B | 34.8 | IGHV1-2 | ARELPPGRMVVPATYWH | IGKV3-20 | QQYGSSPPWT | RBD | IgSeq | PLC |
| | | | | FDL | | (SEQ ID NO: 138) | | | |
| | | | | (SEQ ID NO: 136) | | | | | |
| 3B9 | 3B | 51.4 | IGHV3-30 | ARDGGGYVSY | IGLV3-1 | QAWDSSTVV | NTD | IgSeq | PLC |
| | | | | (SEQ ID NO: 140) | | (SEQ ID NO: 143) | | | |
| 12C130 | 12C | 65.13 | IGHV3-30 | ATGPAVRRGSWFDP | IGKV1-39 | QQSYSTPRT | NTD | IgSeq | YSD |
| | | | | (SEQ ID NO: 92) | | (SEQ ID NO: 213) | | | |
| 4A5 | 4A | 65.7 | IGHV3- | AKASQLFWLGQFTRDG | IGKV3-20 | QQYGSSPPWT | RBD | IgSeq | PLC |
| | | | 30-3 | FDI | | (SEQ ID NO: 138) | | | |
| | | | | (SEQ ID NO: 139) | | | | | |
| N3-63B | N3 | 81.5 | IGHV4-31 | ARGTIYFDRSGYRRVDP | IGLV1-40 | QSYDGSLNDDVI | RBD | YSD | YSD |
| | | | | FHI | | (SEQ ID NO: 145) | | | |
| | | | | (SEQ ID NO: 86) | | | | | |
| 8J127 | 8J | 113 | IGHV3-30 | AKATQLFWLGQFTRDG | IGLV1-40 | QSYDSSLSGWV | RBD | IgSeq | YSD |
| | | | | FDI | | (SEQ ID NO: 466) | | | |
| | | | | (SEQ ID NO: 228) | | | | | |
| B3.1 | B3 | 124 | IGHV3-30 | ARARGGSYYYGMDV | IGKV1-5 | QQYNSYSPWT | S2 | YSD | PLC |
| | | | | (SEQ ID NO: 120) | | (SEQ ID NO: 89) | | | |
| 8K42 | 8K | 124.2 | IGHV3-30 | ARDYGRGGV | IGKV1-39 | QQSYSTRPLT | NTD | IgSeq | YSD |
| | | | | (SEQ ID NO: 218) | | (SEQ ID NO: 215) | | | |
| 8D107 | 8D | 126.9 | IGHV3-30 | AKTSGYNLPDY | IGKV1-39 | QQSYSTPRT | ECD | IgSeq | YSD |
| | | | | (SEQ ID NO: 232) | | (SEQ ID NO: 213) | | | |
| 1D1 | 1D | 173 | IGHV2-70 | ARIPIATHLGSDY | IGKV1-5 | QQYNSYSPWT | RBD | IgSeq | PLC |
| | | | | (SEQ ID NO: 126) | | (SEQ ID NO: 89) | | | |
| 1D9 | 1D | 183 | IGHV2-70 | ARIPIATHLGSDY | IGLV3-1 | QAWDSSTVV | RBD | IgSeq | PLC |
| | | | | (SEQ ID NO: 126) | | (SEQ ID NO: 143) | | | |
| 8L63 | 8L | 212.5 | IGHV3- | ARAYGGGYLTPFDY | IGLV1-40 | NSRDSSGDLVV | ECD | YSD | YSD |
| | | | 30-3 | (SEQ ID NO: 282) | | (SEQ ID NO: 100) | | | |
| N6-2 | N6 | 222 | IGHV3- | ARPYSGSYWGYFDY | IGKV2-28 | MQALQTPPYT | S2 | YSD | PLC |
| | | | 30-3 | (SEQ ID NO: 114) | | (SEQ ID NO: 148) | | | |
| P4D3 | P4 | 242 | IGHV3-30 | AKAPGQWLRFHYYGMDV | IGLV1-40 | QSYGNNQGV | NTD | YSD | YSD |
| | | | | (SEQ ID NO: 150) | | (SEQ ID NO: 153) | | | |
| P4A3 | P4A | 272 | IGHV3-30 | ARDDTGRVGSGWYCPLY | IGKV1-39 | QQSYSTPLS | S2 | YSD | YSD |
| | | | | (SEQ ID NO: 154) | | (SEQ ID NO: 157) | | | |
| 4A7 | 4A | >300 | IGHV3- | AKASQLFWLGQFTRDG | IGLV1-44 | AAWDDSLNGPVV | RBD | IgSeq | PLC |
| | | | 30-3 | FDI | | (SEQ ID NO: 105) | | | |
| | | | | (SEQ ID NO: 139) | | | | | |
| 8I122 | 8I | >300 | IGHV3-30 | AKDRAIFYLNPRYYLDY | IGKV1-5 | QQYNSYPLT | ECD | IgSeq | YSD |
| | | | | (SEQ ID NO: 225) | | (SEQ ID NO: 469) | | | |
| Q318 | Q3 | >300 | IGHV3-30 | AKQAGAYCSGGSCYSSS | IGLV1-40 | QSYGNNQGV | RBD | YSD | YSD |
| | | | | EADY | | (SEQ ID NO: 153) | | | |
| | | | | (SEQ ID NO: 159) | | | | | |
| N5C6 | N5 | >300 | IGHV3-23 | APGRSLY | IGLV1-40 | QSYDSGLSGSI | S2 | YSD | YSD |
| | | | | (SEQ ID NO: 163) | | (SEQ ID NO: 166) | | | |
| N3-63C | N3 | >300 | IGHV4-31 | ARGTIYFDRSGYRRVDP | IGLV3-1 | QAWDSSTVV | RBD | YSD | YSD |
| | | | | FHI | | (SEQ ID NO: 143) | | | |
| | | | | (SEQ ID NO: 86) | | | | | |
| N3-819 | N3 | >300 | IGHV4-31 | ARGTIYFDRSGYRRVDP | IGKV1-39 | QQSYSTRPLT | RBD | YSD | YSD |
| | | | | FHI | | (SEQ ID NO: 215) | | | |
| | | | | (SEQ ID NO: 86) | | | | | |
| 1D5 | 1D | >300 | IGHV2-70 | ARIPIATHLGSDY | IGKV3-20 | QQYGSSPPWT | RBD | IgSeq | PLC |
| | | | | (SEQ ID NO: 126) | | (SEQ ID NO: 138) | | | |
| N6-10B | N6 | >300 | IGHV3- | ARPYSGSYWGYFDY | IGLV3-1 | QAWDSSTF | S2 | YSD | YSD |
| | | | 30-3 | (SEQ ID NO: 114) | | (SEQ ID NO: 161) | | | |
| 9C-118 | 9C | >300 | IGHV3-30 | ASPTVT | IGKV1-39 | QQSYSSSWT | ECD | IgSeq | YSD |
| | | | | (SEQ ID NO: 194) | | (SEQ ID NO: 196) | | | |
| 12C112 | 12C | >300 | IGHV1-24 | ATGPAVRRGSWFDP | IGLV1-40 | QSFDSSLSGPVV | NTD | IgSeq | YSD |
| | | | | (SEQ ID NO: 92) | | (SEQ ID NO: 470) | | | |
| 12C113 | 12C | >300 | IGHV1-24 | ATGPAVRRGSWFDP | IGKV1-39 | QQSYSTRPLT | NTD | IgSeq | YSD |
| | | | | (SEQ ID NO: 92) | | (SEQ ID NO: 215) | | | |

### Example 7—Common Binding Mode of an NTD-Directed Antibody

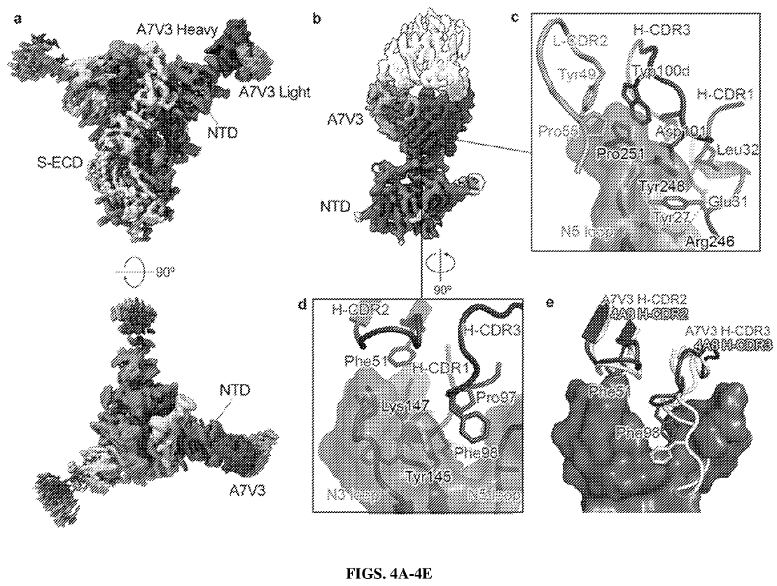

A7V3 was of interest because of the different donor origins of the VH and VL. Its IGHV1-24 was discovered in both IgSeq and YSD from donor 1. However, the IGLV1-51 was mapped to donor 2 and is also related to PLC8. The VH of A7V3 showed a strong preference to PLC8 in ELISA screening, and related light chains appear in four neutralizing VH-VL pairs, commonly pairing with IGHV1-24 VHs. To ascertain its potential binding mode and contributions from both variable regions, a cryo-EM structure of A7V3 Fab complexed with SARS-COV-2 S-ECD was determined. The initial 3D reconstruction from 715,398 particles had a global resolution of 3.0 Å (FIG. 4A). Although the map revealed an A7V3 Fab molecule bound to each NTD of the trimeric spike, only one Fab showed clear density. Further 3D classification was performed to resolve potential heterogeneity in the particle set. One class was obtained consisting of one fourth of the total particles and exhibited defined density for an NTD and its bound Fab. A local refinement of this map focused on the NTD and Fab yielded a reconstruction with a well-resolved binding interface (FIG. 4B). Similar to the first structurally defined NTD-targeted antibody 4A8 (Chi et al., 2020), the heavy chain of A7V3 plays a dominant role in binding the N3 loop (residues 141-156) and N5 loop (residues 246-260) of the NTD. The light chain of A7V3 makes limited contact with the NTD and is therefore likely to play a structural role in positioning the heavy chain. H-CDR1, H-CDR3, and L-CDR2 collectively form a concave surface packed tightly against the N5 loop. Specifically, Trp 100d in H-CDR3, and both Tyr49 and Pro55 in L-CDR2, form a hydrophobic cage to enclose Pro251 at the tip of the N5 loop (FIG. 4C). Glu31 from H-CDR1 is expected to form a salt bridge with Arg246, and Asp101 from H-CDR3 is expected to form a hydrogen bond with the hydroxyl group of Tyr248. Pro97 and Phe98 from H-CDR3 insert into a groove flanked by the N3 and N5 loops (FIG. 4D). As opposed to the major binding interface on the N5 loop, A7V3 only makes a minor contact with the N3 loop, mainly mediated by H-CDR1 and H-CDR2. Akin to a conserved Phe from H-CDR2 of 4A8, Phe51 contacts Lys147 via a π-cation interaction (FIGS. 4D-4E). In stark contrast, H-CDR3 of A7V3 is much shorter than its counterpart in 4A8 (Xiaojie et al., 2021), which contains a Phe that forms a R-T stacking interaction with Trp152 on the N3 loop. Collectively, the structural characterization of A7V3 demonstrates that this semi-synthetic approach is capable of yielding a novel neutralizing antibody that binds to the NTD using an established modality.

### Example 8—an RBD-Targeted Antibody Exhibits a Novel Quaternary Binding Mode

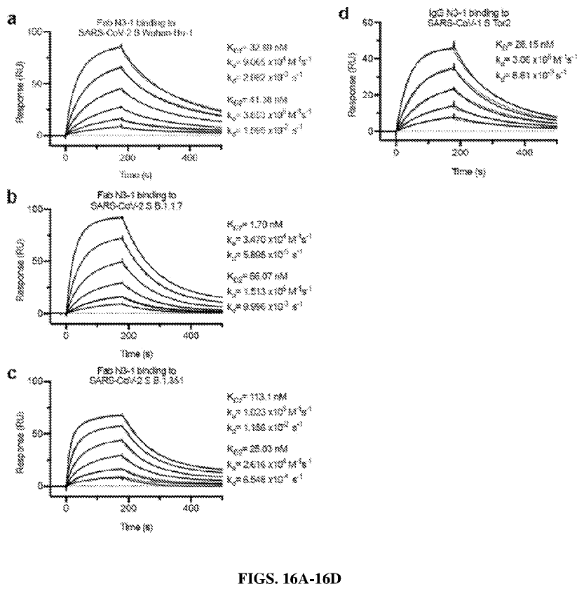

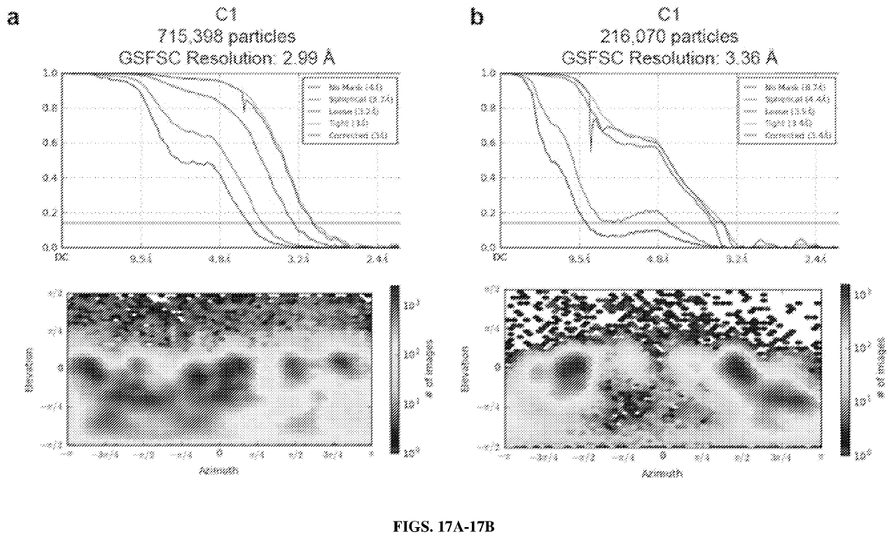

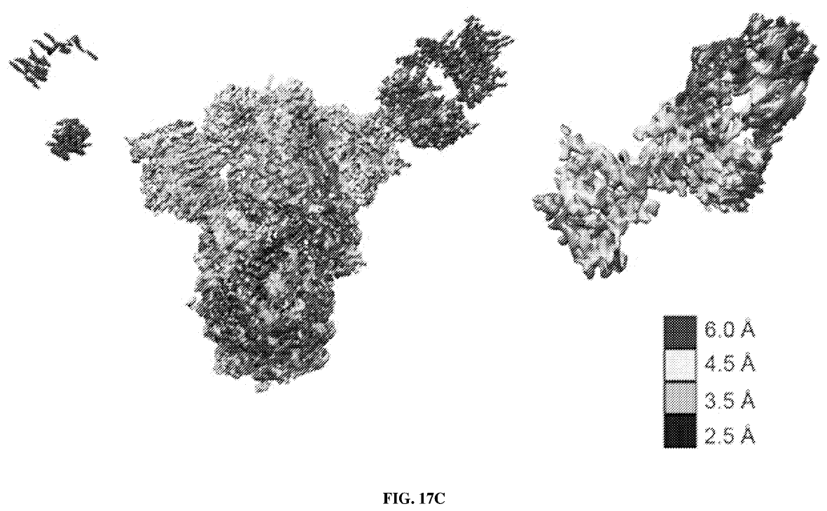

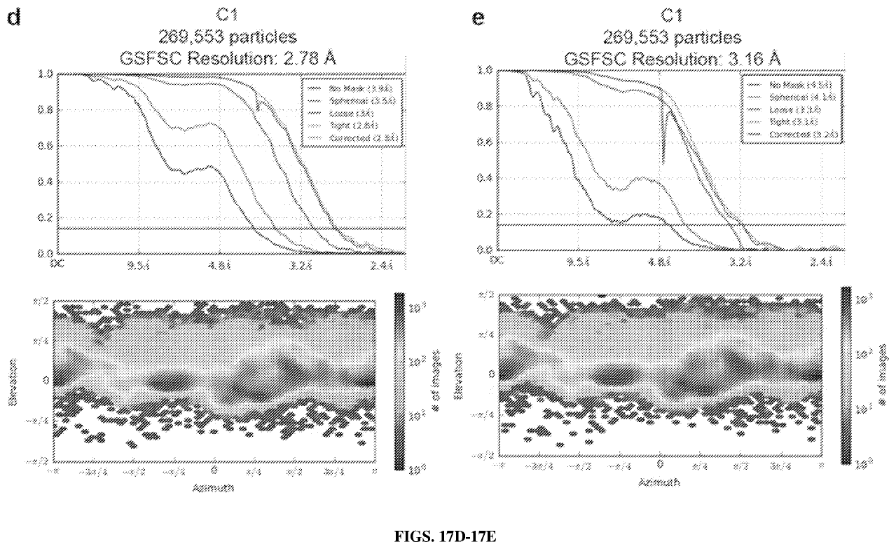

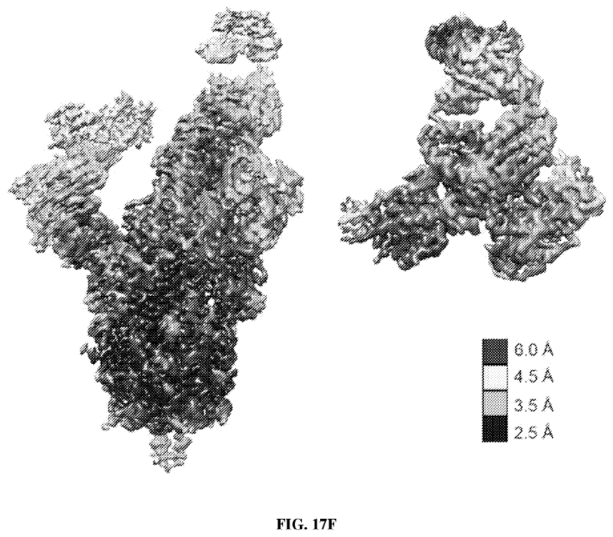

Structurally defined RBD-targeted antibodies or nanobodies complexed with SARS-COV-2-S-protein may be generally grouped into two types based on binding sites. The first type, mainly discovered from the IGHV-53 germline family, recognizes the angiotensin converting enzyme 2 (ACE2)-binding site and directly blocks host receptor engagement (e.g., antibodies CC12.1 (Yuan et al., 2020), C105 (Barnes et al., 2020), and VHH E (Koenig et al., 2021)). The second type, from a more diverse IGHV gene usage, recognizes a relatively conserved region on the RBD that is mostly buried and contacts a neighboring RBD of the S-protein trimer in the down conformation (e.g. C135 (Barnes et al., 2020), S309 (Pinto et al., 2020), and VHH V (Koenig et al., 2021)). From negative stain EM analysis of the IgG N3-1-spike complex, two clear densities of Fab were observed, likely derived from the same IgG molecule, bound to multiple RBDs on a single trimeric spike (FIGS. 15A-15B). The affinity of N3-1 IgG to SARS-COV-2 spike was nearly 1000-fold stronger than N3-1 Fab to spike (FIGS. 16A-16D).

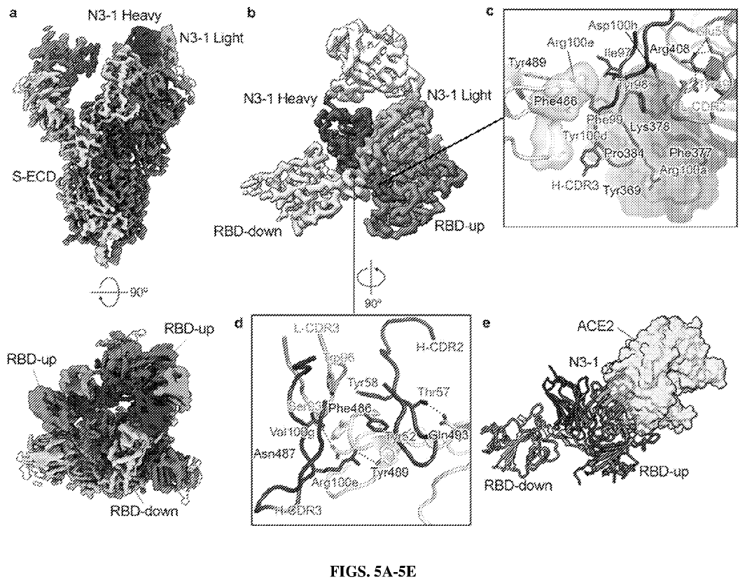

To investigate this unique binding mode, a cryo-EM structure of N3-1 Fab bound to SARS-COV-2 S-ECD was determined to a global resolution of 2.8 Å (FIG. 5A). Two N3-1 Fabs were found to bind to a single trimeric spike with one Fab binding to RBD in the up conformation and the other Fab simultaneously engaging two RBDs: one in up conformation and one in the down conformation. Focused refinement was performed on the Fab bound to the two RBDs (FIG. 5B), which substantially improved the interpretability of the map in this region. The well-resolved Fab-RBD binding interface revealed two completely distinct epitopes on RBD-up and RBD-down (FIG. 5C).

For the Fab binding the RBD-up, contacts are made by the H-CDR1, H-CDR3 and L-CDR2, which together bury 862 Å² surface area. Conversely, the Fab binding the RBD-down creates contacts via the H-CDR2, H-CDR3 and L-CDR3, which buries 696 Å² surface area. Notably, the relatively long H-CDR3 loop (18 a.a.) engages both RBDs via hydrophobic and polar interactions. H-CDR3 residues Tyr98, Phe99 and Arg100a pack against a hydrophobic pocket formed by Tyr369, Phe377, Lys378 and Pro384 on RBD-up, which are highly conserved between SARS-COV and SARS-COV-2. This pocket is barely exposed when RBD is in the down conformation and is part of the shared epitopes targeted by cross-reactive antibodies CR3022 and COVA1-16 (Liu et al., 2020). Lys378, from the upper ridge of the pocket, forms a cation-x interaction with Tyr98 of H-CDR3, and its amine group is expected to form a salt bridge with Asp100h of H-CDR3. In addition, main chain atoms of Cys379 and Tyr369 form hydrogen bonds with Phe99 and Arg 100a from H-CDR3, strengthening this primary Fab binding interface on RBD-up. Furthermore, the sidechain guanidinium of Arg408 on RBD-up likely has polar interactions with Tyr49 and Glu55 from L-CDR2, which along with H-CDR1, constitutes the secondary binding interface on RBD-up.

In contrast, the N3-1 binding site on RBD-down overlaps with the ACE2-binding site, where 11 of 19 N3-1 epitope residues binding to N3-1 are also involved in ACE2 binding (FIG. 5D). Phe486 on RBD-down inserts into a hydrophobic pocket formed by Trp96 (L-CDR3), Tyr52, Tyr58 (H-CDR2), Arg100e and Val100g (H-CDR3). The sidechain guanidinium of Arg100e not only forms a hydrogen bond with Tyr489, but also contacts Phe486 through cation-x interactions. In addition, Asn487 and Gln493 on RBD-down are expected to form polar interactions with Ser93 of L-CDR3 and Thr57 of H-CDR2. The angle of approach of N3-1 prevents ACE2 binding by both trapping one RBD in the down position, thereby preventing exposure of the ACE2 binding site, as well as by sterically inhibiting ACE2 access to the bound ‘up’ RBD. (FIG. 5E). Collectively, the N3-1 antibody engages an extensive quaternary epitope on neighboring RBDs through a novel binding modality.

### Example 9—mAb N3-1 Binds Robustly to Spike Proteins from Emerging Variants of Concern

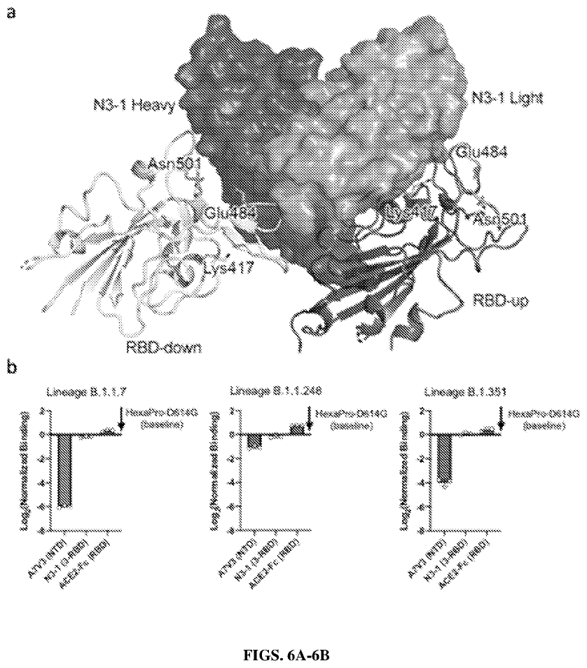

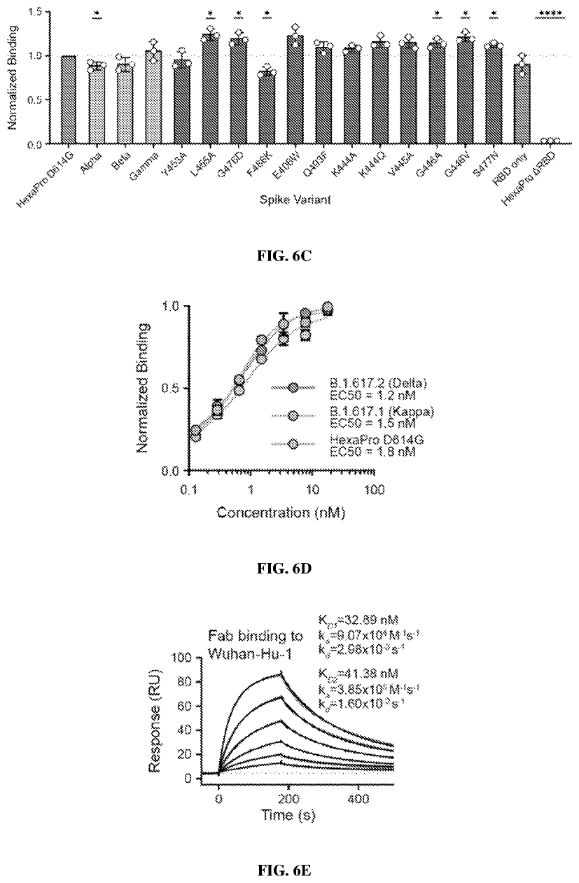

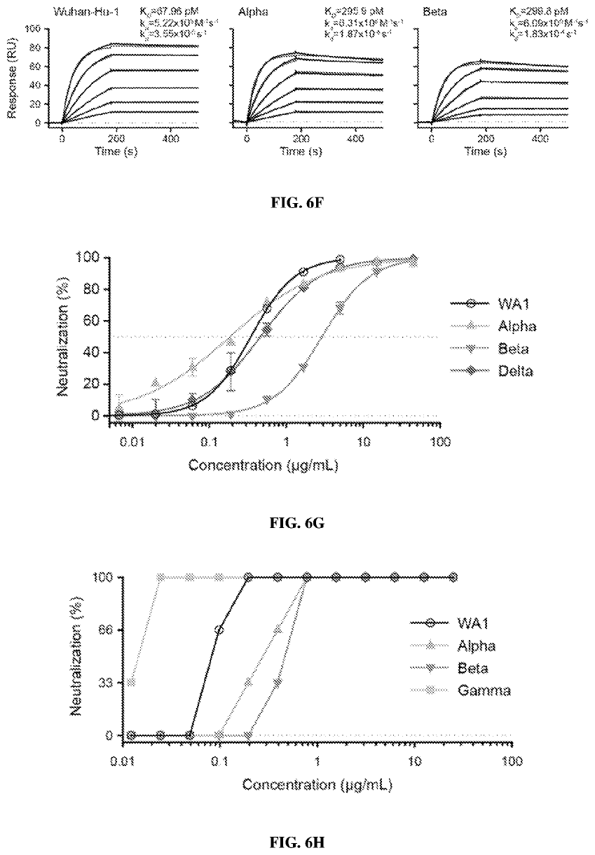

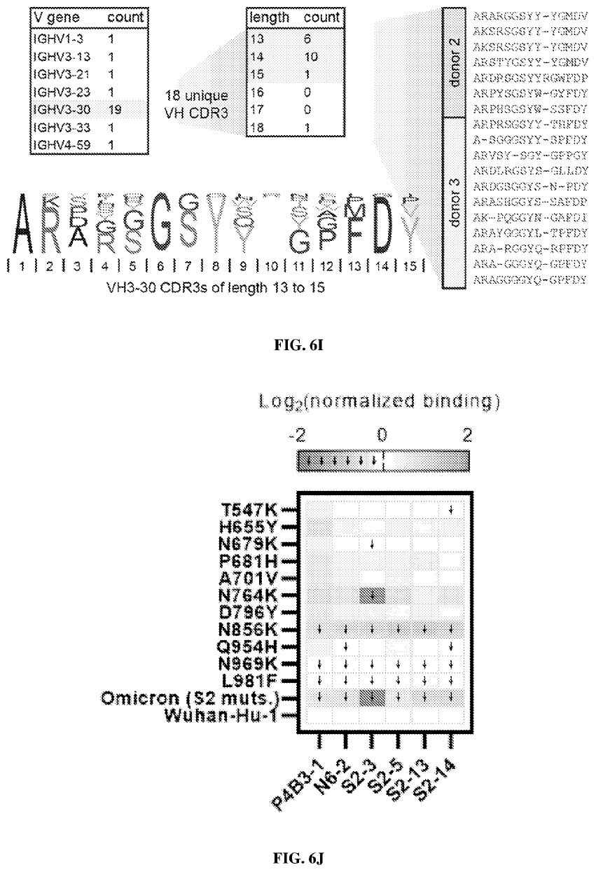

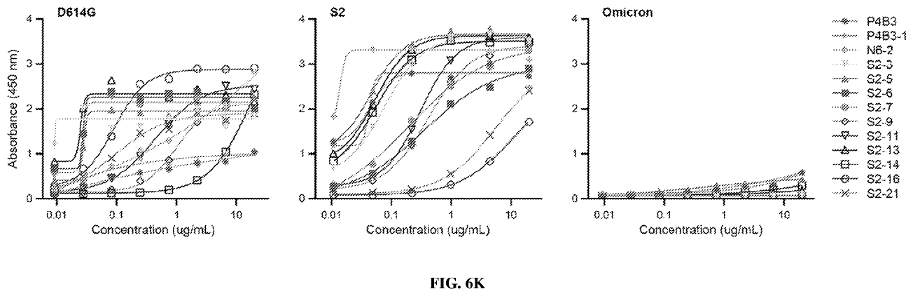

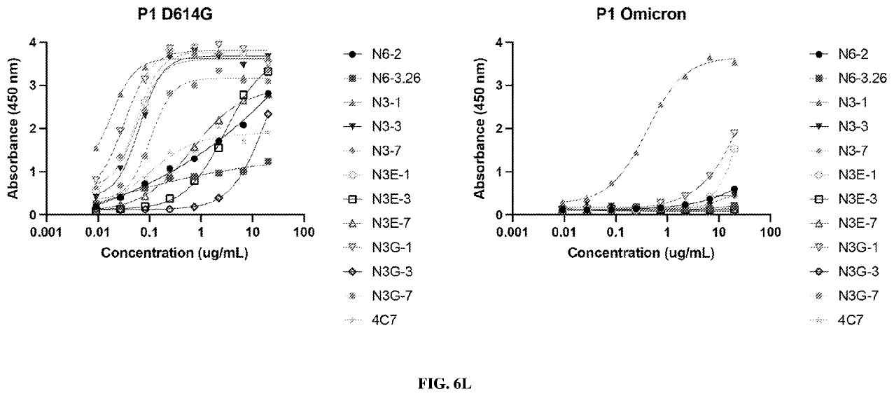

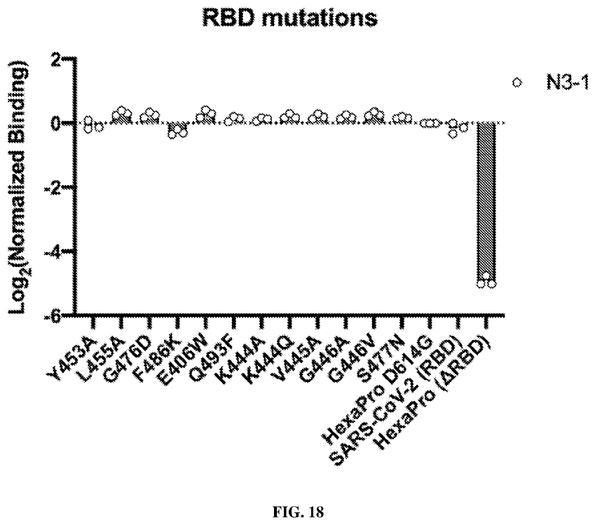

Whether binding of A7V3 (NTD-targeting) or N3-1 (RBD-targeting) was detrimentally affected by mutations emerging in SARS-COV-2 variants of concern was tested. Binding to the 12 most abundant SARS-COV-2 RBD variants from Houston (Long et al., 2020) was assessed and no detrimental losses in N3-a binding avidity were observed (Tables 18 and 19). Next, mammalian surface display (Javanmardi et al., 2021) of spike variants was used to test binding of N3-1 IgG to RBD mutations that were previously shown to reduce or abrogate binding to one or both of the approved therapeutic antibodies (REGN10987 and REG10933) as well as well as the Alpha, Beta, Gamma, and Delta spike protein variants (Tables 5 and 6). Strikingly, N3-1 was able to bind each of the mutant spike variants. N3-1 binding was slightly enhanced by 10 of the 12 mutant spike proteins tested (L455A, G476D, E406W, Q493F, K444A, K444Q, V445A, G446A, G446V, and S477N) using this assay. Variant Y453A was indistinguishable from binding to the prefusion stabilized SARS-COV-2 S D614G, and F486K was the only mutation shown to slightly reduce binding (˜6%) (FIG. 18). Then, binding to B.1.1.7, B.1.1.248, and B.1.351 spike protein variants was tested using the same mammalian surface display assay. Binding of A7V3 was reduced by all three variants, a result predicted from the above structural work. In contrast, N3-1 binding was largely unperturbed by the accumulated mutations (FIG. 6A) of B.1.1.7, B.1.1.248, and B.1.351 spike proteins in this assay (FIG. 6B).

To validate the mammalian display screening results, the binding of N3-1 Fab and full-length IgG to SARS-COV-2 S-protein Wuhan-Hu-1, Alpha and Beta were examined using surface plasmon resonance (SPR). Interestingly, the binding affinity of N3-1 Fab (FIG. 6C) was nearly 1000-fold lower than the full-length IgG (FIG. 6D), which exhibited comparable binding across Wuhan-Hu-1, Alpha and Beta variants. N3-1 Fab binding to each of the variants was only able to be fit to a heterogenous binding model (FIGS. 16A-16D), consistent with the structural studies provided herein. On the other hand, N3-1 IgG exhibited comparable binding to Wuhan-Hu-1 and its variants, with 67.9 pM apparent affinity to WuHan-Hu-1, 296 pM apparent affinity to B.1.1.7, and 300 pM apparent affinity to B.1.351 (FIGS. 6C-6E). Binding via SPR to SARS-COV-1 spike protein was also tested and it was discovered that N3-1 IgG cross-reacts with SARS-COV-1 spike with 28 nM affinity (FIGS. 16A-16D). We corroborated these findings with two orthogonal live virus neutralization assays against circulating VOCs. The results show that the wildtype WA1 strain and all four VOCs were fully neutralized by N3-1 IgG (FIGS. 6E-6F).

TABLE 19
Houston spike variant ELISAs.

| | ACE2 | | CR3022 | | N3-1 | | A7V3 | |
|---|---|---|---|---|---|---|---|---|
| | EC₅₀ | | EC₅₀ | | EC₅₀ | | EC₅₀ | |
| Variant | nM | R² | nM | R² | nM | R² | nM | R² |
| S-2P | 8.5 | 0.99 | 3.6 | 0.99 | 0.8 | 0.96 | 1.8 | 0.97 |
| S-2P | 7.4 | 0.95 | 3.9 | 0.98 | 1.4 | 0.99 | 4.1 | 0.98 |
| D614G | | | | | | | | |
| HexaPro | 8.9 | 0.95 | 7.3 | 0.97 | 1.6 | 0.99 | 8.7 | 0.99 |
| HexaPro | 4.9 | 0.99 | 5.9 | 0.98 | 1.6 | 0.99 | 3.1 | 0.97 |
| D614G | | | | | | | | |
| F338L | 4.5 | 0.97 | 15.9 | 0.99 | 1.5 | 0.93 | 2.5 | 0.98 |
| A352S | 4.4 | 0.99 | 4.3 | 0.97 | 1.6 | 0.98 | 3.7 | 0.98 |
| T385I | 4.8 | 0.97 | 5.2 | 0.96 | 1.0 | 0.99 | 1.6 | 0.98 |
| A419V | 5.4 | 0.99 | 3.9 | 0.96 | 2.1 | 0.95 | 1.9 | 0.96 |
| V445F | 4.2 | 0.95 | 5.4 | 0.98 | 0.9 | 0.99 | 3.1 | 0.99 |
| G446V | 4.0 | 0.96 | 3.3 | 0.99 | 2.0 | 0.97 | 3.1 | 0.99 |
| F456L | 3.8 | 0.99 | 3.9 | 0.98 | 1.5 | 0.98 | 3.9 | 0.98 |
| E484Q | 4.0 | 0.96 | 3.9 | 0.98 | 2.1 | 0.99 | 2.6 | 0.98 |
| A520S | 3.8 | 0.96 | 2.4 | 0.98 | 1.3 | 0.99 | 4.5 | 0.97 |
| K528R | 5.4 | 0.99 | 3.6 | 0.98 | 1.0 | 0.96 | 5.7 | 0.97 |
| S373P | 3.8 | 0.95 | 93.1 | 0.97 | 3.7 | 0.99 | 1.7 | 0.98 |
| R408T | 8.2 | 0.96 | 10.1 | 0.99 | 1.5 | 0.99 | 10.4 | 0.94 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Adams, P. D. et al. PHENIX: building new software for automated crystallographic structure determination. Acta Crystallogr. D Biol. Crystallogr. 58, 1948-1954 (2002).
Banach, B. B. et al. Paired Heavy and Light Chain Signatures Contribute to Potent SARS-CoV-2 in Public Neutralization Antibody Responses. https://papers.ssrn.com/abstract=3754549 (2020) doi: 10.2139/ssrn.3754549.
Barnes, C. O. et al. SARS-COV-2 neutralizing antibody structures inform therapeutic strategies. Nature 588, 682-687 (2020).
Barnes, C. O. et al. Structures of Human Antibodies Bound to SARS-COV-2 Spike Reveal Common Epitopes and Recurrent Features of Antibodies. Cell 182, 828-842.e16 (2020).
Baum, A. et al. Antibody cocktail to SARS-COV-2 spike protein prevents rapid mutational escape seen with individual antibodies. Science 369, 1014-1018 (2020).
Bian, L. et al. Impact of the Delta variant on vaccine efficacy and response strategies. Expert Rev. Vaccines 1-9 (2021) doi: 10.1080/14760584.2021.1976153.
Bolotin, D. A. et al. MiXCR: software for comprehensive adaptive immunity profiling. Nat. Methods 12, 380-381 (2015).
Boutz, D. R. et al. Proteomic Identification of Monoclonal Antibodies from Serum. Anal. Chem. 86, 4758-4766 (2014).
Brown, J. F. et al. Anti-Ebola Virus Antibody Levels in Convalescent Plasma and Viral Load After Plasma Infusion in Patients With Ebola Virus Disease. J. Infect. Dis. 218, 555-562 (2018).
Campos, D. M. de O., Fulco, U. L., de Oliveira, C. B. S. & Oliveira, J. I. N. SARS-COV-2 virus infection: Targets and antiviral pharmacological strategies. J. Evid.-Based Med. 13, 255-260 (2020).
Chi, X. et al. A neutralizing human antibody binds to the N-terminal domain of the Spike protein of SARS-COV-2. Science 369, 650-655 (2020).
Coutinho, R. M. et al. Model-based evaluation of transmissibility and reinfection for the P.1 variant of the SARS-COV-2. medRxiv 2021.03.03.21252706 (2021) doi: 10.1101/2021.03.03.21252706.
Croll, T. I. ISOLDE: a physically realistic environment for model building into low-resolution electron-density maps. Acta Crystallogr. Sect. Struct. Biol. 74, 519-530 (2018).
DeKosky, B. J. et al. In-depth determination and analysis of the human paired heavy- and light-chain antibody repertoire. Nat. Med. 21, 86-91 (2015).
Ehrenmann & Lefranc, Cold Spring Harbor Protoc., 6:737-749, 2011.

### Ehrenmann et al., Nucleic Acids Res., 38: D301-D307, 2010.

Emsley, P. & Cowtan, K. Coot: model-building tools for molecular graphics. Acta Crystallogr. D Biol. Crystallogr. 60, 2126-2132 (2004).
Emsley, P. & Cowtan, K. Coot: model-building tools for molecular graphics. Acta Crystallogr. D Biol. Crystallogr. 60, 2126-2132 (2004).
Farinholt, T. et al. Transmission event of SARS-COV-2 delta variant reveals multiple vaccine breakthrough infections. BMC Med. 19, 255 (2021).
Ferrareze, P. A. G. et al. E484K as an innovative phylogenetic event for viral evolution: Genomic analysis of the E484K spike mutation in SARS-COV-2 lineages from Brazil. bioRxiv 2021.01.27.426895 (2021) doi: 10.1101/2021.01.27.426895.
Goddard, T. D. et al. UCSF ChimeraX: Meeting modern challenges in visualization and analysis. Protein Sci. 27, 14-25 (2018).
Gontu, A. et al. Limited window for donation of convalescent plasma with high live-virus neutralizing antibody titers for COVID-19 immunotherapy. Commun. Biol. 4, 267 (2021).
Grant, T., Rohou, A. & Grigorieff, N. cisTEM, user-friendly software for single-particle image processing. eLife 7.
Hansen, J. et al. Studies in humanized mice and convalescent humans yield a SARS-COV-2 antibody cocktail. Science 369, 1010-1014 (2020).
Henderson, R. et al. Controlling the SARS-COV-2 spike glycoprotein conformation. Nat. Struct. Mol. Biol. 27, 925-933 (2020).
Hoffmann, M. et al. SARS-COV-2 variants B.1.351 and B.1.1.248: Escape from therapeutic antibodies and antibodies induced by infection and vaccination. bioRxiv 2021.02.11.430787 (2021) doi: 10.1101/2021.02.11.430787.
Hsieh, C.-L. et al. Structure-based design of prefusion-stabilized SARS-COV-2 spikes. Science 369, 1501-1505 (2020).

| | | |
|---|---|---|
| Immune | Repertoire | Sequencing. |

https://irweb.irepertoire.com/nir/showsample?sampleid=21746#.

| | | |
|---|---|---|
| Immune | Repertoire | Sequencing. |

https://irweb.irepertoire.com/nir/showsample?sampleid=21745.

| | | |
|---|---|---|
| Immune | Repertoire | Sequencing. |

https://irweb.irepertoire.com/nir/showsample?sampleid=21744.

| | | |
|---|---|---|
| Immune | Repertoire | Sequencing. |

https://irweb.irepertoire.com/nir/showsample?sampleid=21743.

Javanmardi, K. et al. Rapid characterization of spike variants via mammalian cell surface display. bioRxiv 2021.03.30.437622 (2021) doi: 10.1101/2021.03.30.437622.
Ju, B. et al. Human neutralizing antibodies elicited by SARS-COV-2 infection. Nature 584, 115-119 (2020).
Koenig, P.-A. et al. Structure-guided multivalent nanobodies block SARS-COV-2 infection and suppress mutational escape. Science 371, (2021).
Lavinder, J. J. et al. Identification and characterization of the constituent human serum antibodies elicited by vaccination. Proc. Natl. Acad. Sci. 111, 2259-2264 (2014).
Li, W. et al. Angiotensin-converting enzyme 2 is a functional receptor for the SARS coronavirus. Nature 426, 450-454 (2003).
Libster, R. et al. Early High-Titer Plasma Therapy to Prevent Severe Covid-19 in Older Adults. N. Engl. J. Med. 384, 610-618 (2021).
Liu, H. et al. Cross-Neutralization of a SARS-COV-2 Antibody to a Functionally Conserved Site Is Mediated by Avidity. Immunity 53, 1272-1280.e5 (2020).
Liu, L. et al. Potent Neutralizing Antibodies Directed to Multiple Epitopes on SARS-COV-2 Spike. bioRxiv 2020.06.17.153486 (2020) doi: 10.1101/2020.06.17.153486.
Liu, X. et al. Neutralizing Aptamers Block S/RBD-ACE2 Interactions and Prevent Host Cell Infection. Angew. Chem. Int. Ed Engl. (2021) doi: 10.1002/anie.202100345.
Liu, Y. et al. Delta spike P681R mutation enhances SARS-COV-2 fitness over Alpha variant. BioRxiv Prepr. Serv. Biol. 2021.08.12.456173 (2021) doi: 10.1101/2021.08.12.456173.
Long, S. W. et al. Molecular Architecture of Early Dissemination and Massive Second Wave of the SARS-COV-2 Virus in a Major Metropolitan Area. mBio 11, (2020).
Luo, C. H. et al. Infection with the SARS-COV-2 Delta Variant is Associated with Higher Infectious Virus Loads Compared to the Alpha Variant in both Unvaccinated and Vaccinated Individuals. MedRxiv Prepr. Serv. Health Sci. 2021.08.15.21262077 (2021) doi: 10.1101/2021.08.15.21262077.
McCallum, M., Walls, A. C., Bowen, J. E., Corti, D. & Veesler, D. Structure-guided covalent stabilization of coronavirus spike glycoprotein trimers in the closed conformation. Nat. Struct. Mol. Biol. 27, 942-949 (2020).
Pinto, D. et al. Cross-neutralization of SARS-COV-2 by a human monoclonal SARS-COV antibody. Nature 583, 290-295 (2020).
Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
Sanchez-Garcia, R. et al. DeepEMhancer: a deep learning solution for cryo-EM volume post-processing. bioRxiv 2020.06.12.148296 (2020) doi: 10.1101/2020.06.12.148296.
Shen, X. et al. SARS-COV-2 variant B.1.1.7 is susceptible to neutralizing antibodies elicited by ancestral Spike vaccines. bioRxiv 2021.01.27.428516 (2021) doi: 10.1101/2021.01.27.428516.
Shi, R. et al. A human neutralizing antibody targets the receptor-binding site of SARS-COV-2. Nature 584, 120-124 (2020).
Starr, T. N. et al. Prospective mapping of viral mutations that escape antibodies used to treat COVID-19. Science (2021) doi: 10.1126/science.abf9302.
Sui, J. et al. Potent neutralization of severe acute respiratory syndrome (SARS) coronavirus by a human mAb to S1 protein that blocks receptor association. Proc. Natl. Acad. Sci. U.S.A 101, 2536-2541 (2004).
Tai, W. et al. Characterization of the receptor-binding domain (RBD) of 2019 novel coronavirus: implication for development of RBD protein as a viral attachment inhibitor and vaccine. Cell. Mol. Immunol. 17, 613-620 (2020).
Tegally, H. et al. Detection of a SARS-COV-2 variant of concern in South Africa. Nature 592, 438-443 (2021).
Tegunov, D. & Cramer, P. Real-time cryo-electron microscopy data preprocessing with Warp. Nat. Methods 16, 1146-1152 (2019).
Tian, X. et al. Potent binding of 2019 novel coronavirus spike protein by a SARS coronavirus-specific human monoclonal antibody. Emerg. Microbes Infect. 9, 382-385 (2020).
Volz, E. et al. Assessing transmissibility of SARS-COV-2 lineage B.1.1.7 in England. Nature 1-6 (2021) doi: 10.1038/s41586-021-03470-x.
Voss, W. N. et al. Prevalent, protective, and convergent IgG recognition of SARS-COV-2 non-RBD spike epitopes in COVID-19 convalescent plasma. bioRxiv 2020.12.20.423708 (2020) doi: 10.1101/2020.12.20.423708.
Voss, W. N. et al. Prevalent, protective, and convergent IgG recognition of SARS-COV-2 non-RBD spike epitopes. Science (2021) doi: 10.1126/science.abg5268.
Wang, P. et al. Increased Resistance of SARS-COV-2 Variants B.1.351 and B.1.1.7 to Antibody Neutralization. bioRxiv 2021.01.25.428137 (2021) doi: 10.1101/2021.01.25.428137.
Wick, R. R., Judd, L. M. & Holt, K. E. Performance of neural network base calling tools for Oxford Nanopore sequencing. Genome Biol. 20, 129 (2019).
Wrapp, D. et al. Cryo-EM structure of the 2019-nCOV spike in the prefusion conformation. Science 367, 1260-1263 (2020).
Wu, F. et al. A new coronavirus associated with human respiratory disease in China. Nature 579, 265-269 (2020).
Xiaojie, S., Yu, L., lei, Y., Guang, Y. & Min, Q. Neutralizing antibodies targeting SARS-CoV-2 spike protein. Stem Cell Res. 50, 102125 (2021).
Xiong, X. et al. A thermostable, closed SARS-COV-2 spike protein trimer. Nat. Struct. Mol. Biol. 27, 934-941 (2020).
Yan, R. et al. Structural basis for the recognition of SARS-COV-2 by full-length human ACE2. Science 367, 1444-1448 (2020).
Yi, C. et al. Key residues of the receptor binding motif in the spike protein of SARS-COV-2 that interact with ACE2 and neutralizing antibodies. Cell. Mol. Immunol. 17, 621-630 (2020).
Yuan, M. et al. A highly conserved cryptic epitope in the receptor binding domains of SARS-CoV-2 and SARS-COV. Science 368, 630-633 (2020).
Yuan, M. et al. Structural basis of a shared antibody response to SARS-COV-2. Science 369, 1119-1123 (2020).
Zhou, P. et al. A pneumonia outbreak associated with a new coronavirus of probable bat origin. Nature 579, 270-273 (2020).
Zhou, X. et al. Diverse immunoglobulin gene usage and convergent epitope targeting in neutralizing antibody responses to SARS-COV-2. Cell Rep. 109109 (2021) doi: 10.1016/j.celrep.2021.109109.

1. A monoclonal antibody or antibody fragment, wherein the antibody or antibody fragment comprises a heavy chain variable region (VH) comprising VHCDR1, VHCDR2, and VHCDR3 amino acid sequences and a light chain variable region (VL) comprising VLCDR1, VLCDR2, and VLCDR3 amino acid sequences derived from clone-paired VH and VL sequence from Table 1.

2. The monoclonal antibody or antibody fragment of claim 1, wherein the antibody or antibody fragment comprises a heavy chain variable region (VH) comprising VHCDR1, VHCDR2, and VHCDR3 amino acid sequences derived from SEQ ID NO: 1 and a light chain variable region (VL) comprising VLCDR1, VLCDR2, and VLCDR3 amino acid sequences derived from SEQ ID NO: 2.

3. The monoclonal antibody or antibody fragment of claim 1, wherein the antibody or antibody fragment comprises clone-paired heavy and light chain CDR sequences from Table 2.

4. (canceled)

5. The monoclonal antibody or antibody fragment of claim 1, wherein the antibody or antibody fragment comprises clone-paired heavy chain and light chain variable sequences each having at least 95% identity to sequences from Table 1.

6. (canceled)

7. The monoclonal antibody or antibody fragment of claim 1, wherein the antibody fragment is a monovalent scFv (single chain fragment variable) antibody, divalent scFv, Fab fragment, F(ab′)₂ fragment, F(ab′)₃ fragment, Fv fragment, or single chain antibody.

8. The monoclonal antibody or antibody fragment of claim 1, wherein said antibody is a chimeric antibody, bispecific antibody, or BiTE.

9-23. (canceled)

24. The monoclonal antibody or antibody fragment of claim 2, wherein, when bound to a coronavirus spike protein trimer, the monoclonal antibody binds to residues of one subunit of the coronavirus spike protein trimer that correspond to positions 369, 377, 378, and 384 of SEQ ID NO: 298 and to residues of a second subunit of the coronavirus spike protein trimer that correspond to position 486, 487, 489, and 493 of SEQ ID NO: 298.

25. The monoclonal antibody or antibody fragment of claim 24, wherein, when bound to a coronavirus spike protein, the monoclonal antibody additionally binds to at least one residue of a coronavirus spike protein that corresponds to position 379 and 408 of SEQ ID NO: 298.

26. The monoclonal antibody or antibody fragment of claim 1, wherein the antibody is capable of binding to the spike proteins from SARS-COV, SARS-COV-2, and MERS.

27. The monoclonal antibody or antibody fragment of claim 1, wherein the antibody is capable of binding to SARS-COV-2 B.1.1.7, B.1.1.248, and/or B.1.351 spike protein variants.

28. An isolated nucleic acid encoding the antibody heavy and/or light chain variable region of the antibody or antibody fragment of claim 1.

29. (canceled)

30. A hybridoma or engineered cell comprising a nucleic acid encoding an antibody or antibody fragment of claim 1.

31. (canceled)

32. A method of making the monoclonal antibody or antibody fragment of claim 1, the method comprising culturing the hybridoma or engineered cell of claim 30 or 31 under conditions that allow expression of the antibody or antibody fragment and optionally isolating the antibody or antibody fragment from the culture.

33. A pharmaceutical formulation comprising one or more antibody or antibody fragment of claim 1.

34. (canceled)

35. A method of reducing the likelihood of a pathogenic coronavirus infection in a patient at risk of contracting the pathogenic coronavirus, the method comprising delivering to the patient an antibody or antibody fragment of claim 1.

36-39. (canceled)

40. A method of treating a patient infected with a pathogenic coronavirus, the method comprising delivering to the patient an antibody or antibody fragment of claim 1.

41-43. (canceled)

44. A method of detecting a coronavirus infection in a patient, the method comprising:
(a) contacting a sample obtained from the patient with an antibody or antibody fragment of claim 1; and
(b) detecting the coronavirus in the sample by detecting binding of the antibody or antibody fragment to a coronavirus antigen in the sample.

45-50. (canceled)

51. A method of detecting a coronavirus spike protein in an in vitro sample, the method comprising contacting the in vitro sample with an antibody or antibody fragment of claim 1 and detecting the binding of the antibody or antibody fragment to the sample.

52-54. (canceled)

55. A method of identifying an antibody that binds to an antigen of interest, the method comprising:
(a) obtaining antibody V_{H} chain sequence data for at least one antibody that binds to the antigen of interest; and
(b) identifying productive V_{L} pairs for the identified V_{H} chain by pairing the V_{H} chain with a V_{L} repertoire and testing for binding to the antigen of interest.

56-71. (canceled)

72. An antibody generated by the method of claim 55.


SEQUENCE LISTING (470 sequences)

SEQ ID NO: 1 - protein, 128 aa, Artificial sequence [sequence omitted]

SEQ ID NO: 2 - protein, 108 aa, Artificial sequence
  Synthetic polypeptide
DIQMTQSPSTLSASVGDRVTITCRASQSISSWLAWYQQKPGKAPKLLIYDASSLESGVPSRFSGSGSGTEFTLTISSLQPDDFATYYCQQYNSYSPWTFGQGTKVEIK

SEQ ID NO: 3 - protein, 121 aa, Artificial sequence
  Synthetic polypeptide
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATGPAVRRGSWFDPWGQGTLVTVSS

SEQ ID NO: 4 - protein, 110 aa, Artificial sequence
  Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 5 - protein, 122 aa, Artificial sequence
  Synthetic polypeptide
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATGSPFDRTQNWFDPWGQGTLVTVSS

SEQ ID NO: 6 - protein, 110 aa, Artificial sequence
  Synthetic polypeptide
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 7 - protein, 121 aa, Artificial sequence
  Synthetic polypeptide
QVQLVESGAEVKKPGASVKVSCKVSGYTLTELSMHWVRQAPGKGLEWMGGFDPEDGETIYAQKFQGRVTMTEDTSTDTAYMELSSLRSEDTAVYYCATRFAVYGDYLIDYWGQGTLVTVSS

SEQ ID NO: 8 - protein, 108 aa, Artificial sequence
  Synthetic polypeptide
SSELSQEPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKVTVL

SEQ ID NO: 9 - protein, 121 aa, Artificial sequence
  Synthetic polypeptide
QVQLVESGAEVQKPGASVKVSCKVSGYTLPELSMHWVRQAPGKGLEWMGGFDPEDGETFYAQKFQGRVTMTEDSSTDTAYMELSSLGSEDTAVYYCATAAAVRGRGTIDYWGQGTLVTVSS

SEQ ID NO: 10 - protein, 111 aa, Artificial sequence
  Synthetic polypeptide
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVVFGGGTKLTVL

SEQ ID NO: 11 - protein, 123 aa, Artificial sequence
  Synthetic polypeptide
QVQLVESGGGVVQPGGSLRLSCAASGFTFSSYGMHWVRQAPGKGLEWVAFIRYDGSNKYYADSVKGRFTISRDNSKNTLYLQMNSLRAEDTALYYCAKATRANYYYYYGMDVWGQGTTVTVSS

SEQ ID NO: 12 - protein, 114 aa, Artificial sequence
  Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPYTFGQGTKLEIK

SEQ ID NO: 13 - protein, 121 aa, Artificial sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARPYSGSYWGYFDYWGQGTLVTVSS

SEQ ID NO: 14 - protein, 110 aa, Artificial sequence
  Synthetic polypeptide
SYELTQLPSASGTPGQRVTISCSGGSSNIGSNYVYWYQQLPETAPKLLIYRNNKRPSGVPDRFSGSKSGTSASLAISGLRSEDEADYYCAAWDDSLSGPVFGTGTQLTVL

SEQ ID NO: 15 - protein, 108 aa, Artificial sequence [sequence omitted]

SEQ ID NO: 16 - protein, 121 aa, Artificial sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARARGGSYYYGMDVWGQGTTVTVSS

SEQ ID NO: 17 - protein, 109 aa, Artificial sequence
  Synthetic polypeptide
NFMLTQPHSVSESPGKAVTISCTRNSGSIASNFVQWFQQRPGSYPTTIMYENDNRPSGVPDRFSGSKSGTSATLGITGLQAEDEADYFCQSSDDSNQLVFGGGTKVTVL

SEQ ID NO: 18 - protein, 121 aa, Artificial sequence
  Synthetic polypeptide
QVTLKESGPALVKPTQTLTLTCTFSGFSLSTSGMCVTWVRQPPGKALEWLARIDWDDDKYYSTSLKTRLTISKDTSKNQVVLKMTNMDPVDTATYYCARIPIATHLGSDYWGQGTLVTVSS

SEQ ID NO: 19 - protein, 108 aa, Artificial sequence
  Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 20 - protein, 128 aa, Artificial sequence
  Synthetic polypeptide
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDTHSSSWRKYYYYYYGMDVWGQGTTVTVSS

SEQ ID NO: 21 - protein, 110 aa, Artificial sequence
  Synthetic polypeptide
SSELSQEPSFSVSPGGTVTLTCGLSSGSVSTNYYSSWYQQTPGQAPRTLIYSTNTRSSGVPDRFSGSILGNKAALTITGAQADDESDYYCTLYMGGGLLVFGGGTQLTVL

SEQ ID NO: 22 - protein, 127 aa, Artificial sequence
  Synthetic polypeptide
QVQLQESGAEVKKPGASVKVSCKASGYTFTGYYMHWVRQAPGQGLEWMGWINPNSGGTNYAQKFQGRVTMTADRSTNTVYMELSSLKSEDTAVYYCARELPPGRMVVPATYWHFDLWGRGTLVTVSL

SEQ ID NO: 23 - protein, 109 aa, Artificial sequence
  Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 24 - protein, 126 aa, Artificial sequence
  Synthetic polypeptide
QVQLVESGGGVVQPGRSLRLSCAASGFTFSSYAMHWVRQAPGKGLEWVAVISYDGSNKYYADSVKGRFTISRDNSKNTLYLQMDSLRPEDTAVYYCAKASQLFWLGQFTRDGFDIWGQGTMVTVSS

SEQ ID NO: 25 - protein, 117 aa, Artificial sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDGGGYVSYWGQGTLVTVSS

SEQ ID NO: 26 - protein, 106 aa, Artificial sequence
  Synthetic polypeptide
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 27 - protein, 112 aa, Artificial sequence
  Synthetic polypeptide
QSVLTQPPSVSGAPGQRVTISCTGSRSNIGVGYDVHWYQQLPGRAPKLVIYSNNNRPSGVSDRFSGSRSGSSASLAITGLQAEDEADYYCQSYDGSLNDDVIFGGGTQLTVL

SEQ ID NO: 28 - protein, 121 aa, Artificial sequence
  Synthetic polypeptide
QVQLQESGGGVVQPGRSLRLSCAASGFTFSSYAMHWVRQAPGKGLEWVAVISYDGSNKYYADSVKGRFTISRDNSKNTLYLQMNSLRAEDTAVYYCARPYSGSYWGYFDYWGQGTLVTVSS

SEQ ID NO: 29 - protein, 113 aa, Artificial sequence
  Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPYTFGQGTKLEIK

SEQ ID NO: 30 - protein, 124 aa, Artificial sequence
  Synthetic polypeptide
QVQLVESGGGVVQPGRSLRLSCAASGFIFNNYGMHWVRQAPGKGLEWVAVISYDGSNKYYADSVKGRFTISRDNSKNTLYLQMNSLRAEDTAVYYCAKAPGQWLRFHYYGMDVWGQGTTVTVSS

SEQ ID NO: 31 - protein, 109 aa, Artificial sequence
  Synthetic polypeptide
QSVVTQPPSVSGAPGQRVTISCTGSSSNIGAHYDVHWYQQVPGTAPKLLIYGDNNRPSGVPARFSGAKSGTSASLAITGLQADDEAHYYCQSYGNNQGVFGGGTKVTVL

SEQ ID NO: 32 - protein, 124 aa, Artificial sequence
  Synthetic polypeptide
QVQLVESGGGVVQPGRSLRLSCAASGFTFSSYAMHWVRQAPGKGLEWVAVISYDGSNKYYADSVKGRFAISRDISKNTLYLQMNSLRAEDTAVYYCARDDTGRVGSGWYCPLYWGQGTLVTVSS

SEQ ID NO: 33 - protein, 107 aa, Artificial sequence
  Synthetic polypeptide
DIRVTQSPSSLSASVGDRVTITCRASQSISSYLNWYQQKLGKAPKLLIYAASSLQSGVPSRFSGSGSGTDFTLTISSLQPEDFATYYCQQSYSTPLSFGGGTKVEIK

SEQ ID NO: 34 - protein, 128 aa, Artificial sequence
  Synthetic polypeptide
QVQLVQSGGGVVQPGRSLRLSCAASGFIFSSYGMHWVRQAPGKGLEWVAVISYDGSNKYYADSVKGRFTISRDNSKNTLYLQMNSLRAEDTAVYYCAKQAGAYCSGGSCYSSSEADYWGQGTLVTVSS

SEQ ID NO: 35 - protein, 109 aa, Artificial sequence
  Synthetic polypeptide
QSVVTQPPSVSGAPGQRVTISCTGSSSNIGAHYDVHWYQQVPGTAPKLLIYGDNNRPSGVPARFSGAKSGTSASLAITGLQADDEAHYYCQSYGNNQGMFGGGTQLTVL

SEQ ID NO: 36 - protein, 105 aa, Artificial sequence
  Synthetic polypeptide
SYELMQLPSVSVSPGQTASITCSGDKLGDKYACWYQQKPGQSPVLVIYQDSKRPSGIPERFSGSNSGNTATLTISGTQAMDEADYYCQAWDSSTFFGTGTKVTVL

SEQ ID NO: 37 - protein, 106 aa, Artificial sequence
  Synthetic polypeptide
SYELTQPLSVSVALGQTARITCGGNKLGDKYACWYQQKPGQSPVLVIYQDSKRPSGIPERFSGSNSGNTATLTISGTQAMDEADYYCQAWDSSTVVFGGGTKLTVL

SEQ ID NO: 38 - protein, 113 aa, Artificial sequence
  Synthetic polypeptide
QVQLVESGGGLVQPGGSLTLSCAASGFTFSSYAMTWVRQAPGKGLEWVSSISTRGNTYYADSVKGRFTISRDNSKNTLYLQMNSLRGEDTAVYYCAPGRSLYWGQGTLVTVSS

SEQ ID NO: 39 - protein, 111 aa, Artificial sequence
  Synthetic polypeptide
QSVLTQPPSVSGAPGQTVTISCTGSTSNIGADYDVQWYQQLPGRAPKLLIYGNTKRPSGVADRFSASKSGTSASLAITGLQAGDEGDYHCQSYDSGLSGSIFGGGTKLTVL

SEQ ID NO: 40 - protein, 122 aa, Artificial sequence
  Synthetic polypeptide
EVQLVESGGGVVQPGKSLRLSCAASGFTFNNYGMHWVRQAPGKGLEWVALISYDGSNKYYADSVKGRFTISRDNSKNTLYLQMNSLRAEDTAVYYCARDRSGSYYRGWFDPWGQGTLVTVSS

SEQ ID NO: 41 - protein, 114 aa, Artificial sequence
  Synthetic polypeptide
DIQLTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPYTFGQGTKVEIK

SEQ ID NO: 42 - protein, 127 aa, Artificial sequence
  Synthetic polypeptide
EVQLVESGGDLVQPGGSLRLSCAASGFTFSNYAMSWVRQAPGKGLEWVSAISNSGDKTYYADSVKGRFTISRDNSKNTLFLQMNRLRTEDTAVYYCARVRGYSVNWRYYYHYGMDVWGQGTLVTVSS

SEQ ID NO: 43 - protein, 111 aa, Artificial sequence
  Synthetic polypeptide
QPGLTQPHSVSESPGKAVTISCTRNSGSITSNFVQWFQQRPGSYPTTIMYENDKRPTGVPHRFSGSIDSSSNSASLSISGLRAEDEADYYCQSSDDSNQLVFGGGTKLTVL

SEQ ID NO: 44 - protein, 113 aa, Artificial sequence
  Synthetic polypeptide
ETTLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPYTFGQGTKVEIK

SEQ ID NO: 45 - protein, 106 aa, Artificial sequence
  Synthetic polypeptide
SYELIQPPSVSVSPGQTASITCSGDKLGDKYACWYQQKPGQSPVLVIYQDSKRPSGIPERFSGSNSGNTATLTISGTQAMDEADYYCQAWDSSTGVFGGGTKVTVL

SEQ ID NO: 46 - protein, 124 aa, Artificial sequence
  Synthetic polypeptide
QVQLVQSGGGLVQPGGSLRLSCAASGFTFSNYAMNWVRQAPGKGLEWVSGISGSGGSTYYADSVKGRFTISRDNSKNTLYLQMNSLRAEDTAVYYCARDRFQSYTYYYYGMDVWGQGTTVTVSS

SEQ ID NO: 47 - protein, 125 aa, Artificial sequence
  Synthetic polypeptide
QITLKESGPVLVKPTETLTLTCTVSGFSLNNRMGVSWLRQPPGKALEWLAHTFSSGEPSYSTSLKNRVTISKDTSKGQVVLTMANVDPLDTATYYCARVMTSMRLWYWGYWFDPWGQGIRVTVSS

SEQ ID NO: 48 - protein, 112 aa, Artificial sequence
  Synthetic polypeptide
QSVLIQPPSVSGAPGQRVTISCTGSRSNIGVGYDVHWYQQLPGRAPKLVIYSNNNRPSGVSDRFSGSRSGSSASLAITGLQAEDEADYYCQSYDGSLNDDVIFGGGTKLTVL

SEQ ID NO: 49 - protein, 111 aa, Artificial sequence
  Synthetic polypeptide
QSVVTQPPSVSGAPGQTVTISCTGSTSNIGADYDVQWYQQLPGRAPKLLIYGNTKRPSGVADRFSASKSGTSASLAITGLQAGDEGDYHCQSYDSGLSGSIFGGGTQLTVL

SEQ ID NO: 50 - protein, 121 aa, Artificial sequence
  Synthetic polypeptide
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARPYSGSYWGYFDYWGQGTLVTVSS

SEQ ID NO: 51 - protein, 110 aa, Artificial sequence
  Synthetic polypeptide
SSELSQPPSASGTPGQRVTISCSGGSSNIGSNYVYWYQQLPETAPKLLIYRNNKRPSGVPDRFSGSKSGTSASLAISGLRSEDEADYYCAAWDDSLSGPVFGTGTKLTVL

SEQ ID NO: 52 - protein, 109 aa, Artificial sequence
  Synthetic polypeptide
QSVLTQPPSVSGAPGQRVTISCTGSTSNIGATYDVHWYQQRPGAAPKLLIYGNNNRPSGVPDRFSGSKSGTSASLAITGLQADDEAHYYCQSYGNNQGVFGGGTKLTVL

SEQ ID NO: 53 - protein, 123 aa, Artificial sequence
  Synthetic polypeptide
QVTLKESGAEVKKPGASVKVSCKASGYTFTGYYLHWVRQAPGQGLEWMGWINPNSGGTNYAQKFQGRVTMTRDTSISTAYMELSRLRSDDTAVYYCARLRGYSYGHYYGMDVWGQGTTVTVSS

SEQ ID NO: 54 - protein, 108 aa, Artificial sequence
  Synthetic polypeptide
DIVLTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVDIK

SEQ ID NO: 55 - protein, 124 aa, Artificial sequence
  Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDARRFKYYYYYGMDVWGQGTTVTVSS

SEQ ID NO: 56 - protein, 112 aa, Artificial sequence
  Synthetic polypeptide
QSVLTQPPSVSGAPGQRVTISCTGGSSNLGAAYDVHWYQQYPGRAPKLLIYNNNNRPSGVPDRFSGSKSDTSASLVIAGLQAEDEAEYYCQSYDISLSGLYVFGTGTKVTVL

SEQ ID NO: 57 - protein, 111 aa, Artificial sequence
  Synthetic polypeptide
QPVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVVFGGGTKLTVL

SEQ ID NO: 58 - protein, 120 aa, Artificial sequence
  Synthetic polypeptide
QVQLVQSGGGLVQPGGSLRLSCAASGFTFGGYPMHWVRLAPGKGLEWLAVISYDGSSAYYIDSVKGRFTVSRDNSKNTLYLEMSSLGTEDTAVYYCARPRSYRNHRFDYWGQGTLVTVSS

SEQ ID NO: 59 - protein, 124 aa, Artificial sequence
  Synthetic polypeptide
QVQLVQSGGGVVQPGRSLRLSCAASGFIFNNYGMHWVRQAPGKGLEWVAVISYDGSNKYYADSVKGRFTISRDNSKNTLYLQMNSLRAEDTAVYYCAKAPGQWLRFHYYGMDVWGQGTTVTVSS

SEQ ID NO: 60 - protein, 111 aa, Artificial sequence
  Synthetic polypeptide
SSELTQDPAVSVALGQTVKITCQGDSSNIGAHYDVHWYQQVPGTAPKLLIYGKNNRPSGVPARFSGAKSGTSASLAITGLQADDEAHYYCNSRDINSNHVLFGGGTKVTVL

SEQ ID NO: 61 - protein, 133 aa, Artificial sequence
  Synthetic polypeptide
QVQLVESGRRGPAWGSLRLSCAASGFTFSSYGMHWVRQAPGKGLEWVAFIRYDGSNKYYADSVKGRFTISRDNSKNTLYLQMNSLRAEDTAVYYCAKDRRYYDFWSGYVGSPYYYYYYGMDVWGQGTTVTVSS

SEQ ID NO: 62 - protein, 105 aa, Artificial sequence
  Synthetic polypeptide
SYELMHTSVSVSPGQTASITCSGDKLGDKYACWYQQKPGQSPVLVIYSNNKRPSGIPERFLSGSNSGNTATDHQRTQAMDEADYYCQSYGNNQGVFGGGTKVTVL

SEQ ID NO: 63 - protein, 113 aa, Artificial sequence
  Synthetic polypeptide
QVQLVQSGGGVVQPGRSLRLSCAASGFTFSDYGMHWVRQAPGKGLEWVAVVSYDGSNKYYADSVKGRFTISRDNSKNTVYLQMNSLRAEDTAVYYCASPTVTMGHGKLVTVSS

SEQ ID NO: 64 - protein, 107 aa, Artificial sequence
  Synthetic polypeptide
DIQMTQSPSSLSASVGDRVTITCRASQSISTYLNWYQQKPGKAPKLLIYAASRLQSEVPSRFSGSGSGTDFTLTISSLEPEDFATYSCQQSYSSSWTFGQGTKVEIK

SEQ ID NO: 65 - protein, 121 aa, Artificial sequence
  Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGNWGSYYYGMDVWGQGTTVTVSS

SEQ ID NO: 66 - protein, 107 aa, Artificial sequence
  Synthetic polypeptide
SYELMQLPSVSVSPGQTASITCSGDNIGSKSVHWYQQKPGQAPVLVVYDDSKRPSGIPERFSGSNSGNTATLTISGTQAMDEADYYCQSSDDSNQLVFGGGTKVTVL

SEQ ID NO: 67 - protein, 122 aa, Artificial sequence
  Synthetic polypeptide
QVQLVQSGGGLVQPGGSLRLSCAASGFTVSSNYMSWVRQAPGKGLEWVSVIYSGGSTYYADSVKGRFTISRDNSKNTLYLQMNSLRAEDTAVYYCARGGVVDTYYYYGMDVWGQGTTVTVSS

SEQ ID NO: 68 - protein, 109 aa, Artificial sequence
  Synthetic polypeptide
QAVVTQEPSLTVSPGGTVTLTCGSSTGPVTTGHWPYWLQQKTGQAPRTLIYDTVNKHSWTPARFSGSLLGGKAALTLSGAQPEDEADHYCLLSQSGAWVFGGGTKLTVL

SEQ ID NO: 69 - protein, 110 aa, Artificial sequence
  Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSGYVFGTGTKVTVL

SEQ ID NO: 70 - protein, 109 aa, Artificial sequence
  Synthetic polypeptide
QSVLIQPPSVSGAPGQRVTISCTGSSSNIGAHYDVHWYQQVPGTAPKLLIYGDNNRPSGVPARFSGAKSGTSASLAITGLQADDEAHYYCQSYGNNQGVFGGGTKVTVL

SEQ ID NO: 71 - protein, 110 aa, Artificial sequence
  Synthetic polypeptide
QSVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLAFGGGTKLTVL

SEQ ID NO: 72 - protein, 110 aa, Artificial sequence
  Synthetic polypeptide
QSVVTQPPSVSAAPGQKVTISCSGSRSNIGNNYVSWYQQLPGTAPKLLIYDYDKRPSGIPDRFSGSKSGTSATLGITGLQTGDEADYYCGTWDNSLSAGVFGGGTRLTVL

SEQ ID NO: 73 - protein, 121 aa, Artificial sequence
  Synthetic polypeptide
QVQLQESGGGVVQPGRSLRLSCAASGFTFSSYAMHWVRQAPGKGLEWVAVISYDGSNKYYADSVKGRFTISRDNSKNTLYLQMNSLRAEDTAVYYCATGPAVRRGSWFDPWGQGTLVTVSS

SEQ ID NO: 74 - protein, 107 aa, Artificial sequence
  Synthetic polypeptide
AIQLTQSPSSLSASVGDRVTITCRASQSISSYLNWYQQKPGKAPKLLIYAASSMQSGVPSRFSGSGSGTDFTLTISSLQPEDFATYYCQQSYSTPRTFGQGTKVEIK

SEQ ID NO: 75 - protein, 110 aa, Artificial sequence
  Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVL

SEQ ID NO: 76 - protein, 128 aa, Artificial sequence
  Synthetic polypeptide
QVQLQQSGPGLVNPSETLSLTCSVSGGSFATENYYWSWIRQHPGEGLEWIGNIYFSGNTYYNPSLNNRFTISFDTSKNHLSLKLPSVTAADTAVYYCARGTIYFDRSGYRRVDPFHIWGQGTMVIVSS

SEQ ID NO: 77 - protein, 108 aa, Artificial sequence
  Synthetic polypeptide
AIQLTQSPSSLSASVGDRVTITCRASQSISSYLNWYQQKPGKAPKLLIYAASSLQSGVPSRFSGSGSGTDFTLTISSLQPEDFATYYCQQSYSTRPLTFGGGTKVEIK

SEQ ID NO: 78 - protein, 110 aa, Artificial sequence
  Synthetic polypeptide
QSVLTQPPSVSAAPGQKVTISCSGSSSNIGKNYVSWYQQFPGTAPKLLIYSNNQRPSGVPDRFFGSKSGTSASLAISGLQSEDEADYYCAAWDDGLSAVVFGGGTKLTVL

SEQ ID NO: 79 - protein, 116 aa, Artificial sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAIYYCARDYGRGGVWGQGTTVTVSS

SEQ ID NO: 80 - protein, 127 aa, Artificial sequence
  Synthetic polypeptide
EVQLLESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRTYDYVWGSYRYEDYWGQGTLVTVSS

SEQ ID NO: 81 - protein, 106 aa, Artificial sequence
  Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPEFGQGTKVEIK

SEQ ID NO: 82 - protein, 124 aa, Artificial sequence
  Synthetic polypeptide
QVQLVESGGGVVQPGRSLRLSCVVSGFTFNNFGMHWVRQAPGKGLEWVAYISYEGSIKYYAQSLKGRFTISRDSSKNTLYLQMNSLRADDTAMYYCAKDRAIFYLNPRYYLDYWGQGILVTVSS

SEQ ID NO: 83 - protein, 107 aa, Artificial sequence
  Synthetic polypeptide
SYVLTQPPSVSVAPGKTARITCGGNKLGDKYACWYQQKPGQSPVLVIYQDSKRPSGIPERFSGSNSGNTATLTISGTQAMDEADYYCQAWDSSTAVVFGGGTKLTVL

SEQ ID NO: 84 - protein, 10 aa, Artificial sequence
  Synthetic polypeptide
GGSFATENYY

SEQ ID NO: 85 - protein, 7 aa, Artificial sequence
  Synthetic polypeptide
IYFSGNT

SEQ ID NO: 86 - protein, 20 aa, Artificial sequence
  Synthetic polypeptide
ARGTIYFDRSGYRRVDPFHI

SEQ ID NO: 87 - protein, 6 aa, Artificial sequence
  Synthetic polypeptide
QSISSW

SEQ ID NO: 88 - protein, 3 aa, Artificial sequence
  Synthetic polypeptide
DAS

SEQ ID NO: 89 - protein, 10 aa, Artificial sequence
  Synthetic polypeptide
QQYNSYSPWT

SEQ ID NO: 90 - protein, 8 aa, Artificial sequence
  Synthetic polypeptide
GYTLTELS

SEQ ID NO: 91 - protein, 8 aa, Artificial sequence
  Synthetic polypeptide
FDPEDGET

SEQ ID NO: 92 - protein, 14 aa, Artificial sequence
  Synthetic polypeptide
ATGPAVRRGSWFDP

SEQ ID NO: 93 - protein, 8 aa, Artificial sequence
  Synthetic polypeptide
SSNIGNNY

SEQ ID NO: 94 - protein, 3 aa, Artificial sequence
  Synthetic polypeptide
DNN

SEQ ID NO: 95 - protein, 11 aa, Artificial sequence
  Synthetic polypeptide
GTWDSSLSAVV

SEQ ID NO: 96 - protein, 15 aa, Artificial sequence
  Synthetic polypeptide
ATGSPFDRTQNWFDP

SEQ ID NO: 97 - protein, 14 aa, Artificial sequence
  Synthetic polypeptide
ATRFAVYGDYLIDY

SEQ ID NO: 98 - protein, 6 aa, Artificial sequence
  Synthetic polypeptide
SLRIYY

SEQ ID NO: 99 - protein, 3 aa, Artificial sequence
  Synthetic polypeptide
GKH

SEQ ID NO: 100 - protein, 11 aa, Artificial sequence
  Synthetic polypeptide
NSRDSSGDLVV

SEQ ID NO: 101 - protein, 8 aa, Artificial sequence
  Synthetic polypeptide
GYTLPELS

SEQ ID NO: 102 - protein, 14 aa, Artificial sequence
  Synthetic polypeptide
ATAAAVRGRGTIDY

SEQ ID NO: 103 - protein, 8 aa, Artificial sequence
  Synthetic polypeptide
SSNIGSNT

SEQ ID NO: 104 - protein, 3 aa, Artificial sequence
  Synthetic polypeptide
SNN

SEQ ID NO: 105 - protein, 12 aa, Artificial sequence
  Synthetic polypeptide
AAWDDSLNGPVV

SEQ ID NO: 106 - protein, 8 aa, Artificial sequence
  Synthetic polypeptide
GFTFSSYG

SEQ ID NO: 107 - protein, 8 aa, Artificial sequence
  Synthetic polypeptide
IRYDGSNK

SEQ ID NO: 108 - protein, 16 aa, Artificial sequence
  Synthetic polypeptide
AKATRANYYYYYGMDV

SEQ ID NO: 109 - protein, 12 aa, Artificial sequence
  Synthetic polypeptide
QSVLYSSNNKNY

SEQ ID NO: 110 - protein, 3 aa, Artificial sequence
  Synthetic polypeptide
WAS

SEQ ID NO: 111 - protein, 10 aa, Artificial sequence
  Synthetic polypeptide
QQYYSTPPYT

SEQ ID NO: 112 - protein, 8 aa, Artificial sequence
  Synthetic polypeptide
GFTFSSYA

SEQ ID NO: 113 - protein, 8 aa, Artificial sequence
  Synthetic polypeptide
ISYDGSNK

SEQ ID NO: 114 - protein, 14 aa, Artificial sequence
  Synthetic polypeptide
ARPYSGSYWGYFDY

SEQ ID NO: 115 - protein, 8 aa, Artificial sequence
  Synthetic polypeptide
SSNIGSNY

SEQ ID NO: 116 - protein, 3 aa, Artificial sequence
  Synthetic polypeptide
RNN

SEQ ID NO: 117 - protein, 11 aa, Artificial sequence
  Synthetic polypeptide
AAWDDSLSGPV

SEQ ID NO: 118 - protein, 6 aa, Artificial sequence
  Synthetic polypeptide
QSVSSY

SEQ ID NO: 119 - protein, 10 aa, Artificial sequence
  Synthetic polypeptide
QQRSNWPPLT

SEQ ID NO: 120 - protein, 14 aa, Artificial sequence
  Synthetic polypeptide
ARARGGSYYYGMDV

SEQ ID NO: 121 - protein, 8 aa, Artificial sequence
  Synthetic polypeptide
SGSIASNF

SEQ ID NO: 122 - protein, 3 aa, Artificial sequence
  Synthetic polypeptide
END

SEQ ID NO: 123 - protein, 10 aa, Artificial sequence
  Synthetic polypeptide
QSSDDSNQLV

SEQ ID NO: 124 - protein, 10 aa, Artificial sequence
  Synthetic polypeptide
GFSLSTSGMC

SEQ ID NO: 125 - protein, 7 aa, Artificial sequence
  Synthetic polypeptide
IDWDDDK

SEQ ID NO: 126 - protein, 13 aa, Artificial sequence
  Synthetic polypeptide
ARIPIATHLGSDY

SEQ ID NO: 127 - protein, 6 aa, Artificial sequence
  Synthetic polypeptide
QSVSSN

SEQ ID NO: 128 - protein, 3 aa, Artificial sequence
  Synthetic polypeptide
GAS

SEQ ID NO: 129 - protein, 10 aa, Artificial sequence [sequence omitted]

SEQ ID NO: 130 - protein, 21 aa, Artificial sequence
  Synthetic polypeptide
AKDTHSSSWRKYYYYYYGMDV

SEQ ID NO: 131 - protein, 9 aa, Artificial sequence
  Synthetic polypeptide
SGSVSTNYY

SEQ ID NO: 132 - protein, 3 aa, Artificial sequence
  Synthetic polypeptide
STN

SEQ ID NO: 133 - protein, 10 aa, Artificial sequence
  Synthetic polypeptide
TLYMGGGLLV

SEQ ID NO: 134 - protein, 8 aa, Artificial sequence
  Synthetic polypeptide
GYTFTGYY

SEQ ID NO: 135 - protein, 8 aa, Artificial sequence
  Synthetic polypeptide
INPNSGGT

SEQ ID NO: 136 - protein, 20 aa, Artificial sequence
  Synthetic polypeptide
ARELPPGRMVVPATYWHFDL

SEQ ID NO: 137 - protein, 7 aa, Artificial sequence
  Synthetic polypeptide
QSVSSSY

SEQ ID NO: 138 - protein, 10 aa, Artificial sequence
  Synthetic polypeptide
QQYGSSPPWT

SEQ ID NO: 139 - protein, 19 aa, Artificial sequence
  Synthetic polypeptide
AKASQLFWLGQFTRDGFDI

SEQ ID NO: 140 - protein, 10 aa, Artificial sequence
  Synthetic polypeptide
ARDGGGYVSY

SEQ ID NO: 141 - protein, 6 aa, Artificial sequence
  Synthetic polypeptide
KLGDKY

SEQ ID NO: 142 - protein, 3 aa, Artificial sequence
  Synthetic polypeptide
QDS

SEQ ID NO: 143 - protein, 9 aa, Artificial sequence
  Synthetic polypeptide
QAWDSSTVV

SEQ ID NO: 144 - protein, 9 aa, Artificial sequence
  Synthetic polypeptide
RSNIGVGYD

SEQ ID NO: 145 - protein, 12 aa, Artificial sequence
  Synthetic polypeptide
QSYDGSLNDDVI

SEQ ID NO: 146 - protein, 11 aa, Artificial sequence
  Synthetic polypeptide
QSLLHSNGYNY

SEQ ID NO: 147 - protein, 3 aa, Artificial sequence
  Synthetic polypeptide
LGS

SEQ ID NO: 148 - protein, 10 aa, Artificial sequence
  Synthetic polypeptide
MQALQTPPYT

SEQ ID NO: 149 - protein, 8 aa, Artificial sequence
  Synthetic polypeptide
GFIFNNYG

SEQ ID NO: 150 - protein, 17 aa, Artificial sequence
  Synthetic polypeptide
AKAPGQWLRFHYYGMDV

SEQ ID NO: 151 - protein, 9 aa, Artificial sequence
  Synthetic polypeptide
SSNIGAHYD

SEQ ID NO: 152 - protein, 3 aa, Artificial sequence
  Synthetic polypeptide
GDN

SEQ ID NO: 153 - protein, 9 aa, Artificial sequence
  Synthetic polypeptide
QSYGNNQGV

SEQ ID NO: 154 - protein, 17 aa, Artificial sequence
  Synthetic polypeptide
ARDDTGRVGSGWYCPLY

SEQ ID NO: 155 - protein, 6 aa, Artificial sequence
  Synthetic polypeptide
QSISSY

SEQ ID NO: 156 - protein, 3 aa, Artificial sequence
  Synthetic polypeptide
AAS

SEQ ID NO: 157 - protein, 9 aa, Artificial sequence
  Synthetic polypeptide
QQSYSTPLS

SEQ ID NO: 158 - protein, 8 aa, Artificial sequence
  Synthetic polypeptide
GFIFSSYG

SEQ ID NO: 159 - protein, 21 aa, Artificial sequence
  Synthetic polypeptide
AKQAGAYCSGGSCYSSSEADY

SEQ ID NO: 160 - protein, 9 aa, Artificial sequence
  Synthetic polypeptide
QSYGNNQGM

SEQ ID NO: 161 - protein, 8 aa, Artificial sequence
  Synthetic polypeptide
QAWDSSTF

SEQ ID NO: 162 - protein, 7 aa, Artificial sequence
  Synthetic polypeptide
ISTRGNT

SEQ ID NO: 163 - protein, 7 aa, Artificial sequence
  Synthetic polypeptide
APGRSLY

SEQ ID NO: 164 - protein, 9 aa, Artificial sequence
  Synthetic polypeptide
TSNIGADYD

SEQ ID NO: 165 - protein, 3 aa, Artificial sequence
  Synthetic polypeptide
GNT

SEQ ID NO: 166 - protein, 11 aa, Artificial sequence
  Synthetic polypeptide
QSYDSGLSGSI

SEQ ID NO: 167 - protein, 8 aa, Artificial sequence
  Synthetic polypeptide
GFTFNNYG

SEQ ID NO: 168 - protein, 15 aa, Artificial sequence
  Synthetic polypeptide
ARDRSGSYYRGWFDP

SEQ ID NO: 169 - protein, 8 aa, Artificial sequence
  Synthetic polypeptide
GFTFSNYA

SEQ ID NO: 170 - protein, 8 aa, Artificial sequence
  Synthetic polypeptide
ISNSGDKT

SEQ ID NO: 171 - protein, 20 aa, Artificial sequence
  Synthetic polypeptide
ARVRGYSVNWRYYYHYGMDV

SEQ ID NO: 172 - protein, 8 aa, Artificial sequence
  Synthetic polypeptide
SGSITSNF

SEQ ID NO: 173 - protein, 9 aa, Artificial sequence
  Synthetic polypeptide
QAWDSSTGV

SEQ ID NO: 174 - protein, 8 aa, Artificial sequence
  Synthetic polypeptide
ISGSGGST

SEQ ID NO: 175 - protein, 17 aa, Artificial sequence
  Synthetic polypeptide
ARDRFQSYTYYYYGMDV

SEQ ID NO: 176 - protein, 9 aa, Artificial sequence
  Synthetic polypeptide
GFSLNNRMG

SEQ ID NO: 177 - protein, 7 aa, Artificial sequence
  Synthetic polypeptide
TFSSGEP

SEQ ID NO: 178 - protein, 18 aa, Artificial sequence
  Synthetic polypeptide
ARVMTSMRLWYWGYWFDP

SEQ ID NO: 179 - protein, 9 aa, Artificial sequence
  Synthetic polypeptide
TSNIGATYD

SEQ ID NO: 180 - protein, 3 aa, Artificial sequence
  Synthetic polypeptide
GNN

SEQ ID NO: 181 - protein, 16 aa, Artificial sequence
  Synthetic polypeptide
ARLRGYSYGHYYGMDV

SEQ ID NO: 182 - protein, 17 aa, Artificial sequence
  Synthetic polypeptide
ARDARRFKYYYYYGMDV

SEQ ID NO: 183 - protein, 9 aa, Artificial sequence
  Synthetic polypeptide
SSNLGAAYD

SEQ ID NO: 184 - protein, 3 aa, Artificial sequence
  Synthetic polypeptide
NNN

SEQ ID NO: 185 - protein, 12 aa, Artificial sequence
  Synthetic polypeptide
QSYDISLSGLYV

SEQ ID NO: 186 - protein, 8 aa, Artificial sequence
  Synthetic polypeptide
GFTFGGYP

SEQ ID NO: 187 - protein, 8 aa, Artificial sequence
  Synthetic polypeptide
ISYDGSSA

SEQ ID NO: 188 - protein, 13 aa, Artificial sequence
  Synthetic polypeptide
ARPRSYRNHRFDY

SEQ ID NO: 189 - protein, 3 aa, Artificial sequence
  Synthetic polypeptide
GKN

SEQ ID NO: 190 - protein, 11 aa, Artificial sequence
  Synthetic polypeptide
NSRDINSNHVL

SEQ ID NO: 191 - protein, 27 aa, Artificial sequence
  Synthetic polypeptide
AKDRRYYDFWSGYVGSPYYYYYYGMDV

SEQ ID NO: 192 - protein, 8 aa, Artificial sequence
  Synthetic polypeptide
GFTFSDYG

SEQ ID NO: 193 - protein, 8 aa, Artificial sequence
  Synthetic polypeptide
VSYDGSNK

SEQ ID NO: 194 - protein, 6 aa, Artificial sequence
  Synthetic polypeptide
ASPTVT

SEQ ID NO: 195 - protein, 6 aa, Artificial sequence
  Synthetic polypeptide
QSISTY

SEQ ID NO: 196 - protein, 9 aa, Artificial sequence
  Synthetic polypeptide
QQSYSSSWT

SEQ ID NO: 197 - protein, 14 aa, Artificial sequence
  Synthetic polypeptide
ARGNWGSYYYGMDV

SEQ ID NO: 198 - protein, 6 aa, Artificial sequence
  Synthetic polypeptide
NIGSKS

SEQ ID NO: 199 - protein, 3 aa, Artificial sequence
  Synthetic polypeptide
DDS

SEQ ID NO: 200 - protein, 8 aa, Artificial sequence
  Synthetic polypeptide
GFTVSSNY

SEQ ID NO: 201 - protein, 7 aa, Artificial sequence
  Synthetic polypeptide
IYSGGST

SEQ ID NO: 202 - protein, 16 aa, Artificial sequence
  Synthetic polypeptide
ARGGVVDTYYYYGMDV

SEQ ID NO: 203 - protein, 9 aa, Artificial sequence
  Synthetic polypeptide
TGPVTTGHW

SEQ ID NO: 204 - protein, 3 aa, Artificial sequence
  Synthetic polypeptide
DTV

SEQ ID NO: 205 - protein, 9 aa, Artificial sequence
  Synthetic polypeptide
LLSQSGAWV

SEQ ID NO: 206 - protein, 11 aa, Artificial sequence
  Synthetic polypeptide
GTWDSSLSGYV

SEQ ID NO: 207 - protein, 9 aa, Artificial sequence
  Synthetic polypeptide
SSDVGGYNY

SEQ ID NO: 208 - protein, 3 aa, Artificial sequence
  Synthetic polypeptide
EVS

SEQ ID NO: 209 - protein, 10 aa, Artificial sequence
  Synthetic polypeptide
SSYAGSNNLA

SEQ ID NO: 210 - protein, 8 aa, Artificial sequence
  Synthetic polypeptide
RSNIGNNY

SEQ ID NO: 211 - protein, 3 aa, Artificial sequence
  Synthetic polypeptide
DYD

SEQ ID NO: 212 - protein, 11 aa, Artificial sequence
  Synthetic polypeptide
GTWDNSLSAGV

SEQ ID NO: 213 - protein, 9 aa, Artificial sequence
  Synthetic polypeptide
QQSYSTPRT

SEQ ID NO: 214 - protein, 11 aa, Artificial sequence
  Synthetic polypeptide
GTWDSSLSAGV

SEQ ID NO: 215 - protein, 10 aa, Artificial sequence
  Synthetic polypeptide
QQSYSTRPLT

SEQ ID NO: 216 - protein, 8 aa, Artificial sequence
  Synthetic polypeptide
SSNIGKNY

SEQ ID NO: 217 - protein, 11 aa, Artificial sequence
  Synthetic polypeptide
AAWDDGLSAVV

SEQ ID NO: 218 - protein, 9 aa, Artificial sequence
  Synthetic polypeptide
ARDYGRGGV

SEQ ID NO: 219 - protein, 8 aa, Artificial sequence
  Synthetic polypeptide
GFTFSNAW

SEQ ID NO: 220 - protein, 10 aa, Artificial sequence
  Synthetic polypeptide
IKSKTDGGTT

SEQ ID NO: 221 - protein, 18 aa, Artificial sequence
  Synthetic polypeptide
TTDRSYDYVWGSYRYPDY

SEQ ID NO: 222 - protein, 8 aa, Artificial sequence
  Synthetic polypeptide
QQSYSTPE

SEQ ID NO: 223 - protein, 8 aa, Artificial sequence
  Synthetic polypeptide
GFTFNNFG

SEQ ID NO: 224 - protein, 8 aa, Artificial sequence
  Synthetic polypeptide
ISYEGSIK

SEQ ID NO: 225 - protein, 17 aa, Artificial sequence
  Synthetic polypeptide
AKDRAIFYLNPRYYLDY

SEQ ID NO: 226 - protein, 10 aa, Artificial sequence
  Synthetic polypeptide
QAWDSSTAVV

SEQ ID NO: 227 - protein, 15 aa, Artificial sequence
  Synthetic polypeptide
ARDWRVGATTQDVGY

SEQ ID NO: 228 - protein, 19 aa, Artificial sequence
  Synthetic polypeptide
AKATQLFWLGQFTRDGFDI

SEQ ID NO: 229 - protein, 17 aa, Artificial sequence
  Synthetic polypeptide
AKANGFNWKTPENGLDV

SEQ ID NO: 230 - protein, 18 aa, Artificial sequence
  Synthetic polypeptide
ARGVGAMQLWLRGYYFDS

SEQ ID NO: 231 - protein, 22 aa, Artificial sequence
  Synthetic polypeptide
ARRPPQCRNGICQHHLYGALDV

SEQ ID NO: 232 - protein, 11 aa, Artificial sequence
  Synthetic polypeptide
AKTSGYNLPDY

SEQ ID NO: 233 - protein, 9 aa, Artificial sequence
  Synthetic polypeptide
ARRQWTFDY

SEQ ID NO: 234 - protein, 18 aa, Artificial sequence
  Synthetic polypeptide
TTDRTYDYVWGSYRYEDY

SEQ ID NO: 235 - protein, 25 aa, Artificial sequence
  Synthetic polypeptide
ASPEASYYYGDRGHFFPARRIDFDF

SEQ ID NO: 236 - protein, 15 aa, Artificial sequence
  Synthetic polypeptide
TSPRADYGSNSVGNY

SEQ ID NO: 237 - protein, 18 aa, Artificial sequence
  Synthetic polypeptide
ARDGITNSGVVTHFGMDV

SEQ ID NO: 238 - protein, 18 aa, Artificial sequence
  Synthetic polypeptide
ARHMGMQLWLRAHDAFDL

SEQ ID NO: 239 - protein, 8 aa, Artificial sequence
  Synthetic polypeptide
ARLGTGYP

SEQ ID NO: 240 - protein, 13 aa, Artificial sequence
  Synthetic polypeptide
ACSRARGFMFFDY

SEQ ID NO: 241 - protein, 18 aa, Artificial sequence
  Synthetic polypeptide
ARADSCNGGICYYQGMDV

SEQ ID NO: 242 - protein, 20 aa, Artificial sequence
  Synthetic polypeptide
AREQPPGRMVVPATYWHFDL

SEQ ID NO: 243 - protein, 20 aa, Artificial sequence
  Synthetic polypeptide
AKASQLFWLGQFTRDGFDIW

SEQ ID NO: 244 - protein, 19 aa, Artificial sequence
  Synthetic polypeptide
ARDVFDPTAVSPIGGFDYW

SEQ ID NO: 245 - protein, 19 aa, Artificial sequence
  Synthetic polypeptide
ARGVGAMQLWLRGYYFDSW

SEQ ID NO: 246 - protein, 24 aa, Artificial sequence
  Synthetic polypeptide
ARGGRPITILGAVLAGGRNYFDPW

SEQ ID NO: 247 - protein, 19 aa, Artificial sequence
  Synthetic polypeptide
ARDTFDPIDSKEGGWFDPW

SEQ ID NO: 248 - protein, 16 aa, Artificial sequence
  Synthetic polypeptide
ATGSPFDRTQNWFDPW

SEQ ID NO: 249 - protein, 10 aa, Artificial sequence
  Synthetic polypeptide
ARRESPPGYW

SEQ ID NO: 250 - protein, 16 aa, Artificial sequence
  Synthetic polypeptide
ARHLGSGRYLRYLQHW

SEQ ID NO: 251 - protein, 19 aa, Artificial sequence
  Synthetic polypeptide
ARDAHCIKGVCTHHGMDVW

SEQ ID NO: 252 - protein, 17 aa, Artificial sequence
  Synthetic polypeptide
AHRTHALPPSVAGFDYW

SEQ ID NO: 253 - protein, 12 aa, Artificial sequence
  Synthetic polypeptide
AKGRKGAALDYW

SEQ ID NO: 254 - protein, 18 aa, Artificial sequence
  Synthetic polypeptide
AKDYTRTDWYGGEVIDYW

SEQ ID NO: 255 - protein, 20 aa, Artificial sequence
  Synthetic polypeptide
ARTCKGSPGGRDGYNCLDYW

SEQ ID NO: 256 - protein, 12 aa, Artificial sequence
  Synthetic polypeptide
ARAPRGIAGDYW

SEQ ID NO: 257 - protein, 14 aa, Artificial sequence
  Synthetic polypeptide
ARASEGRDIGGGYW

SEQ ID NO: 258 - protein, 12 aa, Artificial sequence
  Synthetic polypeptide
AREGFGVVIDYW

SEQ ID NO: 259 - protein, 19 aa, Artificial sequence
  Synthetic polypeptide
AHRVPRMELWLRGGWFDPW

SEQ ID NO: 260 - protein, 11 aa, Artificial sequence
  Synthetic polypeptide
TKNRGGPSDHW

SEQ ID NO: 261 - protein, 15 aa, Artificial sequence
  Synthetic polypeptide
ATAAGVRGRGTIDYW

SEQ ID NO: 262 - protein, 19 aa, Artificial sequence
  Synthetic polypeptide
ARADSCNGGICYYRGMDVW

SEQ ID NO: 263 - protein, 19 aa, Artificial sequence
  Synthetic polypeptide
ARHMGLQLWLRAHDAFDLW

SEQ ID NO: 264 - protein, 24 aa, Artificial sequence
  Synthetic polypeptide
ARLRGVEDIVVVPAARTYNWFDPW

SEQ ID NO: 265 - protein, 20 aa, Artificial sequence
  Synthetic polypeptide
ATDGYCPGGVCHRLGSFDFW

SEQ ID NO: 266 - protein, 21 aa, Artificial sequence
  Synthetic polypeptide
ARGTIYFDRSGYRRVDPFHIW

SEQ ID NO: 267 - protein, 19 aa, Artificial sequence
  Synthetic polypeptide
ARHGPALYNYGSGAYLDYW

SEQ ID NO: 268 - protein, 14 aa, Artificial sequence
  Synthetic polypeptide
ARIPIATHLGSDYW

SEQ ID NO: 269 - protein, 10 aa, Artificial sequence
  Synthetic polypeptide
ARRQWTFDYW

SEQ ID NO: 270 - protein, 14 aa, Artificial sequence
  Synthetic polypeptide
VRGRRGGYNSPDYW

SEQ ID NO: 271 - protein, 26 aa, Artificial sequence
  Synthetic polypeptide
ASPEASYYYGDRGHFFPARRIDFDFW

SEQ ID NO: 272 - protein, 19 aa, Artificial sequence
  Synthetic polypeptide
ARQNVVLRFLERASRFDPW

SEQ ID NO: 273 - protein, 7 aa, Artificial sequence
  Synthetic polypeptide
ASPTVTM

SEQ ID NO: 274 - protein, 18 aa, Artificial sequence
  Synthetic polypeptide
ARVGTTVTANYYYGMDVW

SEQ ID NO: 275 - protein, 17 aa, Artificial sequence
  Synthetic polypeptide
ATRLEYNTPEKKPFDFW

SEQ ID NO: 276 - protein, 10 aa, Artificial sequence
  Synthetic polypeptide
ARDYGRGGVW

SEQ ID NO: 277 - protein, 16 aa, Artificial sequence
  Synthetic polypeptide
ARRSGSGWTWGYFDYW

SEQ ID NO: 278 - protein, 14 aa, Artificial sequence
  Synthetic polypeptide
ARIPRDGYNFIDFW

SEQ ID NO: 279 - protein, 19 aa, Artificial sequence
  Synthetic polypeptide
ATASPHCSSTSCSINWFDP

SEQ ID NO: 280 - protein, 13 aa, Artificial sequence
  Synthetic polypeptide
ASGGGSYYSPFDY

SEQ ID NO: 281 - protein, 14 aa, Artificial sequence
  Synthetic polypeptide
ARAGGGGYQGPFDY

SEQ ID NO: 282 - protein, 14 aa, Artificial sequence
  Synthetic polypeptide
ARAYGGGYLTPFDY

SEQ ID NO: 283 - protein, 14 aa, Artificial sequence
  Synthetic polypeptide
AKPQGGGYNGAFDI

SEQ ID NO: 284 - protein, 17 aa, Artificial sequence
  Synthetic polypeptide
ATVSPFWSGYYNNWFDP

SEQ ID NO: 285 - protein, 14 aa, Artificial sequence
  Synthetic polypeptide
ARPVTGGYYYYMDV

SEQ ID NO: 286 - protein, 18 aa, Artificial sequence
  Synthetic polypeptide
TTERYYDYIWGSYRYSDY

SEQ ID NO: 287 - protein, 14 aa, Artificial sequence
  Synthetic polypeptide
ARDLRGSYSGLLDY

SEQ ID NO: 288 - protein, 18 aa, Artificial sequence
  Synthetic polypeptide
AKVVRHLYSSGYSNWFDP

SEQ ID NO: 289 - protein, 21 aa, Artificial sequence
  Synthetic polypeptide
ANQVTYYDYIWGSYRPGAFDI

SEQ ID NO: 290 - protein, 18 aa, Artificial sequence
  Synthetic polypeptide
ARDKGRGILFTHRDAFDI

SEQ ID NO: 291 - protein, 12 aa, Artificial sequence
  Synthetic polypeptide
ARRRVYPSGFDY

SEQ ID NO: 292 - protein, 14 aa, Artificial sequence
  Synthetic polypeptide
ARASHGGYSSAFDP

SEQ ID NO: 293 - protein, 19 aa, Artificial sequence
  Synthetic polypeptide
VRSGHCNGGVCYGAEYFQH

SEQ ID NO: 294 - protein, 13 aa, Artificial sequence
  Synthetic polypeptide
ARDGSGGYSNPDY

SEQ ID NO: 295 - protein, 10 aa, Artificial sequence
  Synthetic polypeptide
VKGAGGYFDS

SEQ ID NO: 296 - protein, 14 aa, Artificial sequence
  Synthetic polypeptide
ARAPAVVVGNWFDP

SEQ ID NO: 297 - protein, 15 aa, Artificial sequence
  Synthetic polypeptide
ARGLDIIVVPAALDH

SEQ ID NO: 298 - protein, 1288 aa, Artificial sequence
  Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 299 - protein, 107 aa, Artificial sequence
  Synthetic polypeptide
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPRTFGGGTKLEIK

SEQ ID NO: 300 - protein, 108 aa, Artificial sequence
  Synthetic polypeptide
DIQMTQSPASLSASVGETVTITCGASENIYGALNWYQRKQGKSPQLLIYGATNLADGMSSRFSGSGSGRQYSLKISSLHPDDVATYYCQNVLSTPPWTFGGGTKLEIK

SEQ ID NO: 301 - protein, 108 aa, Artificial sequence
  Synthetic polypeptide
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPYTFGGGTKLEIK

SEQ ID NO: 302 - protein, 108 aa, Artificial sequence
  Synthetic polypeptide
DIQMTQSPASLSVSVGETVTITCRASENIYSNLAWYQQKQGKSPQLLVYAATNLADGVPSRFSGSGSGTQYSLKINSLQSEDFGSYYCQHFWGTPPWTFGGGTKLEIK

SEQ ID NO: 303 - protein, 108 aa, Artificial sequence
  Synthetic polypeptide
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLPWTFGGGTKLEIK

SEQ ID NO: 304 - protein, 108 aa, Artificial sequence
  Synthetic polypeptide
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPLLTFGAGTKLELK

SEQ ID NO: 305 - protein, 108 aa, Artificial sequence
  Synthetic polypeptide
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPLLTFGAGTKLELK

SEQ ID NO: 306 - protein, 112 aa, Artificial sequence
  Synthetic polypeptide
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSWTFGGGTKLEIK

SEQ ID NO: 307 - protein, 113 aa, Artificial sequence
  Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPWTFGGGTKLEIK

SEQ ID NO: 308 - protein, 113 aa, Artificial sequence
  Synthetic polypeptide
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQWTFGGGTKLEIK

SEQ ID NO: 309 - protein, 114 aa, Artificial sequence
  Synthetic polypeptide
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPPYTFGGGTKLEIK

SEQ ID NO: 310 - protein, 6 aa, Artificial sequence
  Synthetic polypeptide
QDISNY

SEQ ID NO: 311 - protein, 6 aa, Artificial sequence
  Synthetic polypeptide
ENIYGA

SEQ ID NO: 312 - protein, 6 aa, Artificial sequence
  Synthetic polypeptide
ENIYSY

SEQ ID NO: 313 - protein, 6 aa, Artificial sequence
  Synthetic polypeptide
ENIYSN

SEQ ID NO: 314 - protein, 6 aa, Artificial sequence
  Synthetic polypeptide
QDINKY

SEQ ID NO: 315 - protein, 6 aa, Artificial sequence
  Synthetic polypeptide
QDVGTA

SEQ ID NO: 316 - protein, 6 aa, Artificial sequence
  Synthetic polypeptide
QNVGTN

SEQ ID NO: 317 - protein, 12 aa, Artificial sequence
  Synthetic polypeptide
QSVLYSSNQKNY

SEQ ID NO: 318 - protein, 11 aa, Artificial sequence
  Synthetic polypeptide
QSIVHSNGNTY

SEQ ID NO: 319 - protein, 11 aa, Artificial sequence
  Synthetic polypeptide
QSLLDSDGKTY

SEQ ID NO: 320 - protein, 12 aa, Artificial sequence
  Synthetic polypeptide
QSLLYSSNQKNY

SEQ ID NO: 321 - protein, 3 aa, Artificial sequence
  Synthetic polypeptide
YTS

SEQ ID NO: 322 - protein, 3 aa, Artificial sequence
  Synthetic polypeptide
GAT

SEQ ID NO: 323 - protein, 3 aa, Artificial sequence
  Synthetic polypeptide
NAK

SEQ ID NO: 324 - protein, 3 aa, Artificial sequence
  Synthetic polypeptide
AAT

SEQ ID NO: 325 - protein, 3 aa, Artificial sequence
  Synthetic polypeptide
SAS

SEQ ID NO: 326 - protein, 3 aa, Artificial sequence
  Synthetic polypeptide
KVS

SEQ ID NO: 327 - protein, 3 aa, Artificial sequence
  Synthetic polypeptide
LVS

SEQ ID NO: 328 - protein, 9 aa, Artificial sequence
  Synthetic polypeptide
QQGNTLPRT

SEQ ID NO: 329 - protein, 10 aa, Artificial sequence
  Synthetic polypeptide
QNVLSTPPWT

SEQ ID NO: 330 - protein, 10 aa, Artificial sequence
  Synthetic polypeptide
QHHYGTPPYT

SEQ ID NO: 331 - protein, 10 aa, Artificial sequence
  Synthetic polypeptide
QHFWGTPPWT

SEQ ID NO: 332 - protein, 10 aa, Artificial sequence
  Synthetic polypeptide
LQYDNLLPWT

SEQ ID NO: 333 - protein, 10 aa, Artificial sequence
  Synthetic polypeptide
QQYSSYPLLT

SEQ ID NO: 334 - protein, 10 aa, Artificial sequence
  Synthetic polypeptide
QQYNSYPLLT

SEQ ID NO: 335 - protein, 8 aa, Artificial sequence
  Synthetic polypeptide
HQYLSSWT

SEQ ID NO: 336 - protein, 10 aa, Artificial sequence
  Synthetic polypeptide
FQGSHVPPWT

SEQ ID NO: 337 - protein, 10 aa, Artificial sequence
  Synthetic polypeptide
WQGTHFPQWT

SEQ ID NO: 338 - protein, 10 aa, Artificial sequence
  Synthetic polypeptide
QQYYSYPPYT

SEQ ID NO: 339 - protein, 121 aa, Artificial Sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSTYGSYYYGMDVWGQGTTVTVSS

SEQ ID NO: 340 - protein, 112 aa, Artificial Sequence
  Synthetic polypeptide
QSVLTQPPSVSGAPGQRVTISCTGGSSNLGAAYDVHWYQQYPGRAPKLLIYNNNNRPSGVPDRFSGSKSDTSASLVIAGLQAEDEAGYYCQSYDISLSGLYVFGTGTKLTVL

SEQ ID NO: 341 - protein, 111 aa, Artificial Sequence
  Synthetic polypeptide
QSVLTQPPSVSGAPGQRVTISCTGSSSNIGAGYDVHWYQQVPGTATKLLIYGNNNRPPGVPDRFSGSKSGTSASLTITGLRAEDEADFFCQSYDRSVSGWVFGGGTKLTVL

SEQ ID NO: 342 - protein, 111 aa, Artificial Sequence
  Synthetic polypeptide
NFMLTQPHSVSESPGKAVTISCTRNSGSIASNFVQWFQQRPGSYPTTIMYENDKRPTGVPHRFSGSIDSSSNSASLSISGLRAEDEADYYCQSSDDSNQLVFGGGTKVTVL

SEQ ID NO: 343 - protein, 108 aa, Artificial Sequence
  Synthetic polypeptide
SYELIQPPSVSVAPGQTARITCGNNIGSKTVHWYQQKPGQAPVLVVYDDSDRPSGIPERFSGSNSGNTATLTISRVEAGDEADYYCQVWESSGDHYWVFGRGTKVTVL

SEQ ID NO: 344 - protein, 108 aa, Artificial Sequence
  Synthetic polypeptide
SSELSQDPAVSVALGQTVRITCQGDSLRSYYASWYQQRPGRAPVLVIYGKNNRPSGIPDRFSGSSSGNTASLTITGAQAEDEADYYCDSRDSSGKRRVFGGGTKLTVL

SEQ ID NO: 345 - protein, 111 aa, Artificial Sequence
  Synthetic polypeptide
QSALIQPPSVSGAPGQRVTISCTGSSSNIGADYDVHWCQLLPGIAPKLLIFANSPRPSGVPDRFSGSKSATSASLAITGLLAEDEASYYCQSYDSILNDSAVGGGTKLTVL

SEQ ID NO: 346 - protein, 112 aa, Artificial Sequence
  Synthetic polypeptide
QSALIQPPSVSGAPGQRVTISCTGSSSNIGADYDVHWCQLLPGIAPKLLIFANSPRPSGVPDRFSGSKSATSASLAITGLLAEDEASYYCQSYDSILNDSGVVGGGTKLTVL

SEQ ID NO: 347 - protein, 111 aa, Artificial Sequence
  Synthetic polypeptide
QSALIQPPSVSGAPGQRVTISCTGSSSNIGADYDVHWCQLLPGIAPKLLIFANSPRPSGVPDRFSGSKSATSASLAITGLLAEDEASYYCQSYDSILNDSVVGGGTKLTVL

SEQ ID NO: 348 - protein, 111 aa, Artificial Sequence
  Synthetic polypeptide
QSVLTQPPSVSGAPGQRVTISCTGGSSNIGAGYDVHWYQQVPGTATKLLIYGNNNRPSGVPDRFSGSKSGTSASLTITGLRAEDEADFFCQSYDRSVSGWVFGGGTQLTVL

SEQ ID NO: 349 - protein, 108 aa, Artificial Sequence
  Synthetic polypeptide
SSELTQEPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKVTVL

SEQ ID NO: 350 - protein, 108 aa, Artificial Sequence
  Synthetic polypeptide
SSELSQDPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKLTVL

SEQ ID NO: 351 - protein, 120 aa, Artificial Sequence
  Synthetic polypeptide
QVQLVQSGGGVVQPGRSLRLSCAASGFTFSSYGMHWVRLAPGKGLEWVAVISYDGSNKYYADSVKGRFTISRDNSKNTLYLQMNSLRAEDTAVYYCAKPQGGYNGAFDIWGQGTMVTVSS

SEQ ID NO: 352 - protein, 108 aa, Artificial Sequence
  Synthetic polypeptide
SSELTQEPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKLTVL

SEQ ID NO: 353 - protein, 125 aa, Artificial Sequence
  Synthetic polypeptide
QVQLVQSGGGVVQPGRSLRLSCAASGVSITSYHWSWIRQSPGKGLEWVAFMYYISGQTSSNPSLKGRVIMSLDTSKNQVSLKLMSVTAADTAIYFCARADSCNGGICYYQGMDVWGQGTAVTVSS

SEQ ID NO: 354 - protein, 106 aa, Artificial Sequence
  Synthetic polypeptide
SYELIQPPSVSVAPGQTARITCGGNKLGDKYACWYQQKPGQSPVLVIYQDSKRPSGIPERFSGSNSGNTATLTISGTQAMDEADYYCQAWDSSTVVFGGGTKLTVL

SEQ ID NO: 355 - protein, 108 aa, Artificial Sequence
  Synthetic polypeptide
SSELTQDPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKLTVL

SEQ ID NO: 356 - protein, 121 aa, Artificial Sequence
  Synthetic polypeptide
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARAGGGGYQGPFDYWGQGTLVTVSS

SEQ ID NO: 357 - protein, 108 aa, Artificial Sequence
  Synthetic polypeptide
SYELTQEPAVSVALGQTVRITCQGDSLRIYYASWYQQKPRQAPVLVIYGKHNRPSGIPERFSGSNSGNTATLTISGTQAMDEADYYCQAWDSSTAWNVFGTGTKVTVL

SEQ ID NO: 358 - protein, 121 aa, Artificial Sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDLRGSYSGLLDYWGQGTLVTVSS

SEQ ID NO: 359 - protein, 111 aa, Artificial Sequence
  Synthetic polypeptide
QSVLTQPPSVSGAPGQRVTISCTGSSSNIGAGYDVHWYQQVPGTATKLLIYGNNNRPSWVPDRFSGSKSGTSASLTITGLRAEDEADFFCQSYDRSVSGWVFGGGTKLTVL

SEQ ID NO: 360 - protein, 124 aa, Artificial Sequence
  Synthetic polypeptide
EVQLVQSGGGLVQPGGSLRLSCAASGFTFSNYAMNWVRQAPGKGLEWVSGISGSGGSTYYADSVKGRFTISRDNSKNTLYLQMNSLRAEDTAVYYCARDRFQSYTYYYYGMDVWGQGTTVTVSS

SEQ ID NO: 361 - protein, 125 aa, Artificial Sequence
  Synthetic polypeptide
AVQLVQSGGGLVQPGGSLRLSCAASGFTFSSYDMHWVRQATGKGLEWVSAIGTAGDTYYPGSVKGRFTISRENAKNSLYLQMNSLRAGDTAVYYCARGSHPRKRSGLSLDAFDIWGQGTMVTVSS

SEQ ID NO: 362 - protein, 110 aa, Artificial Sequence
  Synthetic polypeptide
QSVVTQPPSMSAAPGQRVTISCSGSNSNIGSNYLSWYQQLPGAAPKLLIYENNKRPSGIPDRFSGSKSGTSATLGITGLQTGDEADYYCATWDSSLSAVVFGGGTKLTVL

SEQ ID NO: 363 - protein, 117 aa, Artificial Sequence
  Synthetic polypeptide
QVQLVQSGAEVKKPGASVKVSCKASGYTFTNYGLHWVRQAPGQRLEWMGWISTGSGNTQHSQKFQGRVTITRDTSASTAYMELSSLRSEDTAVYYCVKGAGGYFDSWGQGTLVTVSS

SEQ ID NO: 364 - protein, 108 aa, Artificial Sequence
  Synthetic polypeptide
SYELTQEPAVSVALGQTVRITCQGDSLRIYYASWYQQKPRQAPVLVIYGKHNRPSGIPDRFSGSSSGNTASLTITGAQAEDEADYYCNSRDSSGDLVVFGGGTKLTVL

SEQ ID NO: 365 - protein, 107 aa, Artificial Sequence
  Synthetic polypeptide
DIQMTQSPSSLSASVGDRVTITCRASQSVSSYLNWYQQKPGKAPKLLIYAASGLQSGVPSRFSGRGSGTDFTLTINSLQPEDFATYFCQQSSSIPRTFGQGTKVDIK

SEQ ID NO: 366 - protein, 106 aa, Artificial Sequence
  Synthetic polypeptide
DIQMTQSPSSLSASVGDRVTITCRASQSVSSYLNWYQQNQESPKLLIYAASGLQSGVPSRFSGRGSGTDFTLTINSLQPEDFATYFCQQSSSIPRTFGQGTKVDIK

SEQ ID NO: 367 - protein, 106 aa, Artificial Sequence
  Synthetic polypeptide
DIQMTQSPSSLSASVGDRVTITCRASQSVSSYLNWYQQNQGKPKLLIYAASGLQSGVPSRFSGRGSGTDFTLTINSLQPEDFATYFCQQSSSIPRTFGQGTKVDIK

SEQ ID NO: 368 - protein, 104 aa, Artificial Sequence
  Synthetic polypeptide
SYELTQPPSVSVAPGQTARVTCGGNNIGSKSVHWYQQKPGQAPVLVVYDDSDRPSGIPERFSGSNSGNTATLTISGTQAMDEADYYCQAWDSSVFGGGTKLTVL

SEQ ID NO: 369 - protein, 104 aa, Artificial Sequence
  Synthetic polypeptide
SYELTQPPSVSVAPGQTARVTCGGNNIGSKSVHWYQQKPGQAPVLVVYDDSDRPSGIPERFSGSNSGNTATLTISGTQAMDEADYYCQAWDSVVFGGGTKLTVL

SEQ ID NO: 370 - protein, 105 aa, Artificial Sequence
  Synthetic polypeptide
SYELTQPPSVSVAPGQTARVTCGGNNIGSKSVHWYQQKPGQAPVLVVYDDSDRPSGIPERFSGSNSGNTATLTISGTQAMDEADYYCQAWDSSVEPGGGTKLTVL

SEQ ID NO: 371 - protein, 112 aa, Artificial Sequence
  Synthetic polypeptide
QSVLTQPPSVSAAPGQKVTISCTGSSSNIGAGYNVHWYQQLPGTAPKLLIYGNSNRPSGVPDRFSGSKSGTSASLAITGLQAEDEADYYCQSYDSSLSGSGVFGGGTKLTVL

SEQ ID NO: 372 - protein, 108 aa, Artificial Sequence
  Synthetic polypeptide
SYELTQPPSVSVAPGKTARITCGGNNIGSKSVHWYQQKPGQAPVLVVYDDSDRPSGIPERFSGSNSGNTATLTISRVEAGDEADYYCQVWDSSSDHWVFGGGTKLTVL

SEQ ID NO: 373 - protein, 107 aa, Artificial Sequence
  Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 374 - protein, 113 aa, Artificial Sequence
  Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYSTPPYTFGQGTKLEIK

SEQ ID NO: 375 - protein, 110 aa, Artificial Sequence
  Synthetic polypeptide
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVL

SEQ ID NO: 376 - protein, 107 aa, Artificial Sequence
  Synthetic polypeptide
DIQMTQSPSTLSASVGDRVTITCRASQTIYSWLAWYQQKPGKAPKFLIHKASSLESGVPSRFSGSGSGTEFTLTISSLQPDDFATYYCQQYSSYPRTFGQGTKVEIK

SEQ ID NO: 377 - protein, 107 aa, Artificial Sequence
  Synthetic polypeptide
DIQMTQSPSSMSASVGDRVIITCRASQSISSSLNWYQQKPGKAPNLLIYSASTLQSEVPSRFSGSGSGTDFTLTISSLQPEDFATYYCQQSYSTPPAFGQGTKVEFK

SEQ ID NO: 378 - protein, 112 aa, Artificial Sequence
  Synthetic polypeptide
DIQMTQSPDSLAVSLGERATINCKSGQSVLYSSNNKNYLAWYQQKPGQPPKLLIYWASTRESGVPDRFSGSGSGTDFTLTITSLQAEDVAVYYCQQYYSLYTFGQGTKLEIK

SEQ ID NO: 379 - protein, 107 aa, Artificial Sequence
  Synthetic polypeptide
DIQMTQSPSAMSASVGDRVTITCRASQGISNYLAWFQQKPGKVPKRLIYAASSLQSGVPSRFCGSGSGTDFTLTIASLQPEDFATYYCQQIYSFPITFGQGTRLETK

SEQ ID NO: 380 - protein, 107 aa, Artificial Sequence
  Synthetic polypeptide
DIQMTQSPSSMSASVGDRVIITCRASESLTTWLAWYQQRPGEAPKVLIYKGSTLVRGVPSRFSGRGSGTEFTLTITSLQPDDFATYYCQQYSGFPITFGQGTKVEIK

SEQ ID NO: 381 - protein, 120 aa, Artificial Sequence
  Synthetic polypeptide
QVQLQQSGGGLVKPGGSLRLSCAASGFTFSSYIMKWVRQAPGKGLEWVAVISYDGSNKYYADSVKGRFTISRDNSKNTLYLQMNSLRAEDTAVYYCARVSYSGYGPPGYWGQGTLVTVSS

SEQ ID NO: 382 - protein, 113 aa, Artificial Sequence
  Synthetic polypeptide
DIQMTQSPDSLAVSLGERATINCKSSQSVLYSSNNKNYLAWYQHKPGQPPKLLIYWASTRESGVPDRFSGSGSGTDFTLTISSLQAEDVAVYYCQQCYSSPPTFGQGTKLEIK

SEQ ID NO: 383 - protein, 121 aa, Artificial Sequence
  Synthetic polypeptide
QVQLQQSGGGVVQPGRSLRLSCAASGFTFSSYAMHWVRQAPGKGLEWVAVISYDGDRVYYADSVRGRFNISRDNSKNTLYLQMNSLRPEDTAVYYCAKSRSGSYYYGMDVWGQGTTVTVSS

SEQ ID NO: 384 - protein, 107 aa, Artificial Sequence
  Synthetic polypeptide
DIQMTQSPSSLSASVGDRVTITCRASQSVSSYLNWYQQNQGKAPKLLIYAASGLQSGVPSRFSGRGSGTDFTLTINSLQPEDFATYFCQQSSSIPRTFGQGTKVDIK

SEQ ID NO: 385 - protein, 105 aa, Artificial Sequence
  Synthetic polypeptide
SYELTQPPSVSVAPGQTARVTCGGNNIGSKSVHWYQQKPGQAPVLVVYDDSDRPSGIPERFSGSNSGNTATLTISGTQAMDEADYYCQAWDSSVVFGGGTKLTVL

SEQ ID NO: 386 - protein, 120 aa, Artificial Sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCASGGGSYYSPFDYWGQGTLVTVSS

SEQ ID NO: 387 - protein, 121 aa, Artificial Sequence
  Synthetic polypeptide
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARAYGGGYLTPFDYWGQGTLVTVSS

SEQ ID NO: 388 - protein, 120 aa, Artificial Sequence
  Synthetic polypeptide
QVQLVESGGGVVQPGGSLRLSCAASGFTFSSYAMHWVRQAPGKGLEWVAVISYDGSNKYYADSVKGRFTISRDNSKNTLYLQMNSLRAEDTAVYYCARDGSGGYSNPDYWGQGTLVTVSS

SEQ ID NO: 389 - protein, 108 aa, Artificial Sequence
  Synthetic polypeptide
DIQMTQSPSSLSASVGDRVTITCRASKHVSNYLNWYQQKPGKAPKLLIHAASSLQSGVPSRFSGSGSGTDFTLTISSLQPEDFAIYYCQQSFSTLPYTFGQGTKLKIK

SEQ ID NO: 390 - protein, 121 aa, Artificial Sequence
  Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARASHGGYSSAFDPWGQGTLVTVSS

SEQ ID NO: 391 - protein, 14 aa, Artificial Sequence
  Synthetic polypeptide
ARSTYGSYYYGMDV

SEQ ID NO: 392 - protein, 8 aa, Artificial Sequence
  Synthetic polypeptide
GFTFSSYI

SEQ ID NO: 393 - protein, 13 aa, Artificial Sequence
  Synthetic polypeptide
ARVSYSGYGPPGY

SEQ ID NO: 394 - protein, 9 aa, Artificial Sequence
  Synthetic polypeptide
SSNIGAGYD

SEQ ID NO: 395 - protein, 11 aa, Artificial Sequence
  Synthetic polypeptide
QSYDRSVSGWV

SEQ ID NO: 396 - protein, 8 aa, Artificial Sequence
  Synthetic polypeptide
ISYDGDRV

SEQ ID NO: 397 - protein, 14 aa, Artificial Sequence
  Synthetic polypeptide
AKSRSGSYYYGMDV

SEQ ID NO: 398 - protein, 12 aa, Artificial Sequence
  Synthetic polypeptide
QVWESSGDHYWV

SEQ ID NO: 399 - protein, 13 aa, Artificial Sequence
  Synthetic polypeptide
ASGGGSYYSPFDY

SEQ ID NO: 400 - protein, 6 aa, Artificial Sequence
  Synthetic polypeptide
SLRSYY

SEQ ID NO: 401 - protein, 11 aa, Artificial Sequence
  Synthetic polypeptide
DSRDSSGKRRV

SEQ ID NO: 402 - protein, 9 aa, Artificial Sequence
  Synthetic polypeptide
SSNIGADYD

SEQ ID NO: 403 - protein, 3 aa, Artificial Sequence
  Synthetic polypeptide
ANS

SEQ ID NO: 404 - protein, 11 aa, Artificial Sequence
  Synthetic polypeptide
QSYDSILNDSA

SEQ ID NO: 405 - protein, 12 aa, Artificial Sequence
  Synthetic polypeptide
QSYDSILNDSGV

SEQ ID NO: 406 - protein, 11 aa, Artificial Sequence
  Synthetic polypeptide
QSYDSILNDSV

SEQ ID NO: 407 - protein, 14 aa, Artificial Sequence
  Synthetic polypeptide
ARAYGGGYLTPFDY

SEQ ID NO: 408 - protein, 13 aa, Artificial Sequence
  Synthetic polypeptide
ARDGSGGYSNPDY

SEQ ID NO: 409 - protein, 13 aa, Artificial Sequence
  Synthetic polypeptide
AKPQGGYNGAFDI

SEQ ID NO: 410 - protein, 8 aa, Artificial Sequence
  Synthetic polypeptide
GVSITSYH

SEQ ID NO: 411 - protein, 8 aa, Artificial Sequence
  Synthetic polypeptide
MYYISGQT

SEQ ID NO: 412 - protein, 18 aa, Artificial Sequence
  Synthetic polypeptide
ARADSCNGGICYYQGMDV

SEQ ID NO: 413 - protein, 14 aa, Artificial Sequence
  Synthetic polypeptide
ARAGGGGYQGPFDY

SEQ ID NO: 414 - protein, 11 aa, Artificial Sequence
  Synthetic polypeptide
QAWDSSTAWNV

SEQ ID NO: 415 - protein, 14 aa, Artificial Sequence
  Synthetic polypeptide
ARDLRGSYSGLLDY

SEQ ID NO: 416 - protein, 8 aa, Artificial Sequence
  Synthetic polypeptide
GFTFSSYD

SEQ ID NO: 417 - protein, 7 aa, Artificial Sequence
  Synthetic polypeptide
IGTAGDT

SEQ ID NO: 418 - protein, 19 aa, Artificial Sequence
  Synthetic polypeptide
ARGSHPRKRSGLSLDAFDI

SEQ ID NO: 419 - protein, 8 aa, Artificial Sequence
  Synthetic polypeptide
NSNIGSNY

SEQ ID NO: 420 - protein, 3 aa, Artificial Sequence
  Synthetic polypeptide
ENN

SEQ ID NO: 421 - protein, 11 aa, Artificial Sequence
  Synthetic polypeptide
ATWDSSLSAVV

SEQ ID NO: 422 - protein, 8 aa, Artificial Sequence
  Synthetic polypeptide
GYTFTNYG

SEQ ID NO: 423 - protein, 8 aa, Artificial Sequence
  Synthetic polypeptide
ISTGSGNT

SEQ ID NO: 424 - protein, 10 aa, Artificial Sequence
  Synthetic polypeptide
VKGAGGYFDS

SEQ ID NO: 425 - protein, 9 aa, Artificial Sequence
  Synthetic polypeptide
QQSSSIPRT

SEQ ID NO: 426 - protein, 8 aa, Artificial Sequence
  Synthetic polypeptide
QAWDSSVV

SEQ ID NO: 427 - protein, 7 aa, Artificial Sequence
  Synthetic polypeptide
QAWDSSV

SEQ ID NO: 428 - protein, 7 aa, Artificial Sequence
  Synthetic polypeptide
QAWDSVV

SEQ ID NO: 429 - protein, 8 aa, Artificial Sequence
  Synthetic polypeptide
QAWDSSVE

SEQ ID NO: 430 - protein, 11 aa, Artificial Sequence
  Synthetic polypeptide
AAWDDSLSGWV

SEQ ID NO: 431 - protein, 6 aa, Artificial Sequence
  Synthetic polypeptide
QTIYSW

SEQ ID NO: 432 - protein, 3 aa, Artificial Sequence
  Synthetic polypeptide
KAS

SEQ ID NO: 433 - protein, 9 aa, Artificial Sequence
  Synthetic polypeptide
QQYSSYPRT

SEQ ID NO: 434 - protein, 6 aa, Artificial Sequence
  Synthetic polypeptide
QSISSS

SEQ ID NO: 435 - protein, 3 aa, Artificial Sequence
  Synthetic polypeptide
SAS

SEQ ID NO: 436 - protein, 9 aa, Artificial Sequence
  Synthetic polypeptide
QQSYSTPPA

SEQ ID NO: 437 - protein, 6 aa, Artificial Sequence
  Synthetic polypeptide
QGISNY

SEQ ID NO: 438 - protein, 9 aa, Artificial Sequence
  Synthetic polypeptide
QQIYSFPIT

SEQ ID NO: 439 - protein, 6 aa, Artificial Sequence
  Synthetic polypeptide
ESLTTW

SEQ ID NO: 440 - protein, 3 aa, Artificial Sequence
  Synthetic polypeptide
KGS

SEQ ID NO: 441 - protein, 9 aa, Artificial Sequence
  Synthetic polypeptide
QQYSGFPIT

SEQ ID NO: 442 - protein, 9 aa, Artificial Sequence
  Synthetic polypeptide
SSNIGAGYN

SEQ ID NO: 443 - protein, 3 aa, Artificial Sequence
  Synthetic polypeptide
GNS

SEQ ID NO: 444 - protein, 12 aa, Artificial Sequence
  Synthetic polypeptide
QSYDSSLSGSGV

SEQ ID NO: 445 - protein, 11 aa, Artificial Sequence
  Synthetic polypeptide
QVWDSSSDHWV

SEQ ID NO: 446 - protein, 9 aa, Artificial Sequence
  Synthetic polypeptide
QQSYSTPWT

SEQ ID NO: 447 - protein, 8 aa, Artificial Sequence
  Synthetic polypeptide
QQYYSLYT

SEQ ID NO: 448 - protein, 9 aa, Artificial Sequence
  Synthetic polypeptide
QQCYSSPPT

SEQ ID NO: 449 - protein, 9 aa, Artificial Sequence
  Synthetic polypeptide
QQYSTPPYT

SEQ ID NO: 450 - protein, 6 aa, Artificial Sequence
  Synthetic polypeptide
KHVSNY

SEQ ID NO: 451 - protein, 10 aa, Artificial Sequence
  Synthetic polypeptide
QQSFSTLPYT

SEQ ID NO: 452 - protein, 14 aa, Artificial Sequence
  Synthetic polypeptide
ARPHSGSYWSSFDY

SEQ ID NO: 453 - protein, 14 aa, Artificial Sequence
  Synthetic polypeptide
ARPRSGSYYTHFDY

SEQ ID NO: 454 - protein, 13 aa, Artificial Sequence
  Synthetic polypeptide
ARARGGYQRPFDY

SEQ ID NO: 455 - protein, 13 aa, Artificial Sequence
  Synthetic polypeptide
ARAGGGYQGPFDY

SEQ ID NO: 456 - DNA, 19 bp, Artificial Sequence
  Synthetic polypeptide
gcatcacctgcgactgtgt

SEQ ID NO: 457 - DNA, 34 bp, Artificial Sequence
  Synthetic polypeptide
gcaagcaccaagggcccatagtcgcaggtggcat

SEQ ID NO: 458 - DNA, 19 bp, Artificial Sequence
  Synthetic polypeptide
gcatcacctgcgacttgcc

SEQ ID NO: 459 - DNA, 47 bp, Artificial Sequence
  Synthetic polypeptide
acggtggccgcaccatctgtcttcatcttcccagtcgcaggtggcat

SEQ ID NO: 460 - DNA, 80 bp, Artificial Sequence
  Synthetic polypeptide
ggtcagcccaaggccaaccccacggtcactctgttcccgccctcctctgaggagctccaagccaaagtcgcaggtggcat

SEQ ID NO: 461 - DNA, 15 bp, Artificial Sequence
  Synthetic polypeptide
gtgtagtcgcaggtg

SEQ ID NO: 462 - DNA, 15 bp, Artificial Sequence
  Synthetic polypeptide
cacctgcagtcccat

SEQ ID NO: 463 - DNA, 15 bp, Artificial Sequence
  Synthetic polypeptide
tgccagtcgcaggtg

SEQ ID NO: 464 - DNA, 15 bp, Artificial Sequence
  Synthetic polypeptide
cacctgcagtctccc

SEQ ID NO: 465 - DNA, 15 bp, Artificial Sequence
  Synthetic polypeptide
cacctgcagtcccaa

SEQ ID NO: 466 - protein, 11 aa, Artificial Sequence
  Synthetic polypeptide
QSYDSSLSGWV

SEQ ID NO: 467 - protein, 9 aa, Artificial Sequence
  Synthetic polypeptide
QQSYSTPYT

SEQ ID NO: 468 - protein, 10 aa, Artificial Sequence
  Synthetic polypeptide
QQYNSYSRVT

SEQ ID NO: 469 - protein, 9 aa, Artificial Sequence
  Synthetic polypeptide
QQYNSYPLT

SEQ ID NO: 470 - protein, 12 aa, Artificial Sequence
  Synthetic polypeptide
QSFDSSLSGPVV